(19) United States
(12) Patent Application Publication
(10) Pub. No.: US 2025/0177396 A1
(43) Pub. Date: Jun. 5, 2025

(54) POLO-LIKE KINASE 4 (PLK4) INHIBITORS, PHARMACEUTICAL COMPOSITIONS, METHODS OF PREPARATION AND USES THEREOF

(71) Applicant: Repare Therapeutics Inc., St-Laurent (CA)

(72) Inventors: Frédéric VALLÉE, Montreal [ (CA); Boubacar SOW, Vaudreuil-Dorion (CA); Monica BUBENIK, Laval (CA); Michel GALLANT, Pierrefonds (CA); Philippe MOCHIRIAN, Montreal (CA); Simon SURPRENANT, Boisbriand (CA); Martin DUPLESSIS, St-Laurent (CA); Bingcan LIU, Montreal (CA); Alexander PERRYMAN, Pointe-Claire (CA)

(21) Appl. No.: 18/840,582

(22) PCT Filed: Feb. 23, 2023
(86) PCT No.: PCT/CA2023/050223

§ 371 Date: Aug. 22, 2024

Related U.S. Application Data
(60) Provisional application No. 63/313,035, filed on Feb. 23, 2022.

(51) Int. Cl. A61K 31/513; A61K 31/506; C07D 401/14; C07D 403/14

(52) U.S. Cl. CPC A61K 31/513; A61K 31/506; C07D 401/14; C07D 403/14

(57) ABSTRACT

Disclosed are compounds and pharmaceutically acceptable salts of formula (I), which feature either a N-(1H-pyrazol-3-yl)pyridin-2-amine or a N-(1H-pyrazol-3-yl)pyrimidin-4-amine core structure. These compounds may be used in the treatment of diseases such as cancer or diseases associated with TRIM37. The compounds disclosed herein may be inhibitors of polo-like kinase 4 (PLK4). Also disclosed are pharmaceutical compositions containing the compounds or pharmaceutically acceptable salts thereof, methods of their preparation, and use.

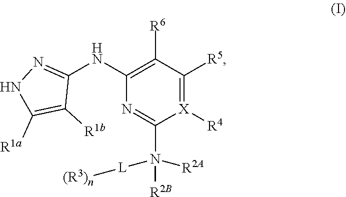

### FIELD OF THE INVENTION

The invention relates to compounds and pharmaceutical compositions, their preparation and their use in the treatment of a disease or condition, e.g., cancer, and, in particular, those diseases or conditions which are sensitive to inhibition of polo-like kinase-4.

### BACKGROUND

Protein kinases are a large group of intracellular and transmembrane signaling proteins in eukaryotic cells. These enzymes are responsible for transfer of the terminal (gamma) phosphate from ATP to specific amino acid residues of target proteins. Phosphorylation of specific amino acid residues in target proteins can modulate their activity leading to profound changes in cellular signaling and metabolism. Protein kinases can be found in the cell membrane, cytosol, organelles and structures such as centrioles and are responsible for mediating multiple cellular functions including metabolism, cellular growth, differentiation, cellular signaling, modulation of immune responses, and cell death. Thus, inhibitors of select kinases or kinase families are expected to be useful in the treatment of cancer and other diseases or conditions.

Centrioles template assembly of microtubules and recruit pericentriolar material to form centrosomes. Centriole duplication is tightly controlled, and normal mitotic cells have precisely two centrosomes. Supernumerary centrosomes are prevalent in cancer and have been postulated to contribute to tumorigenesis. Polo-like kinase 4 (PLK4) is a major player in centriole biogenesis. Depletion or inhibition of its kinase activity prevents centriole formation, while overexpression leads to the formation of multiple centrioles. Cells can survive in the absence of centrioles through use of pericentriolar material for assembly of microtubules. Importantly, loss of both centrioles and pericentriolar material greatly impairs cellular viability. Thus, tumor cells with low levels of pericentriolar material are sensitive to inhibition of PLK4 activity. Select genetic factors, such as overexpression of TRIM37, have been identified that suppress levels of pericentriolar material and which sensitize tumor cells to PLK4 inhibition. Thus, PLK4 inhibitors are expected to have anticancer properties in general and in the specific case of TRIM37 amplification or similar cellular contexts leading to impaired pericentriolar function.

### SUMMARY OF THE INVENTION

In an aspect, the invention provides compound of formula (I):

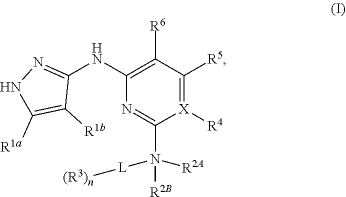

or a pharmaceutically acceptable salt thereof,
wherein
   n is 0, 1, 2, 3, or 4;
   m is 0, 1, or 2;
   L is optionally substituted C₂₋₉ heterocyclyl, optionally substituted C₂₋₉ heteroaryl, optionally substituted C₆₋₁₀ aryl, or optionally substituted C₃₋₈ cycloalkyl, wherein L is further optionally substituted by n occurrences of R³;
   R^{1a} is hydrogen, halogen, optionally substituted C₁₋₆ alkyl, optionally substituted C₁₋₆ alkoxy, optionally substituted C₁₋₆ heteroalkyl, optionally substituted C₂₋₆ alkenyl, optionally substituted C₂₋₆ alkynyl, optionally substituted C₃₋₈ cycloalkyl, or nitrile;
   R^{1b} is hydrogen; or
   R^{1a} and R^{1b}, together with the atoms to which they are attached, are a 3-5-membered cycloalkyl, cycloakylene, cycloalkylyne, heterocycloalkyl, aryl, or heteroaryl;
   A is O or S, and R^{2A} and R^{2B} are both absent; or A is N, R^{2A} is absent, and R^{2B} is hydrogen, optionally substituted C₁₋₆ alkyl, optionally substituted C₁₋₆ heteroalkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl, or R^{2B} and L, together with the atom to which they are attached, combine to form an optionally substituted C₂₋₉ heterocyclyl or optionally substituted C₂₋₉ heteroaryl; or A is C, and each of R^{2A} and R^{2B} are independently hydrogen, optionally substituted C₁₋₆ alkyl, optionally substituted C₁₋₆ heteroalkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl;
   each R³ is independently halogen, cyano, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₆₋₁₀ aryl C₁₋₆ alkyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, —S(O)ₘR^{3A}, —N(R^{3B})₂, or —OR^{3B};
   R^{3A} is optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₆₋₁₀ aryl, optionally substituted C₂₋₉ heterocyclyl, optionally substituted C₁₋₉ heteroaryl, —OR^{3B} or —N(R^{3B})₂.

Each R^{3B} is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl; or two R^{3B} groups, together with the atom to which both are attached, combine to form an optionally substituted C₂₋₉ heterocyclyl;
X is N, and R⁴ is absent; or X is C, and R⁴ is hydrogen, halogen, cyano, optionally substituted amino, optionally substituted acyl, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, or optionally substituted C₁₋₉ heteroaryl;
R⁵ is optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, optionally substituted C₁₋₉ heteroaryl, —CONH₂, or —Z—R^{5A};
Z is optionally substituted amino, optionally substituted C₂₋₉ heterocyclylene, optionally substituted C₂₋₉ heteroarylene, optionally substituted C₆₋₁₀ arylene, or optionally substituted C₃₋₈ cycloalkylene;
R^{5A} is hydrogen, halogen, cyano, optionally substituted C₁₋₆ alkylsulfonyl, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, or optionally substituted C₁₋₉ heteroaryl;
R⁶ is hydrogen, halogen, cyano, optionally substituted C₁₋₆ alkyl, optionally substituted C₂₋₆ alkenyl, optionally substituted C₂₋₆ alkynyl, optionally substituted C₃₋₈ cycloalkyl, or —OR^{6A}; and
   R^{6A} is hydrogen, optionally substituted C₁₋₆ alkyl, optionally substituted C₂₋₆ alkenyl, optionally substituted C₂₋₆ alkynyl, or optionally substituted C₃₋₈ cycloalkyl.

In some embodiments, the compounds is of formula (II):

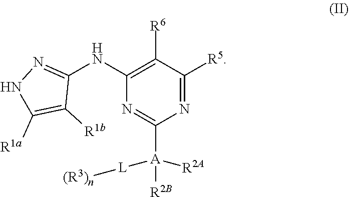

or a pharmaceutically acceptable salt thereof.

In some embodiments, the compound is of formula (III):

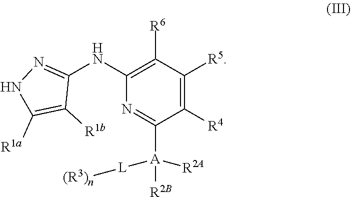

or a pharmaceutically acceptable salt thereof.

In some embodiments, the compound is of formula (II-A):

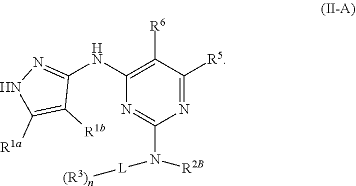

or a pharmaceutically acceptable salt thereof.

In some embodiments, the compound is of formula (III-A):

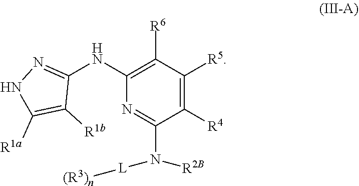

or a pharmaceutically acceptable salt thereof.

In some embodiments, the compound is of formula (II-B):

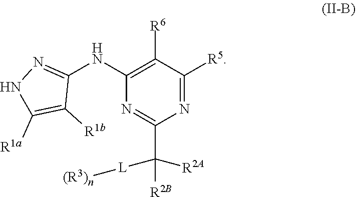

or a pharmaceutically acceptable salt thereof.

In some embodiments, the compound is of formula (III-B):

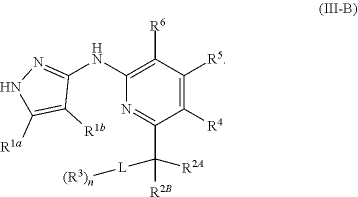

or a pharmaceutically acceptable salt thereof.

In some embodiments, one of R^{2A} and R^{2B} is hydrogen. In some embodiments, one of R^{2A} and R^{2B} is optionally substituted C₁₋₆ alkyl. In some embodiments, one of R^{2A} and R^{2B} is optionally substituted C₁₋₆ heteroalkyl.

In some embodiments, R^{2B} is:

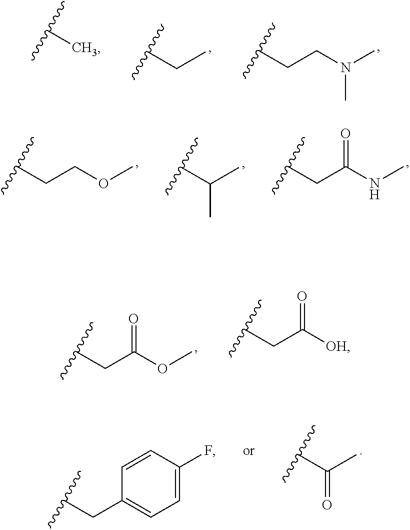

In some embodiments, the compound is of formula (II-C):

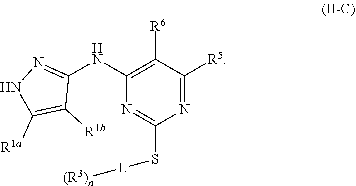

or a pharmaceutically acceptable salt thereof.

In some embodiments, the compound is of formula (III-C):

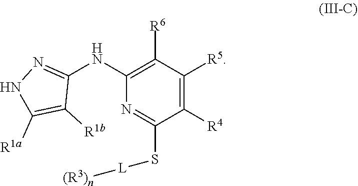

or a pharmaceutically acceptable salt thereof.

In some embodiments, the compound is of formula (II-D):

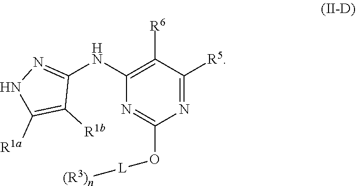

In some embodiments, the compound is of formula (III-D):

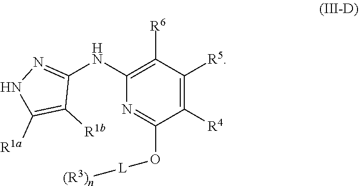

or a pharmaceutically acceptable salt thereof.

In some embodiments, R^{1a} and R^{1b} are independently are optionally substituted C₁₋₆ alkyl, halo, optionally substituted C₁₋₆ alkoxy, optionally substituted alkynyl, or optionally substituted C₃₋₆ cycloalkyl. In some embodiments, R^{1a} and R^{1b} are independently —CH₃—Cl, —OMe, —CH₂OMe, —CN, —CF₂H, —CF₃, —CHF₂, cyclopropyl, or cyclobutyl. In some embodiments, R^{1a} and R^{1b} together with the atoms to which they are attached are a cycloalkyl, cycloalkylene, cycloalkylyne, aryl, heterocyclyl, or heteroaryl. In some embodiments, R^{1a} and R^{1b} together with the atoms to which they are attached are:

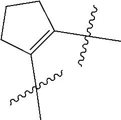 [SMILES: *C1=C(*)CCC1]

In some embodiments, L is optionally substituted C₆₋₁₀ aryl. In some embodiments, the optionally substituted C₆₋₁₀ aryl is optionally substituted phenyl.

In some embodiments, L is optionally substituted C₂₋₉ heteroaryl. In some embodiments, L is optionally substituted C₅ heteroaryl. In more particular embodiments, the optionally substituted C₅ heteroaryl contains one N. In some embodiments, -L-(R³)ₙ is:

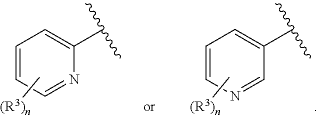

In some embodiments, at least one R³ is halogen. In some embodiments, at least one R₃ is F. In some embodiments, at least one R³ is Cl. In some embodiments, at least one R³ is Br. In some embodiments, at least one R³ is —S(O)ₘR^{3A}. In some embodiments, m is 1. In some embodiments, m is 2. In some embodiments, wherein R^{3A} is optionally substituted C₁₋₆ alkyl. In some embodiments,
R^{3A} is —CH₃. In some embodiments, R^{3A} is optionally substituted C₃₋₈ cycloalkyl. In some embodiments,
R^{3A} is optionally substituted cyclopropyl. In some embodiments, at least one R³ is optionally substituted C₂₋₉ heteroaryl. In some embodiments, at least one R³ is —N(R^{3B})₂. In some embodiments, at least one R³ is —OR^{3B}.

In some embodiments, -L-(R³)ₙ is:

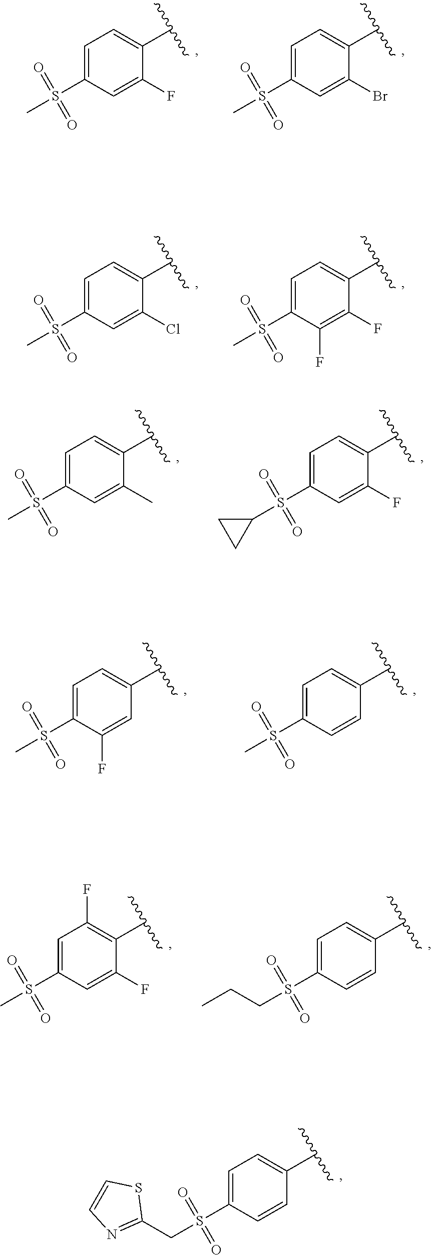

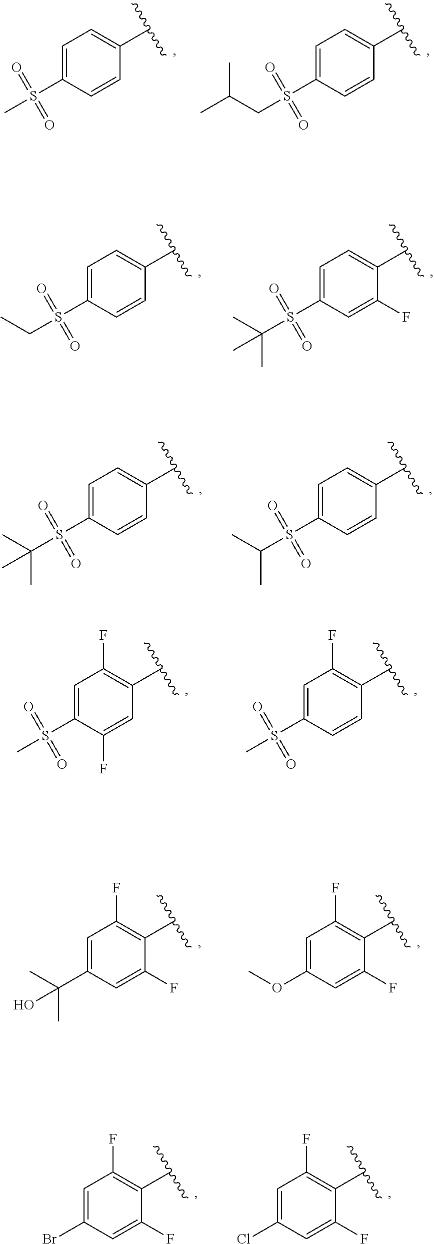

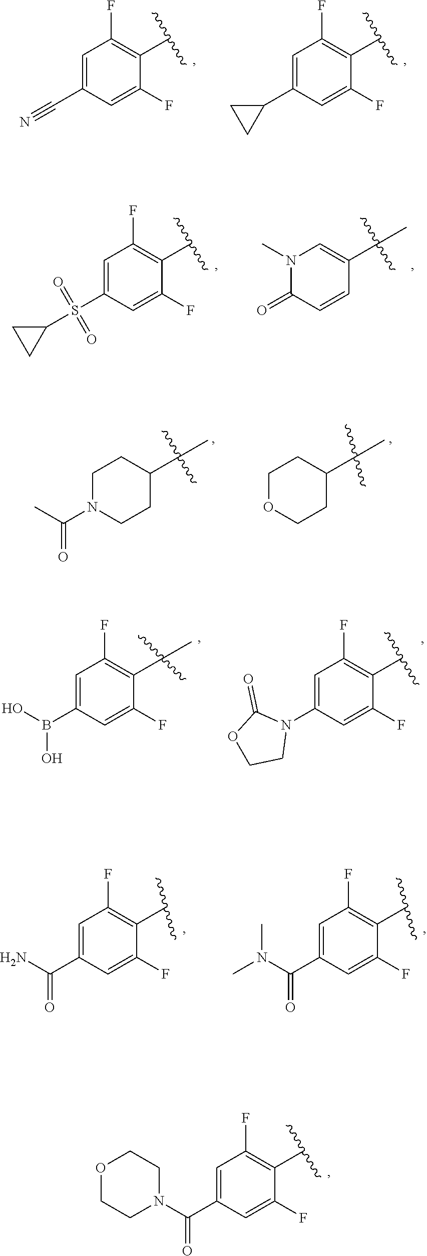

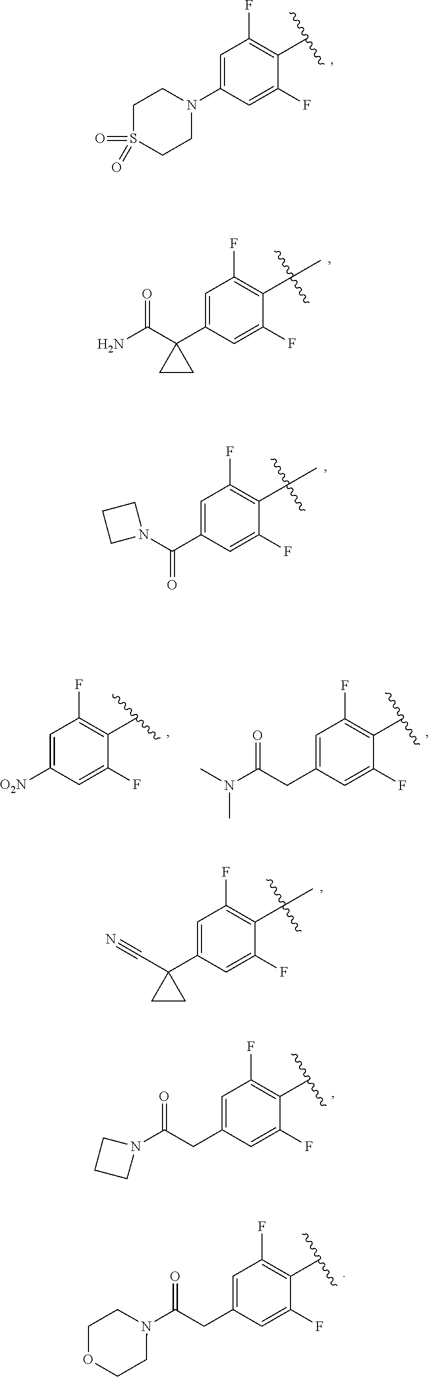

In some embodiments, -L-(R³)ₙ is:

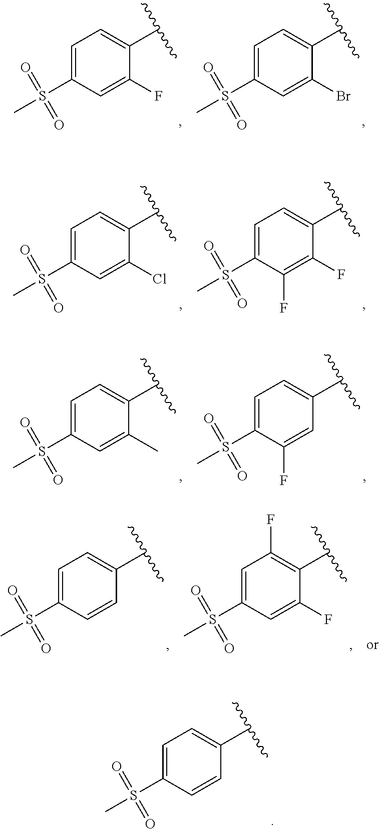

In some embodiments, -L-(R³)ₙ is:

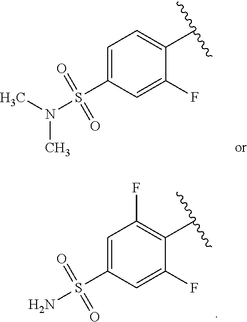 [SMILES: *c1ccc(S(=O)(=O)N(C)C)cc1F ; *c1c(F)cc(S(N)(=O)=O)cc1F]

In some embodiments, -L-(R³)ₙ is:

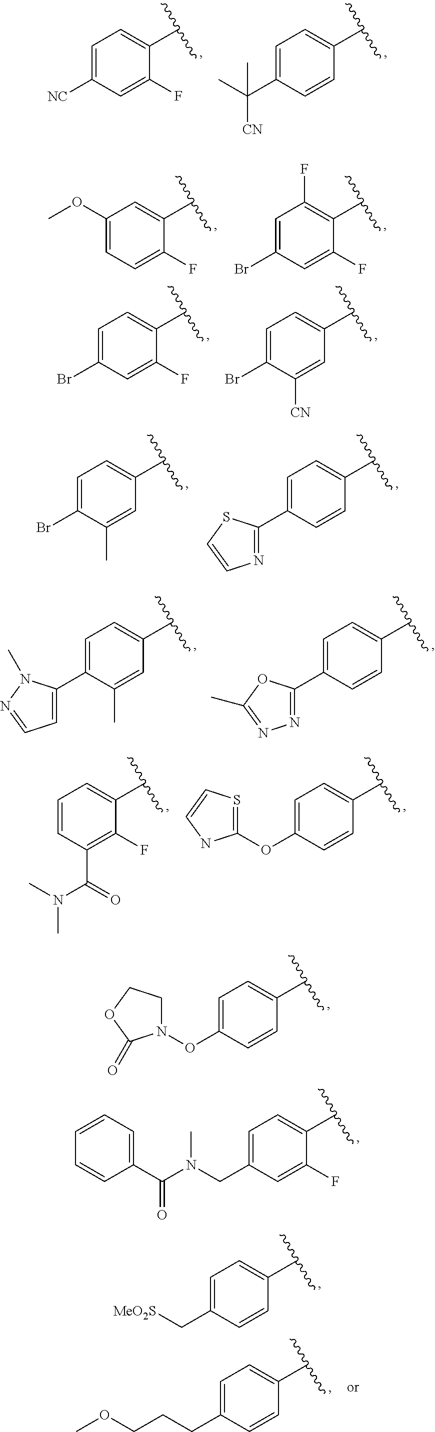

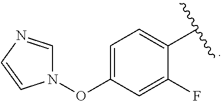 [SMILES: *c1ccc(On2ccnc2)cc1F]

In some embodiments, -L-(R³)ₙ is:

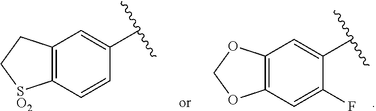 [SMILES: *c1ccc2c(c1)CCS2(=O)=O ; *c1cc2c(cc1F)OCO2]

In some embodiments, -L-(R³)ₙ is:

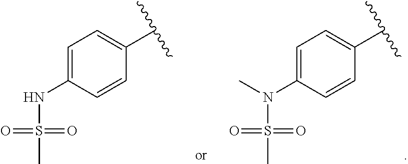 [SMILES: *c1ccc(NS(C)(=O)=O)cc1 ; *c1ccc(N(C)S(C)(=O)=O)cc1]

In some embodiments, -L-(R³)ₙ is:

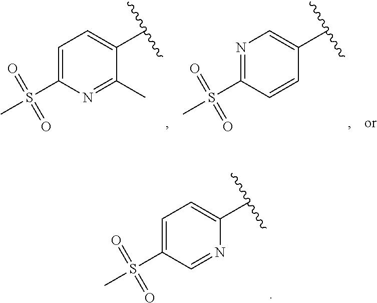

In some embodiments, R⁴ is halogen. In some embodiments, R⁴ is F. In some embodiments, R⁴ is Cl. In some embodiments, R⁴ is cyano. In some embodiments, R⁴ is optionally substituted amino. In some embodiments, R⁴ is —NH₂ or —N(CH₃)₂. In some embodiments, R⁴ is hydrogen. In some embodiments, R⁴ is —CH₃.

In some embodiments, R⁵ is optionally substituted C₁₋₉ heteroaryl. In some embodiments, R⁵ is optionally substituted C₃-C₄ heteroaryl or optionally substituted C₄ heterocycle.

In some embodiments, the optionally substituted C₃-C₄ heteroaryl or optionally substituted C₄ heterocycle comprises 1 to 2 N atoms. In some embodiments, R⁵ is:

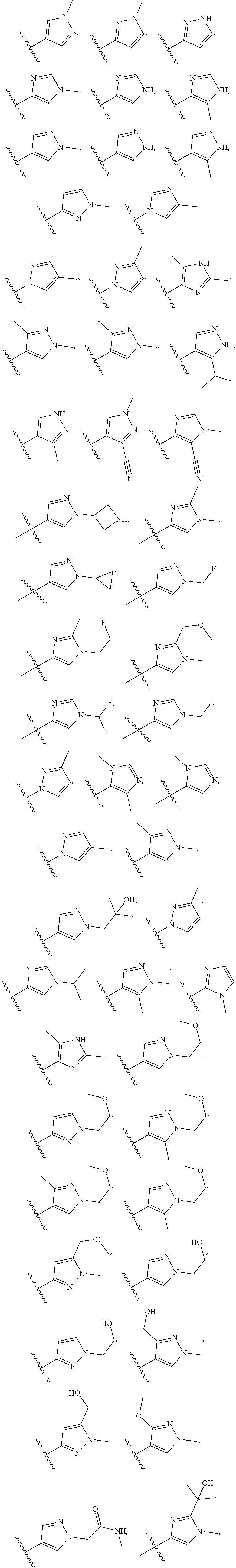

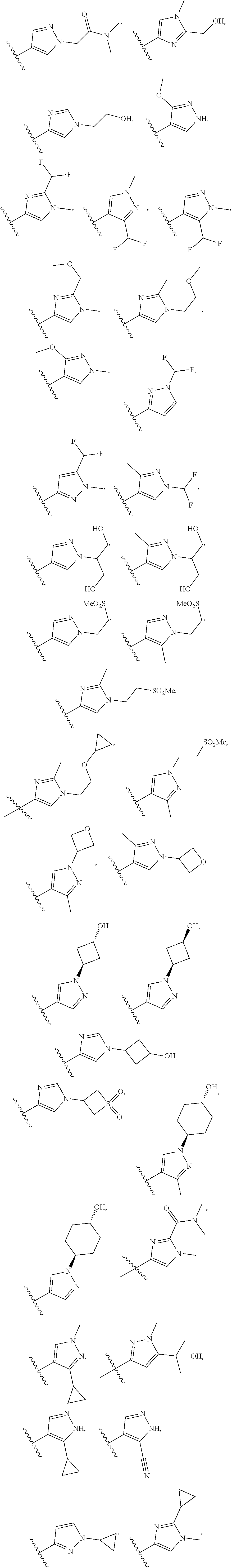

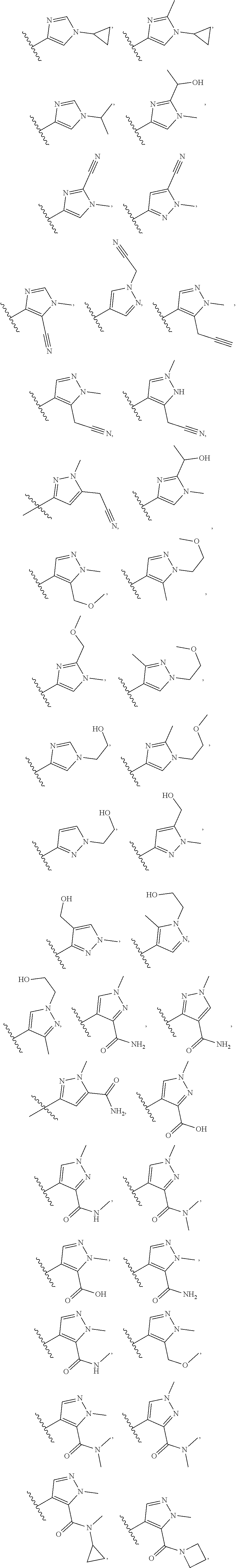

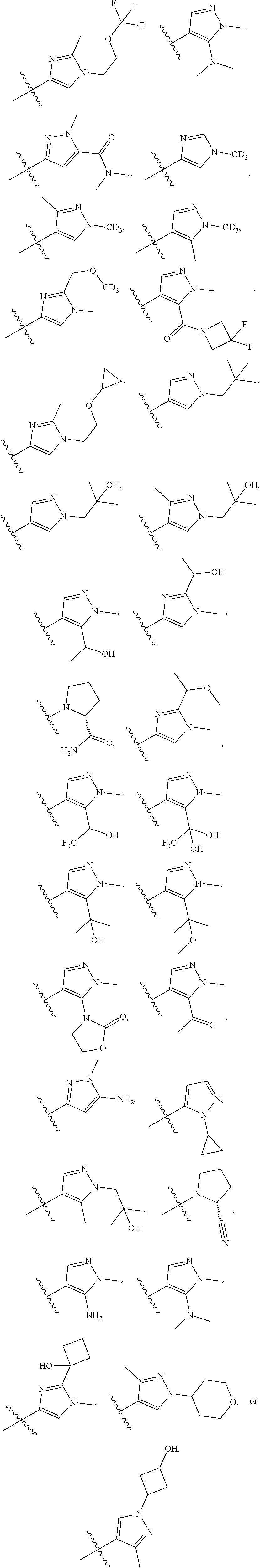

In some embodiments, R⁵ is

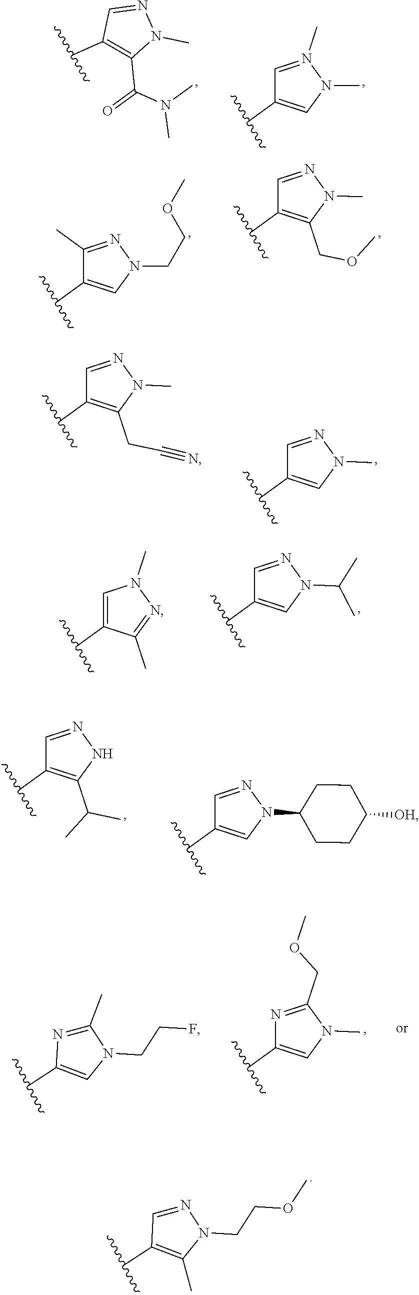

In some embodiments, the optionally substituted C₃ heteroaryl comprises 1 N atom and 1 S atom or 1 O atom. In some embodiments, R⁵ is:

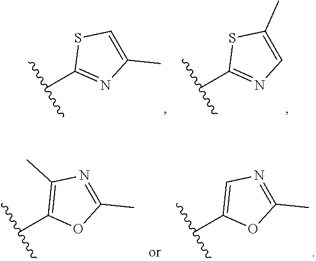

In some embodiments, R⁵ is —Z—R^{5A}. In some embodiments, Z is an optionally substituted amino and R^{5A} is

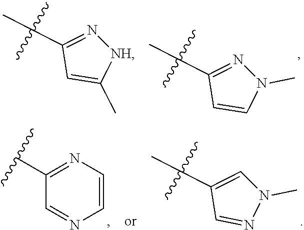

In some embodiments, Z is optionally substituted C₂₋₉ heteroarylene.

In some embodiments, R⁵ is optionally substituted C₂₋₉ heterocyclyl. In some embodiments, R⁵ is:

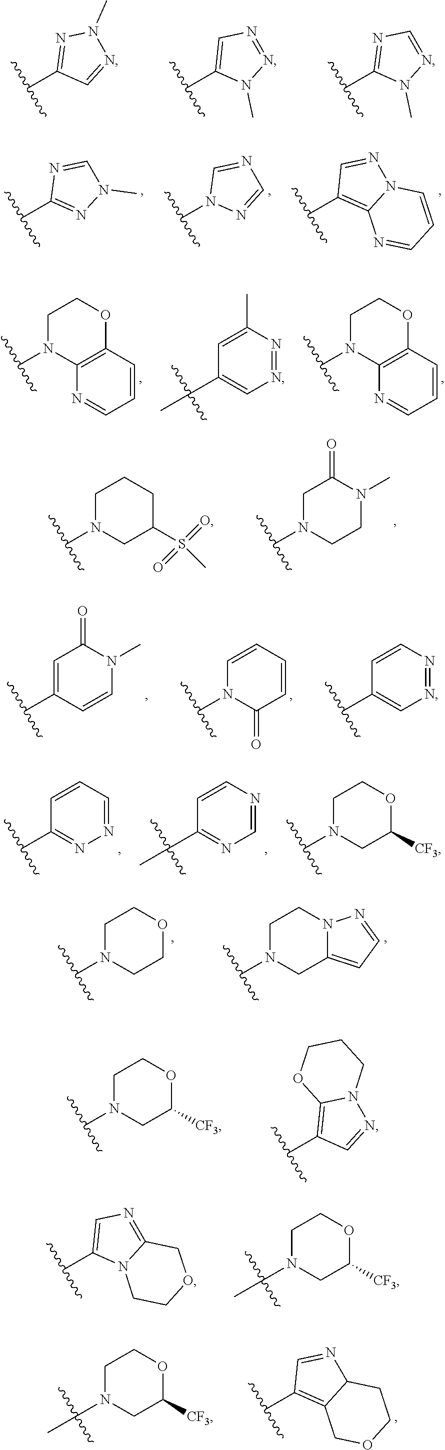

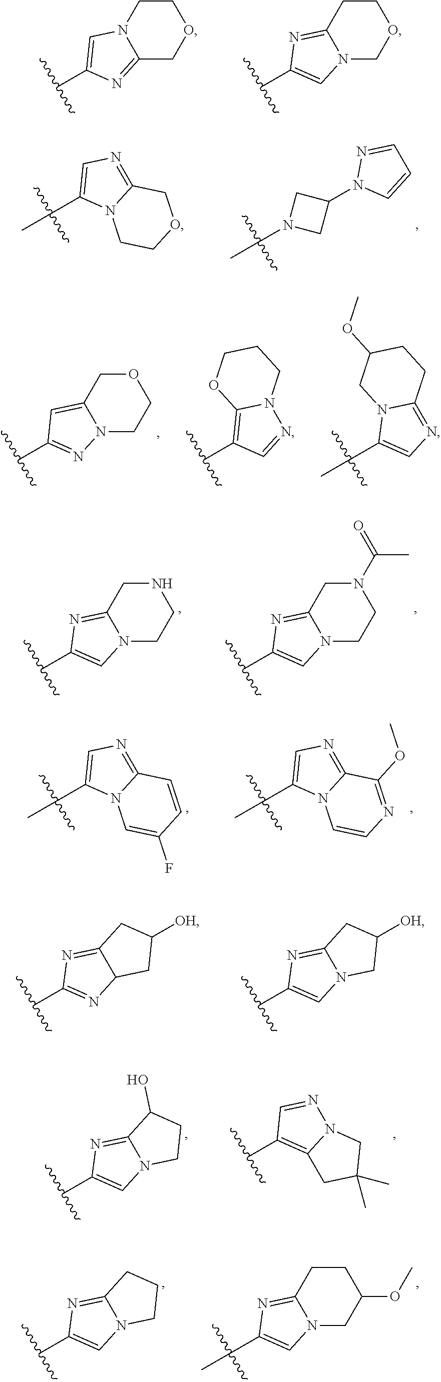

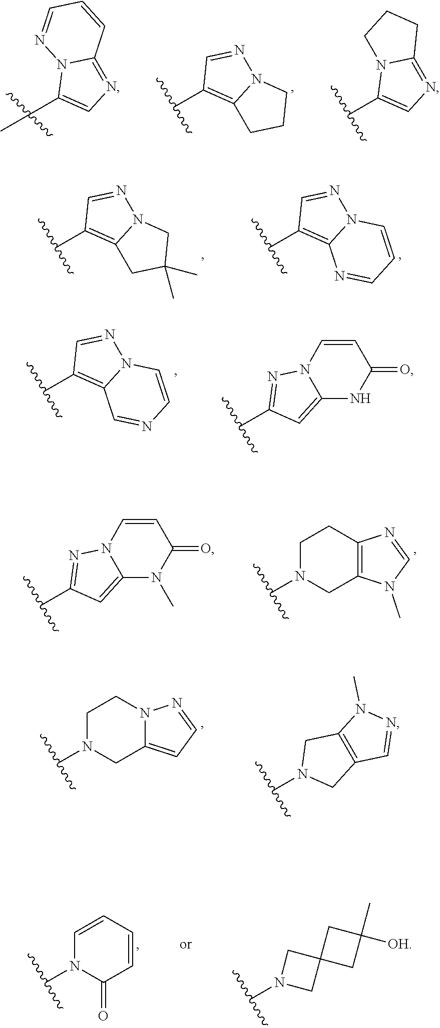

In some embodiments, R⁵ is:

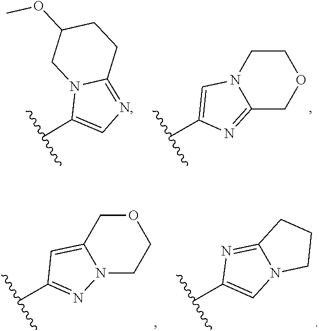 [SMILES: *c1cnc2n1CC(OC)CC2 ; *c1cn2c(n1)COCC2 ; *c1cc2n(n1)CCOC2 ; *c1cn2c(n1)CCC2]

In some embodiments, R^{5A} is:

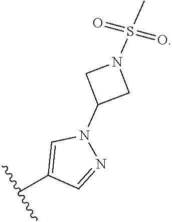 [SMILES: *c1cnn(C2CN(S(C)(=O)=O)C2)c1]

In some embodiments, R⁵ is:

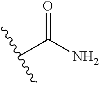 [SMILES: *C(N)=O]

In some embodiments, R⁶ is optionally substituted C₁₋₆ alkyl. In some embodiments, R⁶ is:

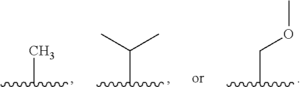

In some embodiments, R⁶ is —OR^{6A}. In some embodiments, R^{6A} is —CH₃. In some embodiments, R⁶ is optionally substituted C₃₋₈ cycloalkyl. In some embodiments, R⁶ is optionally substituted cyclopropyl. In some embodiments, R⁶ is:

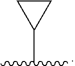 [SMILES: *C1CC1]

In some embodiments, the compound is of formula (IV):

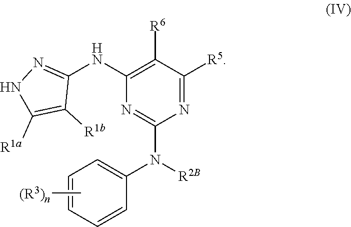

In some embodiments, the compound is of formula (V):

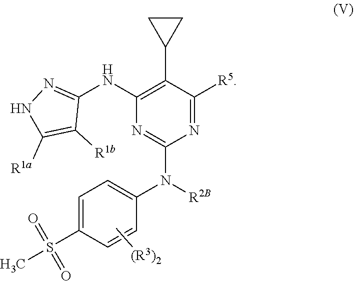

In some embodiments, the compound is selected from the group consisting of compounds 1 to 365, including pharmaceutically acceptable salts thereof.

In another aspect, the invention provides a pharmaceutical composition described herein, or a pharmaceutically acceptable salt thereof, and a pharmaceutically acceptable excipient. In some embodiments, the composition is isotopically enriched in deuterium.

In still another aspect, the invention relates to a method of inhibiting PLK4 expression in a cell, the method including contacting the cell with any compound described herein, or a pharmaceutically acceptable salt thereof. In some embodiments, the cell is overexpressing TRIM37. In some embodiments, the cell is in a subject.

In still another aspect, the invention provides a method of treating a subject in need thereof, the method including administering to the subject a compound described herein, or a pharmaceutically acceptable salt thereof. In some embodiments, the subject is suffering from, and is in need of treatment for, a disease or condition having the symptom of cell hyperproliferation. In some embodiments, the disease is cancer. In some embodiments, the cancer is a cancer overexpressing TRIM37.

In a still further aspect, the invention provides a method of treating cancer in a subject, the method including administering to the subject a therapeutically effective amount of a compound described herein, or a pharmaceutically acceptable salt thereof, or a pharmaceutical composition of any compound described herein including a pharmaceutically acceptable excipient, where the cancer has been previously identified as a cancer overexpressing TRIM37.

In some embodiments, the cancer overexpressing TRIM37 is uterine cancer, ovarian cancer, breast cancer, stomach cancer, esophageal cancer, lung cancer, or endometrial cancer.

In a still further aspect, the invention provides a method of inducing cell death in a cancer cell overexpressing TRIM37, the method including contacting the cell with an effective amount of a PLK4 inhibitor. In some embodiments, the PLK4 inhibitor is a compound described herein, or a pharmaceutically acceptable salt thereof.

In some embodiments, the cell is in a subject.

### ABBREVIATIONS

Abbreviations and terms that are commonly used in the fields of organic chemistry, medicinal chemistry, pharmacology, and medicine and are well known to practitioners in these fields are used herein. Representative abbreviations and definitions are provided below:
Ac is acetyl [CH₃C(O)—];
ACN is acetonitrile;
Ac₂O is acetic anhydride;
AcOH is acetic acid;
APC is antigen-presenting cell;
Ar is aryl;
aq. is aqueous;
9-BBN is 9-borabicyclo[3.3.1]nonane;
BINAP is (2,2′-bis(diphenylphosphino)-1,1′-binaphthyl);
Bn is benzyl;
Boc is tert Butyloxycarbonyl;
n-BuLi is n-butyl lithium;
Br₂ is bromine;
CDI is carbonyldiimidazole;
cmpd is compound;
conc. is concentrated;
DCM is dichloromethane;
DIAD is diisopropylazodicarboxylate;
DIBAL is diisobutylaluminum hydride;
DIPEA is diisopropylethyl amine;
DMA is dimethylacetamide;
DMAP is 4-dimethylaminopyridine;
DME is dimethoxyethane;
DMF is N,N-dimethylformamide;
DMSO is dimethyl sulfoxide;
dppf is 1,1′-bis(diphenylphosphino)ferrocene;
dtbpf is 1,1′-Bis(di-tert-butylphosphino)ferrocene;
EDAC (or EDC) is 1-ethyl-3-[3-(dimethylamino)propyl]-carbodiimide HCl;
ESI is electrospray ionization mass spectrometry;
Et₂O is diethyl ether;
Et₃N is triethylamine;
Et is ethyl;
EtOAc is ethyl acetate;
EtOH is ethanol;
(+ESI) is electronspay ionization in positive mode;
3-F-Ph is 3-fluorophenyl,
h is hour;
hrs is hours;
HATU is (1-[bis(dimethylamino)methylene]-1H-1,2,3-triazolo[4,5-b]pyridinium 3-oxide hexafluorophosphate;
HCl is hydrochloric acid;
Het is heteroaryl;
Hex is hexanes;
HOBt is 1-hydroxybenzotriazole;
HPLC is high performance liquid chromatography;
IPA or iPrOH is isopropanol; 17
IPAc is isopropyl acetate;
I₂ is iodine;
LCMS is HPLC with mass spectral detection;
LiHMDS is lithium bis(trimethylsilyl)amide;
LG is leaving group;
M is molar;
mCPBA is metachloroperbenzoic acid;
mmol is millimole;
Me is methyl;
MeI is iodomethane;
MeCN is acetonitrile;
MeMgBr is methylmagnesium bromide;
MeMgCl is methylmagnesium chloride;
MeOH is methanol;
min is minute;
MOM is methoxymethyl;
Ms is methanesulfonyl;
MS is mass spectrometry;
MTBE is methyl tert-butyl ether;
MW is microwave;
N is normal;
NaBH(OAc)₃ is sodium triacetoxyborohydride
NaH is sodium hydride;
NaHMDS is sodium bis(trimethylsilyl)amide;
NaOAc is sodium acetate;
NaOtBu is sodium tert-butoxide;
NBS is N-bromosuccinimide;
NCS is N-chlorosuccinimide;
NIS is N-iodosuccinimide;
NMO is N-methylmorpholine N-oxide;
NMP is N-methyl pyrrolidinone;
NMR is nuclear magnetic resonance spectroscopy;
PdCl₂(dppf) is [1,1′-Bis(diphenylphosphino)ferrocene]dichloropalladium(II);
PdCl₂(dppf)·CH₂Cl₂ is [1,1′-Bis(diphenylphosphino)ferrocene]dichloropalladium(II), complex with dichloromethane;
Pd₂(dba)₃ is tris(dibenzylideneacetone)dipalladium;
PdCl₂(PPh₃)₂ is dichlorobis-(triphenylphosphene)palladium;
Pd-PEPPSI™-SIPr is (1,3-Bis(2,6-diisopropylphenyl)imidazolidene) (3-chloropyridyl) palladium(II) dichloride;
PE is petroleum ether;
PG Denotes a protecting group;
Ph is phenyl;
PhMe is toluene;
PIV-Cl is pivaloyl chloride, Trimethylacetyl chloride;
PPh₃ is triphenylphosphine;
PMB is para-methoxybenzyl;
Reagent alcohol is a mixture of 90% ethanol, 5% isopropanol and 5% methanol;
rt or RT is room temperature;
RBF is round-bottom flask;
Ruphos is 2-Dicyclohexylphosphino-2′,6′-diisopropoxybiphenyl;
RuPhos Pd G1 is chloro-(2-Dicyclohexylphosphino-2′,6′-diisopropoxy-1,1′-biphenyl)[2-(2-aminoethyl)phenyl]palladium(II);
sat. is saturated;
SEM is [2-(trimethylsilyl)ethoxy]methyl;
SFC is supercritical fluid chromatography;
S_{N}Ar is nucleophilic aromatic substitution;
TBAB is tetrabutyl ammonium bromide;
TBAF is tetrabutyl ammonium fluoride;
TBS is tert-butyldimethylsilyl;
tBu is tert-butyl;
Tf is trifluoromethanesulfonyl;
TFA is trifluoroacetic acid;
THF is tetrahydrofuran;
THP is tetrahydropyran;
TLC is thin layer chromatography;
TMAD is tetramethylazodicarboxamide;
TMS is trimethylsilyl;
TPAP is tetrapropylammonium perruthenate;
Ts is p-toluenesulfonyl;
UPLC is ultra-performance liquid chromatography;
UPLC-MS is UPLC with mass spectral detection;
Xantphos is 4,5-Bis(diphenylphosphino)-9,9-dimethylxanthene:
XPhosPdG₂ is Chloro(2-dicyclohexylphosphino-2′,4′,6′-triisopropyl-1,1′-biphenyl)[2-(2′-amino-1,1′-biphenyl)]palladium(II).
XPhosPdG3 is (2-Dicyclohexylphosphino-2′,4′,6′-triisopropyl-1,1′-biphenyl)[2-(2′-amino-1,1′-biphenyl)]palladium(II) methanesulfonate.

### Definitions

The term “aberrant,” as used herein, refers to different from normal. When used to describe activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g. by administering a compound or using a method as described herein), results in reduction of the disease or one or more disease symptoms.

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C1-7 alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent alkenyl group. An optionally substituted alkenylene is an alkenylene that is optionally substituted as described herein for alkenyl.

The term “alkoxy,” as used herein, represents a chemical substituent of formula-OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; alkoxy; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —C(O)R or —SO₂R, where R is amino; and ═NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)2-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “alkynylene,” as used herein, refers to a divalent alkynyl group. An optionally substituted alkynylene is an alkynylene that is optionally substituted as described herein for alkynyl.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —C(O)OR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; —(CH₂)ₙ—C(O)OR^{A}; —C(O)R; and —SO₂R, where R is amino or alkyl, R^{A} is H or alkyl, and n is 0 or 1. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula-OR, where R is an aryl group, unless otherwise specified. In optionally substituted aryloxy, the aryl group is optionally substituted as described herein for aryl.

The term “azido,” as used herein, represents an —N₃ group.

The term “cancer,” as used herein, refers to all types of cancer, neoplasm or malignant tumors found in mammals (e.g., humans).

The term “carbocyclic,” as used herein, represents an optionally substituted C3-16 monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “carcinoma,” as used herein, refers to a malignant new growth made up of epithelial cells tending to infiltrate the surrounding tissues and give rise to metastases.

The term “cyano,” as used herein, represents —CN group.

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, represents a divalent cycloalkenyl group. An optionally substituted cycloalkenylene is a cycloalkenylene that is optionally substituted as described herein for cycloalkyl.

The term “cycloalkoxy,” as used herein, represents a chemical substituent of formula-OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C_{3-C10} cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is optionally substituted amino; ═NR′, where R′ is H, alkyl, aryl, or heterocyclyl; and —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidinyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, quinazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; —C(O)R or —SO₂R, where R is amino or alkyl; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic).

The term “lymphoma,” as used herein, refers to a cancer arising from cells of immune origin.

The term “melanoma,” as used herein, is taken to mean a tumor arising from the melanocytic system of the skin and other organs.

The term “nitro,” as used herein, represents an —NO₂ group.

The term “oxo,” as used herein, represents a divalent oxygen atom (e.g., the structure of oxo may be shown as ═O).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “PLK4,” as used herein, refers to polo-like kinase 4, also known as serine/threonine-protein kinase PLK4 (Gene name PLK4).

The term “PLK4 inhibitor,” as used herein, represents a compound that upon contacting the enzyme PLK4, whether in vitro, in cell culture, or in an animal, reduces the activity of PLK4, such that the measured PLK4 IC₅₀ is 10 μM or less (e.g., 5 μM or less or 1 μM or less). For certain PLK4 inhibitors, the PLK4 IC₅₀ may be 100 nM or less (e.g., 10 nM or less, or 3 nM or less) and could be as low as 100 pM or 10 pM. Preferably, the PLK IC₅₀ is 1 nM to 1 μM (e.g., 1 nM to 750 nM, 1 nM to 500 nM, or 1 nM to 250 nM). Even more preferably, the PLK4 IC₅₀ is less than 20 nm (e.g., 1 nM to 20 nM).

The term “pre-malignant” or “pre-cancerous,” as used herein, refers to a condition that is not malignant but is poised to become malignant.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone-enol, enamine-imine, amide-imidic acid, nitroso-oxime, ketene-ynol, and amino acid-ammonium carboxylate.

The term “sarcoma” generally refers to a tumor which is made up of a substance like the embryonic connective tissue and is generally composed of closely packed cells embedded in a fibrillar or homogeneous substance.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

The term “TRIM37” refers to the protein tripartite motif containing 37 and its gene.

### DETAILED DESCRIPTION

In general, the invention provides compounds, pharmaceutical compositions containing the same, methods of preparing the compounds, and methods of use. Compounds of the invention may be PLK4 inhibitors. These compounds may be used to inhibit PLK4 in a cell, e.g., a cell in a subject (e.g., a cell overexpressing TRIM37 or having altered centrosomal or centriolar function or number). The subject may be in need of a treatment for a disease or condition, e.g., a disease or condition having a symptom of cell hyperproliferation, e.g., a cancer. The PLK4 inhibitory activity of the compounds disclosed herein is useful for treating a subject in need of a treatment for cancer.

The Polo-like kinase (PLK) family of serine/threonine kinases, characterized by the presence of Polo box domains, play a critical role in the regulation of mitosis. Of the five members described to date, PLK1, PLK2, PLK3, PLK4, and PLK5, PLK1 is the most studied member and multiple PLK1 inhibitors have been described. PLK4 is structurally the most distinct member of the family. Unlike PLK1, PLK2, and PLK3, PLK4 has only one Polo box and an active site with high homology to the Aurora kinases. PLK4 has a restricted tissue distribution, being present only in proliferating tissues. PLK4 localizes to the centrosomes and is a critical regulator of centriole duplication. Deregulation of PLK4 results in loss of centrosome numerical integrity and leads to chromosome instability. It has been shown that PLK4 is up-regulated in breast cancer, specifically in the basal-like subtype, and that high PLK4 levels are associated with poor patient outcomes. Consistent with its key role in centriolar biogenesis, inhibition of PLK4 activity or loss of PLK4 through genetic means has been shown to result in loss of centrioles.

Inhibitors of PLK4 may be particularly useful in the treatment of tumors harboring TRIM37 amplification using a synthetic lethal therapeutic strategy. The TRIM37 locus is found at the border of 17q22 and 17q23—a chromosomal region that is amplified in a number of cancers, most prominently in around 50-60% of neuroblastomas and roughly 10% of breast cancers. TRIM37 is a ubiquitin E3 ligase and plays an important role in regulating the cellular expression of pericentriolar material. Overexpression of TRIM37 leads to enhanced degradation of pericentriolar material. Successful microtubule nucleation is a requirement for cell division and survival. Cells can survive without centrosomes through reliance on pericentriolar material. In contexts where abundance of pericentriolar material is suppressed, such as through overexpression of TRIM37, cells are unable to survive following PLK4 inhibition due to inability to adequately form microtubules.

The compound of the invention may be, e.g., a compound of formula (I):

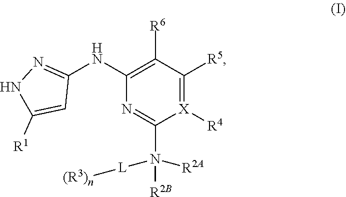

or a pharmaceutically acceptable salt thereof,
where
   n is 0, 1, 2, 3, or 4;
   m is 0, 1, or 2;
   L is optionally substituted C₂₋₉ heterocyclyl, optionally substituted C₂₋₉ heteroaryl, optionally substituted C₆₋₁₀ aryl, or optionally substituted C₃₋₈ cycloalkyl, wherein L is further optionally substituted by n occurrences of R³;
   R¹ is hydrogen, halogen, optionally substituted C₁₋₆ alkyl, optionally substituted C₁₋₆ heteroalkyl, optionally substituted C₂₋₆ alkenyl, optionally substituted C₂₋₆ alkynyl, or optionally substituted C₃₋₈ cycloalkyl;
   A is O or S, and R^{2A} and R^{2B} are both absent; or A is N, R^{2A} is absent, and R^{2B} is hydrogen, optionally substituted C₁₋₆ alkyl, optionally substituted C₁₋₆ heteroalkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl, or R^{2B} and L, together with the atom to which they are attached, combine to form an optionally substituted C₂₋₉ heterocyclyl or optionally substituted C₂₋₉ heteroaryl; or A is C, and each of R^{2A} and R^{2B} are independently hydrogen, optionally substituted C₁₋₆ alkyl, optionally substituted C₁₋₆ heteroalkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl;
   each R³ is independently halogen, cyano, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₆₋₁₀ aryl C₁₋₆ alkyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, —S(O)ₘR^{3A}, —N(R^{3B})₂, or —OR^{3B};
   R^{3A} is optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₆₋₁₀ aryl, optionally substituted C₂₋₉ heterocyclyl, optionally substituted C₁₋₉ heteroaryl, —OR^{3B} or —N(R^{3B})₂.

Each R^{3B} is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl; or two R^{3B} groups, together with the atom to which both are attached, combine to form an optionally substituted C₂₋₉ heterocyclyl;
X is N, and R⁴ is absent; or X is C, and R⁴ is hydrogen, halogen, cyano, optionally substituted amino, optionally substituted acyl, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, or optionally substituted C₁₋₉ heteroaryl;
   R⁵ is optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, optionally substituted C₁₋₉ heteroaryl, or —Z—R^{5A};
   Z is optionally substituted C₂₋₉ heterocyclylene, optionally substituted C₂₋₉ heteroarylene, optionally substituted C₆₋₁₀ arylene, or optionally substituted C₃₋₈ cycloalkylene;
   R^{5A} is hydrogen, halogen, cyano, optionally substituted C₁₋₆ alkylsulfonyl, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, or optionally substituted C₁₋₉ heteroaryl;
   R⁶ is hydrogen, halogen, cyano, optionally substituted C₁₋₆ alkyl, optionally substituted C₂₋₆ alkenyl, optionally substituted C₂₋₆ alkynyl, optionally substituted C₃₋₈ cycloalkyl, or —OR^{6A}; and
   R^{6A} is hydrogen, optionally substituted C₁₋₆ alkyl, optionally substituted C₂₋₆ alkenyl, optionally substituted C₂₋₆ alkynyl, or optionally substituted C₃₋₈ cycloalkyl.

The compound of the invention may be, e.g., a compound listed in Table 1 below or a pharmaceutically acceptable salt thereof.

TABLE 1

| | |
|---|---|
| 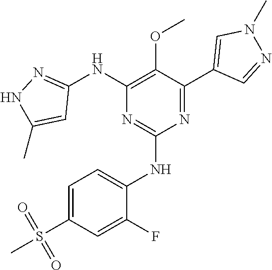 | 1 |
| 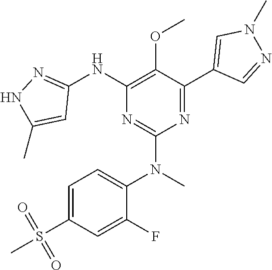 | 2 |
| 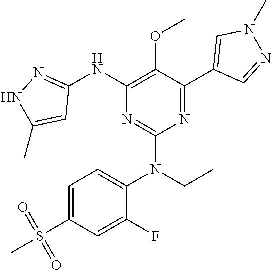 | 3 |
| 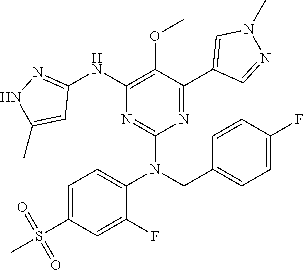 [SMILES: COc1c(Nc2cc(C)[nH]n2)nc(N(Cc2ccc(F)cc2)c2ccc(S(C)(=O)=O)cc2F)nc1-c1cnn(C)c1] | 4 |
| 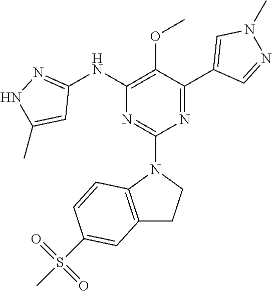 [SMILES: COc1c(Nc2cc(C)[nH]n2)nc(N2CCc3cc(S(C)(=O)=O)ccc32)nc1-c1cnn(C)c1] | 5 |
| 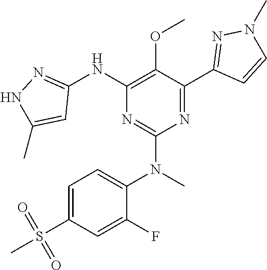 | 6 |
| 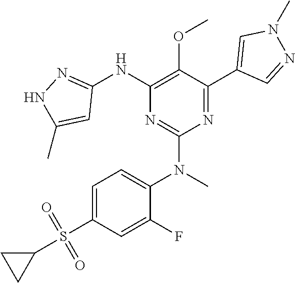 [SMILES: COc1c(Nc2cc(C)[nH]n2)nc(N(C)c2ccc(S(=O)(=O)C3CC3)cc2F)nc1-c1cnn(C)c1] | 7 |
| 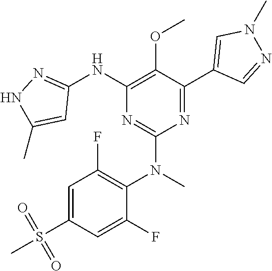 | 8 |
| 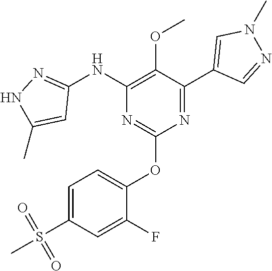 | 9 |
| 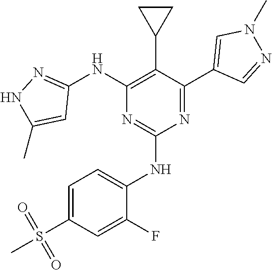 | 10 |
| 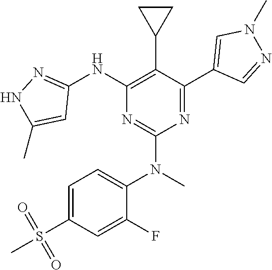 | 11 |
| 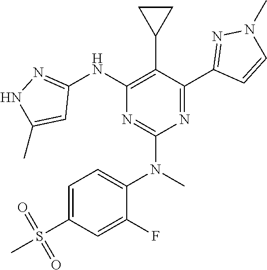 | 12 |
| 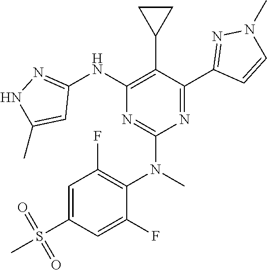 | 13 |
| 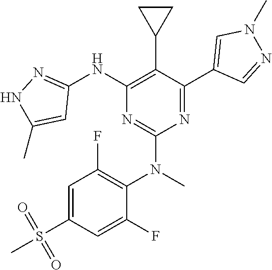 | 14 |
| 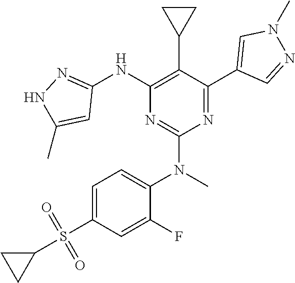 [SMILES: Cc1cc(Nc2nc(N(C)c3ccc(S(=O)(=O)C4CC4)cc3F)nc(-c3cnn(C)c3)c2C2CC2)n[nH]1] | 15 |
| 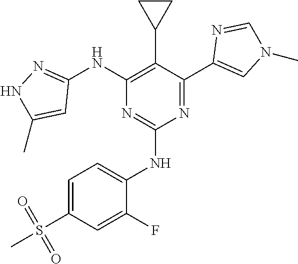 | 16 |
| 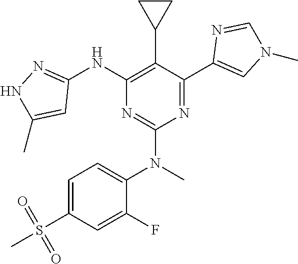 | 17 |
| 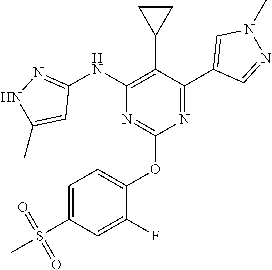 | 18 |
| 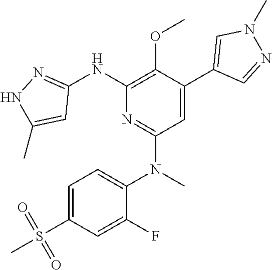 | 19 |
| 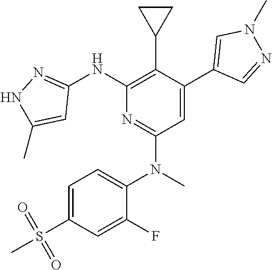 | 20 |
| 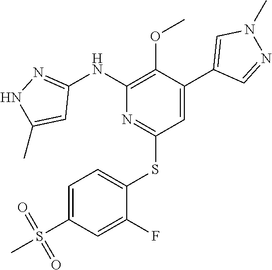 | 21 |
| 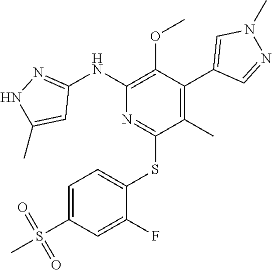 | 22 |
| 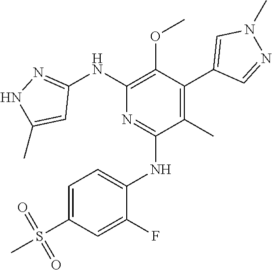 | 23 |
| 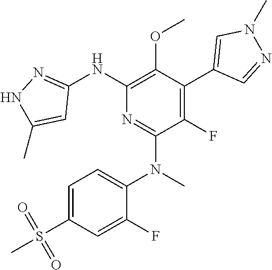 | 24 |
| 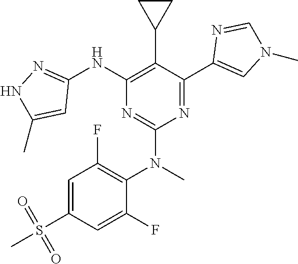 | 25 |
| 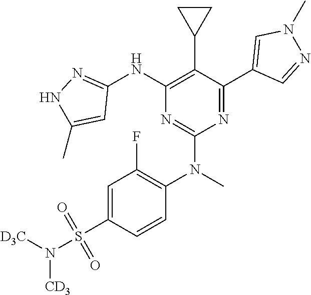 [SMILES: [2H]C([2H])([2H])N(C([2H])([2H])[2H])S(=O)(=O)c1ccc(N(C)c2nc(Nc3cc(C)[nH]n3)c(C3CC3)c(-c3cnn(C)c3)n2)c(F)c1] | 26 |
| 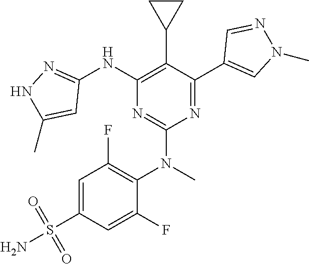 [SMILES: Cc1cc(Nc2nc(N(C)c3c(F)cc(S(N)(=O)=O)cc3F)nc(-c3cnn(C)c3)c2C2CC2)n[nH]1] | 27 |
| 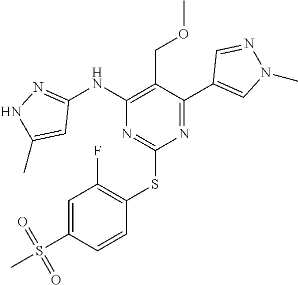 | 28 |
| 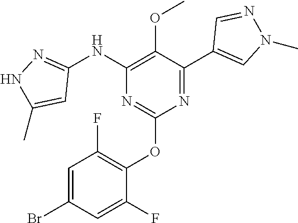 [SMILES: COc1c(Nc2cc(C)[nH]n2)nc(Oc2c(F)cc(Br)cc2F)nc1-c1cnn(C)c1] | 29 |
| 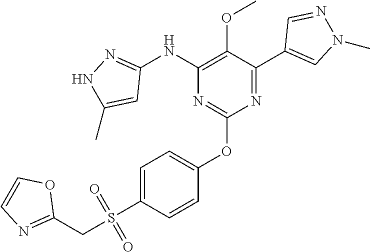 [SMILES: COc1c(Nc2cc(C)[nH]n2)nc(Oc2ccc(S(=O)(=O)Cc3ncco3)cc2)nc1-c1cnn(C)c1] | 30 |
| 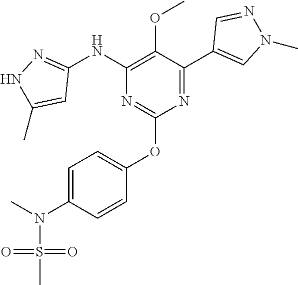 [SMILES: COc1c(Nc2cc(C)[nH]n2)nc(Oc2ccc(N(C)S(C)(=O)=O)cc2)nc1-c1cnn(C)c1] | 31 |
| 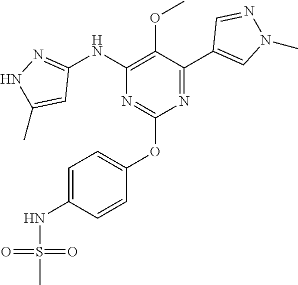 [SMILES: COc1c(Nc2cc(C)[nH]n2)nc(Oc2ccc(NS(C)(=O)=O)cc2)nc1-c1cnn(C)c1] | 32 |
| 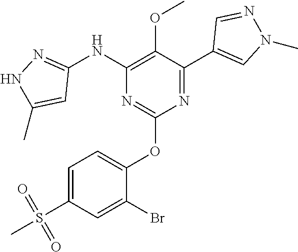 | 33 |
| 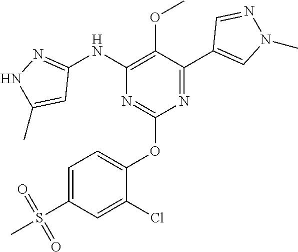 | 34 |
| 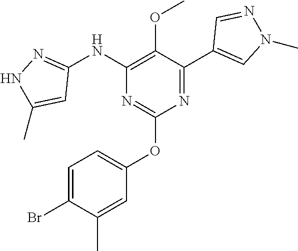 [SMILES: COc1c(Nc2cc(C)[nH]n2)nc(Oc2ccc(Br)c(C)c2)nc1-c1cnn(C)c1] | 35 |
| 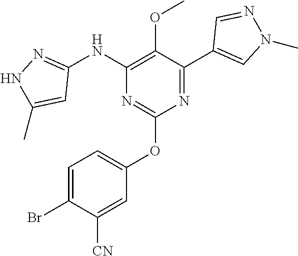 [SMILES: COc1c(Nc2cc(C)[nH]n2)nc(Oc2ccc(Br)c(C#N)c2)nc1-c1cnn(C)c1] | 36 |
| 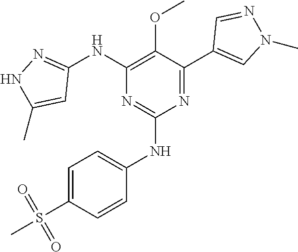 | 37 |
| 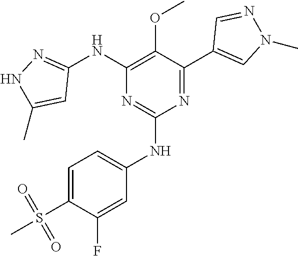 | 38 |
| 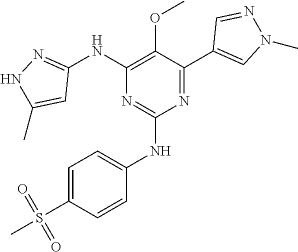 | 39 |
| 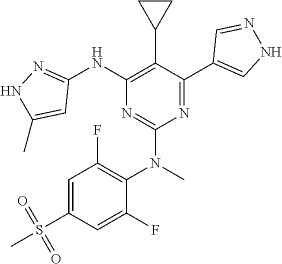 [SMILES: Cc1cc(Nc2nc(N(C)c3c(F)cc(S(C)(=O)=O)cc3F)nc(-c3cn[nH]c3)c2C2CC2)n[nH]1] | 40 |
| 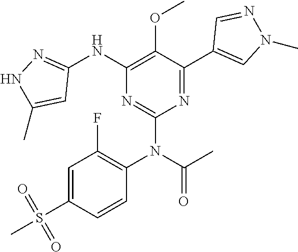 | 41 |
| 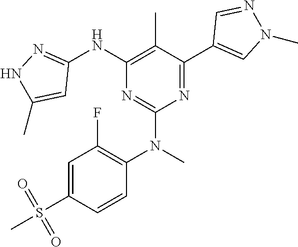 | 42 |
| 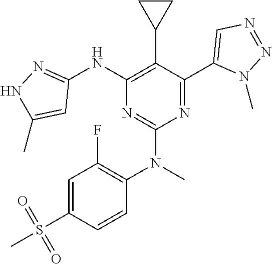 [SMILES: Cc1cc(Nc2nc(N(C)c3ccc(S(C)(=O)=O)cc3F)nc(-c3cnnn3C)c2C2CC2)n[nH]1] | 43 |
| 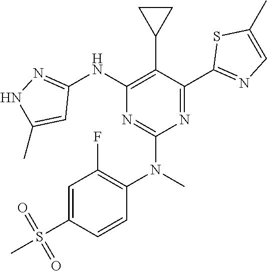 | 44 |
| 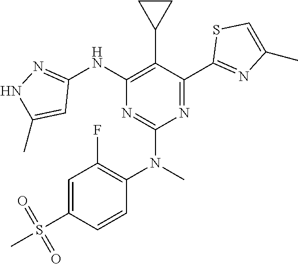 | 45 |
| 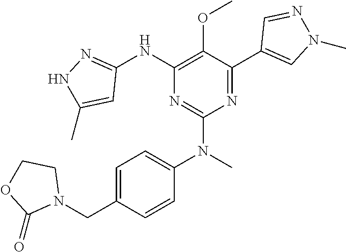 [SMILES: COc1c(Nc2cc(C)[nH]n2)nc(N(C)c2ccc(CN3CCOC3=O)cc2)nc1-c1cnn(C)c1] | 46 |
| 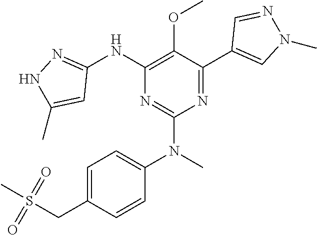 | 47 |
| 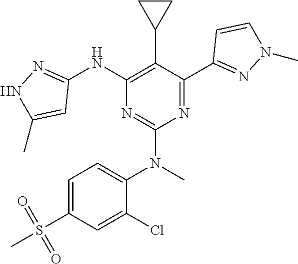 | 48 |
| 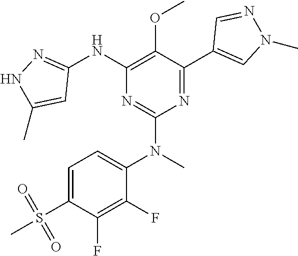 | 49 |
| 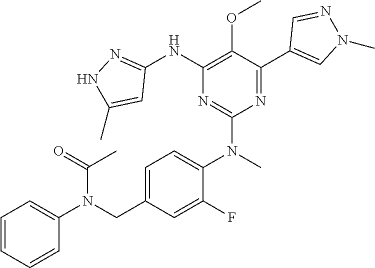 [SMILES: COc1c(Nc2cc(C)[nH]n2)nc(N(C)c2ccc(CN(C(C)=O)c3ccccc3)cc2F)nc1-c1cnn(C)c1] | 50 |
| 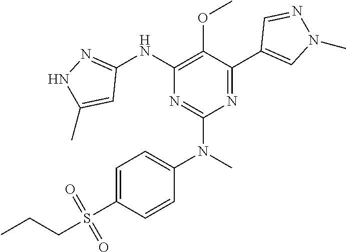 [SMILES: CCCS(=O)(=O)c1ccc(N(C)c2nc(Nc3cc(C)[nH]n3)c(OC)c(-c3cnn(C)c3)n2)cc1] | 51 |
| 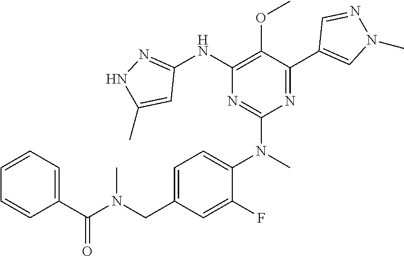 [SMILES: COc1c(Nc2cc(C)[nH]n2)nc(N(C)c2ccc(CN(C)C(=O)c3ccccc3)cc2F)nc1-c1cnn(C)c1] | 52 |
| 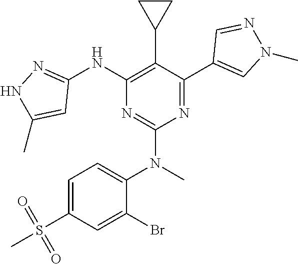 | 53 |
| 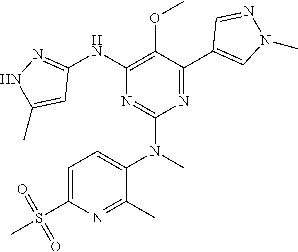 | 54 |
| 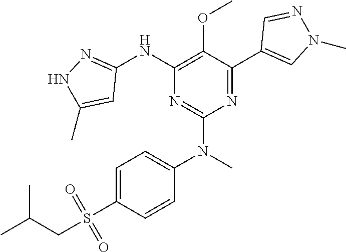 [SMILES: COc1c(Nc2cc(C)[nH]n2)nc(N(C)c2ccc(S(=O)(=O)CC(C)C)cc2)nc1-c1cnn(C)c1] | 55 |
| 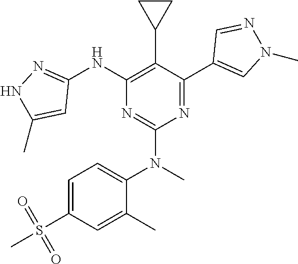 | 56 |
| 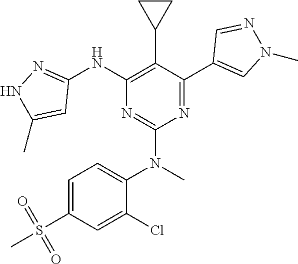 | 57 |
| 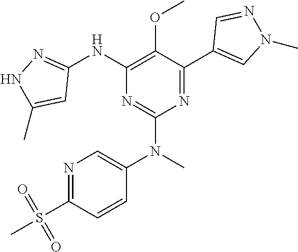 | 58 |
| 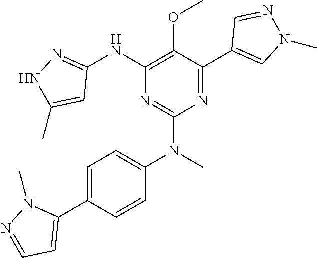 [SMILES: COc1c(Nc2cc(C)[nH]n2)nc(N(C)c2ccc(-c3ccnn3C)cc2)nc1-c1cnn(C)c1] | 59 |
| 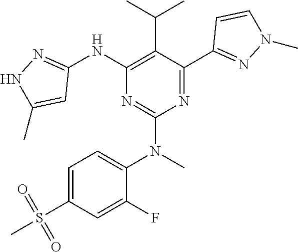 | 60 |
| 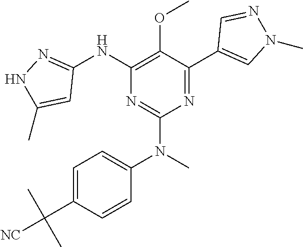 [SMILES: COc1c(Nc2cc(C)[nH]n2)nc(N(C)c2ccc(C(C)(C)C#N)cc2)nc1-c1cnn(C)c1] | 61 |
| 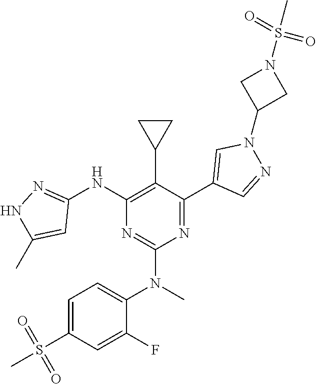 [SMILES: Cc1cc(Nc2nc(N(C)c3ccc(S(C)(=O)=O)cc3F)nc(-c3cnn(C4CN(S(C)(=O)=O)C4)c3)c2C2CC2)n[nH]1] | 62 |
| 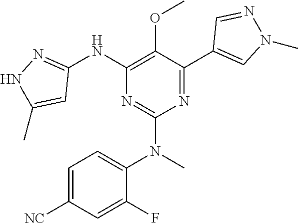 [SMILES: COc1c(Nc2cc(C)[nH]n2)nc(N(C)c2ccc(C#N)cc2F)nc1-c1cnn(C)c1] | 63 |
| 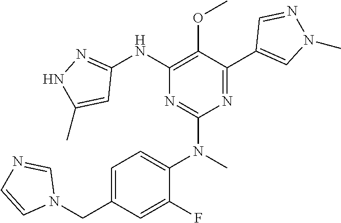 [SMILES: COc1c(Nc2cc(C)[nH]n2)nc(N(C)c2ccc(Cn3ccnc3)cc2F)nc1-c1cnn(C)c1] | 64 |
| 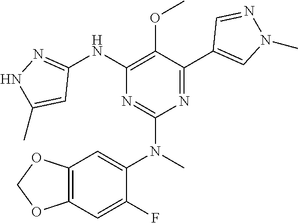 [SMILES: COc1c(Nc2cc(C)[nH]n2)nc(N(C)c2cc3c(cc2F)OCO3)nc1-c1cnn(C)c1] | 65 |
| 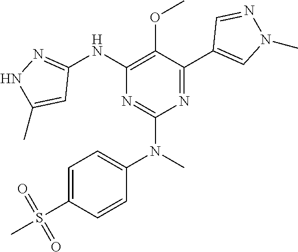 | 66 |
| 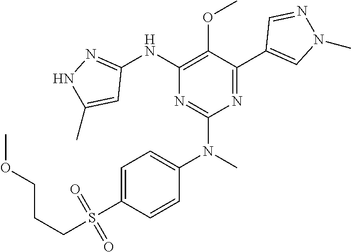 [SMILES: COCCCS(=O)(=O)c1ccc(N(C)c2nc(Nc3cc(C)[nH]n3)c(OC)c(-c3cnn(C)c3)n2)cc1] | 67 |
| 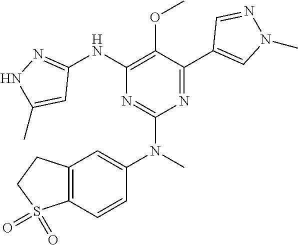 [SMILES: COc1c(Nc2cc(C)[nH]n2)nc(N(C)c2ccc3c(c2)CCS3(=O)=O)nc1-c1cnn(C)c1] | 68 |
| 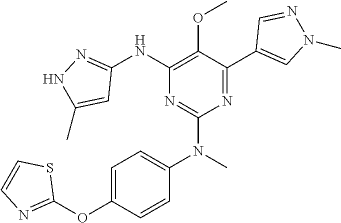 [SMILES: COc1c(Nc2cc(C)[nH]n2)nc(N(C)c2ccc(Oc3nccs3)cc2)nc1-c1cnn(C)c1] | 69 |
| 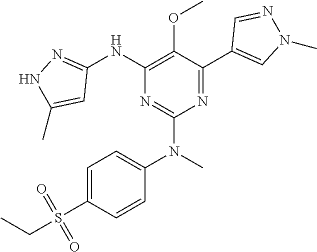 [SMILES: CCS(=O)(=O)c1ccc(N(C)c2nc(Nc3cc(C)[nH]n3)c(OC)c(-c3cnn(C)c3)n2)cc1] | 70 |
| 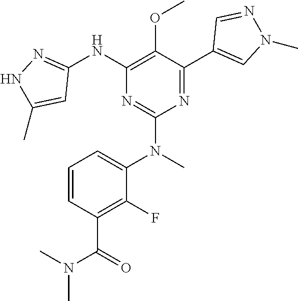 [SMILES: COc1c(Nc2cc(C)[nH]n2)nc(N(C)c2cccc(C(=O)N(C)C)c2F)nc1-c1cnn(C)c1] | 71 |
| 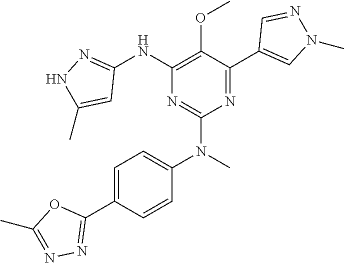 | 72 |
| 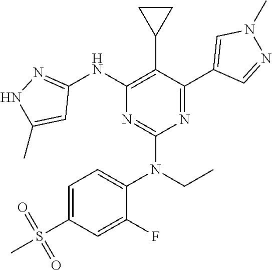 | 73 |
| 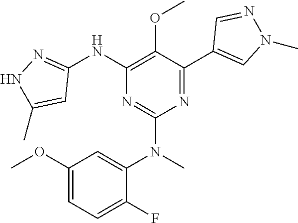 | 74 |
| 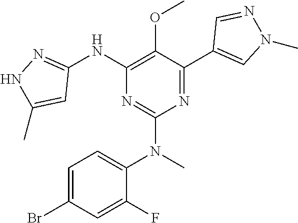 [SMILES: COc1c(Nc2cc(C)[nH]n2)nc(N(C)c2ccc(Br)cc2F)nc1-c1cnn(C)c1] | 75 |
| 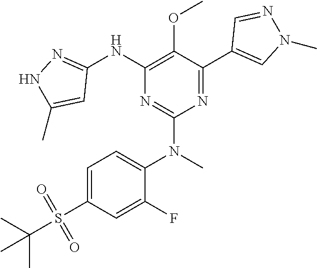 [SMILES: COc1c(Nc2cc(C)[nH]n2)nc(N(C)c2ccc(S(=O)(=O)C(C)(C)C)cc2F)nc1-c1cnn(C)c1] | 76 |
| 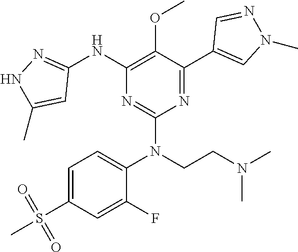 | 77 |
| 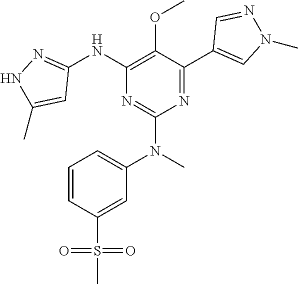 [SMILES: COc1c(Nc2cc(C)[nH]n2)nc(N(C)c2cccc(S(C)(=O)=O)c2)nc1-c1cnn(C)c1] | 78 |
| 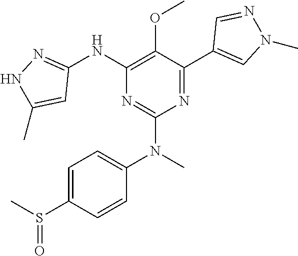 | 79 |
| 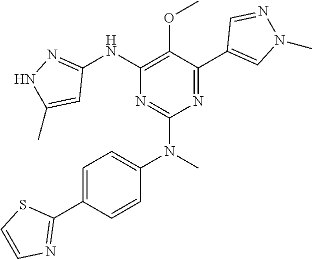 [SMILES: COc1c(Nc2cc(C)[nH]n2)nc(N(C)c2ccc(-c3nccs3)cc2)nc1-c1cnn(C)c1] | 80 |
| 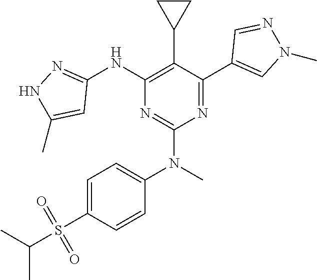 [SMILES: Cc1cc(Nc2nc(N(C)c3ccc(S(=O)(=O)C(C)C)cc3)nc(-c3cnn(C)c3)c2C2CC2)n[nH]1] | 81 |
| 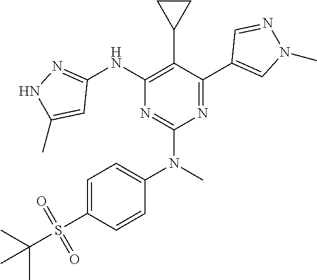 [SMILES: Cc1cc(Nc2nc(N(C)c3ccc(S(=O)(=O)C(C)(C)C)cc3)nc(-c3cnn(C)c3)c2C2CC2)n[nH]1] | 82 |
| 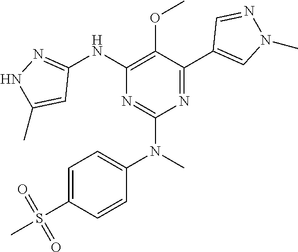 | 83 |
| 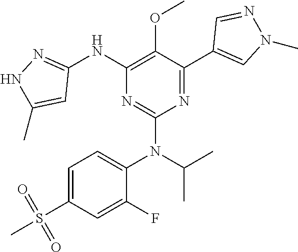 | 84 |
| 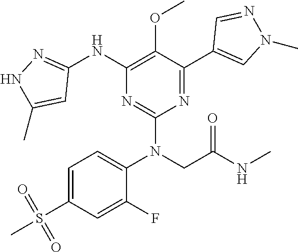 | 85 |
| 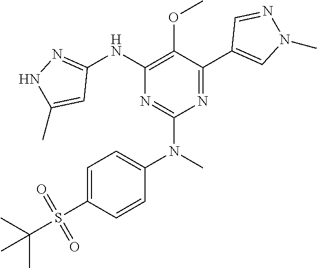 [SMILES: COc1c(Nc2cc(C)[nH]n2)nc(N(C)c2ccc(S(=O)(=O)C(C)(C)C)cc2)nc1-c1cnn(C)c1] | 86 |
| 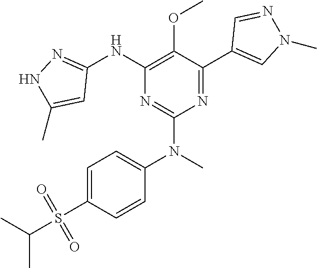 [SMILES: COc1c(Nc2cc(C)[nH]n2)nc(N(C)c2ccc(S(=O)(=O)C(C)C)cc2)nc1-c1cnn(C)c1] | 87 |
| 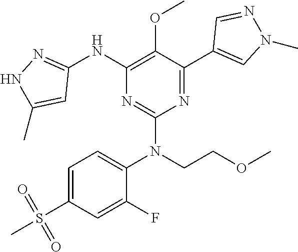 | 88 |
| 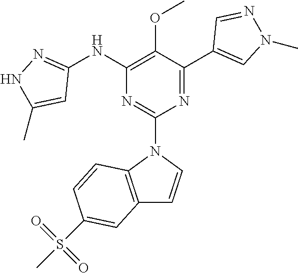 [SMILES: COc1c(Nc2cc(C)[nH]n2)nc(-n2ccc3cc(S(C)(=O)=O)ccc32)nc1-c1cnn(C)c1] | 89 |
| 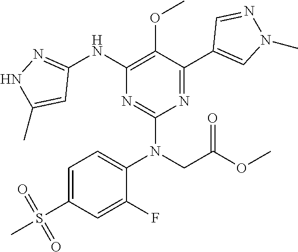 | 90 |
| 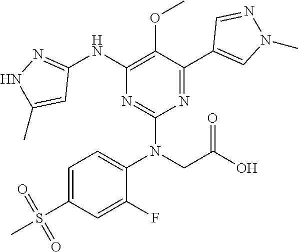 | 91 |
| 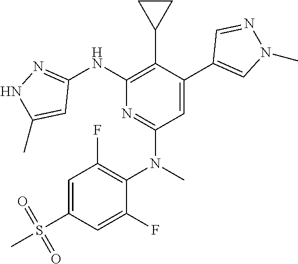 | 92 |
| 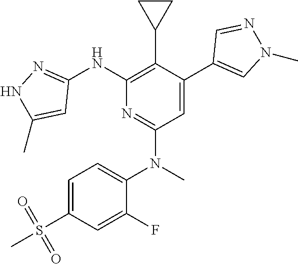 | 93 |
| 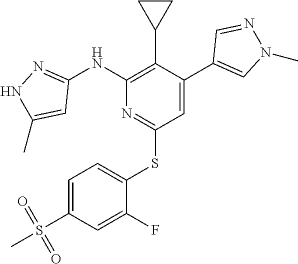 | 94 |
| 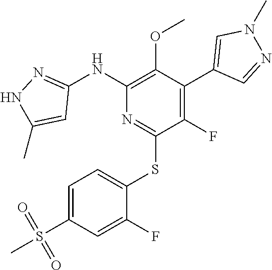 | 95 |
| 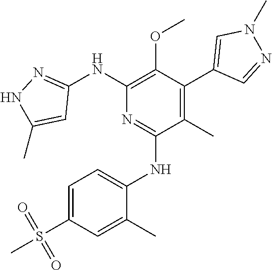 | 96 |
| 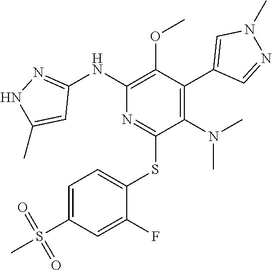 | 97 |
| 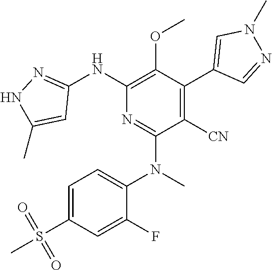 | 98 |
| 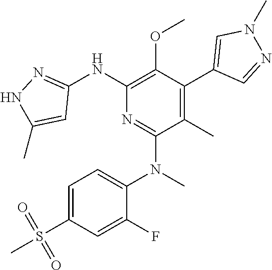 | 99 |
| 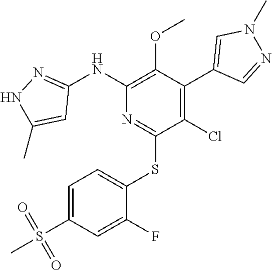 | 100 |
| 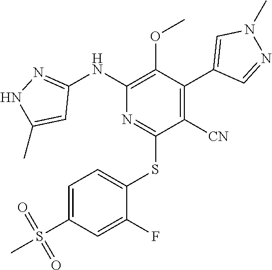 | 101 |
| 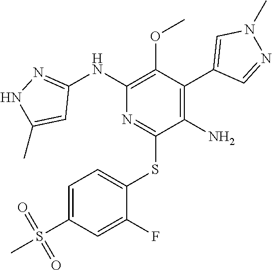 | 102 |
| 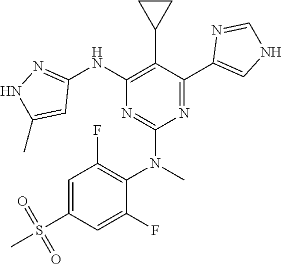 [SMILES: Cc1cc(Nc2nc(N(C)c3c(F)cc(S(C)(=O)=O)cc3F)nc(-c3c[nH]cn3)c2C2CC2)n[nH]1] | 103 |
| 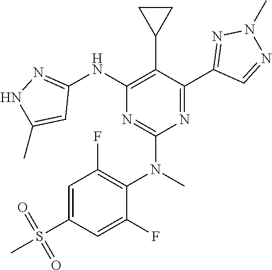 | 104 |
| 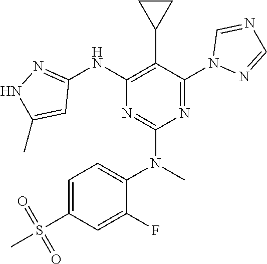 [SMILES: Cc1cc(Nc2nc(N(C)c3ccc(S(C)(=O)=O)cc3F)nc(-n3cncn3)c2C2CC2)n[nH]1] | 105 |
| 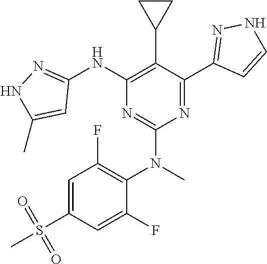 [SMILES: Cc1cc(Nc2nc(N(C)c3c(F)cc(S(C)(=O)=O)cc3F)nc(-c3cc[nH]n3)c2C2CC2)n[nH]1] | 106 |
| 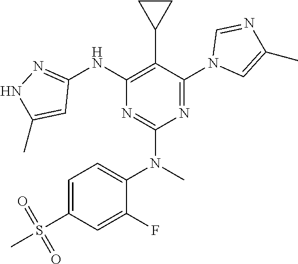 | 107 |
| 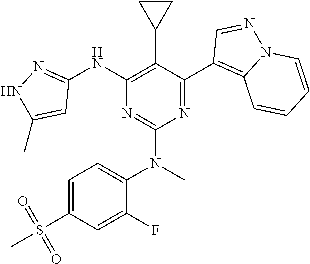 [SMILES: Cc1cc(Nc2nc(N(C)c3ccc(S(C)(=O)=O)cc3F)nc(-c3cnn4ccccc34)c2C2CC2)n[nH]1] | 108 |
| 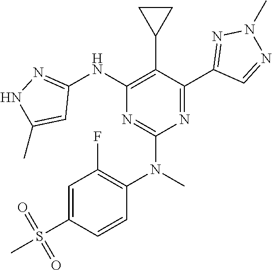 | 109 |
| 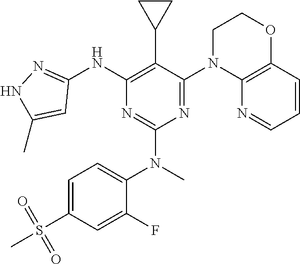 [SMILES: Cc1cc(Nc2nc(N(C)c3ccc(S(C)(=O)=O)cc3F)nc(N3CCOc4cccnc43)c2C2CC2)n[nH]1] | 110 |
| 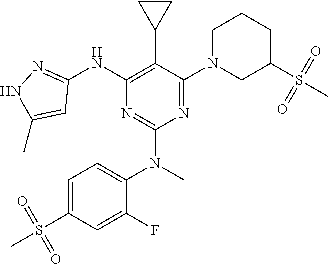 | 111 |
| 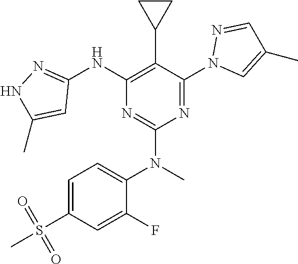 | 112 |
| 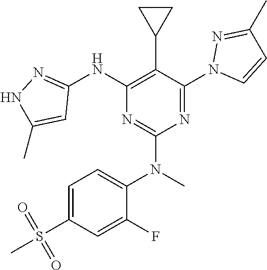 | 113 |
| 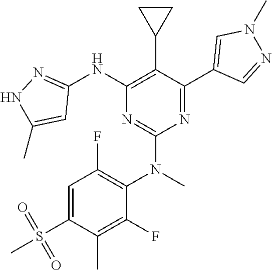 [SMILES: Cc1cc(Nc2nc(N(C)c3c(F)cc(S(C)(=O)=O)c(C)c3F)nc(-c3cnn(C)c3)c2C2CC2)n[nH]1] | 114 |
| 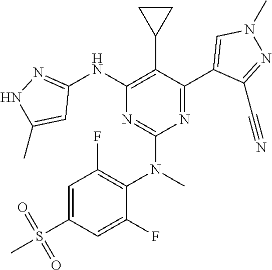 [SMILES: Cc1cc(Nc2nc(N(C)c3c(F)cc(S(C)(=O)=O)cc3F)nc(-c3cn(C)nc3C#N)c2C2CC2)n[nH]1] | 115 |
| 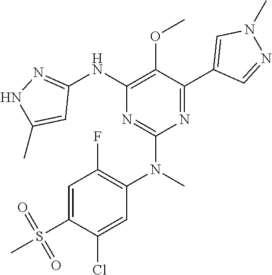 [SMILES: COc1c(Nc2cc(C)[nH]n2)nc(N(C)c2cc(Cl)c(S(C)(=O)=O)cc2F)nc1-c1cnn(C)c1] | 116 |
| 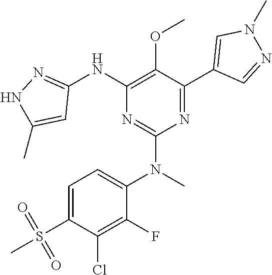 [SMILES: COc1c(Nc2cc(C)[nH]n2)nc(N(C)c2ccc(S(C)(=O)=O)c(Cl)c2F)nc1-c1cnn(C)c1] | 117 |
| 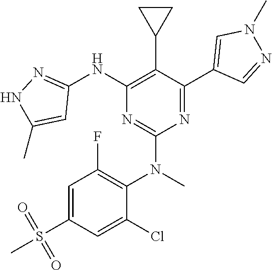 [SMILES: Cc1cc(Nc2nc(N(C)c3c(F)cc(S(C)(=O)=O)cc3Cl)nc(-c3cnn(C)c3)c2C2CC2)n[nH]1] | 118 |
| 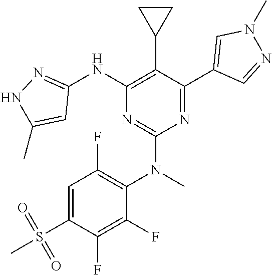 [SMILES: Cc1cc(Nc2nc(N(C)c3c(F)cc(S(C)(=O)=O)c(F)c3F)nc(-c3cnn(C)c3)c2C2CC2)n[nH]1] | 119 |
| 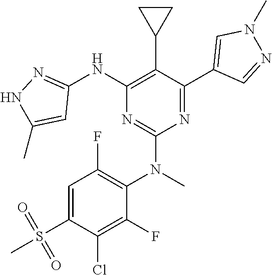 [SMILES: Cc1cc(Nc2nc(N(C)c3c(F)cc(S(C)(=O)=O)c(Cl)c3F)nc(-c3cnn(C)c3)c2C2CC2)n[nH]1] | 120 |
| 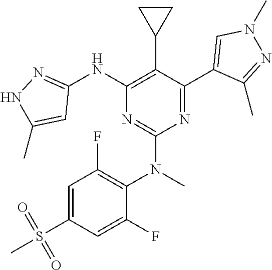 [SMILES: Cc1cc(Nc2nc(N(C)c3c(F)cc(S(C)(=O)=O)cc3F)nc(-c3cn(C)nc3C)c2C2CC2)n[nH]1] | 121 |
| 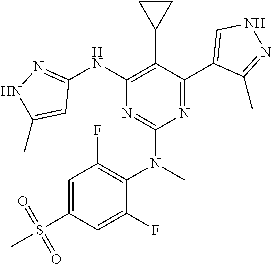 [SMILES: Cc1cc(Nc2nc(N(C)c3c(F)cc(S(C)(=O)=O)cc3F)nc(-c3c[nH]nc3C)c2C2CC2)n[nH]1] | 122 |
| 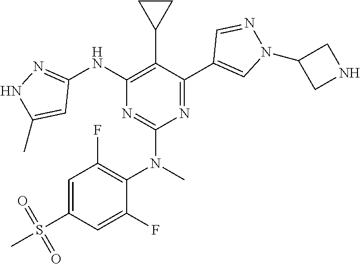 [SMILES: Cc1cc(Nc2nc(N(C)c3c(F)cc(S(C)(=O)=O)cc3F)nc(-c3cnn(C4CNC4)c3)c2C2CC2)n[nH]1] | 123 |
| 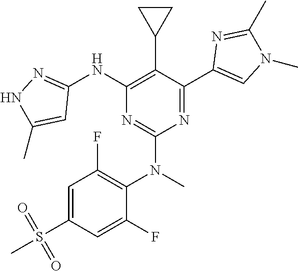 | 124 |
| 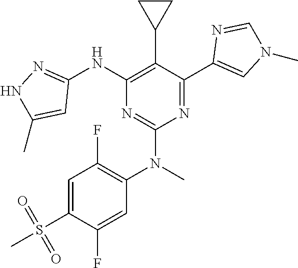 | 125 |
| 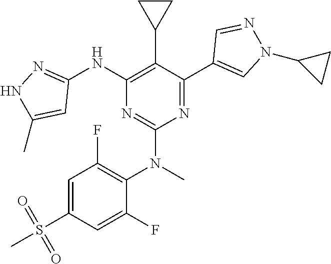 [SMILES: Cc1cc(Nc2nc(N(C)c3c(F)cc(S(C)(=O)=O)cc3F)nc(-c3cnn(C4CC4)c3)c2C2CC2)n[nH]1] | 126 |
| 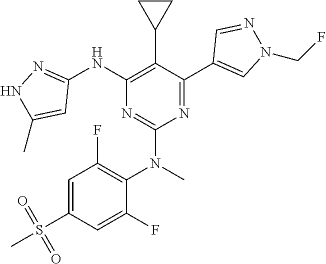 [SMILES: Cc1cc(Nc2nc(N(C)c3c(F)cc(S(C)(=O)=O)cc3F)nc(-c3cnn(CF)c3)c2C2CC2)n[nH]1] | 127 |
| 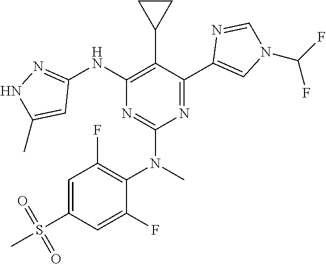 [SMILES: Cc1cc(Nc2nc(N(C)c3c(F)cc(S(C)(=O)=O)cc3F)nc(-c3cn(C(F)F)cn3)c2C2CC2)n[nH]1] | 128 |
| 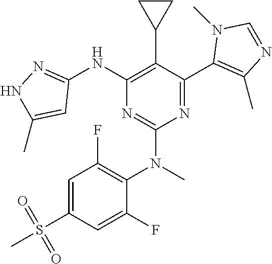 [SMILES: Cc1cc(Nc2nc(N(C)c3c(F)cc(S(C)(=O)=O)cc3F)nc(-c3c(C)ncn3C)c2C2CC2)n[nH]1] | 129 |
| 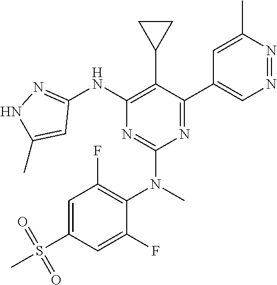 [SMILES: Cc1cc(-c2nc(N(C)c3c(F)cc(S(C)(=O)=O)cc3F)nc(Nc3cc(C)[nH]n3)c2C2CC2)cnn1] | 130 |
| 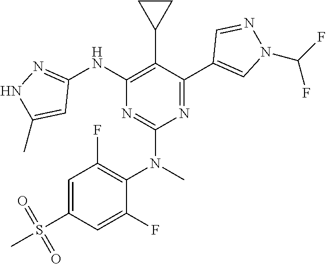 [SMILES: Cc1cc(Nc2nc(N(C)c3c(F)cc(S(C)(=O)=O)cc3F)nc(-c3cnn(C(F)F)c3)c2C2CC2)n[nH]1] | 131 |
| 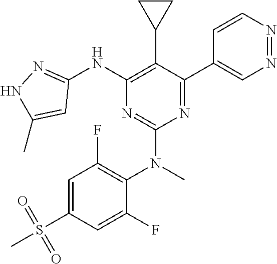 [SMILES: Cc1cc(Nc2nc(N(C)c3c(F)cc(S(C)(=O)=O)cc3F)nc(-c3ccnnc3)c2C2CC2)n[nH]1] | 132 |
| 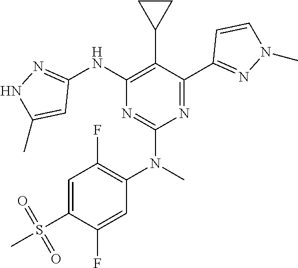 | 133 |
| 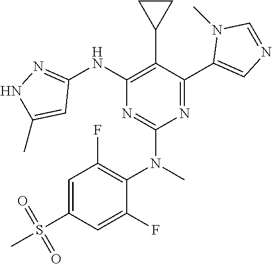 [SMILES: Cc1cc(Nc2nc(N(C)c3c(F)cc(S(C)(=O)=O)cc3F)nc(-c3cncn3C)c2C2CC2)n[nH]1] | 134 |
| 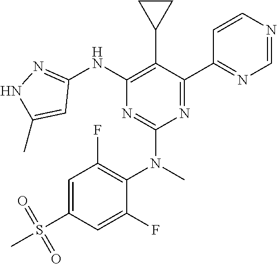 [SMILES: Cc1cc(Nc2nc(N(C)c3c(F)cc(S(C)(=O)=O)cc3F)nc(-c3ccncn3)c2C2CC2)n[nH]1] | 135 |
| 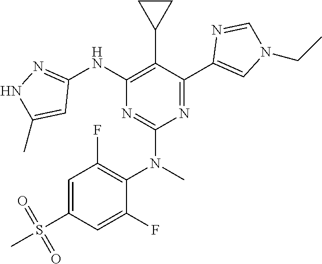 [SMILES: CCn1cnc(-c2nc(N(C)c3c(F)cc(S(C)(=O)=O)cc3F)nc(Nc3cc(C)[nH]n3)c2C2CC2)c1] | 136 |
| 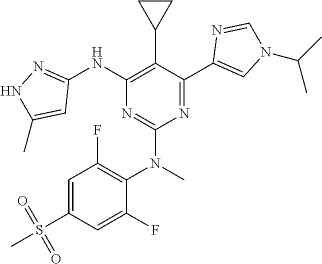 [SMILES: Cc1cc(Nc2nc(N(C)c3c(F)cc(S(C)(=O)=O)cc3F)nc(-c3cn(C(C)C)cn3)c2C2CC2)n[nH]1] | 137 |
| 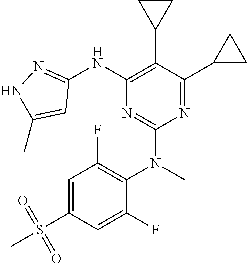 [SMILES: Cc1cc(Nc2nc(N(C)c3c(F)cc(S(C)(=O)=O)cc3F)nc(C3CC3)c2C2CC2)n[nH]1] | 138 |
| 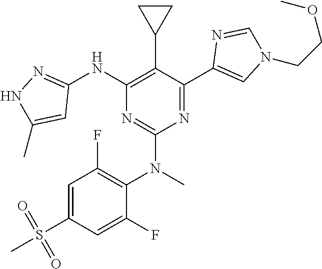 [SMILES: COCCn1cnc(-c2nc(N(C)c3c(F)cc(S(C)(=O)=O)cc3F)nc(Nc3cc(C)[nH]n3)c2C2CC2)c1] | 139 |
| 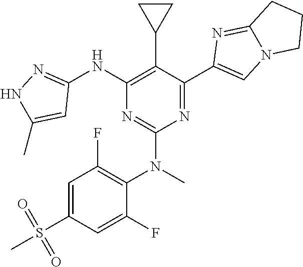 [SMILES: Cc1cc(Nc2nc(N(C)c3c(F)cc(S(C)(=O)=O)cc3F)nc(-c3cn4c(n3)CCC4)c2C2CC2)n[nH]1] | 140 |
| 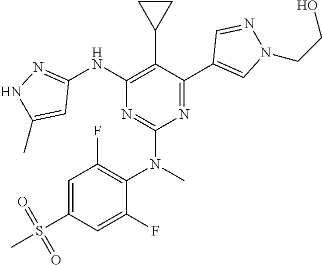 [SMILES: Cc1cc(Nc2nc(N(C)c3c(F)cc(S(C)(=O)=O)cc3F)nc(-c3cnn(CCO)c3)c2C2CC2)n[nH]1] | 141 |
| 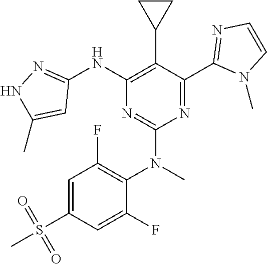 [SMILES: Cc1cc(Nc2nc(N(C)c3c(F)cc(S(C)(=O)=O)cc3F)nc(-c3nccn3C)c2C2CC2)n[nH]1] | 142 |
| 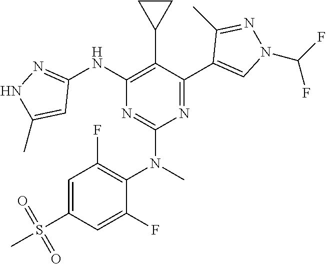 [SMILES: Cc1cc(Nc2nc(N(C)c3c(F)cc(S(C)(=O)=O)cc3F)nc(-c3cn(C(F)F)nc3C)c2C2CC2)n[nH]1] | 143 |
| 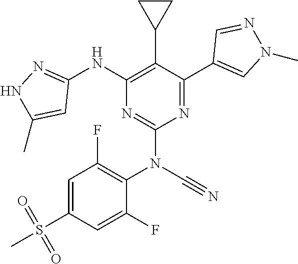 | 144 |
| 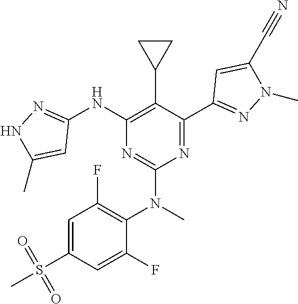 | 145 |
| 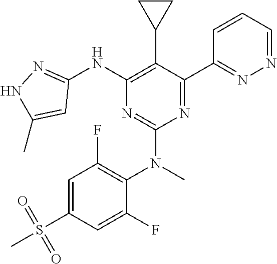 [SMILES: Cc1cc(Nc2nc(N(C)c3c(F)cc(S(C)(=O)=O)cc3F)nc(-c3cccnn3)c2C2CC2)n[nH]1] | 146 |
| 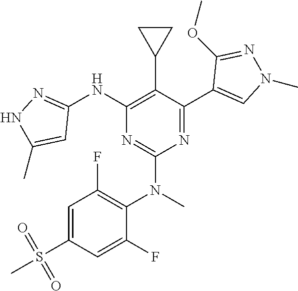 | 147 |
| 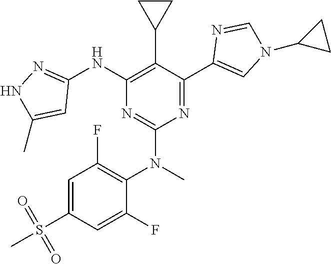 [SMILES: Cc1cc(Nc2nc(N(C)c3c(F)cc(S(C)(=O)=O)cc3F)nc(-c3cn(C4CC4)cn3)c2C2CC2)n[nH]1] | 148 |
| 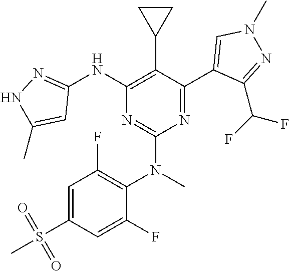 [SMILES: Cc1cc(Nc2nc(N(C)c3c(F)cc(S(C)(=O)=O)cc3F)nc(-c3cn(C)nc3C(F)F)c2C2CC2)n[nH]1] | 149 |
| 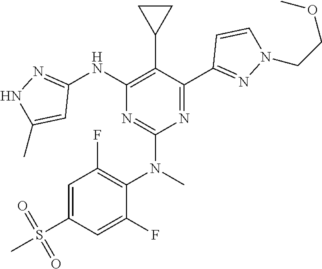 [SMILES: COCCn1ccc(-c2nc(N(C)c3c(F)cc(S(C)(=O)=O)cc3F)nc(Nc3cc(C)[nH]n3)c2C2CC2)n1] | 150 |
| 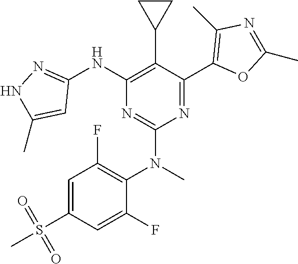 | 151 |
| 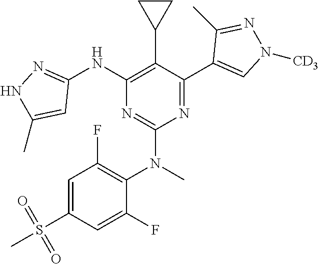 [SMILES: [2H]C([2H])([2H])n1cc(-c2nc(N(C)c3c(F)cc(S(C)(=O)=O)cc3F)nc(Nc3cc(C)[nH]n3)c2C2CC2)c(C)n1] | 152 |
| 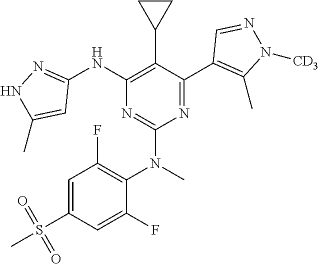 [SMILES: [2H]C([2H])([2H])n1ncc(-c2nc(N(C)c3c(F)cc(S(C)(=O)=O)cc3F)nc(Nc3cc(C)[nH]n3)c2C2CC2)c1C] | 153 |
| 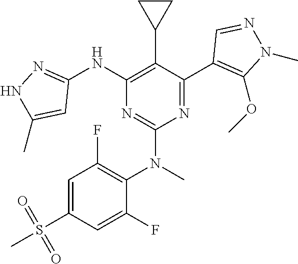 | 154 |
| 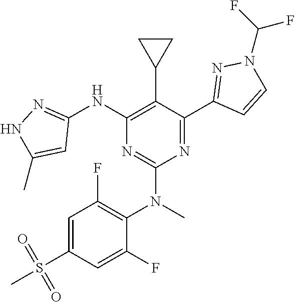 [SMILES: Cc1cc(Nc2nc(N(C)c3c(F)cc(S(C)(=O)=O)cc3F)nc(-c3ccn(C(F)F)n3)c2C2CC2)n[nH]1] | 155 |
| 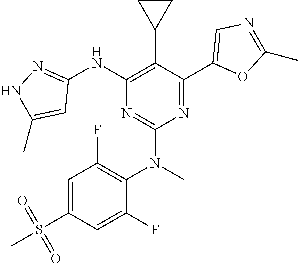 | 156 |
| 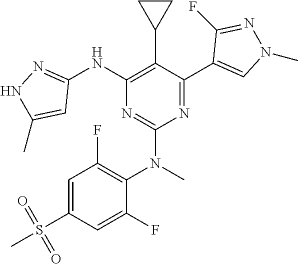 | 157 |
| 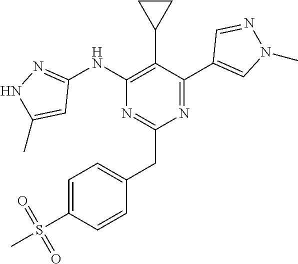 | 158 |
| 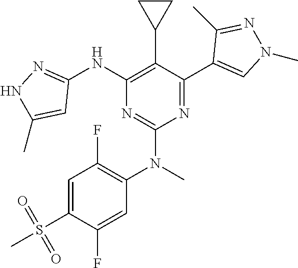 | 159 |
| 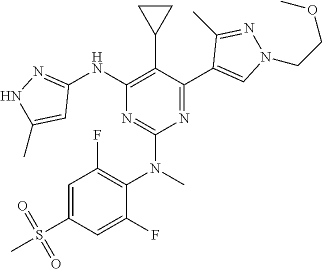 [SMILES: COCCn1cc(-c2nc(N(C)c3c(F)cc(S(C)(=O)=O)cc3F)nc(Nc3cc(C)[nH]n3)c2C2CC2)c(C)n1] | 160 |
| 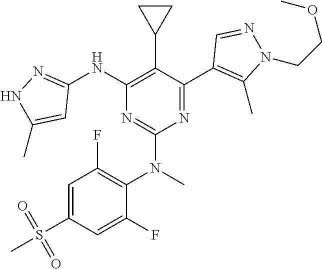 [SMILES: COCCn1ncc(-c2nc(N(C)c3c(F)cc(S(C)(=O)=O)cc3F)nc(Nc3cc(C)[nH]n3)c2C2CC2)c1C] | 161 |
| 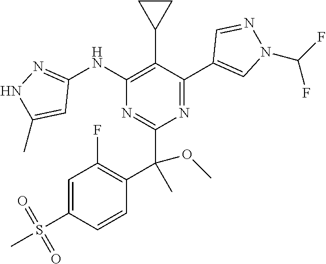 [SMILES: COC(C)(c1nc(Nc2cc(C)[nH]n2)c(C2CC2)c(-c2cnn(C(F)F)c2)n1)c1ccc(S(C)(=O)=O)cc1F] | 162 |
| 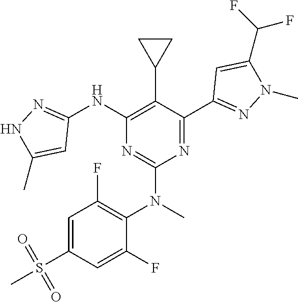 | 163 |
| 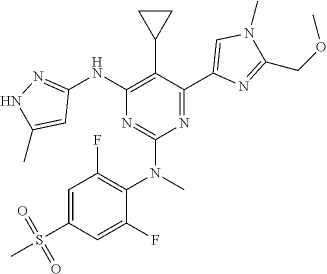 [SMILES: COCc1nc(-c2nc(N(C)c3c(F)cc(S(C)(=O)=O)cc3F)nc(Nc3cc(C)[nH]n3)c2C2CC2)cn1C] | 164 |
| 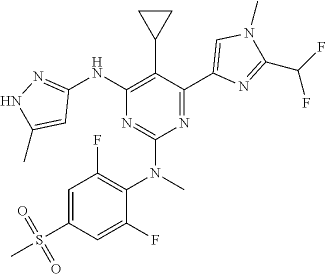 [SMILES: Cc1cc(Nc2nc(N(C)c3c(F)cc(S(C)(=O)=O)cc3F)nc(-c3cn(C)c(C(F)F)n3)c2C2CC2)n[nH]1] | 165 |
| 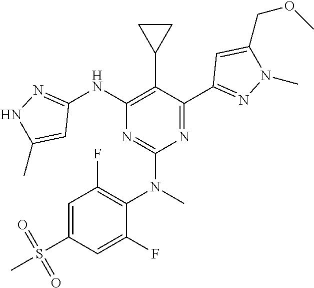 | 166 |
| 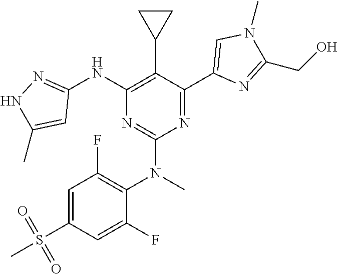 [SMILES: Cc1cc(Nc2nc(N(C)c3c(F)cc(S(C)(=O)=O)cc3F)nc(-c3cn(C)c(CO)n3)c2C2CC2)n[nH]1] | 167 |
| 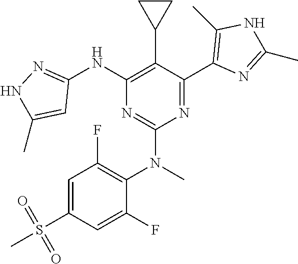 | 168 |
| 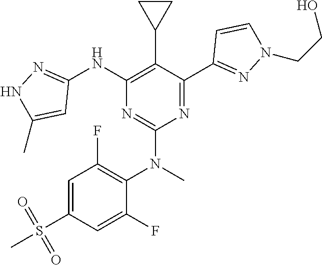 [SMILES: Cc1cc(Nc2nc(N(C)c3c(F)cc(S(C)(=O)=O)cc3F)nc(-c3ccn(CCO)n3)c2C2CC2)n[nH]1] | 169 |
| 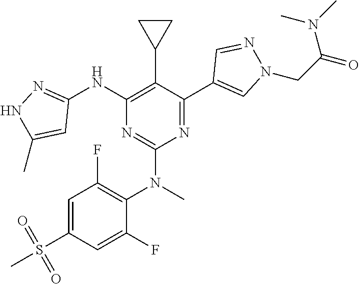 [SMILES: Cc1cc(Nc2nc(N(C)c3c(F)cc(S(C)(=O)=O)cc3F)nc(-c3cnn(CC(=O)N(C)C)c3)c2C2CC2)n[nH]1] | 170 |
| 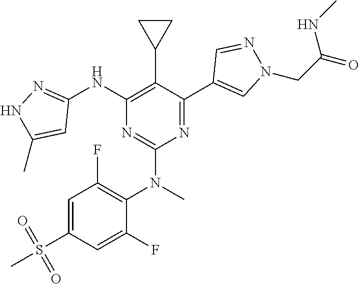 [SMILES: CNC(=O)Cn1cc(-c2nc(N(C)c3c(F)cc(S(C)(=O)=O)cc3F)nc(Nc3cc(C)[nH]n3)c2C2CC2)cn1] | 171 |
| 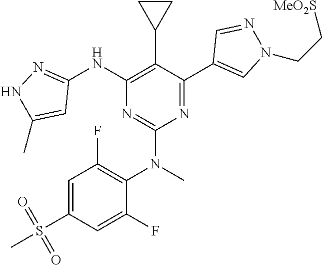 [SMILES: Cc1cc(Nc2nc(N(C)c3c(F)cc(S(C)(=O)=O)cc3F)nc(-c3cnn(CCS(C)(=O)=O)c3)c2C2CC2)n[nH]1] | 172 |
| 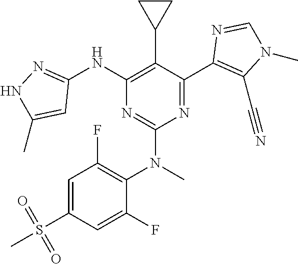 | 173 |
| 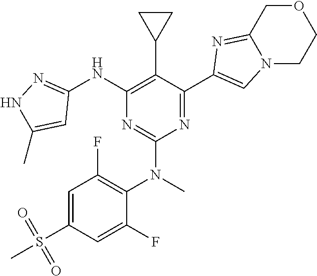 [SMILES: Cc1cc(Nc2nc(N(C)c3c(F)cc(S(C)(=O)=O)cc3F)nc(-c3cn4c(n3)COCC4)c2C2CC2)n[nH]1] | 174 |
| 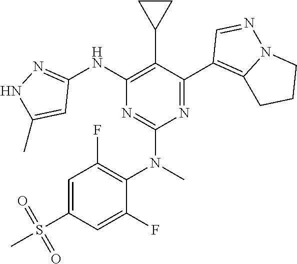 [SMILES: Cc1cc(Nc2nc(N(C)c3c(F)cc(S(C)(=O)=O)cc3F)nc(-c3cnn4c3CCC4)c2C2CC2)n[nH]1] | 175 |
| 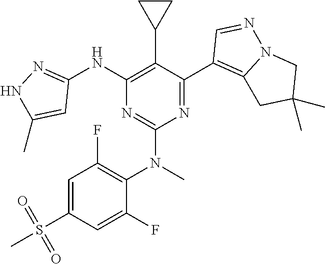 | 176 |
| 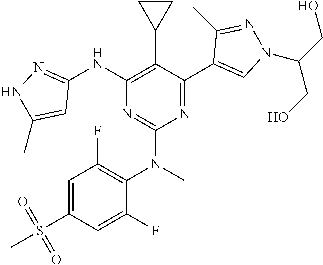 [SMILES: Cc1cc(Nc2nc(N(C)c3c(F)cc(S(C)(=O)=O)cc3F)nc(-c3cn(C(CO)CO)nc3C)c2C2CC2)n[nH]1] | 177 |
| 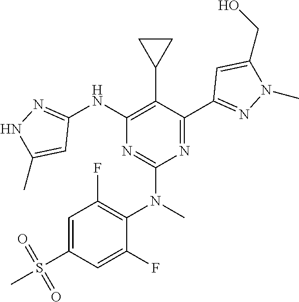 | 178 |
| 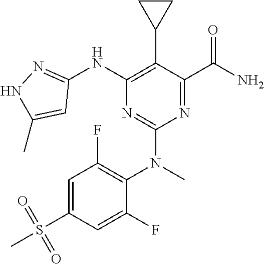 [SMILES: Cc1cc(Nc2nc(N(C)c3c(F)cc(S(C)(=O)=O)cc3F)nc(C(N)=O)c2C2CC2)n[nH]1] | 179 |
| 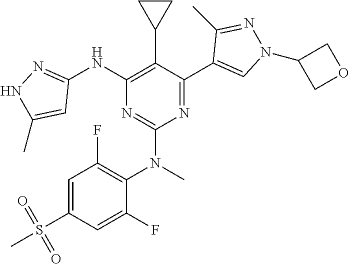 [SMILES: Cc1cc(Nc2nc(N(C)c3c(F)cc(S(C)(=O)=O)cc3F)nc(-c3cn(C4COC4)nc3C)c2C2CC2)n[nH]1] | 180 |
| 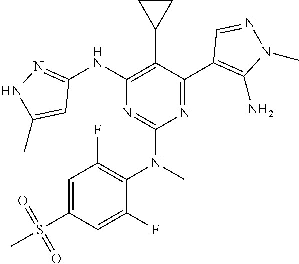 | 181 |
| 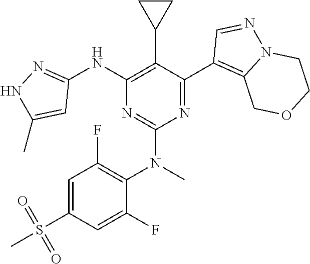 [SMILES: Cc1cc(Nc2nc(N(C)c3c(F)cc(S(C)(=O)=O)cc3F)nc(-c3cnn4c3COCC4)c2C2CC2)n[nH]1] | 182 |
| 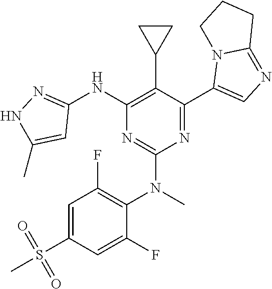 [SMILES: Cc1cc(Nc2nc(N(C)c3c(F)cc(S(C)(=O)=O)cc3F)nc(-c3cnc4n3CCC4)c2C2CC2)n[nH]1] | 183 |
| 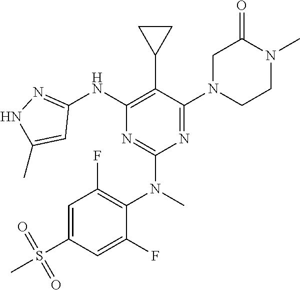 | 184 |
| 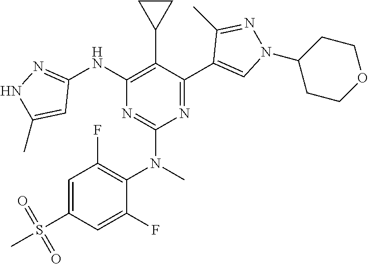 [SMILES: Cc1cc(Nc2nc(N(C)c3c(F)cc(S(C)(=O)=O)cc3F)nc(-c3cn(C4CCOCC4)nc3C)c2C2CC2)n[nH]1] | 185 |
| 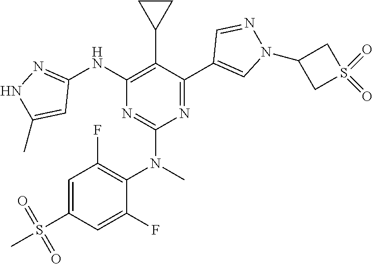 [SMILES: Cc1cc(Nc2nc(N(C)c3c(F)cc(S(C)(=O)=O)cc3F)nc(-c3cnn(C4CS(=O)(=O)C4)c3)c2C2CC2)n[nH]1] | 186 |
| 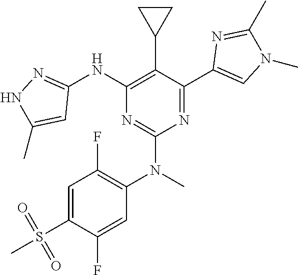 | 187 |
| 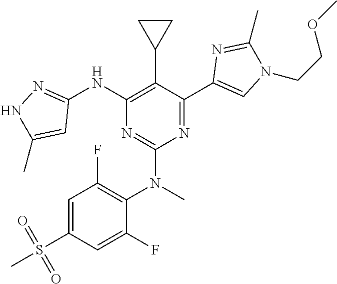 | 188 |
| 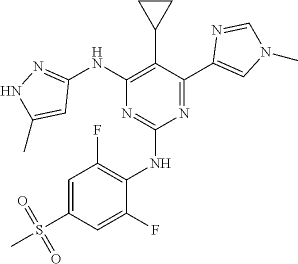 | 189 |
| 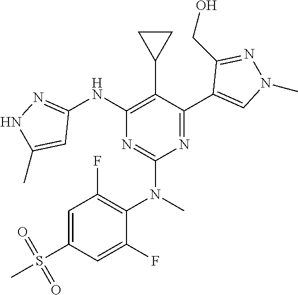 | 190 |
| 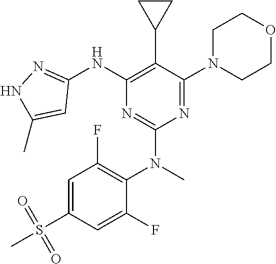 [SMILES: Cc1cc(Nc2nc(N(C)c3c(F)cc(S(C)(=O)=O)cc3F)nc(N3CCOCC3)c2C2CC2)n[nH]1] | 191 |
| 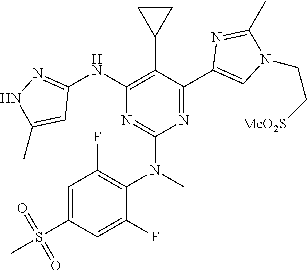 [SMILES: Cc1cc(Nc2nc(N(C)c3c(F)cc(S(C)(=O)=O)cc3F)nc(-c3cn(CCS(C)(=O)=O)c(C)n3)c2C2CC2)n[nH]1] | 192 |
| 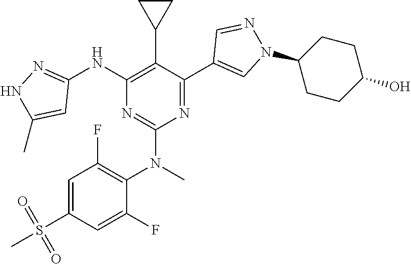 [SMILES: Cc1cc(Nc2nc(N(C)c3c(F)cc(S(C)(=O)=O)cc3F)nc(-c3cnn([C@H]4CC[C@H](O)CC4)c3)c2C2CC2)n[nH]1] | 193 |
| 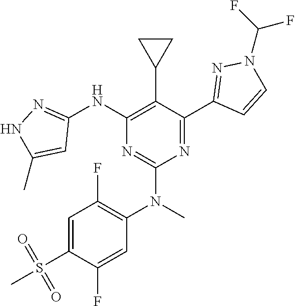 [SMILES: Cc1cc(Nc2nc(N(C)c3cc(F)c(S(C)(=O)=O)cc3F)nc(-c3ccn(C(F)F)n3)c2C2CC2)n[nH]1] | 194 |
| 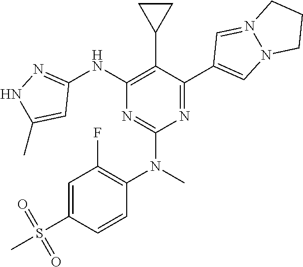 | 195 |
| 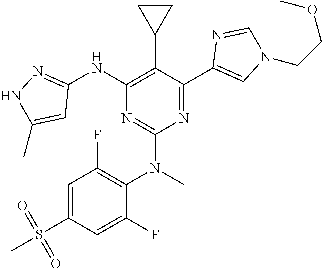 [SMILES: COCCn1cnc(-c2nc(N(C)c3c(F)cc(S(C)(=O)=O)cc3F)nc(Nc3cc(C)[nH]n3)c2C2CC2)c1] | 196 |
| 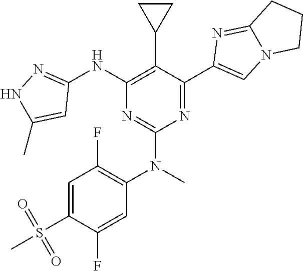 [SMILES: Cc1cc(Nc2nc(N(C)c3cc(F)c(S(C)(=O)=O)cc3F)nc(-c3cn4c(n3)CCC4)c2C2CC2)n[nH]1] | 197 |
| 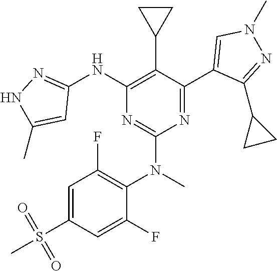 [SMILES: Cc1cc(Nc2nc(N(C)c3c(F)cc(S(C)(=O)=O)cc3F)nc(-c3cn(C)nc3C3CC3)c2C2CC2)n[nH]1] | 198 |
| 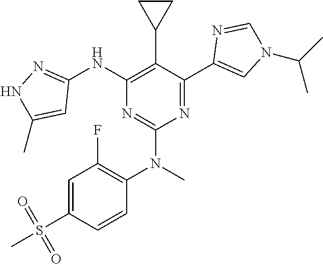 [SMILES: Cc1cc(Nc2nc(N(C)c3ccc(S(C)(=O)=O)cc3F)nc(-c3cn(C(C)C)cn3)c2C2CC2)n[nH]1] | 199 |
| 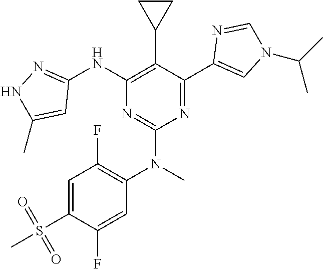 [SMILES: Cc1cc(Nc2nc(N(C)c3cc(F)c(S(C)(=O)=O)cc3F)nc(-c3cn(C(C)C)cn3)c2C2CC2)n[nH]1] | 200 |
| 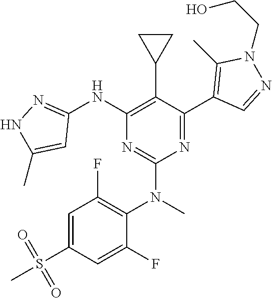 [SMILES: Cc1cc(Nc2nc(N(C)c3c(F)cc(S(C)(=O)=O)cc3F)nc(-c3cnn(CCO)c3C)c2C2CC2)n[nH]1] | 201 |
| 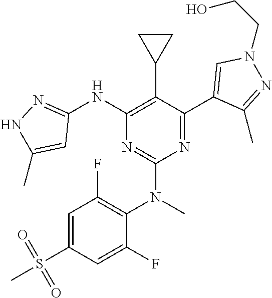 [SMILES: Cc1cc(Nc2nc(N(C)c3c(F)cc(S(C)(=O)=O)cc3F)nc(-c3cn(CCO)nc3C)c2C2CC2)n[nH]1] | 202 |
| 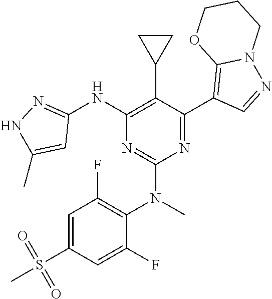 [SMILES: Cc1cc(Nc2nc(N(C)c3c(F)cc(S(C)(=O)=O)cc3F)nc(-c3cnn4c3OCCC4)c2C2CC2)n[nH]1] | 203 |
| 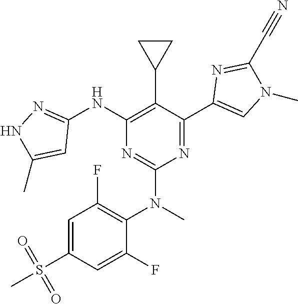 | 204 |
| 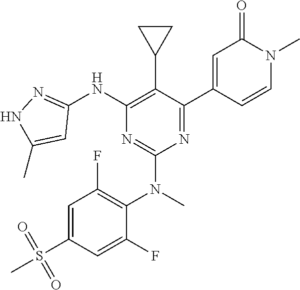 | 205 |
| 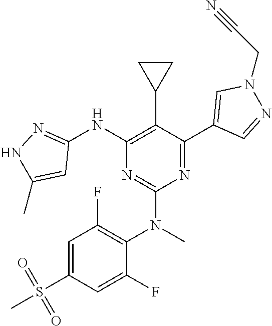 [SMILES: Cc1cc(Nc2nc(N(C)c3c(F)cc(S(C)(=O)=O)cc3F)nc(-c3cnn(CC#N)c3)c2C2CC2)n[nH]1] | 206 |
| 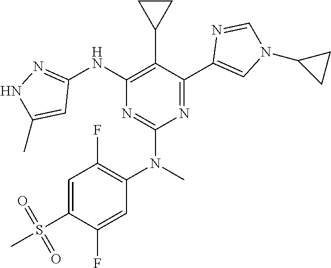 [SMILES: Cc1cc(Nc2nc(N(C)c3cc(F)c(S(C)(=O)=O)cc3F)nc(-c3cn(C4CC4)cn3)c2C2CC2)n[nH]1] | 207 |
| 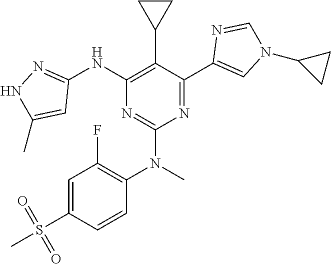 [SMILES: Cc1cc(Nc2nc(N(C)c3ccc(S(C)(=O)=O)cc3F)nc(-c3cn(C4CC4)cn3)c2C2CC2)n[nH]1] | 208 |
| 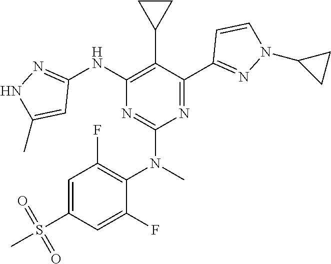 [SMILES: Cc1cc(Nc2nc(N(C)c3c(F)cc(S(C)(=O)=O)cc3F)nc(-c3ccn(C4CC4)n3)c2C2CC2)n[nH]1] | 209 |
| 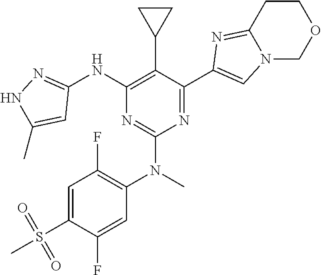 [SMILES: Cc1cc(Nc2nc(N(C)c3cc(F)c(S(C)(=O)=O)cc3F)nc(-c3cn4c(n3)CCOC4)c2C2CC2)n[nH]1] | 210 |
| 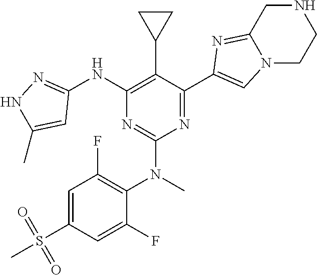 [SMILES: Cc1cc(Nc2nc(N(C)c3c(F)cc(S(C)(=O)=O)cc3F)nc(-c3cn4c(n3)CNCC4)c2C2CC2)n[nH]1] | 211 |
| 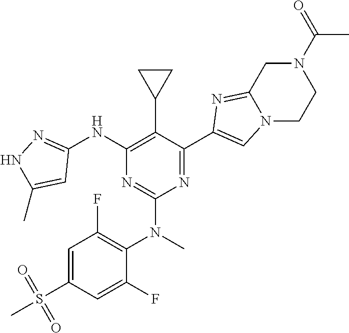 [SMILES: CC(=O)N1CCn2cc(-c3nc(N(C)c4c(F)cc(S(C)(=O)=O)cc4F)nc(Nc4cc(C)[nH]n4)c3C3CC3)nc2C1] | 212 |
| 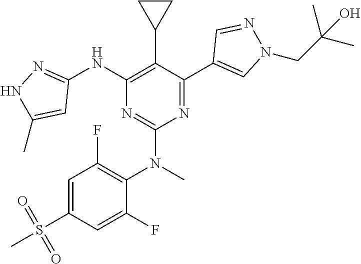 [SMILES: Cc1cc(Nc2nc(N(C)c3c(F)cc(S(C)(=O)=O)cc3F)nc(-c3cnn(CC(C)(C)O)c3)c2C2CC2)n[nH]1] | 213 |
| 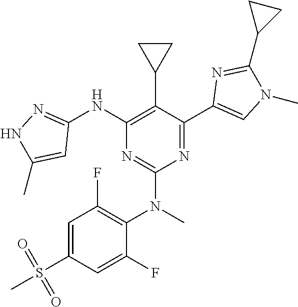 | 214 |
| 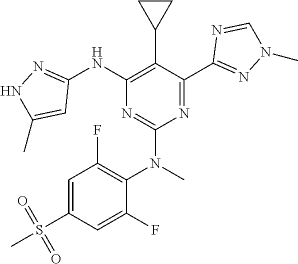 | 215 |
| 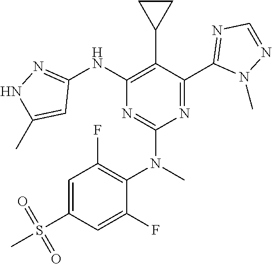 [SMILES: Cc1cc(Nc2nc(N(C)c3c(F)cc(S(C)(=O)=O)cc3F)nc(-c3ncnn3C)c2C2CC2)n[nH]1] | 216 |
| 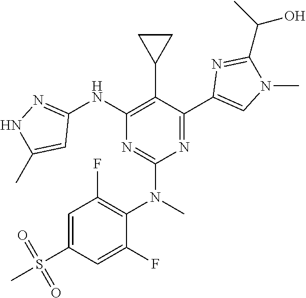 | 217 |
| 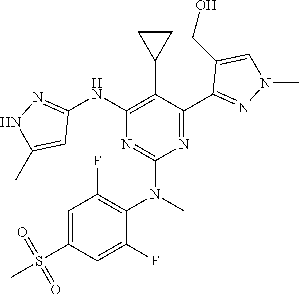 | 218 |
| 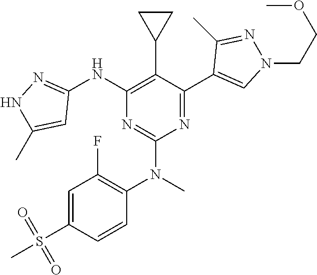 [SMILES: COCCn1cc(-c2nc(N(C)c3ccc(S(C)(=O)=O)cc3F)nc(Nc3cc(C)[nH]n3)c2C2CC2)c(C)n1] | 219 |
| 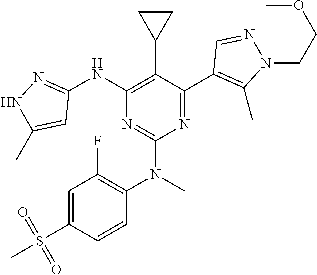 [SMILES: COCCn1ncc(-c2nc(N(C)c3ccc(S(C)(=O)=O)cc3F)nc(Nc3cc(C)[nH]n3)c2C2CC2)c1C] | 220 |
| 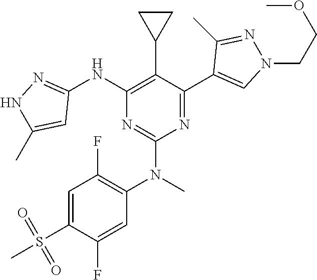 [SMILES: COCCn1cc(-c2nc(N(C)c3cc(F)c(S(C)(=O)=O)cc3F)nc(Nc3cc(C)[nH]n3)c2C2CC2)c(C)n1] | 221 |
| 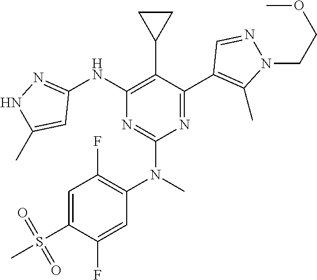 [SMILES: COCCn1ncc(-c2nc(N(C)c3cc(F)c(S(C)(=O)=O)cc3F)nc(Nc3cc(C)[nH]n3)c2C2CC2)c1C] | 222 |
| 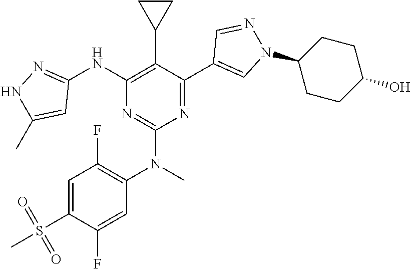 [SMILES: Cc1cc(Nc2nc(N(C)c3cc(F)c(S(C)(=O)=O)cc3F)nc(-c3cnn([C@H]4CC[C@H](O)CC4)c3)c2C2CC2)n[nH]1] | 223 |
| 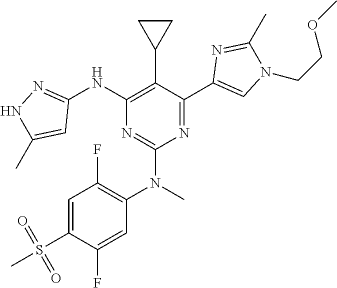 | 224 |
| 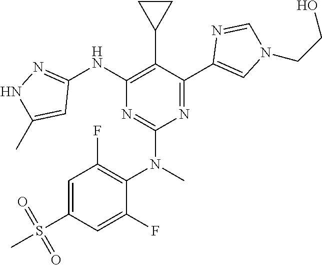 [SMILES: Cc1cc(Nc2nc(N(C)c3c(F)cc(S(C)(=O)=O)cc3F)nc(-c3cn(CCO)cn3)c2C2CC2)n[nH]1] | 225 |
| 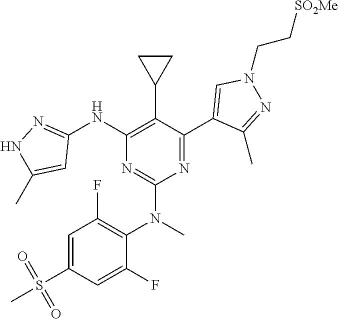 [SMILES: Cc1cc(Nc2nc(N(C)c3c(F)cc(S(C)(=O)=O)cc3F)nc(-c3cn(CCS(C)(=O)=O)nc3C)c2C2CC2)n[nH]1] | 226 |
| 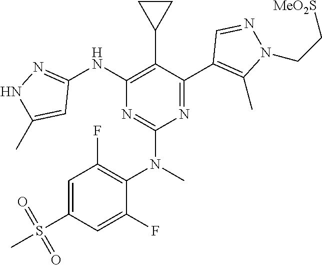 [SMILES: Cc1cc(Nc2nc(N(C)c3c(F)cc(S(C)(=O)=O)cc3F)nc(-c3cnn(CCS(C)(=O)=O)c3C)c2C2CC2)n[nH]1] | 227 |
| 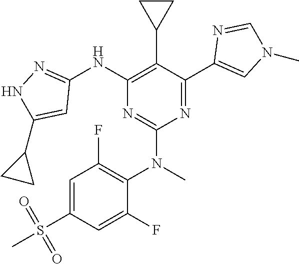 [SMILES: CN(c1nc(Nc2cc(C3CC3)[nH]n2)c(C2CC2)c(-c2cn(C)cn2)n1)c1c(F)cc(S(C)(=O)=O)cc1F] | 228 |
| 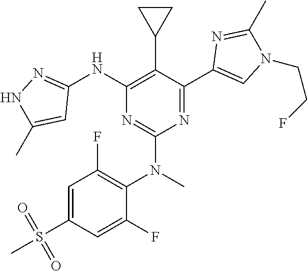 [SMILES: Cc1cc(Nc2nc(N(C)c3c(F)cc(S(C)(=O)=O)cc3F)nc(-c3cn(CCF)c(C)n3)c2C2CC2)n[nH]1] | 229 |
| 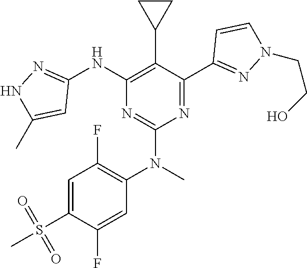 [SMILES: Cc1cc(Nc2nc(N(C)c3cc(F)c(S(C)(=O)=O)cc3F)nc(-c3ccn(CCO)n3)c2C2CC2)n[nH]1] | 230 |
| 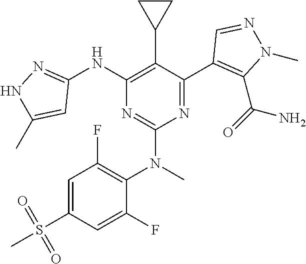 | 231 |
| 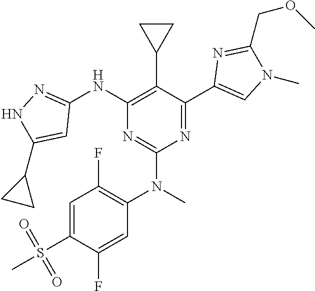 [SMILES: COCc1nc(-c2nc(N(C)c3cc(F)c(S(C)(=O)=O)cc3F)nc(Nc3cc(C4CC4)[nH]n3)c2C2CC2)cn1C] | 232 |
| 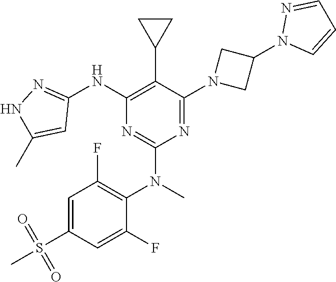 [SMILES: Cc1cc(Nc2nc(N(C)c3c(F)cc(S(C)(=O)=O)cc3F)nc(N3CC(n4cccn4)C3)c2C2CC2)n[nH]1] | 233 |
| 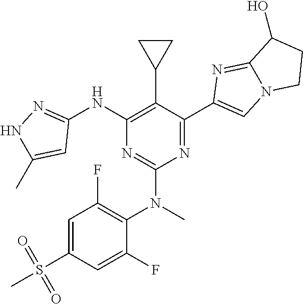 [SMILES: Cc1cc(Nc2nc(N(C)c3c(F)cc(S(C)(=O)=O)cc3F)nc(-c3cn4c(n3)C(O)CC4)c2C2CC2)n[nH]1] | 234 |
| 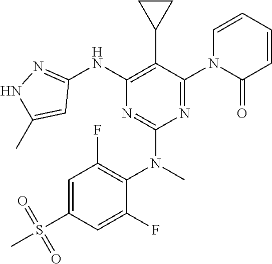 [SMILES: Cc1cc(Nc2nc(N(C)c3c(F)cc(S(C)(=O)=O)cc3F)nc(-n3ccccc3=O)c2C2CC2)n[nH]1] | 235 |
| 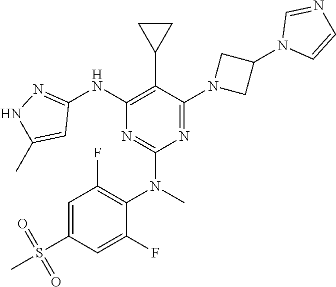 [SMILES: Cc1cc(Nc2nc(N(C)c3c(F)cc(S(C)(=O)=O)cc3F)nc(N3CC(n4ccnc4)C3)c2C2CC2)n[nH]1] | 236 |
| 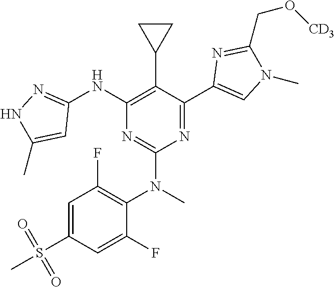 [SMILES: [2H]C([2H])([2H])OCc1nc(-c2nc(N(C)c3c(F)cc(S(C)(=O)=O)cc3F)nc(Nc3cc(C)[nH]n3)c2C2CC2)cn1C] | 237 |
| 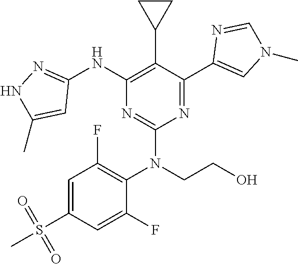 | 238 |
| 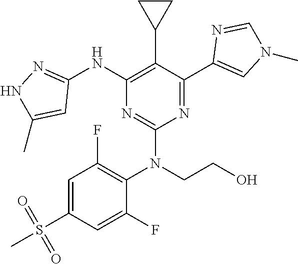 | 239 |
| 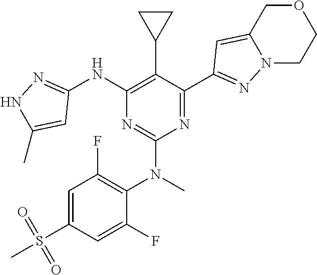 [SMILES: Cc1cc(Nc2nc(N(C)c3c(F)cc(S(C)(=O)=O)cc3F)nc(-c3cc4n(n3)CCOC4)c2C2CC2)n[nH]1] | 240 |
| 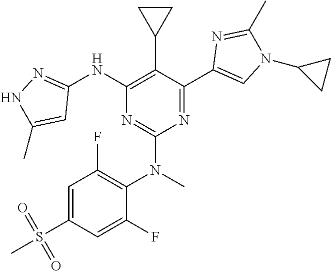 [SMILES: Cc1cc(Nc2nc(N(C)c3c(F)cc(S(C)(=O)=O)cc3F)nc(-c3cn(C4CC4)c(C)n3)c2C2CC2)n[nH]1] | 241 |
| 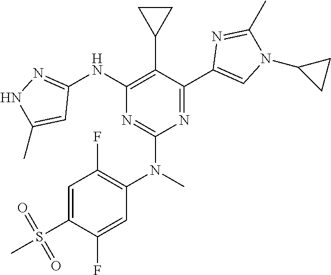 [SMILES: Cc1cc(Nc2nc(N(C)c3cc(F)c(S(C)(=O)=O)cc3F)nc(-c3cn(C4CC4)c(C)n3)c2C2CC2)n[nH]1] | 242 |
| 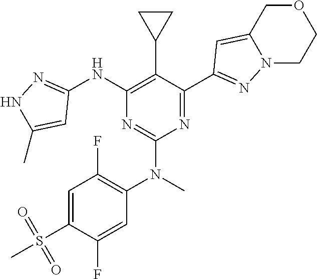 [SMILES: Cc1cc(Nc2nc(N(C)c3cc(F)c(S(C)(=O)=O)cc3F)nc(-c3cc4n(n3)CCOC4)c2C2CC2)n[nH]1] | 243 |
| 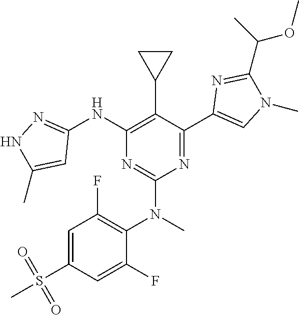 | 244 |
| 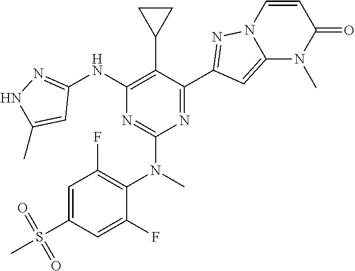 [SMILES: Cc1cc(Nc2nc(N(C)c3c(F)cc(S(C)(=O)=O)cc3F)nc(-c3cc4n(C)c(=O)ccn4n3)c2C2CC2)n[nH]1] | 245 |
| 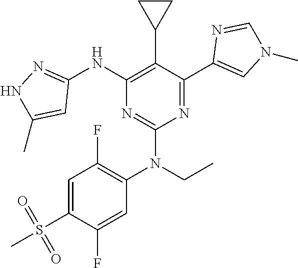 | 246 |
| 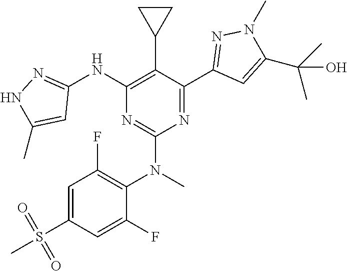 [SMILES: Cc1cc(Nc2nc(N(C)c3c(F)cc(S(C)(=O)=O)cc3F)nc(-c3cc(C(C)(C)O)n(C)n3)c2C2CC2)n[nH]1] | 247 |
| 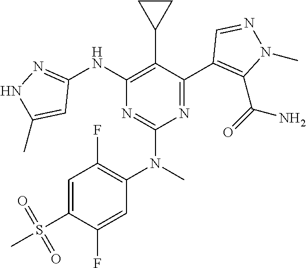 | 248 |
| 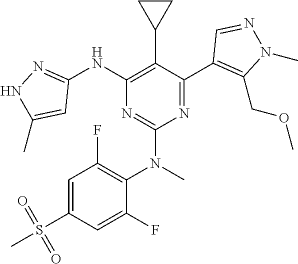 | 249 |
| 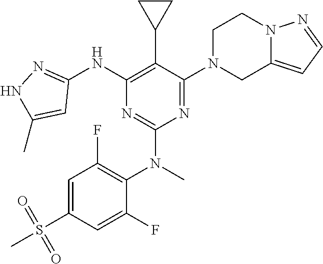 [SMILES: Cc1cc(Nc2nc(N(C)c3c(F)cc(S(C)(=O)=O)cc3F)nc(N3CCn4nccc4C3)c2C2CC2)n[nH]1] | 250 |
| 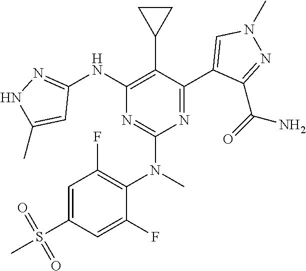 [SMILES: Cc1cc(Nc2nc(N(C)c3c(F)cc(S(C)(=O)=O)cc3F)nc(-c3cn(C)nc3C(N)=O)c2C2CC2)n[nH]1] | 251 |
| 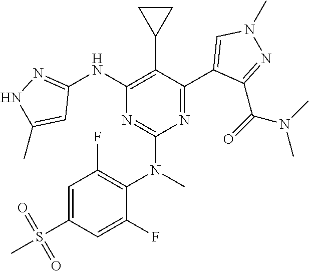 | 252 |
| 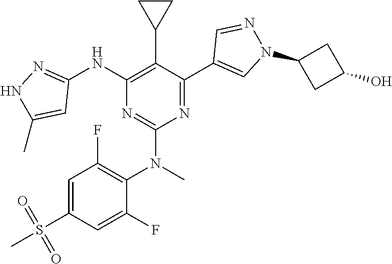 [SMILES: Cc1cc(Nc2nc(N(C)c3c(F)cc(S(C)(=O)=O)cc3F)nc(-c3cnn([C@H]4C[C@H](O)C4)c3)c2C2CC2)n[nH]1] | 253 |
| 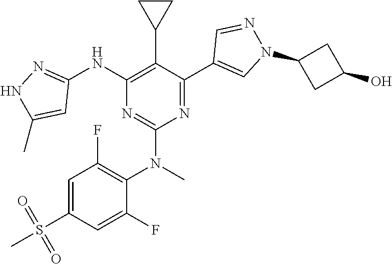 [SMILES: Cc1cc(Nc2nc(N(C)c3c(F)cc(S(C)(=O)=O)cc3F)nc(-c3cnn([C@H]4C[C@@H](O)C4)c3)c2C2CC2)n[nH]1] | 254 |
| 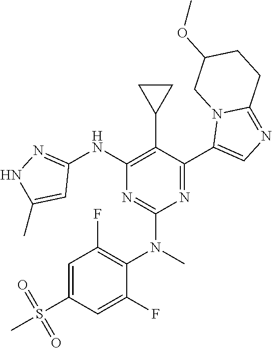 [SMILES: COC1CCc2ncc(-c3nc(N(C)c4c(F)cc(S(C)(=O)=O)cc4F)nc(Nc4cc(C)[nH]n4)c3C3CC3)n2C1] | 255 |
| 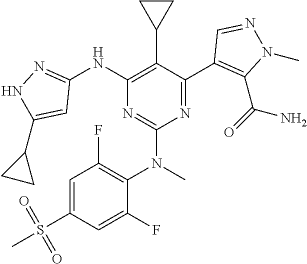 [SMILES: CN(c1nc(Nc2cc(C3CC3)[nH]n2)c(C2CC2)c(-c2cnn(C)c2C(N)=O)n1)c1c(F)cc(S(C)(=O)=O)cc1F] | 256 |
| 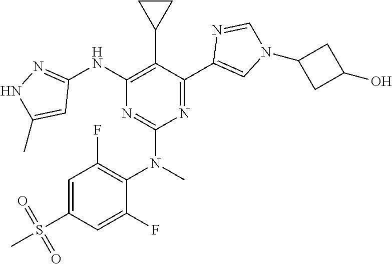 [SMILES: Cc1cc(Nc2nc(N(C)c3c(F)cc(S(C)(=O)=O)cc3F)nc(-c3cn(C4CC(O)C4)cn3)c2C2CC2)n[nH]1] | 257 |
| 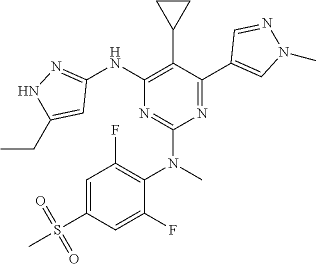 [SMILES: CCc1cc(Nc2nc(N(C)c3c(F)cc(S(C)(=O)=O)cc3F)nc(-c3cnn(C)c3)c2C2CC2)n[nH]1] | 258 |
| 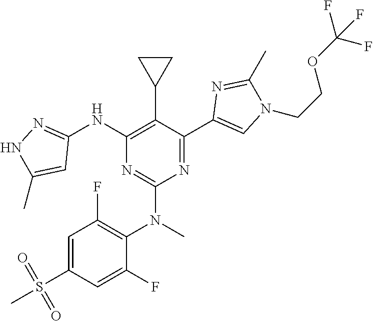 [SMILES: Cc1cc(Nc2nc(N(C)c3c(F)cc(S(C)(=O)=O)cc3F)nc(-c3cn(CCOC(F)(F)F)c(C)n3)c2C2CC2)n[nH]1] | 259 |
| 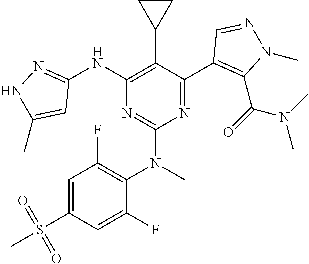 | 260 |
| 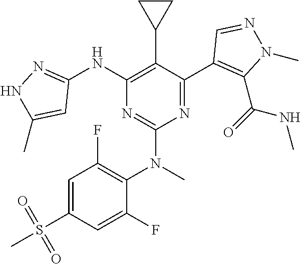 | 261 |
| 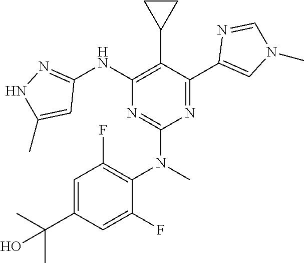 [SMILES: Cc1cc(Nc2nc(N(C)c3c(F)cc(C(C)(C)O)cc3F)nc(-c3cn(C)cn3)c2C2CC2)n[nH]1] | 262 |
| 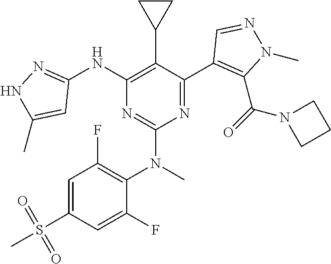 [SMILES: Cc1cc(Nc2nc(N(C)c3c(F)cc(S(C)(=O)=O)cc3F)nc(-c3cnn(C)c3C(=O)N3CCC3)c2C2CC2)n[nH]1] | 263 |
| 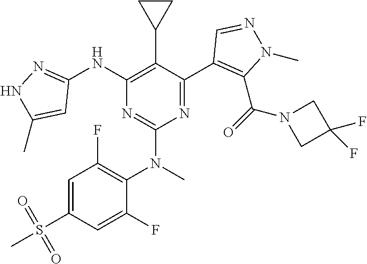 [SMILES: Cc1cc(Nc2nc(N(C)c3c(F)cc(S(C)(=O)=O)cc3F)nc(-c3cnn(C)c3C(=O)N3CC(F)(F)C3)c2C2CC2)n[nH]1] | 264 |
| 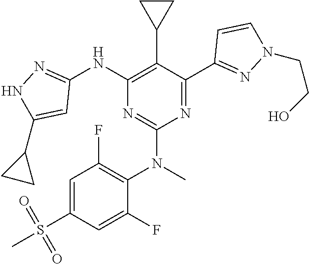 [SMILES: CN(c1nc(Nc2cc(C3CC3)[nH]n2)c(C2CC2)c(-c2ccn(CCO)n2)n1)c1c(F)cc(S(C)(=O)=O)cc1F] | 265 |
| 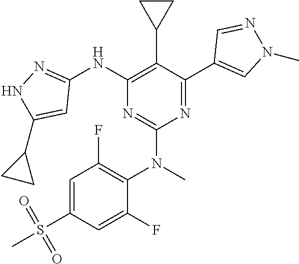 [SMILES: CN(c1nc(Nc2cc(C3CC3)[nH]n2)c(C2CC2)c(-c2cnn(C)c2)n1)c1c(F)cc(S(C)(=O)=O)cc1F] | 266 |
| 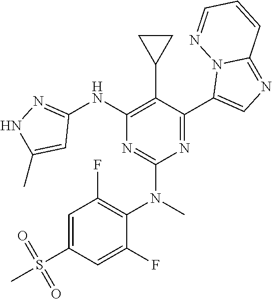 [SMILES: Cc1cc(Nc2nc(N(C)c3c(F)cc(S(C)(=O)=O)cc3F)nc(-c3cnc4cccnn34)c2C2CC2)n[nH]1] | 267 |
| 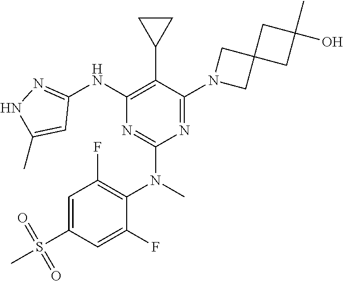 [SMILES: Cc1cc(Nc2nc(N(C)c3c(F)cc(S(C)(=O)=O)cc3F)nc(N3CC4(C3)CC(C)(O)C4)c2C2CC2)n[nH]1] | 268 |
| 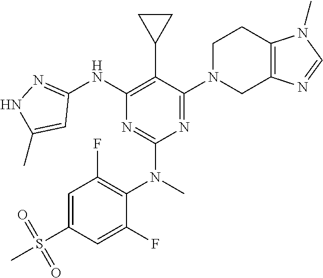 [SMILES: Cc1cc(Nc2nc(N(C)c3c(F)cc(S(C)(=O)=O)cc3F)nc(N3CCc4c(ncn4C)C3)c2C2CC2)n[nH]1] | 269 |
| 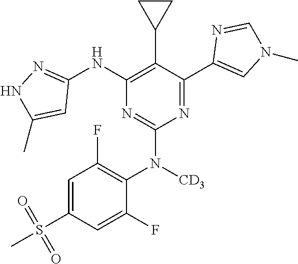 | 270 |
| 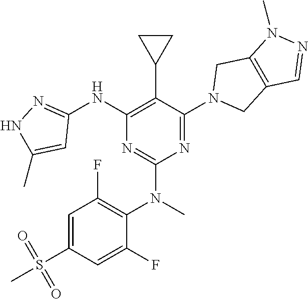 [SMILES: Cc1cc(Nc2nc(N(C)c3c(F)cc(S(C)(=O)=O)cc3F)nc(N3Cc4cnn(C)c4C3)c2C2CC2)n[nH]1] | 271 |
| 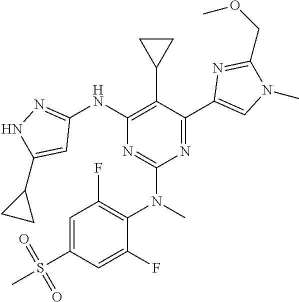 [SMILES: COCc1nc(-c2nc(N(C)c3c(F)cc(S(C)(=O)=O)cc3F)nc(Nc3cc(C4CC4)[nH]n3)c2C2CC2)cn1C] | 272 |
| 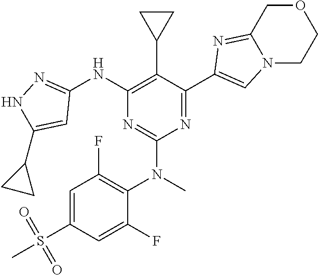 [SMILES: CN(c1nc(Nc2cc(C3CC3)[nH]n2)c(C2CC2)c(-c2cn3c(n2)COCC3)n1)c1c(F)cc(S(C)(=O)=O)cc1F] | 273 |
| 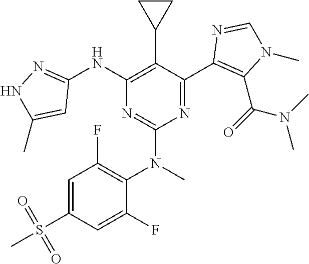 | 274 |
| 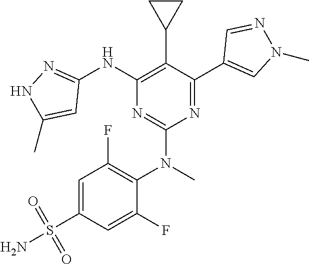 [SMILES: Cc1cc(Nc2nc(N(C)c3c(F)cc(S(N)(=O)=O)cc3F)nc(-c3cnn(C)c3)c2C2CC2)n[nH]1] | 275 |
| 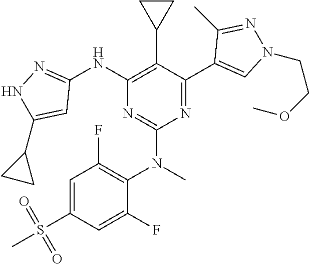 [SMILES: COCCn1cc(-c2nc(N(C)c3c(F)cc(S(C)(=O)=O)cc3F)nc(Nc3cc(C4CC4)[nH]n3)c2C2CC2)c(C)n1] | 276 |
| 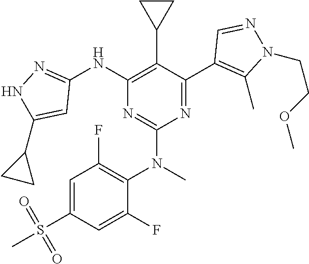 [SMILES: COCCn1ncc(-c2nc(N(C)c3c(F)cc(S(C)(=O)=O)cc3F)nc(Nc3cc(C4CC4)[nH]n3)c2C2CC2)c1C] | 277 |
| 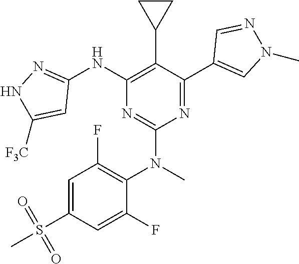 | 278 |
| 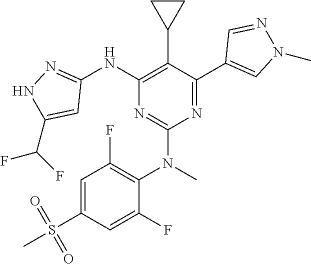 [SMILES: CN(c1nc(Nc2cc(C(F)F)[nH]n2)c(C2CC2)c(-c2cnn(C)c2)n1)c1c(F)cc(S(C)(=O)=O)cc1F] | 279 |
| 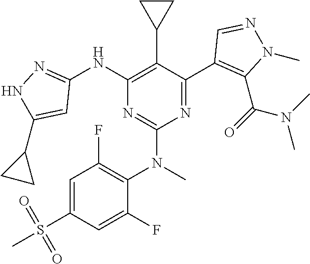 [SMILES: CN(C)C(=O)c1c(-c2nc(N(C)c3c(F)cc(S(C)(=O)=O)cc3F)nc(Nc3cc(C4CC4)[nH]n3)c2C2CC2)cnn1C] | 280 |
| 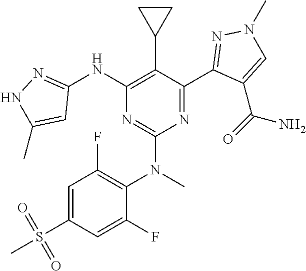 [SMILES: Cc1cc(Nc2nc(N(C)c3c(F)cc(S(C)(=O)=O)cc3F)nc(-c3nn(C)cc3C(N)=O)c2C2CC2)n[nH]1] | 281 |
| 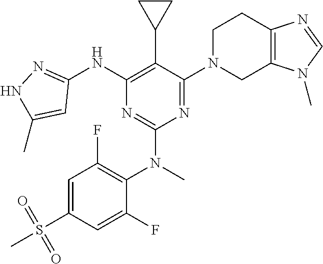 [SMILES: Cc1cc(Nc2nc(N(C)c3c(F)cc(S(C)(=O)=O)cc3F)nc(N3CCc4ncn(C)c4C3)c2C2CC2)n[nH]1] | 282 |
| 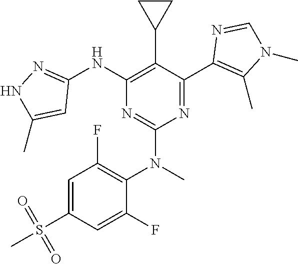 | 283 |
| 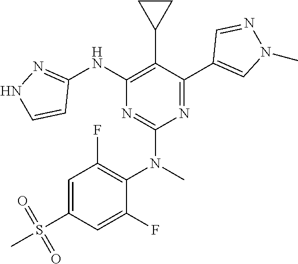 | 284 |
| 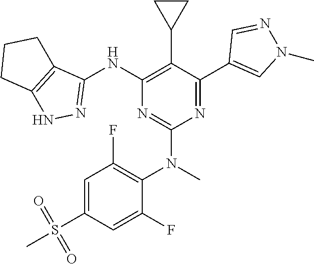 [SMILES: CN(c1nc(Nc2n[nH]c3c2CCC3)c(C2CC2)c(-c2cnn(C)c2)n1)c1c(F)cc(S(C)(=O)=O)cc1F] | 285 |
| 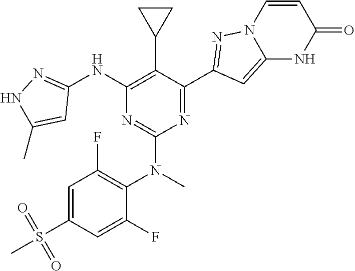 [SMILES: Cc1cc(Nc2nc(N(C)c3c(F)cc(S(C)(=O)=O)cc3F)nc(-c3cc4[nH]c(=O)ccn4n3)c2C2CC2)n[nH]1] | 286 |
| 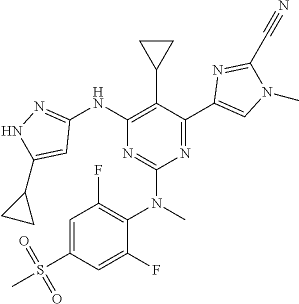 [SMILES: CN(c1nc(Nc2cc(C3CC3)[nH]n2)c(C2CC2)c(-c2cn(C)c(C#N)n2)n1)c1c(F)cc(S(C)(=O)=O)cc1F] | 287 |
| 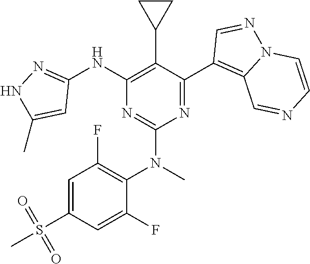 [SMILES: Cc1cc(Nc2nc(N(C)c3c(F)cc(S(C)(=O)=O)cc3F)nc(-c3cnn4ccncc34)c2C2CC2)n[nH]1] | 288 |
| 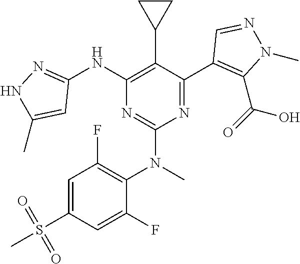 | 289 |
| 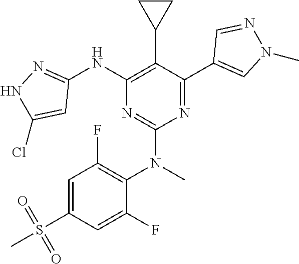 | 290 |
| 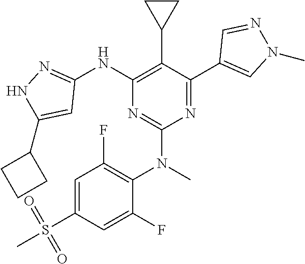 [SMILES: CN(c1nc(Nc2cc(C3CCC3)[nH]n2)c(C2CC2)c(-c2cnn(C)c2)n1)c1c(F)cc(S(C)(=O)=O)cc1F] | 291 |
| 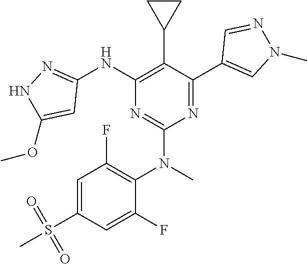 | 292 |
| 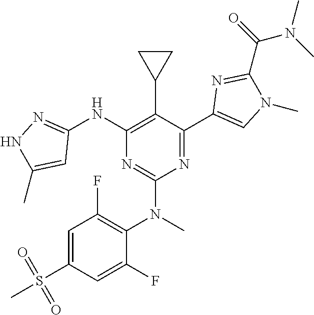 | 293 |
| 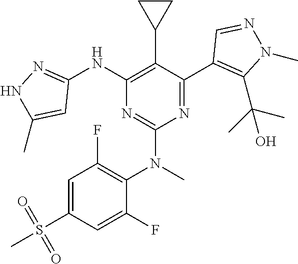 | 294 |
| 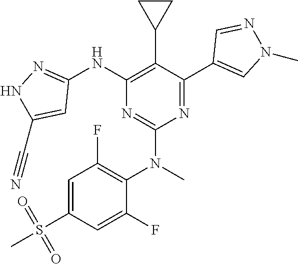 | 295 |
| 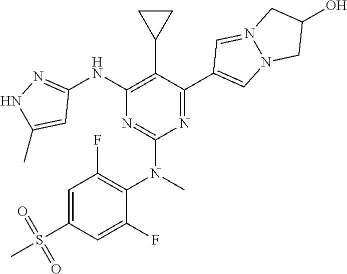 | 296 |
| 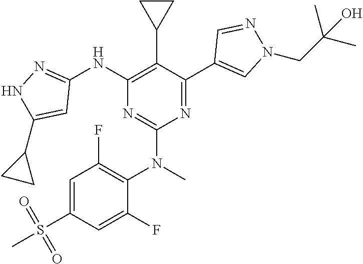 [SMILES: CN(c1nc(Nc2cc(C3CC3)[nH]n2)c(C2CC2)c(-c2cnn(CC(C)(C)O)c2)n1)c1c(F)cc(S(C)(=O)=O)cc1F] | 297 |
| 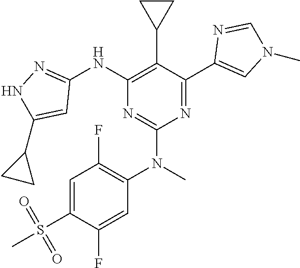 [SMILES: CN(c1nc(Nc2cc(C3CC3)[nH]n2)c(C2CC2)c(-c2cn(C)cn2)n1)c1cc(F)c(S(C)(=O)=O)cc1F] | 298 |
| 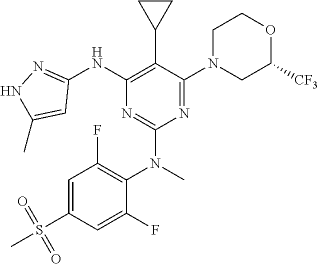 [SMILES: Cc1cc(Nc2nc(N(C)c3c(F)cc(S(C)(=O)=O)cc3F)nc(N3CCO[C@H](C(F)(F)F)C3)c2C2CC2)n[nH]1] | 299 |
| 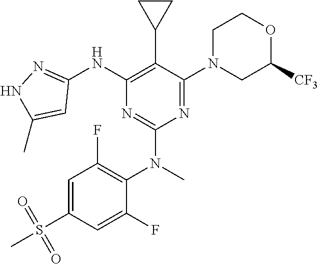 [SMILES: Cc1cc(Nc2nc(N(C)c3c(F)cc(S(C)(=O)=O)cc3F)nc(N3CCO[C@@H](C(F)(F)F)C3)c2C2CC2)n[nH]1] | 300 |
| 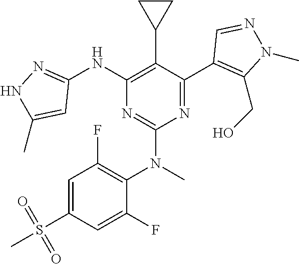 | 301 |
| 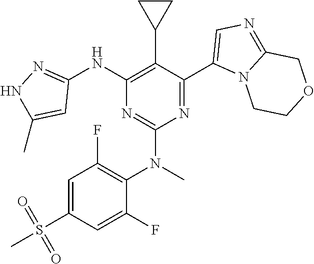 [SMILES: Cc1cc(Nc2nc(N(C)c3c(F)cc(S(C)(=O)=O)cc3F)nc(-c3cnc4n3CCOC4)c2C2CC2)n[nH]1] | 302 |
| 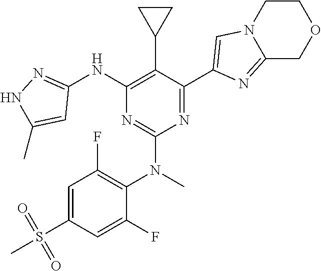 [SMILES: Cc1cc(Nc2nc(N(C)c3c(F)cc(S(C)(=O)=O)cc3F)nc(-c3cn4c(n3)COCC4)c2C2CC2)n[nH]1] | 303 |
| 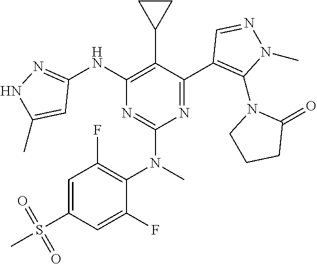 [SMILES: Cc1cc(Nc2nc(N(C)c3c(F)cc(S(C)(=O)=O)cc3F)nc(-c3cnn(C)c3N3CCCC3=O)c2C2CC2)n[nH]1] | 304 |
| 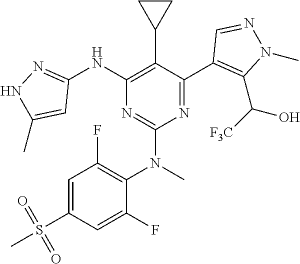 | 305 |
| 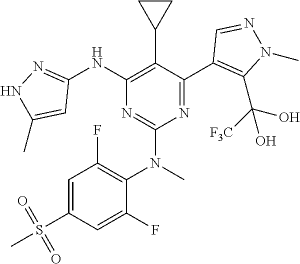 | 306 |
| 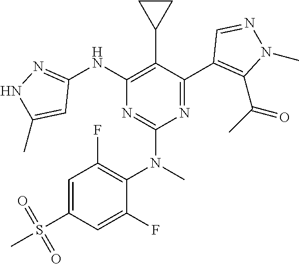 | 307 |
| 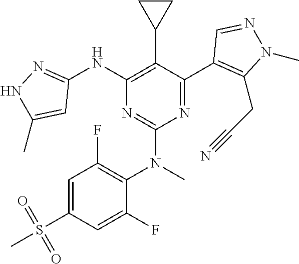 | 308 |
| 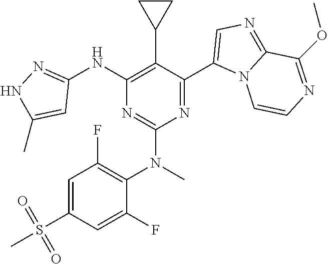 [SMILES: COc1nccn2c(-c3nc(N(C)c4c(F)cc(S(C)(=O)=O)cc4F)nc(Nc4cc(C)[nH]n4)c3C3CC3)cnc12] | 309 |
| 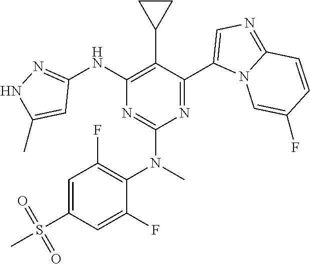 [SMILES: Cc1cc(Nc2nc(N(C)c3c(F)cc(S(C)(=O)=O)cc3F)nc(-c3cnc4ccc(F)cn34)c2C2CC2)n[nH]1] | 310 |
| 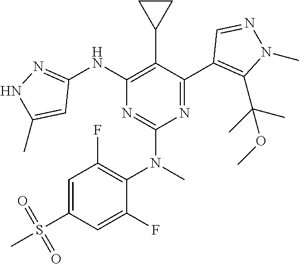 | 311 |
| 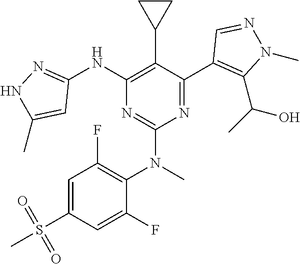 | 312 |
| 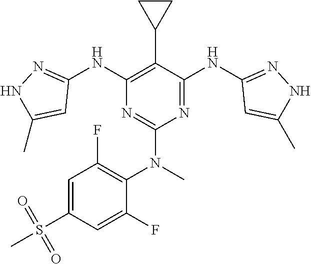 | 313 |
| 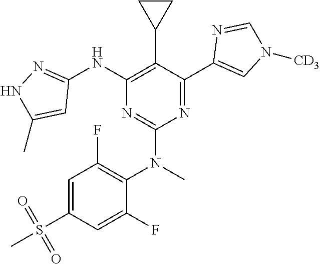 [SMILES: [2H]C([2H])([2H])n1cnc(-c2nc(N(C)c3c(F)cc(S(C)(=O)=O)cc3F)nc(Nc3cc(C)[nH]n3)c2C2CC2)c1] | 314 |
| 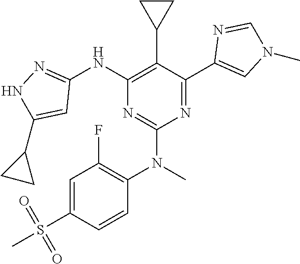 [SMILES: CN(c1nc(Nc2cc(C3CC3)[nH]n2)c(C2CC2)c(-c2cn(C)cn2)n1)c1ccc(S(C)(=O)=O)cc1F] | 315 |
| 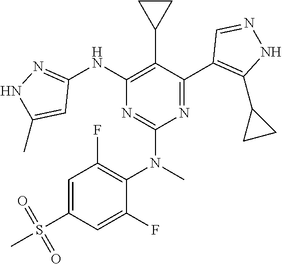 [SMILES: Cc1cc(Nc2nc(N(C)c3c(F)cc(S(C)(=O)=O)cc3F)nc(-c3cn[nH]c3C3CC3)c2C2CC2)n[nH]1] | 316 |
| 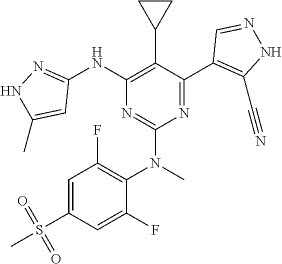 [SMILES: Cc1cc(Nc2nc(N(C)c3c(F)cc(S(C)(=O)=O)cc3F)nc(-c3cn[nH]c3C#N)c2C2CC2)n[nH]1] | 317 |
| 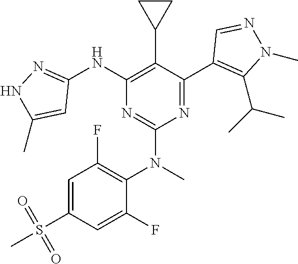 | 318 |
| 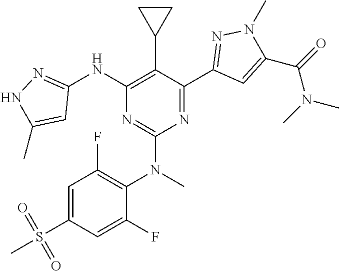 | 319 |
| 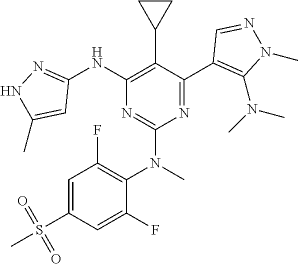 | 320 |
| 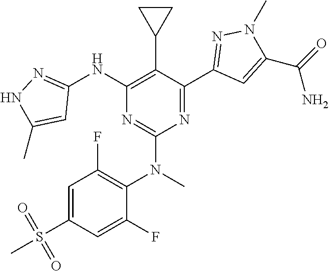 [SMILES: Cc1cc(Nc2nc(N(C)c3c(F)cc(S(C)(=O)=O)cc3F)nc(-c3cc(C(N)=O)n(C)n3)c2C2CC2)n[nH]1] | 321 |
| 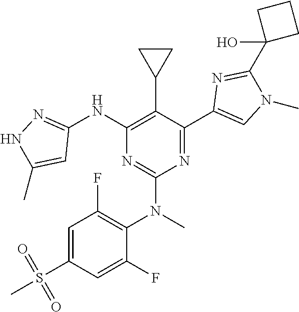 [SMILES: Cc1cc(Nc2nc(N(C)c3c(F)cc(S(C)(=O)=O)cc3F)nc(-c3cn(C)c(C4(O)CCC4)n3)c2C2CC2)n[nH]1] | 322 |
| 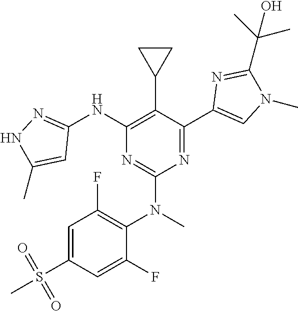 | 323 |
| 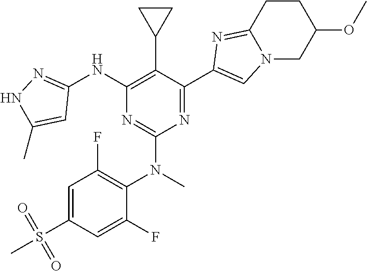 | 324 |
| 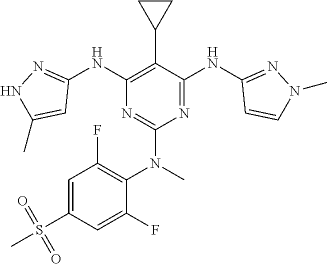 | 325 |
| 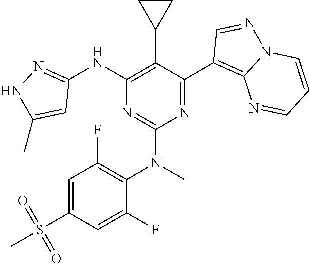 [SMILES: Cc1cc(Nc2nc(N(C)c3c(F)cc(S(C)(=O)=O)cc3F)nc(-c3cnn4cccnc34)c2C2CC2)n[nH]1] | 326 |
| 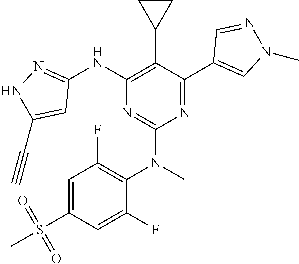 | 327 |
| 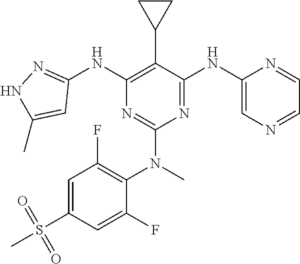 [SMILES: Cc1cc(Nc2nc(N(C)c3c(F)cc(S(C)(=O)=O)cc3F)nc(Nc3cnccn3)c2C2CC2)n[nH]1] | 328 |
| 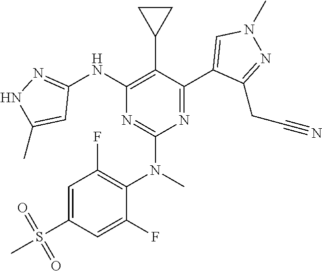 [SMILES: Cc1cc(Nc2nc(N(C)c3c(F)cc(S(C)(=O)=O)cc3F)nc(-c3cn(C)nc3CC#N)c2C2CC2)n[nH]1] | 329 |
| 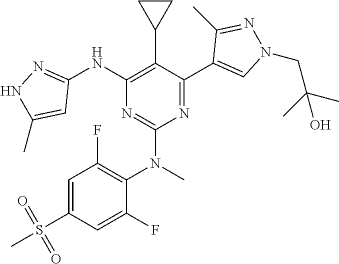 [SMILES: Cc1cc(Nc2nc(N(C)c3c(F)cc(S(C)(=O)=O)cc3F)nc(-c3cn(CC(C)(C)O)nc3C)c2C2CC2)n[nH]1] | 330 |
| 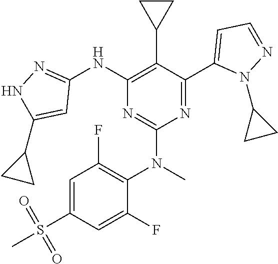 [SMILES: CN(c1nc(Nc2cc(C3CC3)[nH]n2)c(C2CC2)c(-c2ccnn2C2CC2)n1)c1c(F)cc(S(C)(=O)=O)cc1F] | 331 |
| 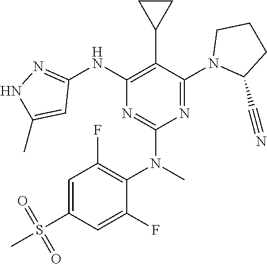 [SMILES: Cc1cc(Nc2nc(N(C)c3c(F)cc(S(C)(=O)=O)cc3F)nc(N3CCC[C@@H]3C#N)c2C2CC2)n[nH]1] | 332 |
| 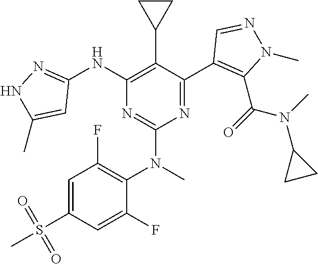 [SMILES: Cc1cc(Nc2nc(N(C)c3c(F)cc(S(C)(=O)=O)cc3F)nc(-c3cnn(C)c3C(=O)N(C)C3CC3)c2C2CC2)n[nH]1] | 333 |
| 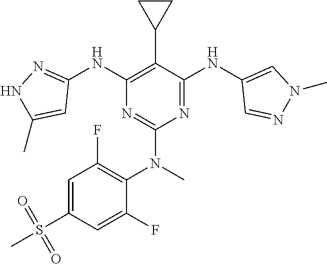 | 334 |
| 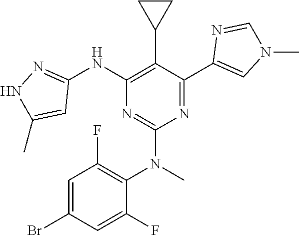 [SMILES: Cc1cc(Nc2nc(N(C)c3c(F)cc(Br)cc3F)nc(-c3cn(C)cn3)c2C2CC2)n[nH]1] | 335 |
| 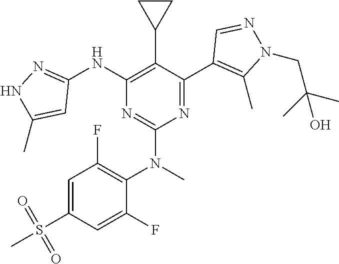 [SMILES: Cc1cc(Nc2nc(N(C)c3c(F)cc(S(C)(=O)=O)cc3F)nc(-c3cnn(CC(C)(C)O)c3C)c2C2CC2)n[nH]1] | 336 |
| 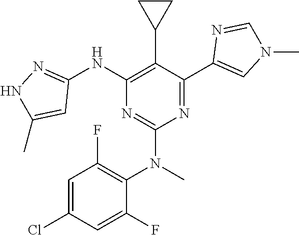 [SMILES: Cc1cc(Nc2nc(N(C)c3c(F)cc(Cl)cc3F)nc(-c3cn(C)cn3)c2C2CC2)n[nH]1] | 337 |
| 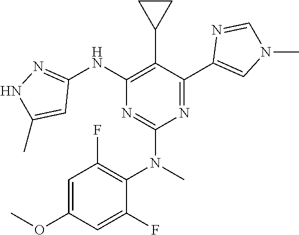 | 338 |
| 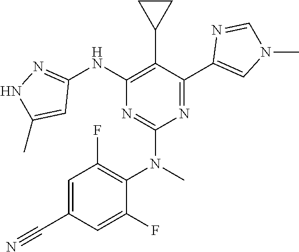 [SMILES: Cc1cc(Nc2nc(N(C)c3c(F)cc(C#N)cc3F)nc(-c3cn(C)cn3)c2C2CC2)n[nH]1] | 339 |
| 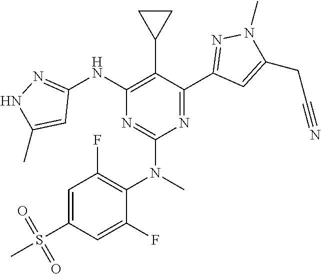 [SMILES: Cc1cc(Nc2nc(N(C)c3c(F)cc(S(C)(=O)=O)cc3F)nc(-c3cc(CC#N)n(C)n3)c2C2CC2)n[nH]1] | 340 |
| 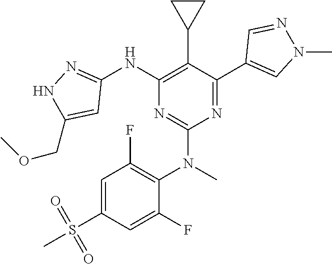 | 341 |
| 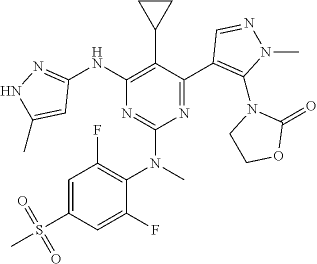 [SMILES: Cc1cc(Nc2nc(N(C)c3c(F)cc(S(C)(=O)=O)cc3F)nc(-c3cnn(C)c3N3CCOC3=O)c2C2CC2)n[nH]1] | 342 |
| 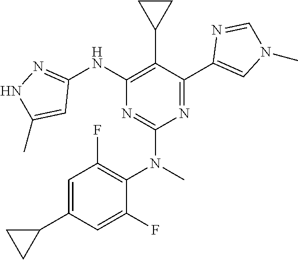 [SMILES: Cc1cc(Nc2nc(N(C)c3c(F)cc(C4CC4)cc3F)nc(-c3cn(C)cn3)c2C2CC2)n[nH]1] | 343 |
| 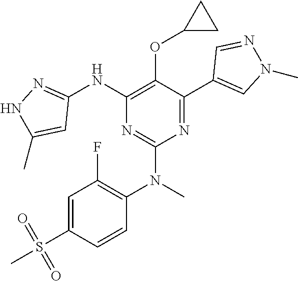 | 344 |
| 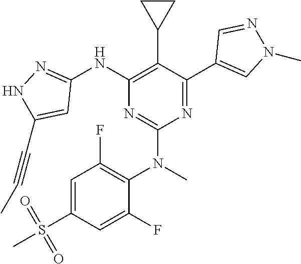 [SMILES: CC#Cc1cc(Nc2nc(N(C)c3c(F)cc(S(C)(=O)=O)cc3F)nc(-c3cnn(C)c3)c2C2CC2)n[nH]1] | 345 |
| 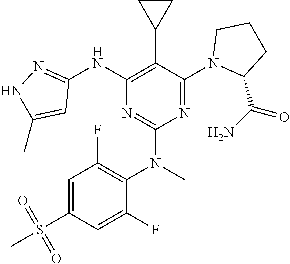 [SMILES: Cc1cc(Nc2nc(N(C)c3c(F)cc(S(C)(=O)=O)cc3F)nc(N3CCC[C@@H]3C(N)=O)c2C2CC2)n[nH]1] | 346 |
| 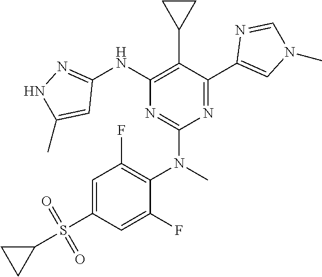 [SMILES: Cc1cc(Nc2nc(N(C)c3c(F)cc(S(=O)(=O)C4CC4)cc3F)nc(-c3cn(C)cn3)c2C2CC2)n[nH]1] | 347 |
| 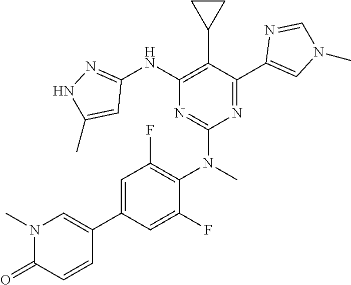 [SMILES: Cc1cc(Nc2nc(N(C)c3c(F)cc(-c4ccc(=O)n(C)c4)cc3F)nc(-c3cn(C)cn3)c2C2CC2)n[nH]1] | 348 |
| 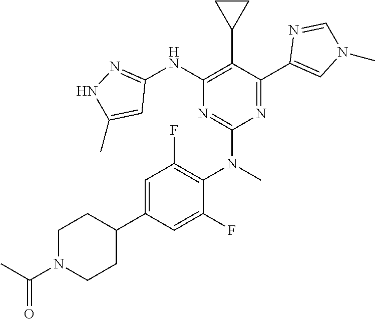 [SMILES: CC(=O)N1CCC(c2cc(F)c(N(C)c3nc(Nc4cc(C)[nH]n4)c(C4CC4)c(-c4cn(C)cn4)n3)c(F)c2)CC1] | 349 |
| 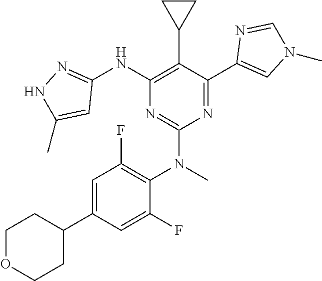 [SMILES: Cc1cc(Nc2nc(N(C)c3c(F)cc(C4CCOCC4)cc3F)nc(-c3cn(C)cn3)c2C2CC2)n[nH]1] | 350 |
| 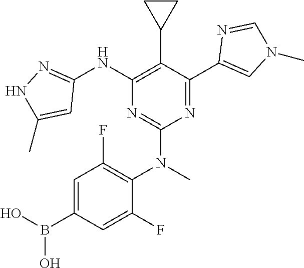 [SMILES: Cc1cc(Nc2nc(N(C)c3c(F)cc(B(O)O)cc3F)nc(-c3cn(C)cn3)c2C2CC2)n[nH]1] | 351 |
| 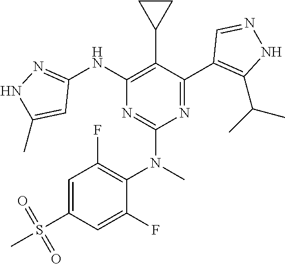 [SMILES: Cc1cc(Nc2nc(N(C)c3c(F)cc(S(C)(=O)=O)cc3F)nc(-c3cn[nH]c3C(C)C)c2C2CC2)n[nH]1] | 352 |
| 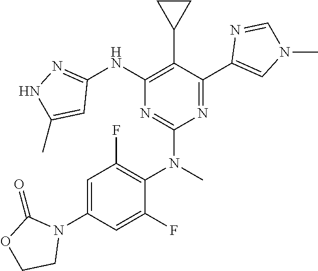 [SMILES: Cc1cc(Nc2nc(N(C)c3c(F)cc(N4CCOC4=O)cc3F)nc(-c3cn(C)cn3)c2C2CC2)n[nH]1] | 353 |
| 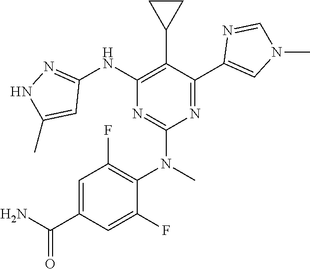 [SMILES: Cc1cc(Nc2nc(N(C)c3c(F)cc(C(N)=O)cc3F)nc(-c3cn(C)cn3)c2C2CC2)n[nH]1] | 354 |
| 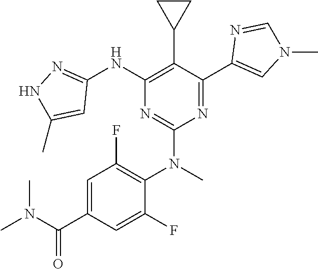 | 355 |
| 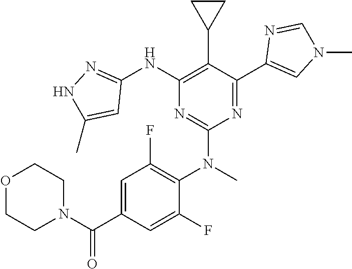 [SMILES: Cc1cc(Nc2nc(N(C)c3c(F)cc(C(=O)N4CCOCC4)cc3F)nc(-c3cn(C)cn3)c2C2CC2)n[nH]1] | 356 |
| 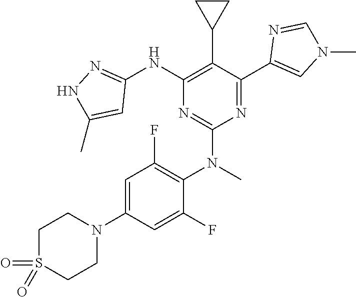 [SMILES: Cc1cc(Nc2nc(N(C)c3c(F)cc(N4CCS(=O)(=O)CC4)cc3F)nc(-c3cn(C)cn3)c2C2CC2)n[nH]1] | 357 |
| 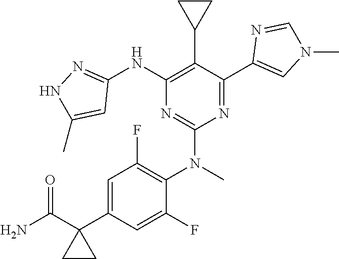 [SMILES: Cc1cc(Nc2nc(N(C)c3c(F)cc(C4(C(N)=O)CC4)cc3F)nc(-c3cn(C)cn3)c2C2CC2)n[nH]1] | 358 |
| 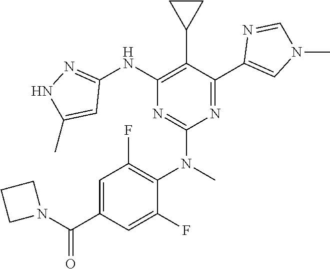 [SMILES: Cc1cc(Nc2nc(N(C)c3c(F)cc(C(=O)N4CCC4)cc3F)nc(-c3cn(C)cn3)c2C2CC2)n[nH]1] | 359 |
| 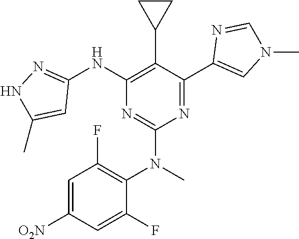 [SMILES: Cc1cc(Nc2nc(N(C)c3c(F)cc([N+](=O)[O-])cc3F)nc(-c3cn(C)cn3)c2C2CC2)n[nH]1] | 360 |
| 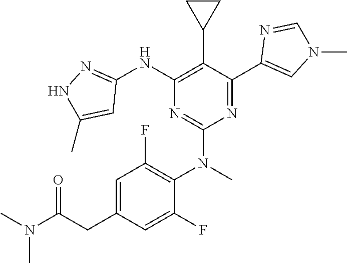 | 361 |
| 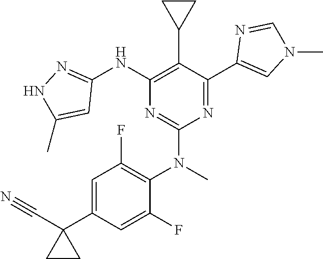 [SMILES: Cc1cc(Nc2nc(N(C)c3c(F)cc(C4(C#N)CC4)cc3F)nc(-c3cn(C)cn3)c2C2CC2)n[nH]1] | 362 |
| 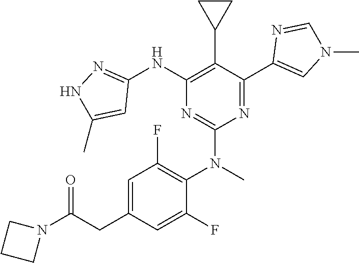 [SMILES: Cc1cc(Nc2nc(N(C)c3c(F)cc(CC(=O)N4CCC4)cc3F)nc(-c3cn(C)cn3)c2C2CC2)n[nH]1] | 363 |
| 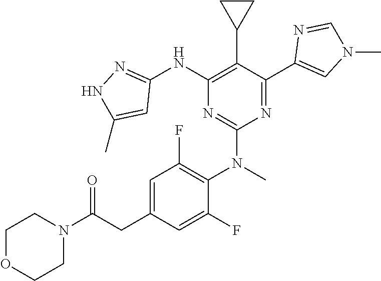 [SMILES: Cc1cc(Nc2nc(N(C)c3c(F)cc(CC(=O)N4CCOCC4)cc3F)nc(-c3cn(C)cn3)c2C2CC2)n[nH]1] | 364 |
| 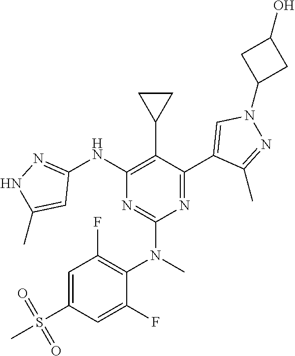 [SMILES: Cc1cc(Nc2nc(N(C)c3c(F)cc(S(C)(=O)=O)cc3F)nc(-c3cn(C4CC(O)C4)nc3C)c2C2CC2)n[nH]1] | 365 |

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating PLK4-mediated diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions comprising the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions comprising (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

### Optical Isomers-Diastereomers-Geometric Isomers-Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

### Metabolites-Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

### Isotopically Enriched Derivatives

The invention includes molecules which have been isotopically enriched at one or more position within the molecule. Thus, compounds enriched for deuterium fall within the scope of the claims.

### Methods of Preparing a Compound of the Invention

Compounds of the present invention may be prepared using reactions and techniques known in the art and those described herein. One of skill in the art will appreciate that methods of preparing compounds of the invention described herein are non-limiting and that steps within the methods may be interchangeable without affecting the structure of the end product.

### Method A

Compounds of Type I, in which A is O or S or A-R^{2B} is NH and X—R⁴ is N, can be prepared in 5 steps from as described in Scheme A. SnAr reaction under basic condition between appropriately protected amino pyrazole IV and substituted 4,6-dichloro-2-(methylthio)pyrimidine V would generate the amino pyrazole pyrimidine of type VI. Oxidation of the methylthiol VI followed by Suzuki or Stille coupling with the appropriate boronic acid (R⁵—B(OH)₂) or stannane (R⁵—SnBu₃) would afford the advanced intermediate methyl sulfone pyrimidine VIII. SnAr type addition of properly substituted aniline ((R³)ₙ-L-NH₂), phenol ((R³)ₙ-L-OH), or thiophenol ((R³)ₙ-L-SH) followed by removal of the protecting group PG¹ can finally afford tetrasubstituted pyrimidine of Type I.

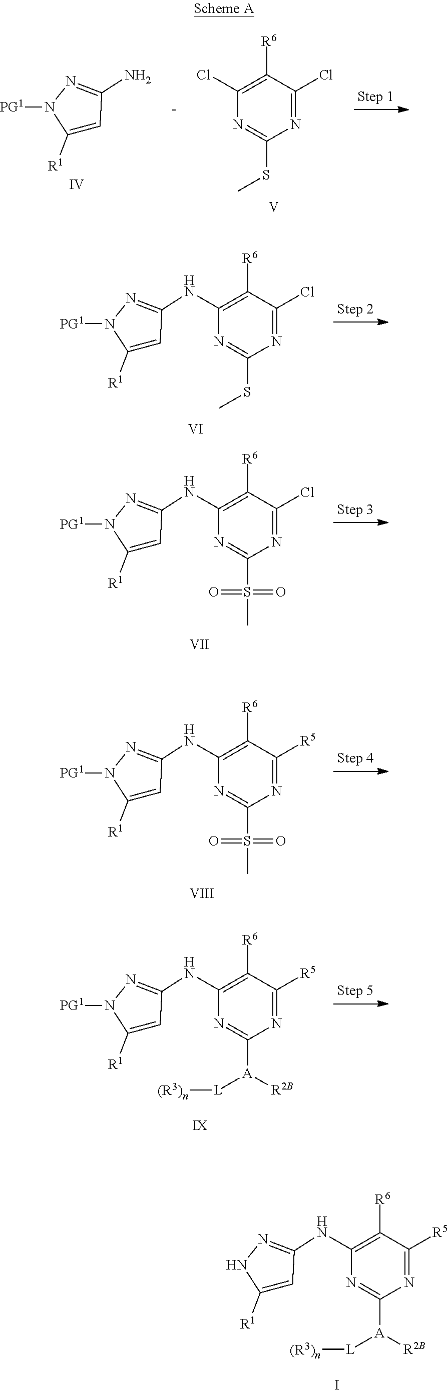

### Method B

Compounds of Type I, in which A-R^{2B} is, N—R^{2B} and X—R⁴ is N, can be prepared in 7 steps as described in Scheme B. Introduction of a protecting group (e.g., SEM, PMB, etc.) of the amino pyrazole pyrimidine of type VI described in Method A followed by oxidation of the methylthiol can generate the bis-protected amino-pyrazole pyrimidine XI. Suzuki or Stille coupling with the appropriate boronic acid boronic acid (R⁵—B(OH)₂) or stannane (R⁵—SnBu₃) can afford the advanced intermediate methyl sulfone pyrimidine XII. SnAr type addition of properly substituted aniline ((R³)ₙ-L-NH₂) to XII followed by alkylation of the aniline gives the intermediate XIV. Removal of the protecting group PG¹ and PG² can finally afford tetrasubstituted pyrimidine of Type I as exemplified in Scheme B.

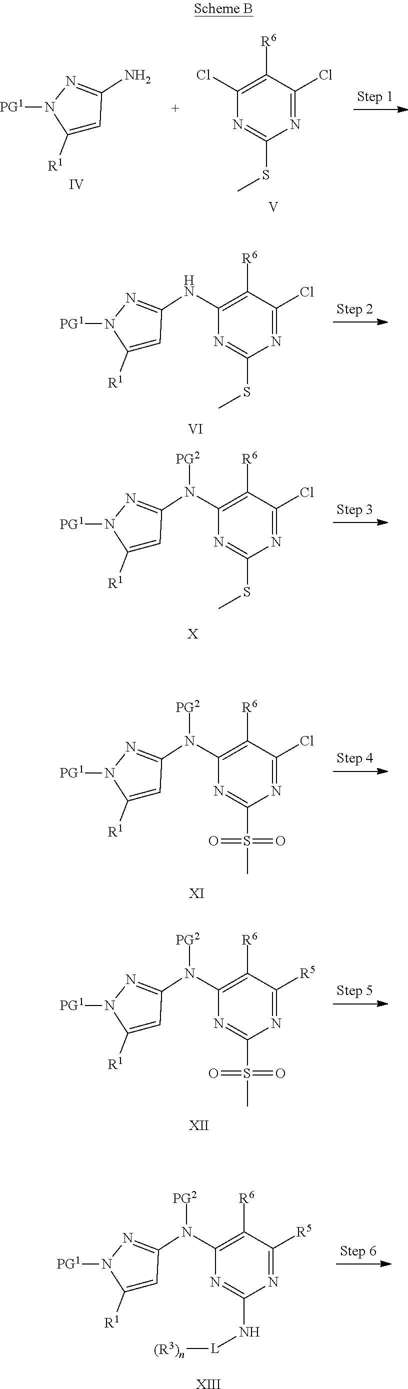

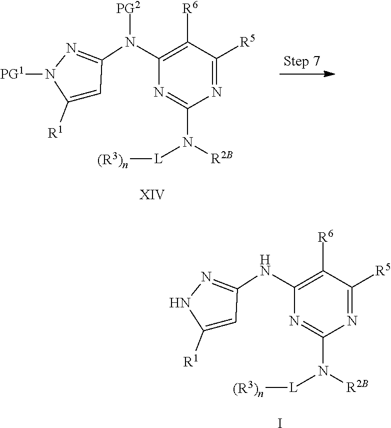

### Method C

Compounds of Type I, in which A is O or S or A-R^{2B} is NR^{2B}, X is C and R⁴ is H can be prepared in 6 steps from as described in Scheme C. Suzuki coupling between appropriate substituted boronic acid (R⁵—B(OH)₂) and 4-bromo-6-chloro-2-fluoropyridin-3-ol gives regioselectively pyridine of type XV. The phenol moiety is then transformed into the corresponding triflate XVI followed by a second Suzuki coupling to afford the pyridine of type XVII. SnAr type addition of amino-pyrazole IV on fluoro-chloro pyridine XVII, regioselectively gives the chloro-pyridine XVIII. A second SnAr type reaction of properly substituted aniline ((R³)ₙ-L-NH₂), phenol ((R³)ₙ-L-OH), or thiophenol ((R³)ₙ-L-SH) on the chloro-pyridine XVIII and subsequent deprotection of PG¹ can finally afford tetrasubstituted pyridine of Type I as exemplified in Scheme C.

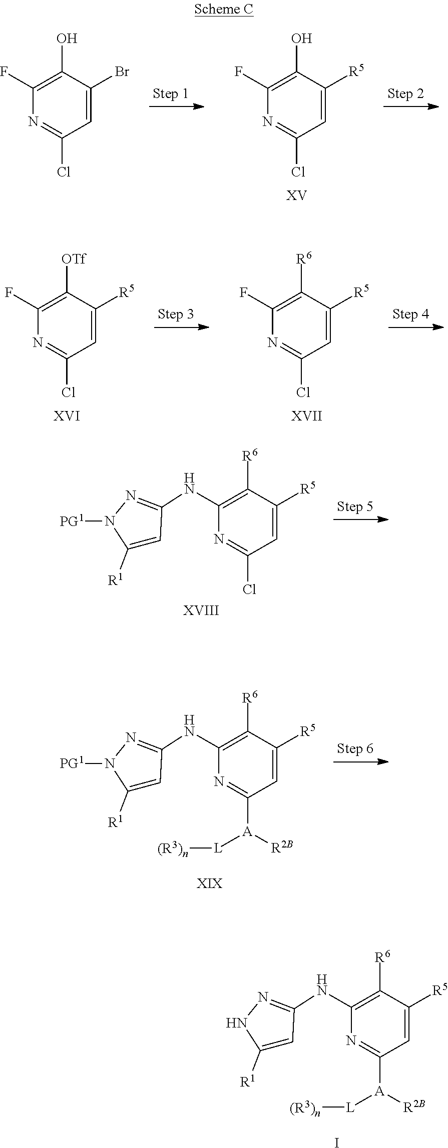

### Method D

Compounds of Type I, in which R⁶ is OMe, A is O or S or A-R^{2B} is NR^{2B}, X is C and R⁴ is not H can be prepared in 6 steps from as described in Scheme D. Bromination of commercially available phenol of type XX followed by methylation of the phenol moiety can afford the intermediate dibromo-chloro pyridine of type XXII. Buchwald-Hartwig amination of XXI with amino-pyrazole IV followed by Suzuki coupling with appropriate substituted boronic acid (R⁵—B(OH)₂) can afford the chloro-pyridine of type XXIV. SnAr type reaction of properly substituted aniline ((R³)ₙ-L-NH₂), phenol ((R³)ₙ-L-OH), or thiophenol ((R³)ₙ-L-SH) on the chloro-pyridine XXIV and subsequent deprotection of PG¹ can finally afford tetrasubstituted pyridine of Type I as exemplified in Scheme D.

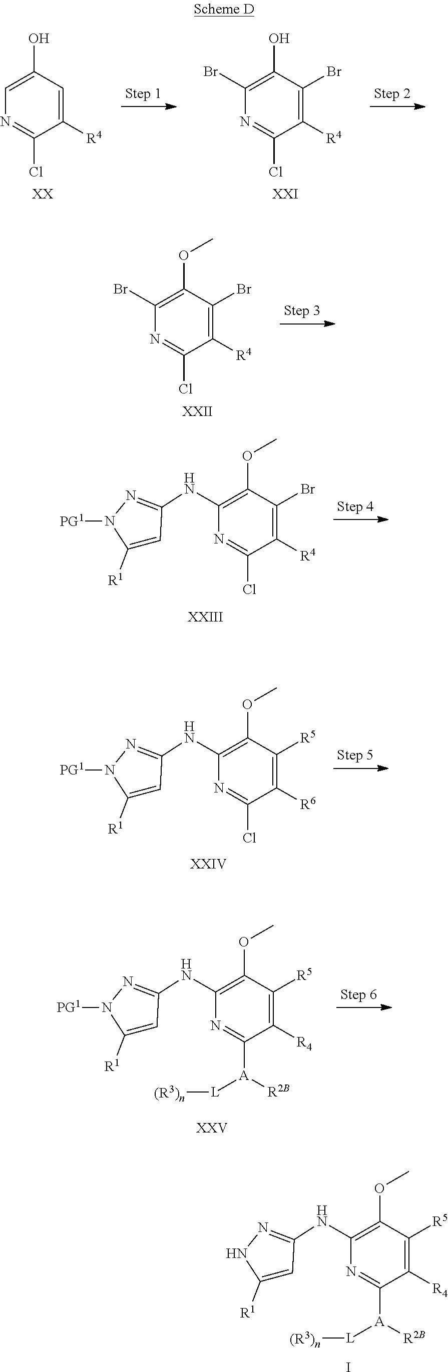

### Method E

Compounds of Type I, in which A is O or S or A-R^{2B} is NR^{2B}, and X—R⁴ is N can be prepared, alternatively from Method B previously described, in 5 steps as depicted in Scheme E starting by addition of amino-pyrazole IV on trichloro pyrimidine XXVI via SnAr type reaction. Protection of the amino group followed by SnAr type reaction of properly substituted aniline ((R³)ₙ-L-NHR^{2B}), phenol ((R³)ₙ-L-OH), or thiophenol ((R³)ₙ-L-SH) on the dichloro-pyrimidine XXVIII can generate the intermediate XXIX. Suzuki coupling with appropriately substituted boronic acid (R⁵—B(OH)₂) can afford the previously described tetra substituted pyrimidine of type XIV. Subsequent deprotection of PG¹ and PG² can finally afford the desired pyrimidine of Type I as exemplified in Scheme E.

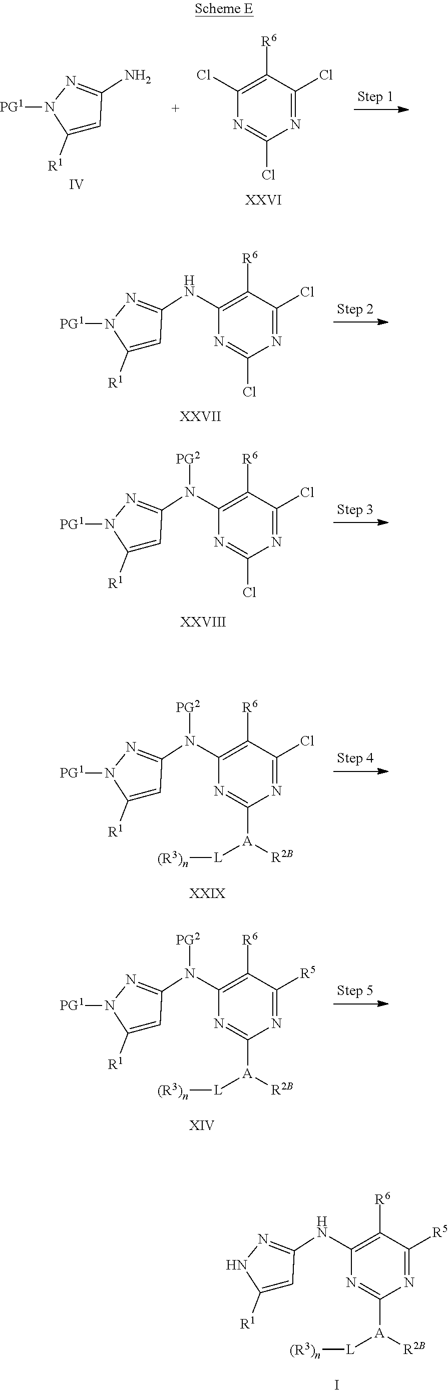

### Method F

Compounds of Type I, in which A-R^{2B} is NR^{2B}, and X—R⁴ is N and R⁵ is linked to the core pyrimidine by a C—N bond can be prepared in 4 steps from the common intermediate XI as depicted in Scheme F. SnAr type reaction of properly substituted aniline ((R³)ₙ-L-NH₂) on the chloro-pyrimidine XI is performed first to generate the pyrimidine of type XXX. The resulting aniline is then substituted to afford XXXI followed by the introduction of R⁵ via a Buchwald-Hartwig amination to generate XIV type pyrimidine. Subsequent deprotection of PG¹ and PG² can finally afford the desired pyrimidine of Type I in which R⁵ is linked to the core pyrimidine by a carbon-nitrogen bond.

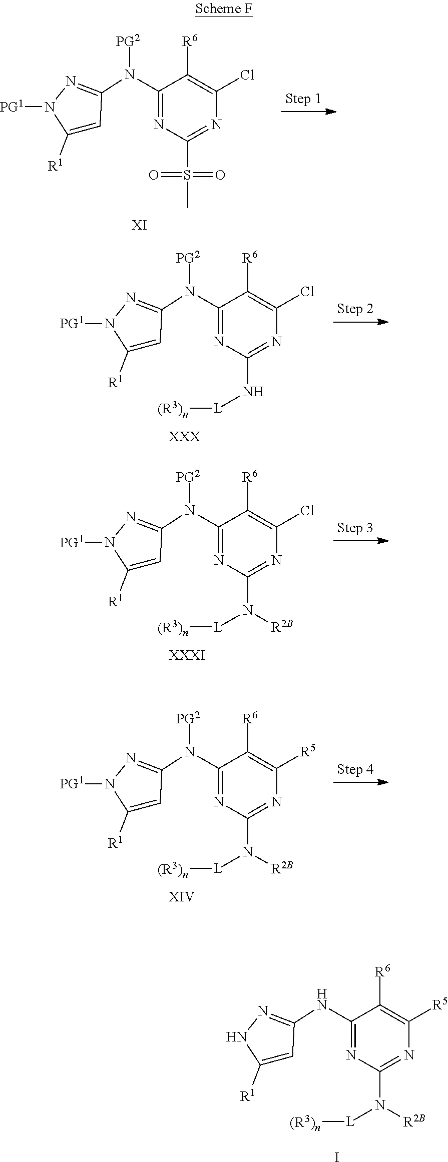

### Method G

Compounds of Type I, in which A-R^{2B} is NR^{2B}, X—R⁴ is N, and R⁵ is linked to the core pyrimidine by either a C—N or C—C bond can be prepared in 5 steps from the common intermediate X as depicted in Scheme G similarly to Method B but with an alternative reaction sequence. Buchwald-Hartwig amination, Suzuki or Stille coupling are performed first on thiomethyl pyrimidine XXXII before the oxidation step leading to XII. The remaining three steps from XII to the final compound of type I were described in Method B.

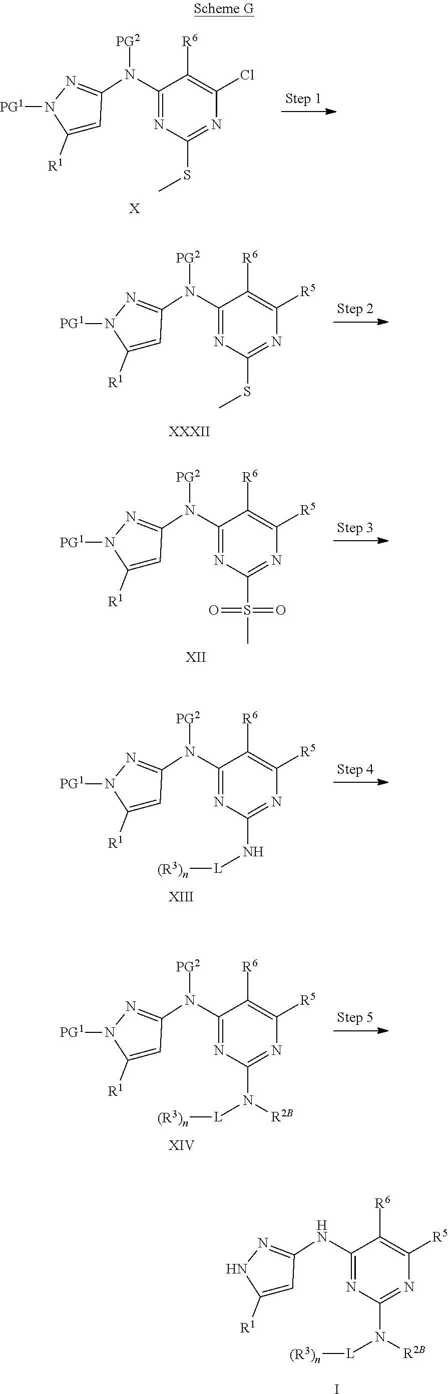

### Method H

Compounds of Type I, in which A-R^{2B} is NR^{2B}, X—R⁴ is N, and R⁵ is linked to the core pyrimidine by a C—C bond can be prepared in 2 steps as depicted in Scheme G, from intermediate XXXI, previously described. R⁵ is introduced via Suzuki or Stille coupling on the chloro-pyrimidine XXXI to generate XXXIII type pyrimidine. Subsequent deprotection of PG¹ and PG² can finally afford the desired pyrimidine of Type I as in which R⁵ is linked to the core pyrimidine by a carbon-carbon bond.

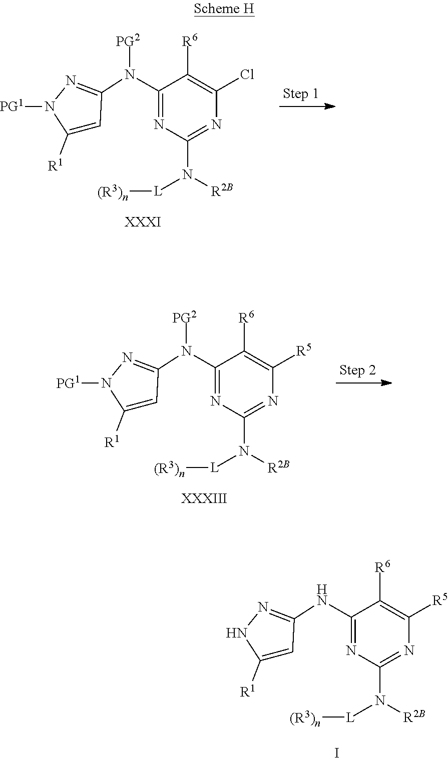

### Method I

Compounds of Type I, in which A-R^{2B} is CR^{2B}, X—R⁴ is N, and R⁵ is linked to the core pyrimidine by a C—C bond can be prepared in 2 steps from intermediate XII, previously described. SnAr type addition of aryl methyl acetate or aryl acetonitrile on intermediate XII under basic condition can afford pyrimidine of Type XXXIV. Subsequent decarboxylation following saponification of the ester and deprotection of PG¹ and PG² can finally afford the desired pyrimidine of Type I in which R^{2B} is H,H. For intermediate of type XXXIV in which R^{2B} is nitrile, direct deprotection of PG¹ and PG² can finally afford the desired pyrimidine of Type I in which R^{2B} is nitrile and A is carbon.

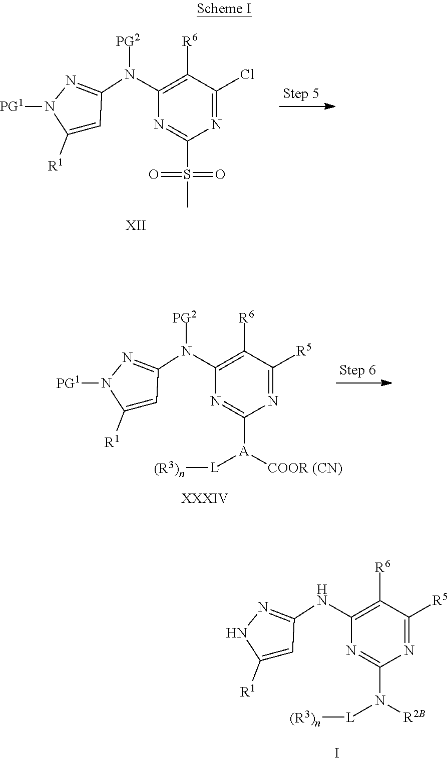

### Methods of Treatment

Compounds of the invention may be used for the treatment of a disease or condition (e.g., a cancer overexpressing TRIM37) which depend on the activity of PLK4.

The disease or condition may have the symptom of cell hyperproliferation. For example, the disease or condition may be a cancer (e.g., a cancer overexpressing TRIM37).

Cancers which have a high incidence of TRIM37 overexpression include e.g., uterine cancer, ovarian cancer, breast cancer, stomach cancer, esophageal cancer, lung cancer, and endometrial cancer.

A compound of the invention may be administered by a route selected from the group consisting of oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, intratumoral, and topical administration.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers including excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In some embodiments, the compounds are administered to the patient over a time period that is, for example, 4-11 months or 1-30 years. In some embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a disease or condition. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) Drug Injection: a liquid preparation that is a drug substance (e.g., a compound of the invention), or a solution thereof;
(2) Drug for Injection: the drug substance (e.g., a compound of the invention) as a dry solid that will be combined with the appropriate sterile vehicle for parenteral administration as a drug injection;
(3) Drug Injectable Emulsion: a liquid preparation of the drug substance (e.g., a compound of the invention) that is dissolved or dispersed in a suitable emulsion medium;
(4) Drug Injectable Suspension: a liquid preparation of the drug substance (e.g., a compound of the invention) suspended in a suitable liquid medium; and
(5) Drug for Injectable Suspension: the drug substance (e.g., a compound of the invention) as a dry solid that will be combined with the appropriate sterile vehicle for parenteral administration as a drug injectable suspension.

Formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Combinations

Compounds of the present invention may be administered to a subject in combination with one or more additional agents, e.g.:
(a) a cytotoxic agent;
(b) an antimetabolite;
(c) an alkylating agent;
(d) an anthracycline;
(e) an antibiotic;
(f) an anti-mitotic agent;
(g) a hormone therapy;
(h) a signal transduction inhibitor;
(i) a gene expression modulator;
(j) an apoptosis inducer;
(k) an angiogenesis inhibitor;
(l) an immunotherapy agent;
(m) a DNA damage repair inhibitor;
or
a combination thereof.

The cytotoxic agent may be, e.g., actinomycin-D, alemtuzumab, alitretinoin, allopurinol, altretamine, amifostine, amphotericin, amsacrine, arsenic trioxide, asparaginase, azacitidine, azathioprine, Bacille Calmette-Guérin (BCG), bendamustine, bexarotene, bevacizumab, bleomycin, bortezomib, busulphan, capecitabine, carboplatin, carfilzomib, carmustine, cetuximab, cisplatin, chlorambucil, cladribine, clofarabine, colchicine, crisantaspase, cyclophosphamide, cyclosporine, cytarabine, cytochalasin B, dacarbazine, dactinomycin, darbepoetin alfa, dasatinib, daunorubicin, 1-dehydrotestosterone, denileukin, dexamethasone, dexrazoxane, dihydroxy anthracin dione, disulfiram, docetaxel, doxorubicin, emetine, epirubicin, erlotinib, epigallocatechin gallate, epoetin alfa, estramustine, ethidium bromide, etoposide, everolimus, filgrastim, finasunate, floxuridine, fludarabine, flurouracil (5-FU), fulvestrant, ganciclovir, geldanamycin, gemcitabine, glucocorticoids, gramicidin D, histrelin acetate, hydroxyurea, ibritumomab, idarubicin, ifosfamide, imatinib, irinotecan, interferons, interferon alfa-2a, interferon alfa-2b, ixabepilone, lactate dehydrogenase A (LDH-A), lenalidomide, letrozole, leucovorin, levamisole, lidocaine, lomustine, mechlorethamine, melphalan, 6-mercaptopurine, mesna, methotrexate, methoxsalen, metoprine, metronidazole, mithramycin, mitomycin-C, mitoxantrone, nandrolone, nelarabine, nilotinib, nofetumomab, oprelvekin, oxaliplatin, paclitaxel, pemetrexed, pentostatin, palifermin, pamidronate, pegademase, pegaspargase, pegfilgrastim, pemetrexed disodium, plicamycin, porfimer sodium, procaine, procarbazine, propranolol, puromycin, quinacrine, radicicol, radioactive isotopes, raltitrexed, rapamycin, rasburicase, salinosporamide A, sargramostim, sunitinib, temozolomide, teniposide, tetracaine, 6-thioguanine, thiotepa, topotecan, toremifene, trastuzumab, treosulfan, tretinoin, valrubicin, vinblastine, vincristine, vindesine, vinorelbine, zoledronate, or a combination thereof.

The antimetabolites may be, e.g., methotrexate, 6-mercaptopurine, 6-thioguanine, cytarabine, 5-fluorouracil decarbazine, cladribine, pemetrexed, gemcitabine, capecitabine, hydroxyurea, mercaptopurine, fludarabine, pralatrexate, clofarabine, cytarabine, decitabine, floxuridine, nelarabine, trimetrexate, thioguanine, pentostatin, or a combination thereof.

The alkylating agent may be, e.g., mechlorethamine, thiotepa, chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, altretamine, cyclophosphamide, ifosfamide, hexamethylmelamine, altretamine, procarbazine, dacarbazine, temozolomide, streptozocin, carboplatin, cisplatin, oxaliplatin, uramustine, bendamustine, trabectedin, semustine, or a combination thereof.

The anthracycline may be, e.g., daunorubicin, doxorubicin, aclarubicin, aldoxorubicin, amrubicin, annamycin, carubicin, epirubicin, idarubicin, mitoxantrone, valrubicin, or a combination thereof.

The antibiotic may be, e.g., dactinomycin, bleomycin, mithramycin, anthramycin (AMC), ampicillin, bacampicillin, carbenicillin, cloxacillin, dicloxacillin, flucloxacillin, mezlocillin, nafcillin, oxacillin, piperacillin, pivampicillin, pivmecillinam, ticarcillin, aztreonam, imipenem, doripenem, ertapenem, meropenem, cephalosporins, clarithromycin, dirithromycin, roxithromycin, telithromycin, lincomycin, pristinamycin, quinupristin, amikacin, gentamicin, kanamycin, neomycin, netilmicin, paromomycin, tobramycin, streptomycin, sulfamethizole, sulfamethoxazole, sulfisoxazole, demeclocycline, minocycline, oxytetracycline, tetracycline, penicillin, amoxicillin, cephalexin, erythromycin, clarithromycin, azithromycin, ciprofloxacin, levofloxacin, ofloxacin, doxycycline, clindamycin, metronidazole, tigecycline, chloramphenicol, metronidazole, tinidazole, nitrofurantoin, vancomycin, teicoplanin, telavancin, linezolid, cycloserine, rifamycins, polymyxin B, bacitracin, viomycin, capreomycin, quinolones, daunorubicin, doxorubicin, 4′-deoxydoxorubicin, epirubicin, idarubicin, plicamycin, mitomycin-c, mitoxantrone, or a combination thereof.

The anti-mitotic agent may be, e.g., vincristine, vinblastine, vinorelbine, docetaxel, estramustine, ixabepilone, paclitaxel, maytansinoid, a dolastatin, a cryptophycin, or a combination thereof.

The signal transduction inhibitor may be, e.g., imatinib, trastuzumab, erlotinib, sorafenib, sunitinib, temsirolimus, vemurafenib, lapatinib, bortezomib, cetuximab panitumumab, matuzumab, gefitinib, STI 571, rapamycin, flavopiridol, imatinib mesylate, vatalanib, semaxinib, motesanib, axitinib, afatinib, bosutinib, crizotinib, cabozantinib, dasatinib, entrectinib, pazopanib, lapatinib, vandetanib, or a combination thereof.

The gene expression modulator may be, e.g., a siRNA, a shRNA, an antisense oligonucleotide, an HDAC inhibitor, or a combination thereof. An HDAC inhibitor may be, e.g., trichostatin A, trapoxin B, valproic acid, vorinostat, belinostat, LAQ824, panobinostat, entinostat, tacedinaline, mocetionstat, givinostat, resminostat, abexinostat, quisinostat, rocilinostat, practinostat, CHR-3996, butyric acid, phenylbutyric acid, 4SC202, romidepsin, sirtinol, cambinol, EX-527, nicotinamide, or a combination thereof. An antisense oligonucleotide may be, e.g., custirsen, apatorsen, AZD9150, trabadersen, EZN-2968, LErafAON-ETU, or a combination thereof. An siRNA may be, e.g., ALN-VSP, CALAA-01, Atu-027, SPC2996, or a combination thereof.

The hormone therapy may be, e.g., a luteinizing hormone-releasing hormone (LHRH) antagonist. The hormone therapy may be, e.g., firmagon, leuproline, goserelin, buserelin, flutamide, bicalutadmide, ketoconazole, aminoglutethimide, prednisone, hydroxyl-progesterone caproate, medroxy-progesterone acetate, megestrol acetate, diethylstil-bestrol, ethinyl estradiol, tamoxifen, testosterone propionate, fluoxymesterone, flutamide, raloxifene, droloxifene, iodoxyfene, 4-hydroxytamoxifen, trioxifene, keoxifene, LY117018, onapristone, toremifine citrate, megestrol acetate, exemestane, fadrozole, vorozole, letrozole, anastrozole, nilutamide, tripterelin, histerelin, arbiraterone, medroxyprogesterone acetate, diethylstilbestrol, premarin, fluoxymesterone, tretinoin, fenretinide, troxacitabine, or a combination thereof.

The apoptosis inducers may be, e.g., a recombinant human TNF-related apoptosis-inducing ligand (TRAIL), camptothecin, bortezomib, etoposide, tamoxifen, or a combination thereof.

The angiogenesis inhibitors may be, e.g., sorafenib, sunitinib, pazopanib, everolimus or a combination thereof.

The immunotherapy agent may be, e.g., a monoclonal antibody, cancer vaccine (e.g., a dendritic cell (DC) vaccine), oncolytic virus, cytokine, adoptive T cell therapy, Bacille Calmette-Guérin (BCG), GM-CSF, thalidomide, lenalidomide, pomalidomide, imiquimod, or a combination thereof. The monoclonal antibody may be, e.g., anti-CTLA4, anti-PD1, anti-PD-L1, anti-LAG3, anti-KIR, or a combination thereof. The monoclonal antibody may be, e.g., alemtuzumab, trastuzumab, ibritumomab tiuxetan, brentuximab vedotin, trastuzumab, ado-trastuzumab emtansine, blinatumomab, bevacizumab, cetuximab, pertuzumab, panitumumab, ramucirumab, obinutuzumab, ofatumumab, rituximab, pertuzumab, tositumomab, gemtuzumab ozogamicin, tositumomab, or a combination thereof. The cancer vaccine may be, e.g., Sipuleucel-T, BioVaxID, NeuVax, DCVax, SuVaxM, CIMAvax®, Provenge®, hsp110 chaperone complex vaccine, CDX-1401, MIS416, CDX-110, GVAX Pancreas, HyperAcute™ Pancreas, GTOP-99 (MyVax®), or Imprime PGG®. The oncolytic virus may be, e.g., talimogene laherparepvec. The cytokine may be, e.g., IL-2, IFNα, or a combination thereof. The adoptive T cell therapy may be, e.g., tisagenlecleucel, axicabtagene ciloleucel, or a combination thereof.

The DNA damage repair inhibitor may be, e.g., a PARP inhibitor, a cell checkpoint kinase inhibitor, or a combination thereof. The PARP inhibitor may be, e.g., olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338 or a combination thereof. The cell checkpoint kinase inhibitor may be, e.g., MK-1775 or AZD1775, AZD7762, LY2606368, PF-0477736, AZD0156, GDC-0575, ARRY-575, CCT245737, PNT-737 or a combination thereof.

### EXAMPLES

The following examples are meant to illustrate the invention. They are not meant to limit the invention in any way.

Reactions were typically performed at room temperature (rt or RT) under a nitrogen atmosphere using dry solvents (Sure/Seal™) if not described otherwise in the Examples below. Reactions were monitored by TLC or by injection of a small aliquot on a Waters Acquity-H UPLC® Class system using an Acquity® UPLC HSS C18 2.1×30 mm column eluting with a gradient (1.86 min) of acetonitrile (15% to 98%) in water (both containing 0.1% formic acid). Purifications by preparative HPLC were performed on a Teledyne Isco Combi Flash® EZ Prep system using either Phenomenex Gemini® 5 μm NX-C18 110 Å 150×21.2 mm column at a flow of 40 mL/min over 12 min (<100 mg or multiple injections of <100 mg) or HP C18 RediSep® Rf gold column (>100 mg) eluting with an appropriate gradient of acetonitrile in water (both containing 0.1% formic acid) unless otherwise specified. The gradient was selected based on the retention time observed by reaction monitoring on the Waters Acquity-H UPLC® Class system (see above). Fractions containing the desired compounds were combined and finally lyophilized. Purifications by silica gel chromatography were performed on a Teledyne Isco Combi Flash® Rf system using RediSep® Rf silica gel columns of appropriate sizes. Purity of final Compounds was assessed by injection of a small aliquot on a Waters Acquity-H UPLC® Class system using an Acquity® UPLC BEH C18 2.1×50 mm column eluting with a gradient (7 min) of acetonitrile (2% to 98%) in water (both containing 0.1% formic acid).

### Example 1. Preparation of Compounds

### Intermediates

The Intermediates exemplified in table 2 were used to prepare the Compounds described herein.

TABLE 2

| | |
|---|---|
| 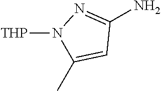 | Intermediate 1 |
| 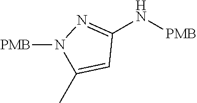 | Intermediate 2 |
| 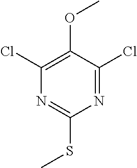 | Intermediate 3 |
| 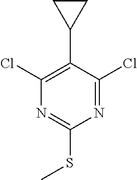 | Intermediate 4 |
| 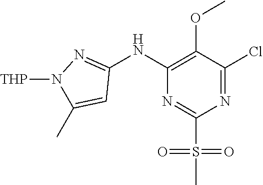 | Intermediate 5 |
| 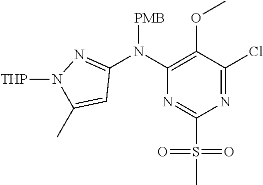 | Intermediate 6 |
| 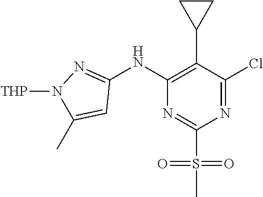 | Intermediate 7 |
| 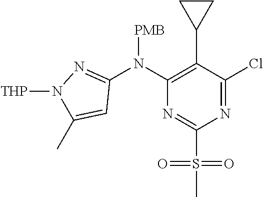 | Intermediate 8 |
| 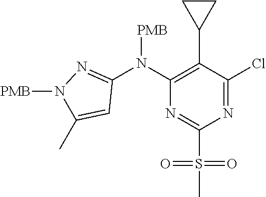 | Intermediate 9 |
| 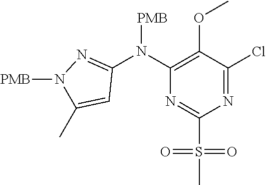 | Intermediate 10 |
| 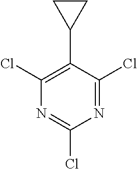 [SMILES: Clc1nc(Cl)c(C2CC2)c(Cl)n1] | Intermediate 11 |
| 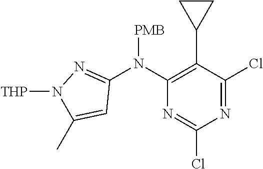 | Intermediate 12 |
| 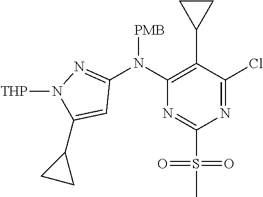 | Intermediate 13 |

### Intermediate 1/5-methyl-1-(tetrahydro-2H-pyran-2-yl)-1H-pyrazol-3-amine

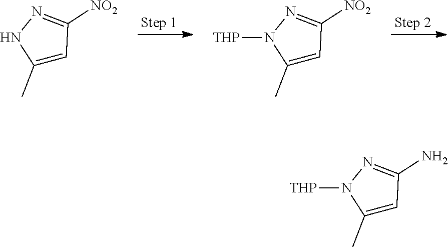

### Step 1/5-methyl-3-nitro-1-(tetrahydro-2H-pyran-2-yl)-1H-pyrazole

To 5-methyl-3-nitro-1H-pyrazole (50.0 g, 393 mmol) in toluene (1.0 L) was added TFA (6.0 mL, 78.7 mmol) and 3,4-dihydro-2H-pyran 3,4-dihydro-2H-pyran (39.5 mL, 433 mmol) in one portion. The reaction mixture was heated to 90° C. Reaction mixture was cooled to rt then poured on 10% KHCO₃ (1.0 L). Phases were separated, the aqueous phase was back extracted twice with EtOAc (2×200 mL). The combined organic phases were washed with brine (200 mL). Activated charcoal (7.5 g, 15% w) was added and the suspension was stirred for 15 minutes then anhydrous Na₂SO₄ (50 g) was added, and the suspension was stirred for 30 minutes. The resulting suspension was filtered over a Celite® pad and the cake was rinsed twice with EtOAc (2×100 mL). The combined organic phases were concentrated in vacuo to afford 5-methyl-3-nitro-1-(tetrahydro-2H-pyran-2-yl)-1H-pyrazole (88.04 g, 89% pure, 95% yield corrected). ¹H NMR (400 MHz, CDCL₃) δ ppm 1.46-1.72 (m, 3H), 1.79-1.89 (m, 1H), 1.98-2.08 (m, 1H), 2.21 (s, 3H), 2.28-2.45 (m, 1H), 3.40-3.72 (m, 3H), 3.96-4.08 (m, 1H), 5.03 (dd, J=10.3, 2.4 Hz, 1H), 5.42 (s, 1H).

### Step 2/Intermediate 1

5-methyl-3-nitro-1-(tetrahydro-2H-pyran-2-yl)-1H-pyrazole (78.7 g, 373 mmol) was suspended in methanol (1.25 L) then this suspension was charged in a 2-gallon Parr pressure reactor. 10% Palladium on charcoal (wet support) (7.9 g, 10% w) was added then the reactor was purged three times with 30 PSI of nitrogen and three times with 20 PSI of hydrogen. Hydrogen pressure was set at 35 PSI and the reaction was stirred at rt for 90 minutes. Reactor and filter were rinsed with methanol (2×500 mL) then solvent was concentrated to dryness to gives 71.9 g of crude desired compound as a brown oil. The crude product was pre-absorbed on silica gel (80 g) then purified by silica gel chromatography eluting with a gradient of MeOH (0 to 4%) in DCM. Pure fractions were pooled and concentrated to dryness to afford Intermediate 1 (57.7 g, 97% pure by HPLC at 215 nm, 86% corrected yield) as an amber oil. ¹H NMR (400 MHz, CDCl₃) δ ppm 1.46-1.72 (m, 3H), 1.79-1.89 (m, 1H), 1.98-2.08 (m, 1H), 2.21 (s, 3H), 2.28-2.45 (m, 1H), 3.40-3.72 (m, 3H), 3.96-4.08 (m, 1H), 5.03 (dd, J=10.3, 2.4 Hz, 1H), 5.42 (s, 1H).

### Intermediate 2/N, 1-bis(4-methoxybenzyl)-5-methyl-1H-pyrazol-3-amine

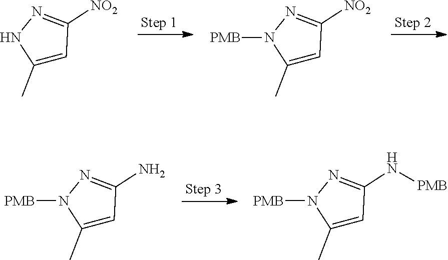

### Step 1/1-(4-methoxybenzyl)-5-methyl-3-nitro-1H-pyrazole

A mixture of 5-methyl-3-nitro-1H-pyrazole (19.2 g, 151 mmol), 1-(chloromethyl)-4-methoxy-benzene (21.6 mL, 159 mmol), K₂CO₃ (41.8 g, 302 mmol) in MeCN (200 mL) was heated at 80° C. for 3 hrs. The reaction mixture was filtered, and the filtrate was concentrated in vacuo. The resulting yellow solid was dissolved in DCM and purified by silica gel chromatography eluting with EtOAc (0 to 40%) in hexanes. The relevant fractions were combined and evaporated to give 1-[(4-methoxyphenyl)methyl]-5-methyl-3-nitro-pyrazole (23.0 g, 62% yield).

### Step 2/1-(4-methoxybenzyl)-5-methyl-1H-pyrazol-3-amine

To 1-(4-methoxybenzyl)-5-methyl-3-nitro-1H-pyrazole (21.5 g, 87.1 mmol) in MeOH (550 mL) was added zinc (56.9 g, 871 mmol) and NH₄HCO₂ (54.9 g, 871 mmol) at 0° C. The reaction mixture was stirred for 30 min, filtered through celite, and evaporated in vacuo to afford 1-(4-methoxybenzyl)-5-methyl-1H-pyrazol-3-amine (15.57 g, 82% yield) which was used in the next step without further purification.

### Step 3/Intermediate 2

A mixture of 1-(4-methoxybenzyl)-5-methyl-1H-pyrazol-3-amine (11.67 g, 53.71 mmol) and 4-methoxybenzaldehyde (6.53 mL, 53.7 mmol,) in DCM (200 mL) was stirred 30 min at rt. NaBH(OAc)₃ (12.52 g, 59.08 mmol) was added and the reaction mixture was stirred at rt for 2 h. The reaction mixture was quenched with aqueous saturated NaHCO₃ and extracted with DCM. The organic layer was evaporated in vacuo and purified by silica gel chromatography eluting with EtOAc. The relevant fractions were combined to give Intermediate 2 (12.57 g, 69% yield) as a white solid. UPLC-MS (+ESI) m/z=338.3 (M+H)⁺.

### Intermediate 3/4,6-dichloro-5-methoxy-2-(methylthio)pyrimidine

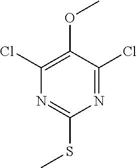

Intermediate 3 was prepared according to the procedure described in WO2016/166604 A1

### Intermediate 4/4,6-dichloro-5-cyclopropyl-2-(methylthio)pyrimidine

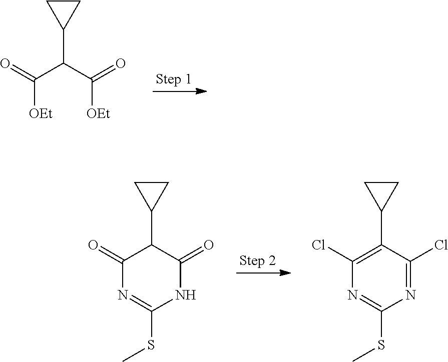

### Step 1/5-cyclopropyl-2-(methylthio)pyrimidine-4,6(1H,5H)-dione

A mixture of 1,3-diethyl 2-cyclopropylpropanedioate (111 g, 80% pure, 435 mmol), thiourea (40.0 g, 526 mmol) in MeOH (420 mL) was stirred for 10 min then a MeOH solution of NaOMe (25% w/w, 108 g, 500 mmol) was added. The final mixture was heated to 50° C. for 16 hrs. The mixture was cooled to rt and MeI (74.0 g, 522 mmol) was added. The final mixture was stirred for 22 hrs at rt, then quenched with water (500 mL) and acidified to pH˜1 by addition of conc. HCl solution (10 mL). The mixture was concentrated under reduced pressure to remove most of the methanol. The suspension was cooled to 20° C. and filtered on a Büchner funnel with a paper filter. The cake was rinsed with water (2×100 mL). The solid was dried under vacuum for 3 hrs affording 5-cyclopropyl-2-(methylthio)pyrimidine-4,6(1H,5H)-dione (80.0 g, 80% yield, 86% pure by w/w quantitative NMR) as a white powder which was used in the next step without further purification. UPLC-MS (+ESI) m/z=199.1 (M+H)⁺.

### Step 2/Intermediate 4

To 5-cyclopropyl-2-(methylthio)pyrimidine-4,6(1H,5H)-dione (76.5 g, 332 mmol) in MeCN (300 mL) and N,N-dimethylformamide (38 mL) was added POCl₃ (60 mL, 644 mmol) diluted with MeCN (100 mL) and added gradually over 35 min. The final mixture was heated to 70° C. for 3 hrs, then at 20° C. for 16 h. The resulting reaction mixture was cooled in a water bath and then quenched by addition of water (750 mL) over 20 min. The resulting solid was filtered on a Büchner funnel with a paper filter and rinsed with water (2×200 mL). After removal of most of the water by suction, the cake was dried under vacuum at 50° C. for 45 hrs affording Intermediate 4 (77.0 g, 89% yield, 89.0% purity by HPLC @ 215 nm) as a yellow solid. UPLC-MS (+ESI) m/z=235.0 (M+H)⁺.

### Intermediate 5/6-chloro-5-methoxy-N-(5-methyl-1-(tetrahydro-2H-pyran-2-yl)-1H-pyrazol-3-yl)-2-(methylsulfonyl)pyrimidin-4-amine

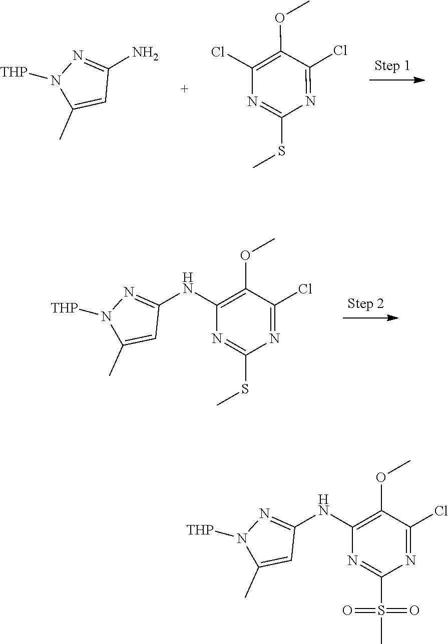

### Step 1/6-chloro-5-methoxy-N-(5-methyl-1-(tetrahydro-2H-pyran-2-yl)-1H-pyrazol-3-yl)-2-(methylthio)pyrimidin-4-amine

A solution of NaHMDS (159.9 mL, 159.9 mmol) was added over 30 min to a solution of Intermediate 1 (9.66 g, 53.3 mmol) in THF (320 mL) at rt, under N₂ atmosphere. Intermediate 3 (12.00 g, 53.31 mmol) was then added and the reaction mixture was stirred at 80° C. for 3 h, at which time UPLC-MS showed complete consumption of starting material. The mixture was allowed to cool to rt and then poured into 500 ml of saturated aqueous NaHCO₃. The resulting mixture was extracted with EtOAc (3×). The combined organics were washed with brine, dried over Na₂SO₄, filtered and concentrated. The residue was purified by silica gel chromatography eluting with EtOAc (0 to 80%) in Hep to afford 6-chloro-5-methoxy-N-(5-methyl-1-(tetrahydro-2H-pyran-2-yl)-1H-pyrazol-3-yl)-2-(methylthio)pyrimidin-4-amine (10.0 g, 51% yield) as a light-yellow solid. UPLC-MS (+ESI) m/z=370.0 (M+H)^{+ 1}H NMR (400 MHz, DMSO-d₆) δ ppm 1.44-1.56 (m, 2H), 1.58-1.74 (m, 1H), 1.83 (dd, J=13.0, 2.2 Hz, 1H), 1.98 (d, J=13.0 Hz, 1H), 2.21-2.28 (m, 1H), 2.30 (s, 3H), 2.45 (s, 3H), 3.58-3.68 (m, 1H), 3.74 (s, 3H), 3.90 (d, J=11.5 Hz, 1H), 5.32 (dd, J=9.8, 2.2 Hz, 1H), 6.46 (s, 1H), 9.78 (s, 1H).

### Step 2/Intermediate 5

Hydrogen peroxide 35% (w/w) in water (12.0 mL, 124 mmol) was added to a mixture of 6-chloro-5-methoxy-N-(5-methyl-1-(tetrahydro-2H-pyran-2-yl)-1H-pyrazol-3-yl)-2-(methylthio)pyrimidin-4-amine (6.54 g, 17.7 mmol), tetrabutylammonium hydrogen sulfate (960.0 mg, 2.83 mmol) and sodium tungstate dihydrate (583 mg, 1.77 mmol) in EtOAc (106 mL) and THF (106 mL). The mixture was heated at 50° C. for 2 hrs. The reaction mixture was then cooled to 0° C., diluted with EtOAc and washed with 10% NaHSO3 (260 mL). The layers were separated, and the aqueous layer was back extracted with EtOAc (2×). The combined organic layers were washed with brine, dried over MgSO₄, filtered, and concentrated to give crude Intermediate 5 (7.10 g, 88% purity by HPLC @ 254 nm).) as a light-orange solid, UPLC-MS (+ESI) m/z=402.0 (M+H)^{+ 1}H NMR (400 MHz, DMSO-d₆) δ ppm 1.45-1.57 (m, 2H), 1.59-1.75 (m, 1H), 1.85 (dd, J=13.0, 2.4 Hz, 1H), 1.94-2.03 (m, 1H), 2.21-2.30 (m, 1H), 2.32 (s, 3H), 3.31-3.32 (m, 3H), 3.64 (td, J=11.0, 3.4 Hz, 1H), 3.87 (s, 3H), 3.90 (dd, J=11.2, 1.7 Hz, 1H), 5.35 (dd, J=10.0, 2.4 Hz, 1H), 6.55 (s, 1H), 10.50 (s, 1H).

### Intermediate 6/6-chloro-5-methoxy-N-(4-methoxybenzyl)-N-(5-methyl-1-(tetrahydro-2H-pyran-2-yl)-1H-pyrazol-3-yl)-2-(methylsulfonyl)pyrimidin-4-amine

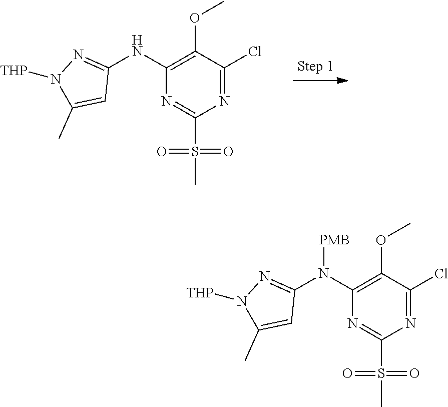

4-Methoxybenzyl chloride (5.18 mL, 35.1 mmol) was added to a mixture of Intermediate 5 (7.05 g, 17.6 mmol) and K₂CO₃ (7.28 g, 52.6 mmol) in MeCN (35 mL) under nitrogen atmosphere. The mixture was stirred at 80° C. for 18 hours then cooled to rt and diluted with water. The mixture was extracted with EtOAc (3×) and the combined organic layers were dried over Na₂SO₄, filtered and concentrated. The residue was purified by silica gel column chromatography eluting with Hep/EtOAc (0 to 100%) to afford Intermediate 6 (5.90 g, 65%) as a white solid. UPLC-MS (+ESI) m/z=522.0 (M+H)^{+ 1}H NMR (400 MHz, DMSO-d₆) δ ppm 1.46-1.56 (m, 2H), 1.57-1.70 (m, 1H), 1.80 (dd, J=13.3, 2.8 Hz, 1H), 1.90-2.00 (m, 1H), 2.08-2.21 (m, 1H), 2.28 (s, 3H), 3.28 (s, 3H), 3.32 (s, 3H), 3.56-3.65 (m, 1H), 3.70 (s, 3H), 3.78-3.87 (m, 1H), 5.03 (s, 2H), 5.38 (dd, J=9.0, 2.2 Hz, 1H), 6.01 (s, 1H), 6.83 (d, J=8.6 Hz, 2H), 7.31 (d, J=8.6 Hz, 2H).

### Intermediate 7/6-chloro-5-cyclopropyl-N-(5-methyl-1-(tetrahydro-2H-pyran-2-yl)-1H-pyrazol-3-yl)-2-(methylsulfonyl)pyrimidin-4-amine

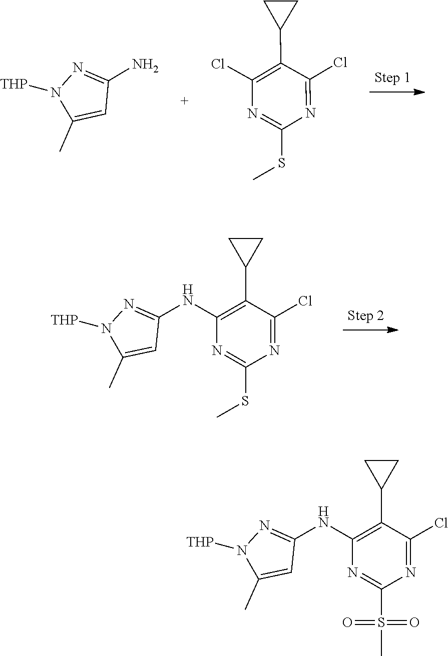

### Step 1/6-chloro-5-cyclopropyl-N-(5-methyl-1-(tetrahydro-2H-pyran-2-yl)-1H-pyrazol-3-yl)-2-(methylthio)pyrimidin-4-amine

To a solution of Intermediate 1 (10.9 g, 46.4 mmol) was dissolved in THF (270 mL) was added NaHMDS (104 mL, 104 mmol, 1M in THF) then Intermediate 4 (8.40 g, 46.4 mmol, corrected for purity: 89%). The resulting solution was heated to reflux for 30 minutes, cooled to rt and concentrated to dryness. The residue was dissolved in EtOAc (500 mL) and wash with water (250 mL). The aqueous phase was back extracted twice with EtOAc (2×100 mL) then the pooled organic phases were successively washed with water (100 mL), brine (100 mL), dried over Na₂SO₄, filtered. At this stage, crude material from a previous test on 1.12 g of Intermediate 4 was added to the batch. The combined organic phases were partially evaporated to 80 mL and Hep (190 mL) was added to crystallize the product. Solid was collected by filtration, then the cake was washed with heptane (100 mL). Solid was dried under vacuum at 45-50° C. until constant weight was observed to afford 6-chloro-5-cyclopropyl-N-(5-methyl-1-(tetrahydro-2H-pyran-2-yl)-1H-pyrazol-3-yl)-2-(methylthio)pyrimidin-4-amine (14.06 g, 73% yield corrected) as an off-white solid. ¹H NMR (400 MHz, CDCL₃) δ ppm 0.73 (d, J=4.2 Hz, 2H), 1.18 (dd, J=7.9, 1.6 Hz, 2H), 1.42-1.81 (m, 5H), 1.89 (br d, J=12.2 Hz, 1H), 2.04-2.18 (m, 1H), 2.36 (s, 4H), 2.55 (s, 3H), 3.57-3.75 (m, 1H), 4.11 (br d, J=11.5 Hz, 1H), 5.19 (dd, J=10.4, 2.3 Hz, 1H), 6.67 (s, 1H), 7.84 (s, 1H).

### Step 2/Intermediate 7

6-chloro-5-cyclopropyl-N-(5-methyl-1-(tetrahydro-2H-pyran-2-yl)-1H-pyrazol-3-yl)-2-(methylthio)pyrimidin-4-amine (450 mg, 1.2 mmol) was dissolved in MeOH (10 mL) and water (5 mL). Oxone (398 mg, 2.4 mmol) was added at rt. The resulting mixture was stirred for 16 hrs at rt. The resulting mixture was poured in water, then extracted with EtOAc (3×). The organic layers were dried over anhydrous Na₂SO₄, filtered, and concentrated under reduced pressure. The residue was purified by Prep-TLC (DCM/MeOH=15:1) to afford Intermediate 7 (165 mg, 34% yield) as a white solid. UPLC-MS (+ESI) m/z=412.0 (M+H)⁺.

### Intermediate 8/6-chloro-5-cyclopropyl-N-(4-methoxybenzyl)-N-(5-methyl-1-(tetrahydro-2H-pyran-2-yl)-1H-pyrazol-3-yl)-2-(methylsulfonyl)pyrimidin-4-amine

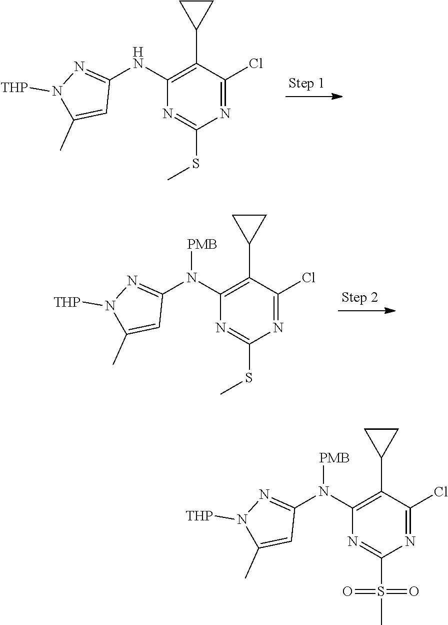

### Step 1/6-chloro-5-cyclopropyl-N-(4-methoxybenzyl)-N-(5-methyl-1-(tetrahydro-2H-pyran-2-yl)-1H-pyrazol-3-yl)-2-(methylthio)pyrimidin-4-amine

6-chloro-5-cyclopropyl-N-(5-methyl-1-(tetrahydro-2H-pyran-2-yl)-1H-pyrazol-3-yl)-2-(methylthio)pyrimidin (see Intermediate 7, Step 1) (14.0 g, 36.9 mmol) and K₂CO₃ (10.2 g, 73.7 mmol) were suspended in N,N-dimethylacetamide (140 mL) then 4-methoxybenzyl chloride (6.93 g, 44.2 mmol) was added in one portion. The suspension was heated to 90° C. for 9 hrs. The reaction mixture was cooled to rt and poured over a mixture of EtOAc (100 mL) and water (700 mL). Phases were separated and the aqueous phase was back extracted twice with EtOAc (2×100 mL). The pooled organic phases were successively washed with water (2×100 mL) and brine (100 mL), dried over Na₂SO₄, filtered, and concentrated to dryness. The crude product was suspended in diisopropyl ether (100 mL) and heated to reflux for 30 minutes. The resulting slurry was slowly cooled to rt then heptane (150 mL) was added. The solids were collected by filtration, washed with a mixture of 15% diisopropyl ether in heptane (50 mL). Solids were dried under vacuum at 45-50° C. until constant weight was observed to afford 6-chloro-5-cyclopropyl-N-(4-methoxybenzyl)-N-(5-methyl-1-(tetrahydro-2H-pyran-2-yl)-1H-pyrazol-3-yl)-2-(methylthio)pyrimidin-4-amine (14.72 g, 80% yield) as an off-white solid. ¹H NMR (400 MHz, CDCl₃) δ ppm 0.34 (br d, J=5.4 Hz, 2H), 0.53 (td, J=8.4, 4.9 Hz, 1H), 0.58-0.68 (m, 1H), 1.02-1.12 (m, 1H), 1.51-1.77 (m, 5H), 1.79-1.91 (m, 1H), 2.04-2.17 (m, 1H), 2.24-2.39 (m, 4H), 2.44 (s, 3H), 3.57-3.68 (m, 1H), 3.78 (s, 3H), 4.00 (br d, J=11.5 Hz, 1H), 5.05-5.14 (m, 1H), 5.18 (dd, J=9.5, 2.4 Hz, 1H), 5.21-5.29 (m, 1H), 5.70 (s, 1H), 6.81 (d, J=8.8 Hz, 2H), 7.34 (d, J=8.6 Hz, 2H).

### Step 2/Intermediate 8

To a solution of 6-chloro-5-cyclopropyl-N-(4-methoxybenzyl)-N-(5-methyl-1-(tetrahydro-2H-pyran-2-yl)-1H-pyrazol-3-yl)-2-(methylthio)pyrimidin-4-amine (11.71 g, 23.42 mmol) in EtOAc (210 mL) and THF (210 mL) was added successively tetrabutylammonium hydrogen sulfate (1.27 g, 3.75 mmol), sodium tungstate dihydrate (772 mg, 3.35 mmol) and hydrogen peroxide 35% in water (15.9 g, 164 mmol). The resulting solution was heated to 50° C. for 4.5 hrs. The resulting reaction mixture was cooled to 5-10° C. and poured over 10% NaHSO₃ (0.8 L). Phases were separated then aqueous phase was back extracted twice with ethyl acetate (2×100 mL). The pooled organic phases were successively washed with water (100 mL), brine (100 mL), dried over Na₂SO₄, filtered, and concentrated almost to dryness. Heptane (100 mL) was added and concentrated to dryness to give 12.5 g of crude product as a pale-yellow solid. The solid was suspended in diisopropyl ether (70 mL) and heated to reflux for 45 minutes. Heating was stopped and the slurry was stirred for 0.5 hours at rt. Heptane (70 mL) was added and then solid was collected by filtration. The cake was washed with heptane (35 mL) and finally dried under vacuum at 45-50° C. to afford Intermediate 8 (11.85 g, 94% yield) as an off-white solid. ¹H NMR (400 MHz, CDCl₃) δ ppm 0.31-0.43 (m, 2H), 0.62 (td, J=8.5, 4.5 Hz, 1H), 0.65-0.75 (m, 1H), 1.14 (tt, J=8.4, 5.8 Hz, 1H), 1.53-1.77 (m, 3H), 1.85 (br dd, J=13.1, 2.6 Hz, 1H), 2.03-2.16 (m, 1H), 2.32 (s, 4H), 3.20 (s, 3H), 3.59-3.69 (m, 1H), 3.77 (s, 3H), 4.02 (br d, J=11.2 Hz, 1H), 5.05-5.13 (m, 1H), 5.16-5.27 (m, 2H), 5.79 (s, 1H), 6.81 (d, J=8.8 Hz, 2H), 7.39 (d, J=8.8 Hz, 2H).

### Intermediate 9/6-chloro-5-cyclopropyl-N-(4-methoxybenzyl)-N-(1-(4-methoxybenzyl)-5-methyl-1H-pyrazol-3-yl)-2-(methylsulfonyl)pyrimidin-4-amine

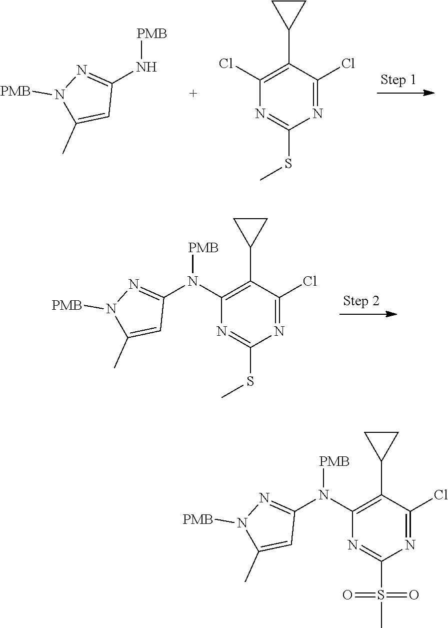

### Step 1/6-chloro-5-cyclopropyl-N-(4-methoxybenzyl)-N-(1-(4-methoxybenzyl)-5-methyl-1H-pyrazol-3-yl)-2-(methylthio)pyrimidin-4-amine

NaHMDS (1 M, 6.41 mL, 6.41 mmol) was added to a solution of Intermediate 2 (2.16 g, 6.41 mmol) in THF (10 mL) and stirred at rt for 10 min. Intermediate 4 (1.37 g, 5.83 mmol) was then added and the resulting mixture was stirred 80° C. for 30 min. The reaction mixture was concentrated in vacuo the residue was purified by silica gel chromatography eluting with EtOAc (70%) in Hep. The relevant fractions were combined to afford 6-chloro-5-cyclopropyl-N-(4-methoxybenzyl)-N-(1-(4-methoxybenzyl)-5-methyl-1H-pyrazol-3-yl)-2-(methylthio)pyrimidin-4-amine (1.47 g, 47% yield). UPLC-MS (+ESI) m/z=536.2 (M+H)⁺.

### Step 2/Intermediate 9

To 6-chloro-5-cyclopropyl-N-(4-methoxybenzyl)-N-(1-(4-methoxybenzyl)-5-methyl-1H-pyrazol-3-yl)-2-(methylthio)pyrimidin-4-amine (1.47 g, 2.74 mmol) in DCM (10 mL) at 0° C., was added mCPBA (946 mg, 5.48 mmol). The reaction was warmed to rt for 1 h. An additional 950 mg of mCPBA was added to complete the reaction. The reaction mixture was concentrated in vacuo the residue was purified by silica gel chromatography eluting with EtOAc (70 to 100%) in Hep. The relevant fractions were combined to give Intermediate 9 (1.13 g, 73% yield). UPLC-MS (+ESI) m/z=568.2 (M+H)⁺.

### Intermediate 10/6-chloro-5-methoxy-N-(4-methoxybenzyl)-N-(1-(4-methoxybenzyl)-5-methyl-1H-pyrazol-3-yl)-2-(methylsulfonyl)pyrimidin-4-amine

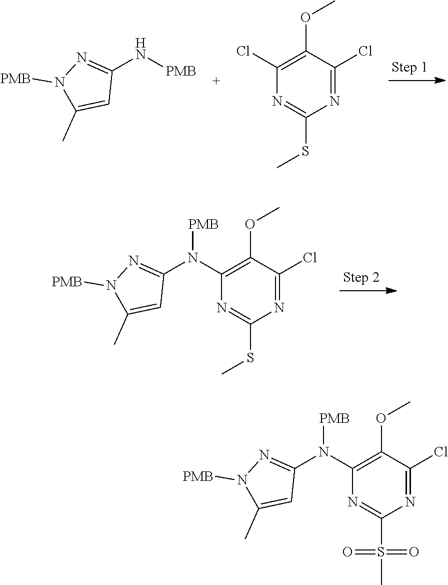

### Step 1/6-chloro-5-methoxy-N-(4-methoxybenzyl)-N-(1-(4-methoxybenzyl)-5-methyl-1H-pyrazol-3-yl)-2-(methylthio)pyrimidin-4-amine

NaHMDS (9.18 mL, 9.18 mmol, 1M) was added to a solution of Intermediate 2 (3.1 g, 9.19 mmol) in THF (20 mL) and stirred at rt for 10 min. The resulting mixture was added dropwise via syringe to Intermediate 3 (2.07 g, 9.18 mmol) in THF (20 mL) at rt. The reaction mixture was evaporated in vacuo and purified by silica gel chromatography on eluting with EtOAc (70%) in hexanes. The relevant fractions were combined to give 6-chloro-5-methoxy-N-(4-methoxybenzyl)-N-(1-(4-methoxybenzyl)-5-methyl-1H-pyrazol-3-yl)-2-(methylthio)pyrimidin-4-amine (1.82 g, 38% yield). UPLC-MS (+ESI) m/z=526.2 (M+H)⁺.

### Step 2/Intermediate 10

To 6-chloro-5-methoxy-N-(4-methoxybenzyl)-N-(1-(4-methoxybenzyl)-5-methyl-1H-pyrazol-3-yl)-2-(methylthio)pyrimidin-4-amine (1.82 g, 3.46 mmol) in DCM (10 mL) at 0° C., was added mCPBA (1.19 g, 6.92 mmol). The reaction was warmed to rt, stirred for 4 h and left in the fridge overnight. The reaction mixture was concentrated in vacuo and the residue was purified by silica gel chromatography eluting with EtOAc (50-100%) in Hex. The relevant fractions were combined and concentrated to give Intermediate 10 (1.14 g, 59% yield). UPLC-MS (+ESI) m/z=558.2 (M+H)⁺.

### Intermediate 11/2,4,6-trichloro-5-cyclopropylpyrimidine

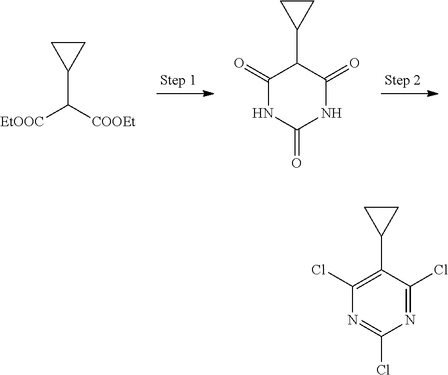 [SMILES: CCOC(=O)C(C(=O)OCC)C1CC1 ; O=C1NC(=O)C(C2CC2)C(=O)N1 ; Clc1nc(Cl)c(C2CC2)c(Cl)n1]

### Step 1/5-cyclopropylpyrimidine-2,4,6 (1H,3H,5H)-trione

A solution of diethyl 2-cyclopropylmalonate (15.6 g, 77.9 mmol) and urea (4.68 g, 77.9 mmol) in MeOH (390 mL) was maintained at 23° C. for 20 min. Sodium methoxide (25% in MeOH, 17.8 mL, 77.9 mmol) was then added to the reaction mixture. After the addition was complete, the reaction mixture was heated at reflux for 24 h. The resulting mixture was stirred at rt overnight and concentrated to dryness. The reaction mixture was cooled to 0° C. and water (25 mL) was slowly added. The precipitate was collected by filtration and washed with water (4 mL). The white solid was then dried under high vacuum. The filtrate, still containing the desired product, was concentrated to 5 mL and extracted with Me-THF (3×20 mL). The organic layers were combined and washed with brine, dried over Na₂SO₄, filtered, concentrated to dryness in vacuo was combined with the precipitate collected by filtration to afford 5-cyclopropylpyrimidine-2,4,6 (1H,3H,5H)-trione (7.40 g, 56% yield). UPLC-MS (+ESI): m/z=169.1 [M+H]⁺.

### Step 2/Intermediate 11

A mixture of 5-cyclopropylpyrimidine-2,4,6 (1H,3H,5H)-trione (7.40 g, 44.0 mmol) and N,N-dimethylaniline (16.9 mL, 132 mmol) in POCl₃ (82.0 mL, 880 mmol) was heated at reflux for 3 hrs, then cooled to rt. The reaction mixture was poured slowly on ice in an Erlenmeyer flask with vigorous stirring. After the addition was complete, the mixture was extracted with DCM (3×75 mL) and the combined organic layers were washed with brine, dried over Na₂SO₄, and concentrated to dryness in vacuo. The residue was purified by silica gel chromatography eluting with EtOAc (0-10%) in hexanes. The desired fractions were combined and concentrated to dryness in vacuo to afford Intermediate 11 (6.0 g, 61% yield). UPLC-MS (+ESI): m/z=223.9 [M+H]⁺.

### Intermediate 12/2,6-dichloro-5-cyclopropyl-N-(4-methoxybenzyl)-N-(5-methyl-1-(tetrahydro-2H-pyran-2-yl)-1H-pyrazol-3-yl)pyrimidin-4-amine

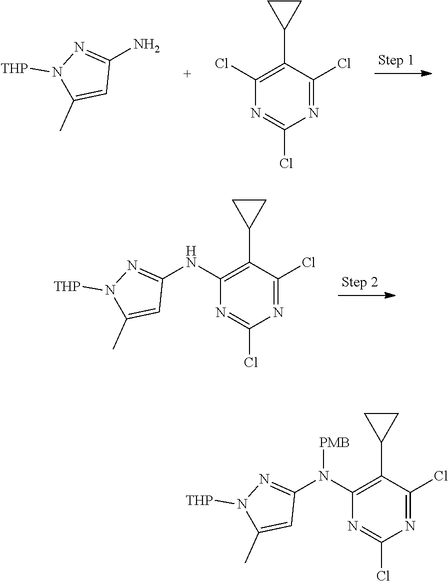

### Step 1/2,6-dichloro-5-cyclopropyl-N-(5-methyl-1-(tetrahydro-2H-pyran-2-yl)-1H-pyrazol-3-yl)pyrimidin-4-amine

To solution of Intermediate 11 (6.00 g, 26.8 mmol) and Intermediate 1 (5.35, 29.5 mmol) in dry THF (67 mL) at −78° C. under inert atmosphere was added NaHMDS solution (1 M in THF, 26.8 mL, 26.8 mmol). The resulting reaction mixture was stirred at this temperature for 30 minutes and then poured in water (100 mL) at 0° C. and diluted with DCM (100 mL). The layers were partitioned. Brine was added to the aqueous layer and back extracted with DCM (2×100 mL). The combined organic layers were washed with brine, dried over Na₂SO₄, filtered, and concentrated in vacuo. The crude material was purified by silica gel chromatography eluting with EtOAc (0 to 30%) in hexanes. The desired fractions were combined and concentrated to dryness in vacuo to afford 2,6-dichloro-5-cyclopropyl-N-(5-methyl-1-(tetrahydro-2H-pyran-2-yl)-1H-pyrazol-3-yl)pyrimidin-4-amine (4.10 g, 41% yield). UPLC-MS (+ESI): m/z=368.1 [M+H]⁺.

### Step 2/Intermediate 12

To a solution of 2,6-dichloro-5-cyclopropyl-N-(5-methyl-1-(tetrahydro-2H-pyran-2-yl)-1H-pyrazol-3-yl)pyrimidin-4-amine (1.70 g, 4.62 mmol) in DMA (10 mL) was added K₂CO₃ (1.91 g, 13.9 mmol). The mixture was purged with nitrogen for 5 min at rt. 1-(chloromethyl)-4-methoxy-benzene (1.45 g, 9.23 mmol, 1.25 mL) was added and the mixture was heated to 85° C. for 18 h. The resulting mixture was filtered and purified by preparative HPLC eluting with a gradient of MeCN (10 to 100%) in water both containing 0.1% formic acid. Appropriate fractions were combined and lyophilized to afford Intermediate 12 (1.69 g, 75% yield). UPLC-MS (+ESI) m/z=488.1 (M+H)⁺.

### Intermediate 13/6-chloro-5-cyclopropyl-N-(5-cyclopropyl-1-(tetrahydro-2H-pyran-2-yl)-1H-pyrazol-3-yl)-N-(4-methoxybenzyl)-2-(methylsulfonyl)pyrimidin-4-amine

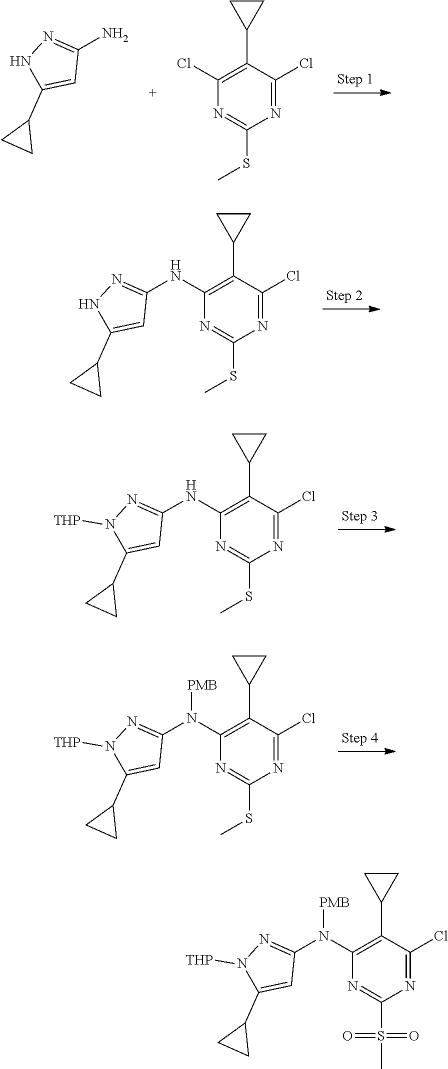

### Step 1/6-chloro-5-cyclopropyl-N-(5-cyclopropyl-1H-pyrazol-3-yl)-2-(methylthio)pyrimidin-4-amine

To a solution of Intermediate 4 (705 mg, 3.00 mmol) in DMF (5 mL) were added 5-cyclopropyl-1H-pyrazol-3-amine (517 mg, 4.20 mmol) and NaI (630 mg, 4.20 mmol). The mixture was stirred at 60° C. for 72 hrs. The reaction was quenched by addition of water, extracted with EtOAc (3×15 mL). The combined organic extracts were washed with brine, dried over Na₂SO₄, filtered, and concentrated to dryness. The residue was purified on silica gel column eluting with EtOAc (50-100%) in heptane. Appropriate fraction were combined and concentrated in vacuo to afford 6-chloro-5-cyclopropyl-N-(5-cyclopropyl-1H-pyrazol-3-yl)-2-(methylthio)pyrimidin-4-amine (586 mg, 61% yield) as a white solid.

### Step 2/6-chloro-5-cyclopropyl-N-(5-cyclopropyl-1-(tetrahydro-2H-pyran-2-yl)-1H-pyrazol-3-yl)-2-(methylthio)pyrimidin-4-amine

A mixture of 6-chloro-5-cyclopropyl-N-(5-cyclopropyl-1H-pyrazol-3-yl)-2-(methylthio)pyrimidin-4-amine (530 mg, 1.65 mmol), 4-methylbenzenesulfonic acid hydrate (60.0 mg, 315 μmol), 3,4-dihydro-2H-pyran (600 μL, 6.61 mmol,) in EtOAc (12 mL) was refluxed for 18 h. The reaction mixture was diluted with EtOAc (50 mL), washed with saturated aqueous NaHCO₃ (2 mL), and brine (15 mL), dried over Na₂SO₄, filtered, and concentrated to dryness. The residue was purified by flash chromatography eluting with a gradient of EtOAc (10-70%) in heptane. The appropriate fractions were combined and concentrated to afford 6-chloro-5-cyclopropyl-N-(5-cyclopropyl-1-(tetrahydro-2H-pyran-2-yl)-1H-pyrazol-3-yl)-2-(methylthio)pyrimidin-4-amine (668 mg, 100% yield) as an off-white solid. UPLC-MS (+ESI): m/z=406.5 [M+H]⁺.

### Step 3/6-chloro-5-cyclopropyl-N-(5-cyclopropyl-1-(tetrahydro-2H-pyran-2-yl)-1H-pyrazol-3-yl)-N-(4-methoxybenzyl)-2-(methylthio)pyrimidin-4-amine

To a solution of 6-chloro-5-cyclopropyl-N-(5-cyclopropyl-1-(tetrahydro-2H-pyran-2-yl)-1H-pyrazol-3-yl)-2-(methylthio)pyrimidin-4-amine (685 mg, 1.69 mmol) in DMF (8 mL) was added NaH (60% dispersion) (84.0 mg, 2.19 mmol, 60% purity) at 0° C. The mixture was stirred at 0° C. for 10 min then (chloromethyl)-4-methoxy-benzene (300 μL, 2.21 mmol,) and NEt₄I (31 mg, 84 μmol) were added. The final mixture was stirred at rt for 18 hrs. The mixture was diluted with EtOAc (100 mL), washed with water (20 mL) and brine (20 mL), dried over Na₂SO₄, filtered, and concentrated to dryness. The residue was purified on silica gel column eluting with a gradient of EtOAc (0-60%) in heptane. Combining and concentrating appropriate fractions afforded 6-chloro-5-cyclopropyl-N-(5-cyclopropyl-1-(tetrahydro-2H-pyran-2-yl)-1H-pyrazol-3-yl)-N-(4-methoxybenzyl)-2-(methylthio)pyrimidin-4-amine (650 mg, 73% yield) as a semi-solid. UPLC-MS (+ESI): m/z=526.6 [M+H]⁺.

### Step 4/Intermediate 13

6-chloro-5-cyclopropyl-N-(5-cyclopropyl-1-(tetrahydro-2H-pyran-2-yl)-1H-pyrazol-3-yl)-N-(4-methoxybenzyl)-2-(methylthio)pyrimidin-4-amine (650 mg, 1.24 mmol) was charged in a flask and dissolved in THF (10 mL) and EtOAc (10 mL). Tetrabutylammonium hydrogen sulfate (67.0 mg, 197 μmol), sodium tungstate dihydrate (41.0 mg, 124 μmol) and H₂O₂ (35% in water, 760 UL, 8.60 mmol) were successively added and the resulting solution was heated to 50° C. for 5 hrs. The reaction mixture was cooled to 5-10° C., poured over 10% NaHSO3 aqueous solution (10 mL). The organic phase was separated then aqueous phase was back extracted twice with ethyl acetate (20 mL). The pooled organic phases were washed with brine (20 mL), dried over anhydrous Na₂SO₄, filtered, and concentrated to dryness. The residue was purified on silica gel column eluting with a gradient of EtOAc (20-80%) in heptane. Appropriate fractions were combined and concentrated in vacuo to provide Intermediate 13 (432 mg, 63% yield) as a white solid. UPLC-MS (+ESI): m/z=558.6 [M+H]⁺.

### Compounds

### Compound 1/Method A/N²-(2-fluoro-4-(methylsulfonyl)phenyl)-5-methoxy-N⁴-(5-methyl-1H-pyrazol-3-yl)-6-(1-methyl-1H-pyrazol-4-yl)pyrimidine-2,4-diamine

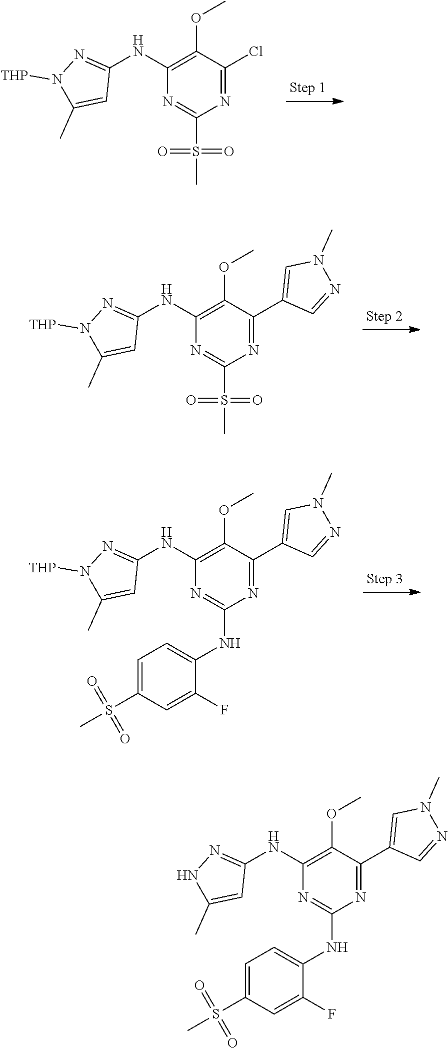

### Step 1/5-methoxy-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine

A solution of Intermediate 5 (1.00 g, 2.40 mmol) and 1-methylpyrazol-4-ylboronic acid (454 mg, 3.6 mmol) in 1,4-dioxane (30 mL) was added XPhosPdG₂ (189 mg, 0.24 mmol), K₃PO₄ (51 mg, 0.24 mmol) and water (3 mL). The resulting mixture was stirred at 90° C. for 16 hrs under nitrogen atmosphere. The solid was filtered out and the crude supernatant was purified by Prep-TLC (ethyl acetate/petroleum ether) to afford 5-methoxy-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (562 mg, 51% yield) as a pink solid. UPLC-MS (+ESI) m/z=448.1 (M+H)⁺. ¹H NMR (400 MHz, DMSO-d₆) δ 10.11 (s, 1H), 8.46 (s, 1H), 8.09-8.11 (s, 1H), 6.61 (s, 1H), 5.33-5.36 (m, 1H), 3.82-3.95 (m, 4H), 3.67-3.77 (m, 3H), 3.37-3.62 (m, 1H), 3.31-3.37 (m, 3H), 2.50-2.51 (m, 4H), 2.20-2.33 (m, 1H), 1.97-2.00 (m, 1H), 1.84-1.87 (m, 1H), 1.74-1.84 (m, 2H).

### Step 2/N²-(2-fluoro-4-(methylsulfonyl)phenyl)-5-methoxy-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To 4-methylsulfonylaniline (13.2 uL, 112 umol) in DMF (0.5 mL) was added NaH (8.90 mg, 223 umol, 60% purity) and stirred at rt for 10 min. To this mixture was added 5-methoxy-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (50 mg, 112 umol) and the resulting mixture was stirred at 80° C. for 1 h then heated to 120° C. for 15 hrs to complete the reaction. The crude reaction mixture was diluted with water then EtOAc and saturated aqueous NaHCO₃. The organic phase was separated, washed with brine, dried over Na₂SO₄, filtrated, and finally evaporated in vacuo to afford N²-(2-fluoro-4-(methylsulfonyl)phenyl)-5-methoxy-N⁴-(5-methyl-1-(tetrahydro-2H-pyran-2-yl)-1H-pyrazol-3-yl)-6-(1-methyl-1H-pyrazol-4-yl)pyrimidine-2,4-diamine (45 mg, 75% yield) as brown solid which was used as such in the next step without further purification. UPLC-MS (+ESI) m/z=539.2 (M+H)⁺.

### Step 3/Compound 1

To N²-(2-fluoro-4-(methylsulfonyl)phenyl)-5-methoxy-N⁴-(5-methyl-1-(tetrahydro-2H-pyran-2-yl)-1H-pyrazol-3-yl)-6-(1-methyl-1H-pyrazol-4-yl)pyrimidine-2,4-diamine (46 mg, 82 umol) was added 4M HCl in dioxane (2.0 mL). The reaction mixture was stirred at rt for 1 h. The crude reaction mixture was concentrated. The residue was dissolved in DMSO and purified by preparative HPLC eluting with a gradient of MeCN (25 to 55%) in water both containing 0.1% formic acid. Appropriate fractions were combined and lyophilized to afford Compound 1 (6.0 mg, 15% yield) as white solid. UPLC-MS (+ESI) m/z=473.1 (M+H)⁺. ¹H NMR (400 MHz, DMSO-d₆) δ 8.90 (s, 1H), 8.37 (s, 1H), 8.08 (s, 1H), 7.85-7.73 (m, 2H), 6.00 (s, 1H), 3.96 (s, 3H), 3.68 (s, 3H), 3.23 (s, 3H), 2.18 (s, 3H).

### Compound 2/Method B/N²-(2-fluoro-4-(methylsulfonyl)phenyl)-5-methoxy-N²-methyl-N⁴-(5-methyl-1H-pyrazol-3-yl)-6-(1-methyl-1H-pyrazol-4-yl)pyrimidine-2,4-diamine

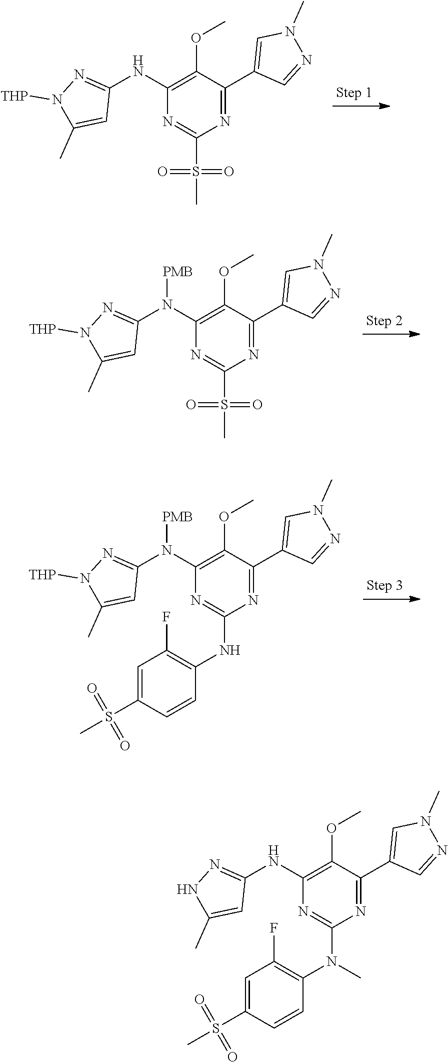

### Step 1/5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine

To a solution of 5-methoxy-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 1, Step 1) (5.30 g, 11.8 mmol) in MeCN (20 mL) at rt was added 1-(chloromethyl)-4-methoxy-benzene (2.40 mL, 17.8 mmol,) and K₂CO₃ (4.91 g, 35.5 mmol). The reaction mixture was stirred at 80° C. for 3 hrs. The crude reaction mixture was diluted with EtOAc, quenched with saturated aqueous NaHCO₃, then extracted with EtOAc. The organic layer was evaporated in vacuo and the residue purified on silica gel chromatography eluting with EtOAc (50-100%) in Heptane. The relevant fractions were evaporated to give 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (6.40 g, 95% yield) as a off-white foam. UPLC-MS (+ESI) m/z=568.3 (M+H)⁺.

### Step 2/N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To a solution of 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (1.4 g, 2.47 mmol) in DMPU (7 mL) was added 2-fluoro-4-methylsulfonyl-aniline (933 mg, 4.93 mmol) and NaH (197 mg, 4.93 mmol, 60% purity). The reaction mixture was stirred at 120° C. for 15 hrs. The crude reaction mixture was quenched with water, filtrated, and purified by preparative HPLC eluting with a gradient of MeCN in water both containing 0.1% formic acid. Appropriate fractions were combined and lyophilized to afford the desired product N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (761 mg, 46% yield) as a yellow solid. UPLC-MS (+ESI) m/z=677.3 (M+H)⁺.

### Step 3/Compound 2

To a solution of N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (98.0 mg, 145 umol) in DMF (1 mL) was added NaH (10.4 mg, 434 umol, 60% dispersion), then after 10 min MeI (27 uL, 434 umol,) was added and the reaction mixture was stirred at rt for 1 h. The crude reaction mixture was quenched with water, filtrated, and purified by preparative HPLC eluting with a gradient of MeCN in water both containing 0.1% formic acid. Appropriate fractions were combined and lyophilized to afford the intermediate N²-(2-fluoro-4-methylsulfonyl-phenyl)-5-methoxy-N⁴-[(4-methoxyphenyl)methyl]-N²-methyl-6-(1-methylpyrazol-4-yl)-N⁴-(5-methyl-1-tetrahydropyran-2-yl-pyrazol-3-yl)pyrimidine-2,4-diamine as brown solid. To this intermediate was added TFA (0.5 mL). The reaction mixture was stirred at 80° C. for 1 h. The crude reaction mixture was evaporated, diluted in DMSO, filtrated and purified by preparative HPLC eluting with a gradient of MeCN (10 to 50%) in water both containing 0.1% formic acid. Appropriate fractions were combined and lyophilized to afford Compound 2 (18 mg, 24% yield, 94% purity) as white solid. UPLC-MS (+ESI) m/z=487.2 (M+H)⁺. ¹H NMR (400 MHz, DMSO-d₆) δ 11.88 (s, 1H), 8.84 (s, 1H), 8.27 (s, 1H), 7.97 (d, J=0.6 Hz, 1H), 7.89-7.79 (m, 2H), 7.79-7.73 (m, 1H), 5.65 (s, 1H), 3.92 (s, 3H), 3.62 (s, 3H), 3.49 (s, 3H), 3.31 (s, 3H), 2.04 (s, 3H).

### Compound 3/Method B/N²-ethyl-N²-(2-fluoro-4-(methylsulfonyl)phenyl)-5-methoxy-N⁴-(5-methyl-1H-pyrazol-3-yl)-6-(1-methyl-1H-pyrazol-4-yl)pyrimidine-2,4-diamine

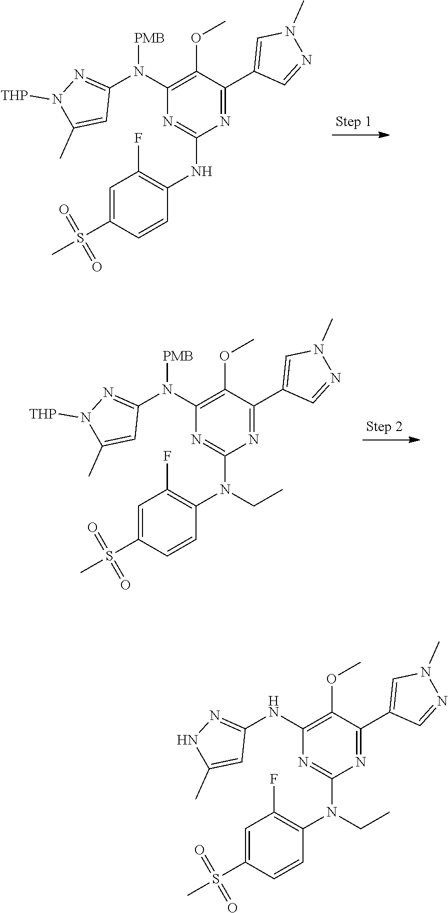

### Step 1/N²-ethyl-N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To a solution of N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (see Compound 2, Step 2) (37 mg, 54.67 umol) in DMF (0.5 mL) was added NaH (3.9 mg, 164 μmol), after stirring for 10 min iodoethane (8.8 μL, 109 μmol) was added and the reaction mixture was stirred at rt for 1 h. The crude reaction mixture was quenched with water, filtrated, and purified by preparative HPLC eluting with a gradient of MeCN in water both containing 0.1% formic acid. Appropriate fractions were combined and lyophilized to afford the intermediate N²-ethyl-N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine as brown solid which was use directly in the next step without purification.

### Step 2/Compound 3

To crude N²-ethyl-N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine was added TFA (0.5 mL). The reaction mixture was stirred at 80° C. for 1 h. The crude reaction mixture was evaporated, diluted in DMSO, filtrated, and purified by preparative HPLC eluting with a gradient of MeCN (10 to 50%) in water both containing 0.1% formic acid. Appropriate fractions were combined and lyophilized to afford Compound 3 (7 mg, 26% yield from Step 1) as white solid. UPLC-MS (+ESI) m/z=501.2 (M+H)⁺. ¹H NMR (400 MHz, DMSO-d₆) δ 11.86 (s, 1H), 8.84 (s, 1H), 8.26 (s, 1H), 7.95 (s, 1H), 7.86 (ddd, J=13.2, 8.9, 2.1 Hz, 2H), 7.74 (t, J=7.8 Hz, 1H), 5.54 (s, 1H), 4.02 (q, J=7.0 Hz, 2H), 3.93 (s, 3H), 3.61 (s, 3H), 2.02 (s, 3H), 1.19 (t, J=7.0 Hz, 3H).

### Compound 4/Method B/N²-(2-fluoro-4-(methylsulfonyl)phenyl)-N²-(4-fluorobenzyl)-5-methoxy-N⁴-(5-methyl-1H-pyrazol-3-yl)-6-(1-methyl-1H-pyrazol-4-yl)pyrimidine-2,4-diamine

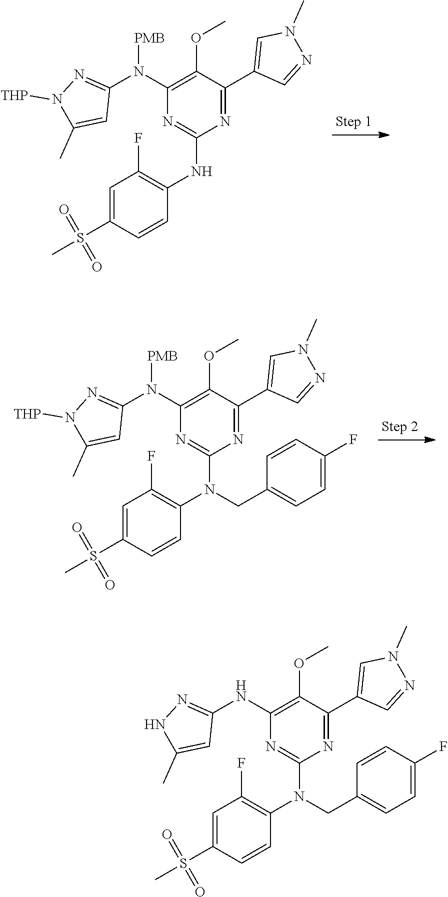

### Step 1/N²-(2-fluoro-4-(methylsulfonyl)phenyl)-N²-(4-fluorobenzyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To a solution of N²-(2-fluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (see Compound 2, Step 2) (33.0 mg, 48.7 μmol) in DMF (0.5 mL) was added NaH (5.1 mg, 146 μmol, 60% dispersion), after stirring for 10 min, 1-(bromomethyl)-4-fluoro-benzene (18 uL, 146 μmol,) was added and the reaction mixture was stirred at rt for 15 h. The crude reaction mixture was quenched with water, filtrated, and purified by preparative HPLC eluting with a gradient of MeCN in water both containing 0.1% formic acid. Appropriate fractions were combined and lyophilized to afford the intermediate N²-(2-fluoro-4-(methylsulfonyl)phenyl)-N²-(4-fluorobenzyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine as brown solid which was used in the next step without further purification.

### Step 2/Compound 4

To crude N²-(2-fluoro-4-(methylsulfonyl)phenyl)-N²-(4-fluorobenzyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine from step one was added TFA (0.5 mL). The reaction mixture was stirred at 80° C. for 1 h. The crude reaction mixture was evaporated, diluted in DMSO, filtrated, and purified by preparative HPLC eluting with a gradient of MeCN (10 to 50%) in water both containing 0.1% formic acid. Appropriate fractions were combined and lyophilized to afford the desired product Compound 4 (5.1 mg, 18% yield from Step 1) as white solid. UPLC-MS (+ESI) m/z=581.3 (M+H)⁺. ¹H NMR (400 MHz, DMSO-d₆) δ 11.86 (s, 1H), 8.84 (s, 1H), 8.24 (s, 1H), 7.91 (s, 1H), 7.86-7.76 (m, 2H), 7.72 (t, J=7.7 Hz, 1H), 7.37 (dd, J=8.6, 5.7 Hz, 2H), 7.17-7.06 (m, 2H), 5.49 (s, 1H), 5.27 (s, 2H), 3.91 (s, 3H), 3.62 (s, 3H), 3.28 (s, 3H), 2.00 (s, 3H).

### Compound 5/Method A/5-methoxy-N-(5-methyl-1H-pyrazol-3-yl)-6-(1-methyl-1H-pyrazol-4-yl)-2-(5-(methylsulfonyl) indolin-1-yl)pyrimidin-4-amine

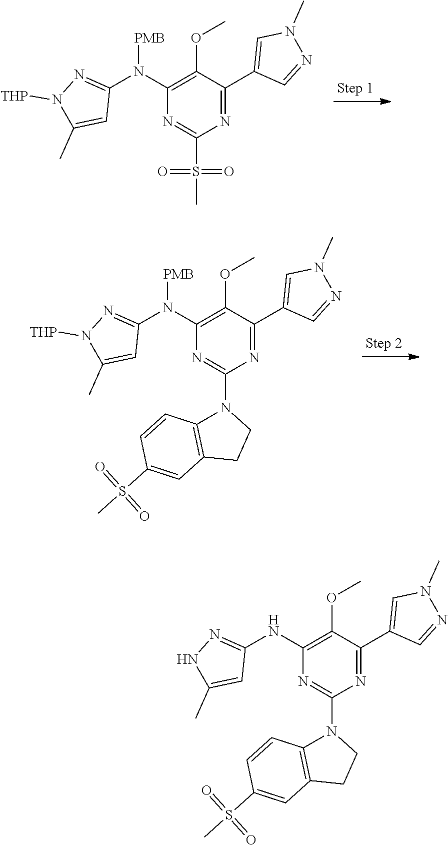

### Step 1/5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(5-(methylsulfonyl) indolin-1-yl)pyrimidin-4-amine

To a solution of 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 2, Step 2) (20.0 mg, 35.2 μmol) in DMPU (0.5 mL) was added NaH (2.8 mg, 71 mmol, 60% purity). The reaction mixture was stirred at 120° C. for 15 hrs. The crude reaction mixture was quenched with water, filtrated, and purified by preparative HPLC eluting with a gradient of MeCN (40 to 70%) in water both containing 0.1% formic acid. Appropriate fractions were combined and lyophilized to afford the 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(5-(methylsulfonyl) indolin-1-yl)pyrimidin-4-amine as yellow solid which was used in the next step without further purification.

### Step 2/Compound 5

To crude 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(5-(methylsulfonyl) indolin-1-yl)pyrimidin-4-amine was added TFA (0.5 mL). The reaction mixture was stirred at 80° C. for 1 h. The crude reaction mixture was evaporated, diluted in DMSO, filtrated, and purified by preparative HPLC eluting with a gradient of MeCN (10 to 50%) in water both containing 0.1% formic acid. Appropriate fractions were combined and lyophilized to afford Compound 5 (3.0 mg, 18% yield from Step 1) as white solid. UPLC-MS (+ESI) m/z=481.2 (M+H)⁺. ¹H NMR (400 MHz, DMSO-d₆) δ 9.10 (s, 1H), 8.55 (d, J=8.6 Hz, 1H), 8.38 (s, 1H), 8.09 (s, 1H), 7.73 (dd, J=8.6, 2.1 Hz, 1H), 7.66 (d, J=1.9 Hz, 1H), 6.74 (s, 1H), 6.37 (s, 1H), 4.30 (t, J=8.9 Hz, 2H), 3.96 (s, 3H), 3.68 (s, 3H), 3.22 (t, J=8.8 Hz, 2H), 3.13 (s, 3H), 2.27 (s, 3H).

### Compound 6/Method B/N²-(2-fluoro-4-(methylsulfonyl)phenyl)-5-methoxy-N²-methyl-6-(1-methyl-1H-pyrazol-3-yl)-N⁴-(5-methyl-1H-pyrazol-3-yl)pyrimidine-2,4-diamine

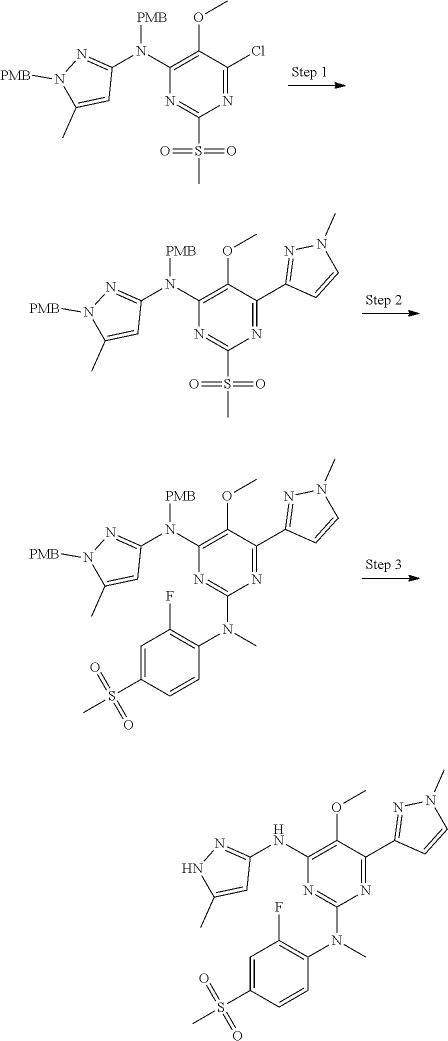

### Step 1/5-methoxy-N-(4-methoxybenzyl)-N-(1-(4-methoxybenzyl)-5-methyl-1H-pyrazol-3-yl)-6-(1-methyl-1H-pyrazol-3-yl)-2-(methylsulfonyl)pyrimidin-4-amine

To a solution of Intermediate 10 (208 mg, 372 μmol) in Dioxane (2 mL) was added tributyl-(1-methylpyrazol-3-yl) stannane (152 mg, 410 μmol) and Pd(PPh₃)₄ (43 mg, 37 μmol). The reaction mixture was stirred at 100° C. for 5 hrs. The crude reaction mixture was evaporated, dissolved in 2 mL DMSO, filtered and purified by preparative HPLC eluting with a gradient of MeCN in water (40-70%) both containing 0.1% formic acid. Appropriate fractions were combined and evaporated to afford 5-methoxy-N-(4-methoxybenzyl)-N-(1-(4-methoxybenzyl)-5-methyl-1H-pyrazol-3-yl)-6-(1-methyl-1H-pyrazol-3-yl)-2-(methylsulfonyl)pyrimidin-4-amine (106 mg, 47% yield). UPLC-MS (+ESI) m/z=604.3 (M+H)⁺.

### Step 2/N²-(2-fluoro-4-(methylsulfonyl)phenyl)-5-methoxy-N⁴-(4-methoxybenzyl)-N⁴-(1-(4-methoxybenzyl)-5-methyl-1H-pyrazol-3-yl)-N²-methyl-6-(1-methyl-1H-pyrazol-4-yl)pyrimidine-2,4-diamine

To 2-fluoro-4-methylsulfonyl-aniline (37 mg, 193 μmol) in DMPU (1 mL) was added NaH (4.4 mg, 193 μmol, 60% dispersion). The mixture was stirred at rt for 10 min. To this mixture was added 5-methoxy-N-(4-methoxybenzyl)-N-(1-(4-methoxybenzyl)-5-methyl-1H-pyrazol-3-yl)-6-(1-methyl-1H-pyrazol-3-yl)-2-(methylsulfonyl)pyrimidin-4-amine (106 mg, 176 umol) and the reaction heated at 130° C. for 5 hrs. The mixture was then cooled down to rt and MeI (56 uL, 897 umol,) was added and the mixture was stirred overnight at rt. The crude mixture was filtered and purified by preparative HPLC eluting with a gradient of MeCN in water (40-70%) both containing 0.1% formic acid. Appropriate fractions were combined and evaporated to afford N²-(2-fluoro-4-(methylsulfonyl)phenyl)-5-methoxy-N⁴-(4-methoxybenzyl)-N⁴-(1-(4-methoxybenzyl)-5-methyl-1H-pyrazol-3-yl)-N²-methyl-6-(1-methyl-1H-pyrazol-4-yl)pyrimidine-2,4-diamine (10 mg) and the non-methylated intermediate N²-(2-fluoro-4-(methylsulfonyl)phenyl)-5-methoxy-N⁴-(4-methoxybenzyl)-N⁴-(1-(4-methoxybenzyl)-5-methyl-1H-pyrazol-3-yl)-6-(1-methyl-1H-pyrazol-3-yl)pyrimidine-2,4-diamine (48 mg). To the later dissolved in DMF (1.5 mL) was added NaH (3.2 mg, 135 μmol, 60% dispersion) then MeI (8.4 μL, 135 μmol). The reaction was then stirred at rt for 15 min. The crude was purified by preparative HPLC eluting with a gradient of MeCN in water (40-70%) both containing 0.1% formic acid. Appropriate fractions were combined and evaporated to afford N²-(2-fluoro-4-(methylsulfonyl)phenyl)-5-methoxy-N⁴-(4-methoxybenzyl)-N⁴-(1-(4-methoxybenzyl)-5-methyl-1H-pyrazol-3-yl)-N²-methyl-6-(1-methyl-1H-pyrazol-4-yl)pyrimidine-2,4-diamine which was combined with the 10 mg isolated previously.

### Step 3/Compound 6

The combined N²-(2-fluoro-4-(methylsulfonyl)phenyl)-5-methoxy-N⁴-(4-methoxybenzyl)-N⁴-(1-(4-methoxybenzyl)-5-methyl-1H-pyrazol-3-yl)-N²-methyl-6-(1-methyl-1H-pyrazol-4-yl)pyrimidine-2,4-diamine from the previous step dissolved in TFA (2 mL) and heated overnight at 120° C. The reaction mixture was evaporated in vacuo, dissolved in DMSO, and purified by preparative HPLC eluting with a gradient of MeCN in water (20-50%) both containing 0.1% formic acid. Appropriate fractions were combined and evaporated to afford Compound 6 (1.7 mg, 5.2% yield for Step 2 and 3). UPLC-MS (+ESI) m/z=487.2 (M+H)⁺. ¹H NMR (400 MHz, DMSO-d₆) δ 7.88-7.77 (m, 2H), 7.74 (q, J=2.8 Hz, 2H), 7.71 (s, OH), 6.72 (d, J=2.2 Hz, 1H), 5.63 (s, 1H), 3.91 (s, 3H), 3.62 (s, 3H), 3.45 (s, 3H), 3.27 (s, 4H), 2.01 (s, 3H).

### Compound 7/Method B/N²-(4-(cyclopropylsulfonyl)-2-fluorophenyl)-5-methoxy-N²-methyl-N⁴-(5-methyl-1H-pyrazol-3-yl)-6-(1-methyl-1H-pyrazol-4-yl)pyrimidine-2,4-diamine

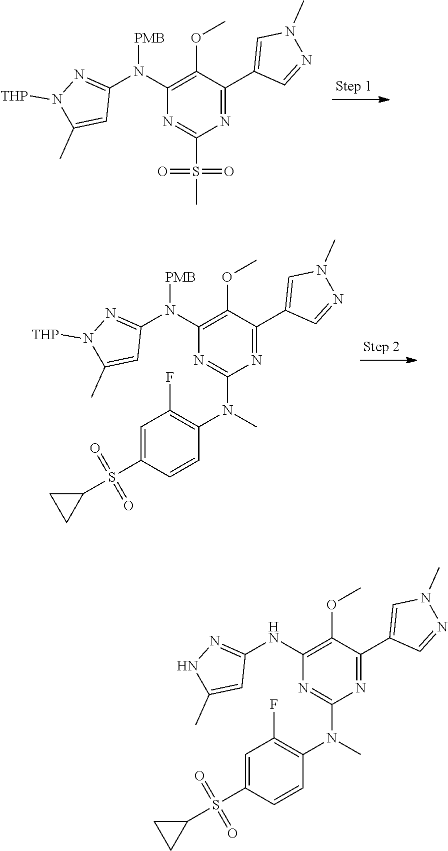

### Step 1/N²-(4-(cyclopropylsulfonyl)-2-fluorophenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 2, Step 1) (87 mg, 153 μmol) in DMPU (1 mL) was added 4-cyclopropylsulfonyl-2-fluoro-aniline (73 mg, 339 umol). Nitrogen gas was bubbled for 5 minutes and then NaH (22 mg, 550 μmol, 60% purity) was added at rt. The vial was closed, and the mixture was heated to 125° C. for 18 hrs. The mixture was cooled to rt and MeI (100 μL, 1.61 mmol,) was added and the mixture was stirred for 1 h at rt. 1 ml of MeOH was added to quench the reaction and the mixture was filtered on a celite cartridge. The MeOH was removed under reduced pressure. The crude reaction mixture was filtered and purified by preparative HPLC eluting with a gradient of MeCN (35 to 65%) in water, both containing 0.1% formic acid. Appropriate fractions were combined and lyophilized to afford N²-(4-cyclopropylsulfonyl-2-fluoro-phenyl)-5-methoxy-N⁴-[(4-methoxyphenyl)methyl]-N²-methyl-6-(1-methylpyrazol-4-yl)-N⁴-(5-methyl-1-tetrahydropyran-2-yl-pyrazol-3-yl)pyrimidine-2,4-diamine (39 mg, 36% yield).

### Step 2/Compound 7

TFA (3.0 mL, 39.2 mmol,) was added to N²-(4-cyclopropylsulfonyl-2-fluoro-phenyl)-5-methoxy-N⁴-[(4-methoxyphenyl)methyl]-N²-methyl-6-(1-methylpyrazol-4-yl)-N⁴-(5-methyl-1-tetrahydropyran-2-yl-pyrazol-3-yl)pyrimidine-2,4-diamine (39 mg, 54.4 umol) and the mixture was stirred at 90° C. for 2 h. The volatiles were removed under reduced pressure, the residue was dissolved in DMSO purified by preparative HPLC eluting with a gradient of MeCN (25 to 55%) in water, both containing 0.1% formic acid. Appropriate fractions were combined and lyophilized to afford Compound 7 (14.6 mg, 19% yield). UPLC-MS (+ESI) m/z=513.2 (M+H)⁺. ¹H NMR (400 MHz, DMSO-d₆) δ11.83 (s, 1H), 8.77 (s, 1H), 8.23 (s, 1H), 7.92 (s, 1H), 7.81-7.62 (m, 3H), 5.61 (s, 1H), 3.89 (s, 3H), 3.58 (s, 3H), 3.46 (s, 3H), 2.01 (s, 3H), 1.20-1.12 (m, 2H), 1.10-1.00 (m, 2H).

### Compound 8/Method B/N²-(2,6-difluoro-4-(methylsulfonyl)phenyl)-5-methoxy-N²-methyl-N⁴-(5-methyl-1H-pyrazol-3-yl)-6-(1-methyl-1H-pyrazol-4-yl)pyrimidine-2,4-diamine

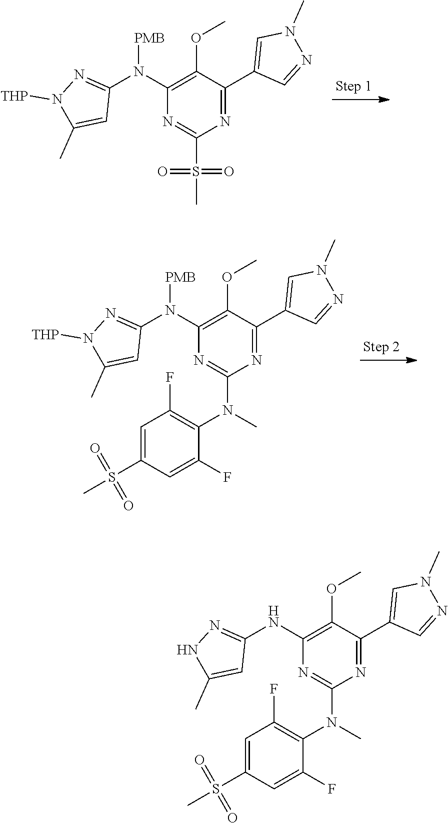

### Step 1/N²-(2,6-difluoro-4-(methylsulfonyl)phenyl)-5-methoxy-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To a solution of 5-methoxy-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 2, Step 1) (50 mg, 88 μmol) and 2,6-difluoro-4-methylsulfonyl-aniline (110 mg, 529 umol) in DMPU (0.5 mL) at rt was added NaH (21 mg, 529 μmol, 60% dispersion). The reaction mixture was stirred at 80° C. for 15 hrs. The reaction was quenched with water (0.2 mL). The resulting reaction mixture was filtrated and purified by preparative HPLC eluting with a gradient of MeCN in water both containing 0.1% formic acid. Appropriate fractions were combined and evaporated to afford N²-(2,6-difluoro-4-(methylsulfonyl)phenyl)-5-methoxy-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (19 mg, 31% yield, 27 μmol) as yellow solid. The later was dissolved in DMF (0.5 mL) before adding NaH (10 mg, 250 μmol, 60% dispersion), followed by MeI (16.0 μL, 264 μmol) at rt. The reaction was stirred for 30 min then was quenched with water 0.2 mL. The crude reaction mixture was filtrated and purified by preparative HPLC eluting with a gradient of MeCN in water both containing 0.1% formic acid. Appropriate fractions were combined and evaporated to afford N²-(2,6-difluoro-4-(methylsulfonyl)phenyl)-5-methoxy-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (13 mg, 53% yield) as yellow solid. UPLC-MS (+ESI) m/z=709.3 (M+H)⁺.

### Step 2/Compound 8

To N²-(2,6-difluoro-4-(methylsulfonyl)phenyl)-5-methoxy-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (11 mg, 15.5 μmol) was added TFA (0.5 mL). The reaction mixture was stirred at 80° C. for 1 h. The crude reaction mixture was concentrated, the residue dissolved in DMSO and purified by preparative HPLC eluting with a gradient of MeCN in water both containing 0.1% formic acid. Appropriate fractions were combined and lyophilized to afford Compound 8 (4.3 mg, 55% yield) as white solid. UPLC-MS (+ESI) m/z=505.1 (M+H)⁺. ¹H NMR (400 MHz, DMSO-d₆) δ 11.89 (s, 1H), 8.92 (s, 1H), 8.28 (s, 1H), 7.98 (s, 1H), 7.85 (d, J=6.9 Hz, 2H), 5.45 (s, 1H), 3.92 (s, 3H), 3.62 (s, 3H), 3.42 (s, 3H), 3.37 (s, 3H), 2.04 (s, 3H).

### Compound 9/Method A/2-(2-fluoro-4-(methylsulfonyl) phenoxy)-5-methoxy-N-(5-methyl-1H-pyrazol-3-yl)-6-(1-methyl-1H-pyrazol-4-yl)pyrimidin-4-amine

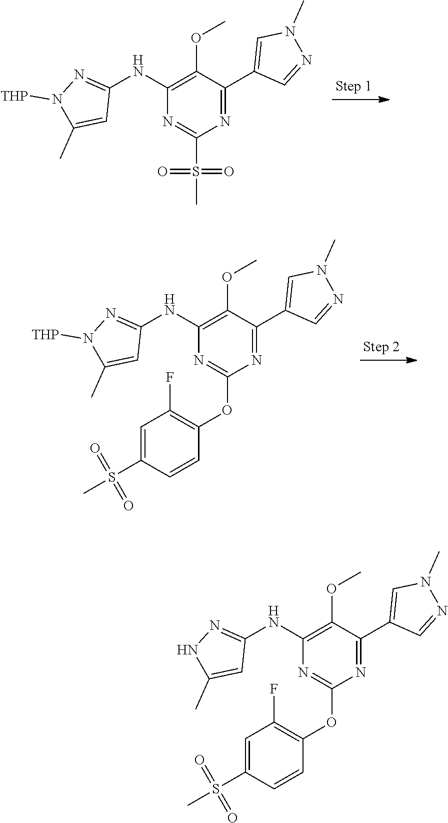

### Step 1/2-(2-fluoro-4-(methylsulfonyl) phenoxy)-5-methoxy-N-(5-methyl-1-(tetrahydro-2H-pyran-2-yl)-1H-pyrazol-3-yl)-6-(1-methyl-1H-pyrazol-4-yl)pyrimidin-4-amine

To 2-fluoro-4-methylsulfonyl-phenol (102 mg, 536 umol) in MeCN (3 mL) was added NaH (22 mg, 550 μmol, 60% dispersion) and the mixture was stirred at rt for 15 min. To this mixture was added 5-methoxy-N-(5-methyl-1-(tetrahydro-2H-pyran-2-yl)-1H-pyrazol-3-yl)-6-(1-methyl-1H-pyrazol-4-yl)-2 (methylsulfonyl)pyrimidin-4-amine (see Compound 1, Step 1) (120 mg, 268 μmol) and the reaction heated in microwave for 90 min at 170° C. The reaction mixture was evaporated in vacuo dissolved in DMSO and purified by preparative HPLC eluting with a gradient of MeCN in water both containing 0.1% formic acid. Appropriate fractions were combined and lyophilized to afford 2-(2-fluoro-4-(methylsulfonyl) phenoxy)-5-methoxy-N-(5-methyl-1-(tetrahydro-2H-pyran-2-yl)-1H-pyrazol-3-yl)-6-(1-methyl-1H-pyrazol-4-yl)pyrimidin-4-amine (18.6 mg, 13% yield).

### Step 2/Compound 9

To 2-(2-fluoro-4-(methylsulfonyl) phenoxy)-5-methoxy-N-(5-methyl-1-(tetrahydro-2H-pyran-2-yl)-1H-pyrazol-3-yl)-6-(1-methyl-1H-pyrazol-4-yl)pyrimidin-4-amine (18.6 mg, 33.4 μmol) in dioxane (1 mL) was added HCl in dioxane (0.1 mL, 4N) then the mixture was heated at 50° C. overnight. The reaction mixture was evaporated in vacuo to give the desired product Compound 9 (13.3 mg, 81% yield). UPLC-MS (+ESI) m/z=474.1 (M+H)⁺. ¹H NMR (400 MHz, DMSO-d₆) δ 9.80 (s, 1H), 8.29 (s, 1H), 7.99 (dd, J=9.7, 2.2 Hz, 1H), 7.91 (d, J=0.7 Hz, 1H), 7.84 (dt, J=8.4, 1.4 Hz, 1H), 7.68 (dd, J=8.4, 7.6 Hz, 1H), 5.63 (d, J=0.8 Hz, 1H), 3.89 (s, 3H), 3.64 (s, 3H), 3.29 (s, 3H), 2.16-1.95 (m, 3H).

### Compound 10/Method A/5-cyclopropyl-N²-(2-fluoro-4-(methylsulfonyl)phenyl)-N⁴-(5-methyl-1H-pyrazol-3-yl)-6-(1-methyl-1H-pyrazol-4-yl)pyrimidine-2,4-diamine

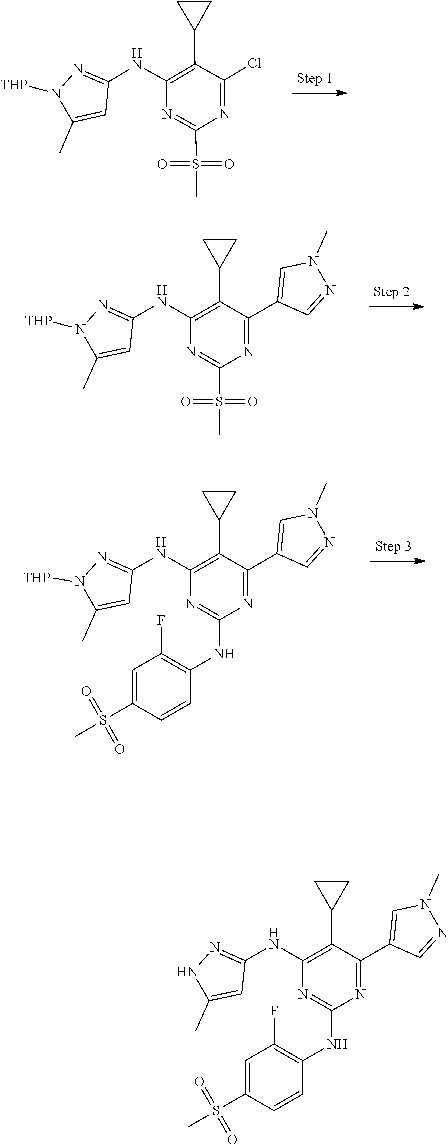

### Step 1/5-cyclopropyl-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine

To solution of Intermediate 7 (523 mg, 1.27 mmol) in dioxane/water (5 mL, 9/1) was added 1-methyl-4-(4,4,5,5-tetramethyl-1,3,2-dioxaborolan-2-yl) pyrazole (396 mg, 1.90 mmol), K₂CO₃ (526 mg, 3.81 mmol), Pd(dppf)Cl₂ (93 mg, 127 μmol) and the mixture was degassed with nitrogen for 2 min. The reaction mixture was then stirred at 120° C. for 5 h. The crude reaction mixture was diluted with EtOAc, quenched with NaHCO₃ sat., then extracted with EtOAc. The combined organic phases were washed with brine, dried with Na₂SO₄, filtrated, and concentrated in vacuo. The crude mixture was purified by silica gel chromatography eluting with a gradient of MeOH (0-10%) in DCM. Appropriate fractions were combined and concentrated to afford the 5-cyclopropyl-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (388 mg, 67% yield) as a yellow solid. UPLC-MS (+ESI) m/z=458.2 (M+H)⁺.

### Step 2, 3/Compound 10

To a solution of 5-cyclopropyl-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (20.0 mg, 43.7 μmol) and 2-fluoro-4-methylsulfonyl-aniline (16.5 mg, 87.4 μmol) in DMPU (0.5 mL) was added NaH (3.5 mg, 87 umol, 60% purity). The reaction mixture was stirred at 120° C. for 15 hrs. The crude reaction mixture was quenched with water, filtered, and purified by preparative HPLC eluting with a gradient of MeCN in water both containing 0.1% formic acid. Appropriate fractions were combined and lyophilized to afford the intermediate 5-cyclopropyl-N²-(2-fluoro-4-methylsulfonyl-phenyl)-6-(1-methylpyrazol-4-yl)-N⁴-(5-methyl-1-tetrahydropyran-2-yl-pyrazol-3-yl)pyrimidine-2,4-diamine as yellow solid. To the later was added HCl in Dioxane (2 mL, 4M) and MeOH (0.1 mL). The reaction mixture was stirred at rt for 5 hrs. The crude reaction mixture was concentrated, diluted in DMSO, filtrated, and purified by preparative HPLC eluting with a gradient of MeCN (15 to 45%) in water both containing 0.1% formic acid. Appropriate fractions were combined and lyophilized to afford Compound 10 (8.0 mg, 38% yield) as white solid. UPLC-MS (+ESI) m/z=483.1 (M+H)⁺. ¹H NMR (400 MHz, DMSO-d₆) δ 8.90 (s, 1H), 8.33 (s, 1H), 8.06 (s, 1H), 7.86-7.70 (m, 2H), 5.89 (s, 1H), 3.94 (s, 3H), 3.23 (s, 3H), 2.19 (s, 3H), 1.80 (td, J=7.8, 3.8 Hz, 1H), 1.18 (d, J=6.6 Hz, 2H), 0.23 (d, J=5.1 Hz, 2H).

### Compound 11/Method B/5-cyclopropyl-N²-(2-fluoro-4-(methylsulfonyl)phenyl)-N²-methyl-N⁴-(5-methyl-1H-pyrazol-3-yl)-6-(1-methyl-1H-pyrazol-4-yl)pyrimidine-2,4-diamine

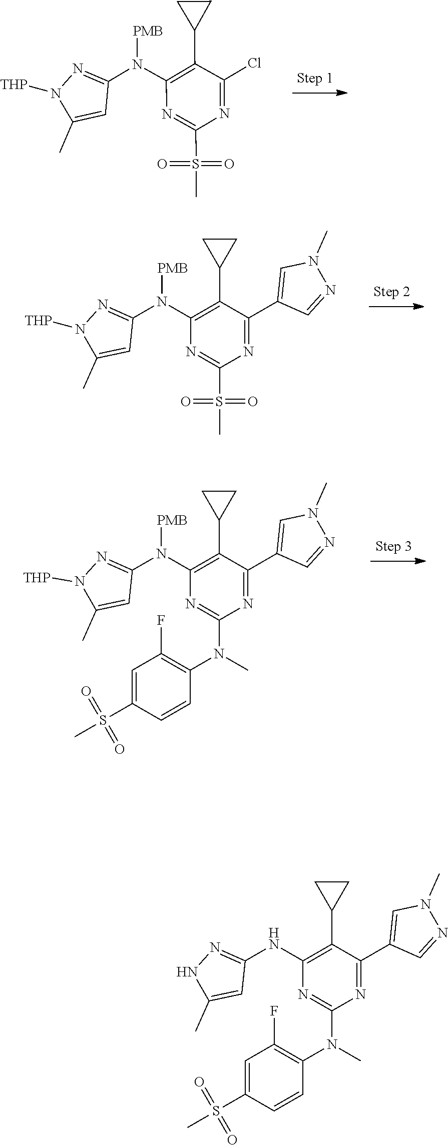

### Step 1/5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine

To the solution of Intermediate 8 (2.00 g, 3.76 mmol) in Dioxane (10 mL) and H₂O (3 mL) was added 1-methyl-4-(4,4,5,5-tetramethyl-1,3,2-dioxaborolan-2-yl) pyrazole (939 mg, 4.51 mmol), Pd(dppf)Cl₂ (550 mg, 752 μmol) and Cs₂CO₃ (3.06 g, 9.40 mmol). The resulting mixture was degassed in vacuo and then backfilled with nitrogen. The resulting reaction mixture was stirred at 90° C. for 3 hrs, then cooled to rt and diluted with EtOAc. The organic phase was extracted, washed with brine, dried over Na₂SO₄, filtered, and concentrated in vacuo. The residue was purified by preparative HPLC eluting with a gradient of MeCN (10 to 100%) in water both containing 0.1% formic acid. Appropriate fractions were combined and lyophilized to afford 5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (1.45 g, 51% yield, 77% purity). UPLC-MS (+ESI) m/z=578.2 (M+H)⁺.

### Step 2/5-cyclopropyl-N²-(2-fluoro-4-(methylsulfonyl)phenyl)-N⁴-(4-methoxybenzyl)-N²-methyl-N4-(5-methyl-1-(tetrahydro-2H-pyran-2-yl)-1H-pyrazol-3-yl)-6-(1-methyl-1H-pyrazol-4-yl)pyrimidine-2,4-diamine

To 5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (690 mg, 1.05 mmol) in DMPU (6 mL) was added 2-fluoro-4-methylsulfonyl-aniline (259 mg, 1.37 mmol). Nitrogen was bubbled for 5 min in the reaction mixture and then LiHMDS (1M in THF, 3.15 mL, 3.15 mmol) was slowly added at rt. The vial was closed, and the mixture was heated to 80° C. for 1 h. The mixture was cooled to rt and MeI (327 μL, 5.26 mmol,) was added and the mixture was stirred for 1 h at rt. 1 mL of MeOH was added to quench the reaction and the mixture was filtrated on a celite cartridge and evaporated to dryness. The filtrate was purified by preparative HPLC eluting with a gradient of MeCN (10 to 100%) in water both containing 0.1% formic acid. Appropriate fractions were combined and lyophilized to afford 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (358 mg, 49% yield). UPLC-MS (+ESI) m/z=701.4 (M+H)⁺.

### Step 3/Compound 11

To 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (938 mg, 1.34 mmol) was added TFA (21 mL, 268 mmol) and the reaction mixture was stirred at 120° C. for 6 h. The TFA was removed under reduced pressure and the residue was purified by preparative HPLC eluting with a gradient of MeCN (10 to 100%) in water both containing 0.1% formic acid. Appropriate fractions were combined and lyophilized to afford Compound 11 (430 mg, 65% yield). UPLC-MS (+ESI) m/z=497.2 (M+H)⁺. ¹H NMR (400 MHz, DMSO-d₆) δ 8.21 (s, 1H), 7.92 (d, J=0.7 Hz, 1H), 7.85-7.77 (m, 2H), 7.71 (dd, J=8.2, 7.3 Hz, 1H), 5.58 (s, 1H), 3.87 (s, 3H), 3.45 (s, 3H), 3.27 (s, 3H), 2.05-1.95 (m, 3H), 1.77-1.65 (m, 1H), 1.16-1.00 (m, 2H), 0.18-0.12 (m, 2H).

### Compound 12/Method B/5-cyclopropyl-N²-(2-fluoro-4-(methylsulfonyl)phenyl)-N²-methyl-6-(1-methyl-1H-pyrazol-3-yl)-N⁴-(5-methyl-1H-pyrazol-3-yl)pyrimidine-2,4-diamine

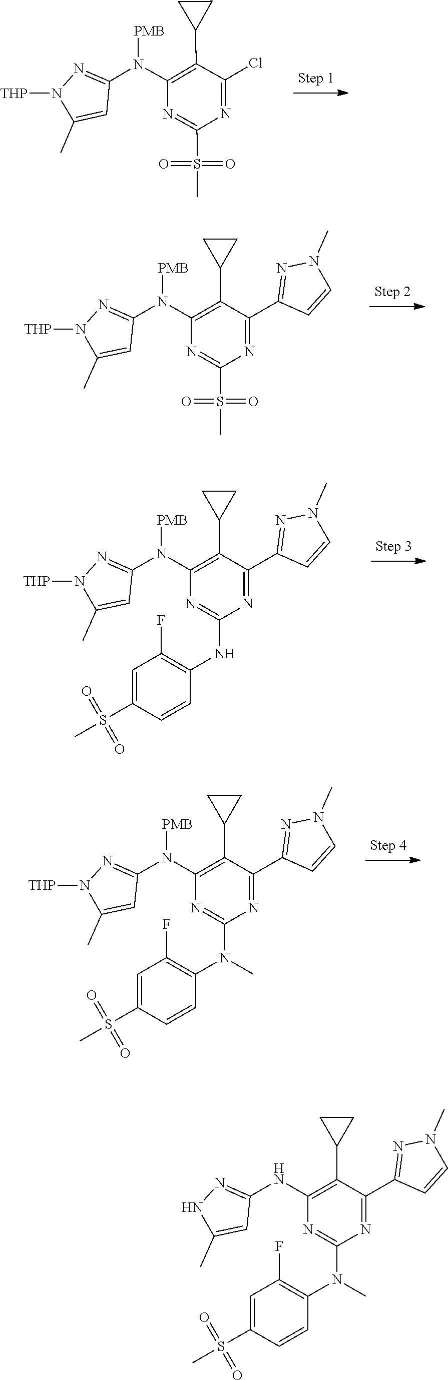

### Step 1/5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-3-yl)-2-(methylsulfonyl)pyrimidin-4-amine

To a solution of Intermediate 8 (312 mg, 586 μmol) in Dioxane (2 mL) was added tributyl-(1-methylpyrazol-3-yl) stannane (239 mg, 645 μmol), Pd(PPh₃)₄ (68 mg, 59 μmol). The reaction mixture was then stirred overnight at 100° C. The crude was purified by preparative HPLC eluting with a gradient of MeCN in water (15-45%), both containing 0.1% formic acid. Appropriate fractions were combined and lyophilized to afford the desired product 5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-3-yl)-2-(methylsulfonyl)pyrimidin-4-amine (142 mg, 42% yield). UPLC-MS (+ESI) m/z=578.3 (M+H)⁺.

### Step 2/5-cyclopropyl-N²-(2-fluoro-4-(methylsulfonyl)phenyl)-N⁴-(4-methoxybenzyl)-N⁴-(5-methyl-1-(tetrahydro-2H-pyran-2-yl)-1H-pyrazol-3-yl)-6-(1-methyl-1H-pyrazol-3-yl)pyrimidine-2,4-diamine

A mixture of 2-fluoro-4-methylsulfonyl-aniline (70.0 mg, 370 μmol) and NaH (9.0 mg, 391 umol, 60% dispersion) in DMPU (1.5 mL) was stirred at rt for 10 min. 5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-3-yl)-2-(methylsulfonyl)pyrimidin-4-amine (142 mg, 246 μmol) was added and the reaction mixture was heated overnight at 130° C. The reaction mixture was cooled to rt, quenched with a drop of water, and purified by preparative HPLC eluting with a gradient of MeCN in water both containing 0.1% formic acid. Appropriate fractions were combined and lyophilized to afford 5-cyclopropyl-N²-(2-fluoro-4-(methylsulfonyl)phenyl)-N⁴-(4-methoxybenzyl)-N⁴-(5-methyl-1-(tetrahydro-2H-pyran-2-yl)-1H-pyrazol-3-yl)-6-(1-methyl-1H-pyrazol-3-yl)pyrimidine-2,4-diamine (50 mg, 30% yield).

### Step 3/5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-3-yl)pyrimidine-2,4-diamine

To 5-cyclopropyl-N²-(2-fluoro-4-(methylsulfonyl)phenyl)-N⁴-(4-methoxybenzyl)-N⁴-(5-methyl-1-(tetrahydro-2H-pyran-2-yl)-1H-pyrazol-3-yl)-6-(1-methyl-1H-pyrazol-3-yl)pyrimidine-2,4-diamine (50 mg, 73 μmol) in DMF (1.5 mL) was added NaH (9.0 mg, 391 μmol, 60% dispersion) and MeI (40 μL, 643 μmol) and the reaction was stirred overnight at rt. The reaction mixture was quenched with water (0.2 mL), filtered and purified by preparative HPLC eluting with a gradient of MeCN (30-80%) in water both containing 0.1% formic acid. Appropriate fractions were combined and lyophilized to afford 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-3-yl)pyrimidine-2,4-diamine (35 mg, 69% yield). UPLC-MS (+ESI) m/z=701.4 (M+H)⁺.

### Step 4/Compound 12

To 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-3-yl)pyrimidine-2,4-diamine (35 mg, 50 μmol) was added TFA (3 mL) and the mixture was heated at 90° C. for 2 h. The reaction mixture was concentrated, the residue was dissolved in DMSO and purified by preparative HPLC eluting with a gradient of MeCN (20-70%) in water both containing 0.1% formic acid. Appropriate fractions were combined and lyophilized to afford the desired product Compound 12 (13 mg, 52% yield). UPLC-MS (+ESI) m/z=497.2 (M+H)⁺. ¹H NMR (400 MHz, DMSO-d₆) δ 11.84 (s, 1H), 8.22 (s, 1H), 7.89-7.77 (m, 2H), 7.77-7.62 (m, 2H), 6.55 (d, J=2.2 Hz, 1H), 5.59 (s, 1H), 3.87 (s, 3H), 3.43 (s, 3H), 2.01 (s, 3H), 1.85-1.58 (m, 1H), 0.95-0.73 (m, 2H), 0.00 (dt, J=5.7, 3.0 Hz, 2H).

### Compound 13/Method B/5-cyclopropyl-N²-(2,6-difluoro-4-(methylsulfonyl)phenyl)-N²-methyl-6-(1-methyl-1H-pyrazol-3-yl)-N⁴-(5-methyl-1H-pyrazol-3-yl)pyrimidine-2,4-diamine

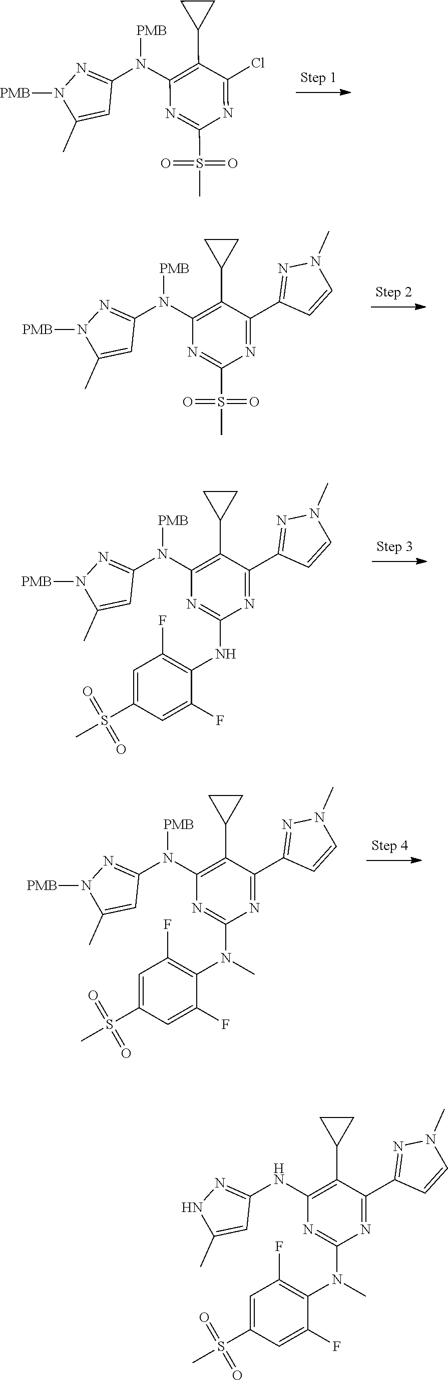

### Step 1/5-cyclopropyl-N-(4-methoxybenzyl)-N-(1-(4-methoxybenzyl)-5-methyl-1H-pyrazol-3-yl)-6-(1-methyl-1H-pyrazol-3-yl)-2-(methylsulfonyl)pyrimidin-4-amine

To a solution of Intermediate 9 (1.15 g, 2.02 mmol) in Dioxane (10 mL) was added tributyl-(1-methylpyrazol-3-yl) stannane (790 mg, 2.13 mmol), Pd(PPh₃)₄ (240 mg, 208 μmol). The reaction mixture was then stirred overnight at 85° C. The crude mixture was purified by silica gel column chromatography eluting with of EtOAc (70%) in heptane. Appropriate fractions were combined and evaporated to afford the desired product 5-cyclopropyl-N-(4-methoxybenzyl)-N-(1-(4-methoxybenzyl)-5-methyl-1H-pyrazol-3-yl)-6-(1-methyl-1H-pyrazol-3-yl)-2-(methylsulfonyl)pyrimidin-4-amine (217 mg, 18% yield). UPLC-MS (+ESI) m/z=614.0 (M+H)⁺.

### Step 2/5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-3-yl)pyrimidine-2,4-diamine

A mixture of 2,6-difluoro-4-methylsulfonyl-aniline (90 mg, 434 μmol) and NaH (18.0 mg, 470 μmol, 60% dispersion) in DMPU (1 mL) was stirred at rt for 10 min. To the resulting mixture was added 5-cyclopropyl-N-(4-methoxybenzyl)-N-(1-(4-methoxybenzyl)-5-methyl-1H-pyrazol-3-yl)-6-(1-methyl-1H-pyrazol-3-yl)-2-(methylsulfonyl)pyrimidin-4-amine (217 mg, 353 μmol) and the reaction was heated overnight at 130° C. The reaction mixture was purified by preparative HPLC eluting with a gradient of MeCN (30-70%) in water both containing 0.1% formic acid. Appropriate fractions were combined and lyophilized to afford the intermediate 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-3-yl)pyrimidine-2,4-diamine (65 mg, 25% yield). UPLC-MS (+ESI) m/z=741.3 (M+H)⁺.

### Step 3/5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-N²-methyl-6-(1-methyl-1H-pyrazol-3-yl)pyrimidine-2,4-diamine

To 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-3-yl)pyrimidine-2,4-diamine (65 mg, 87 μmol) in DMF (1 mL) was added NaH (4.0 mg, 104 μmol, 60% dispersion), MeI (20 μL, 321 umol). The reaction mixture was stirred for 1.5 h at rt. The reaction mixture was quenched with water (0.2 ml) and purified by preparative HPLC eluting with a gradient of MeCN (30-70%) in water both containing 0.1% formic acid. Appropriate fractions were combined and lyophilized to afford 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-3-yl)pyrimidine-2,4-diamine which was used directly in the next step.

### Step 4/Compound 13

To 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-3-yl)pyrimidine-2,4-diamine from Step 3 was added TFA (2 mL) and the mixture was heated at 120° C. for 15 hrs. The reaction mixture was evaporated, diluted in DMSO and purified by preparative HPLC eluting with a gradient of MeCN (30-70%) in water both containing 0.1% formic acid. Appropriate fractions were combined and lyophilized to afford the desired product Compound 13 (5 mg, 3% yield). UPLC-MS (+ESI) m/z=515.2 (M+H)⁺. ¹H NMR (400 MHz, DMSO-d₆) δ 11.86 (s, 1H), 8.29 (d, J=26.1 Hz, 1H), 7.82 (d, J=6.8 Hz, 2H), 7.68 (s, 1H), 6.56 (s, 0.3H), 5.33 (s, 1H), 3.88 (s, 3H), 3.35 (s, 3H), 3.33 (s, 3H), 2.00 (s, 3H), 1.78-1.67 (m, 1H), 0.93-0.83 (m, 2H), 0.04-−0.04 (m, 2H).

### Compound 14/Method B/5-cyclopropyl-N²-(2,6-difluoro-4-(methylsulfonyl)phenyl)-N²-methyl-N⁴-(5-methyl-1H-pyrazol-3-yl)-6-(1-methyl-1H-pyrazol-4-yl)pyrimidine-2,4-diamine

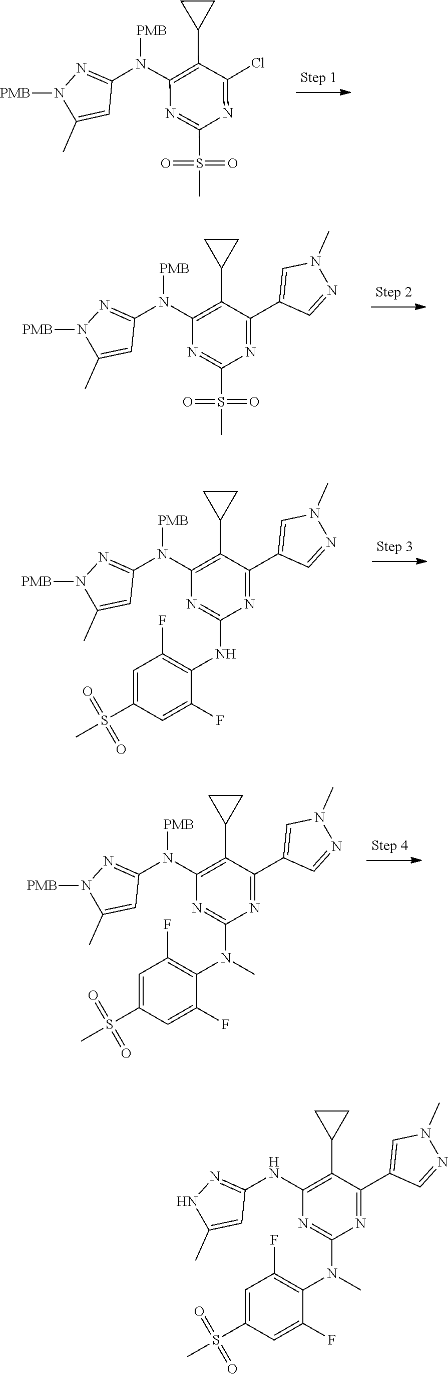

### Step 1/5-cyclopropyl-N-(4-methoxybenzyl)-N-(1-(4-methoxybenzyl)-5-methyl-1H-pyrazol-3-yl)-6-(1-methyl-1H-pyrazol-4-yl)-2-(methylsulfonyl)pyrimidin-4-amine

To a solution of Intermediate 9 (1.60 g, 2.82 mmol) in Dioxane (40 mL) was added 1-methyl-4-(4,4,5,5-tetramethyl-1,3,2-dioxaborolan-2-yl) pyrazole (704 mg, 3.38 mmol), Pd(dppf)Cl₂ (206 mg, 282 μmol), Cs₂CO₃ (1.84 g, 5.63 mmol) and water (10 mL). The reaction mixture was then stirred for 1.5 hrs at 80° C. A second portion of 1-methyl-4-(4,4,5,5-tetramethyl-1,3,2-dioxaborolan-2-yl) pyrazole (250 mg, 1.20 mmol) and Pd(dppf)Cl₂ (100 mg, 39 μmol) were added and the reaction mixture was stirred for 1.5 hrs at 80° C. The reaction mixture was extracted with EtOAc and the organic phase evaporated in vacuo. The crude was purified by silica gel chromatography eluting with of EtOAc (100%). Appropriate fractions were combined and evaporated to afford 5-cyclopropyl-N-(4-methoxybenzyl)-N-(1-(4-methoxybenzyl)-5-methyl-1H-pyrazol-3-yl)-6-(1-methyl-1H-pyrazol-4-yl)-2-(methylsulfonyl)pyrimidin-4-amine (800 mg, 46% yield). UPLC-MS (+ESI) m/z=614.3 (M+H)⁺.

### Step 2/5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-4-yl)pyrimidine-2,4-diamine

A mixture of 2,6-difluoro-4-methylsulfonyl-aniline (122 mg, 589 μmol) and NaH (24.0 mg, 626 μmol, 60% dispersion) in DMPU (1 mL) was stirred at rt for 10 min. To this was added 5-cyclopropyl-N-(4-methoxybenzyl)-N-(1-(4-methoxybenzyl)-5-methyl-1H-pyrazol-3-yl)-6-(1-methyl-1H-pyrazol-4-yl)-2-(methylsulfonyl)pyrimidin-4-amine (300 mg, 489 μmol) and the reaction was heated overnight at 130° C. The reaction mixture was purified by preparative HPLC eluting with a gradient of MeCN (30-70%) in water both containing 0.1% formic acid. Appropriate fractions were combined and lyophilized to afford 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-4-yl)pyrimidine-2,4-diamine (29 mg, 8% yield). UPLC-MS (+ESI) m/z=741.3 (M+H)⁺.

### Step 3,4/5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-N²-methyl-6-(1-methyl-1H-pyrazol-4-yl)pyrimidine-2,4-diamine

To 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-6-(1-methyl-1H-pyrazol-4-yl)pyrimidine-2,4-diamine (29 mg, 39 μmol) in DMF (1 mL) was added NaH (3 mg, 75 μmol, 60% dispersion), MeI (10 μL, 161 umol) and the reaction stirred for 1 h at rt. The reaction mixture was purified by preparative HPLC eluting with a gradient of MeCN (30-70%) in water both containing 0.1% formic acid. Appropriate fractions were combined and lyophilized to afford the intermediate 5-cyclopropyl-N²-(2,6-difluoro-4-(methylsulfonyl)phenyl)-N⁴-(4-methoxybenzyl)-N⁴-(1-(4-methoxybenzyl)-5-methyl-1H-pyrazol-3-yl)-N²-methyl-6-(1-methyl-1H-pyrazol-4-yl)pyrimidine-2,4-diamine. UPLC-MS (+ESI) m/z=755.9 (M+H)⁺. To the later intermediate was added TFA (2 mL) and the mixture was heated overnight at 120° C. The reaction mixture was evaporated, diluted in DMSO and purified by preparative HPLC eluting with a gradient of MeCN (20-70%) in water both containing 0.1% formic acid. Appropriate fractions were combined and lyophilized to afford Compound 14 (1.4 mg, 7% yield for two steps). UPLC-MS (+ESI) m/z=515.2 (M+H)⁺. ¹H NMR (400 MHz, DMSO-d₆) δ 11.83 (s, 1H), 8.26 (d, J=16.6 Hz, 2H), 7.98 (d, J=25.5 Hz, 1H), 7.82 (d, J=6.8 Hz, 2H), 6.58 (s, 0.2H), 5.29 (s, 1H), 3.87 (s, 3H), 3.37 (s, 3H), 3.33 (s, 3H), 2.03 (s, 3H), 1.72 (tt, J=7.9, 5.5 Hz, 1H), 1.19-0.86 (m, 2H), 0.34-0.07 (m, 2H).

### Compound 15/Method B/5-cyclopropyl-N²-(4-(cyclopropylsulfonyl)-2-fluorophenyl)-N²-methyl-N⁴-(5-methyl-1H-pyrazol-3-yl)-6-(1-methyl-1H-pyrazol-4-yl)pyrimidine-2,4-diamine

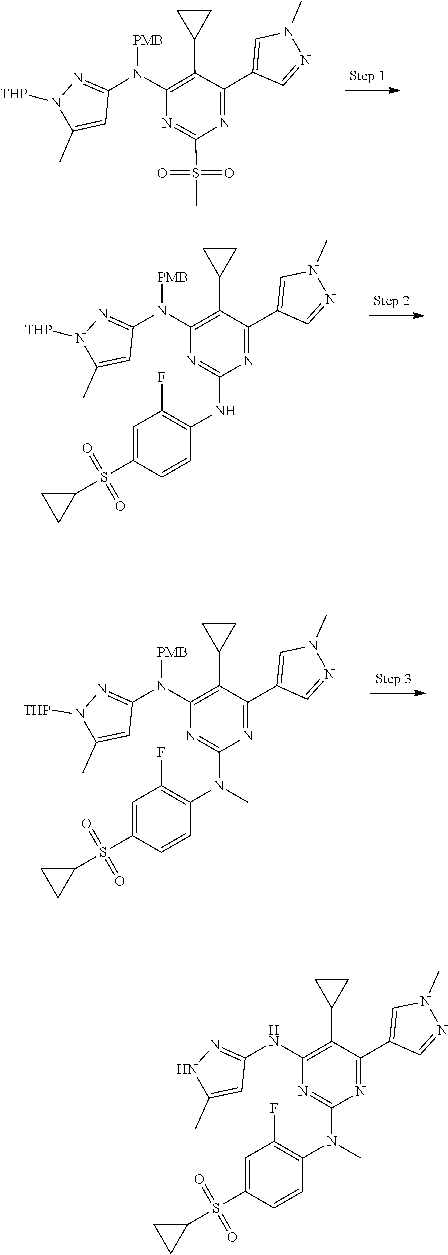

### Step 1/5-cyclopropyl-N²-(4-(cyclopropylsulfonyl)-2-fluorophenyl)-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To a solution of 5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 11, Step 1) (50.0 mg, 86.6 μmol) and 4-cyclopropylsulfonyl-2-fluoro-aniline (55.9 mg, 260 μmol) in DMPU (0.5 mL) at rt was added NaH (10.4 mg, 260 μmol, 60% dispersion). The reaction mixture was stirred at 120° C. for 3 hrs. The crude reaction mixture was quenched with water, diluted with EtOAc, quenched with NaHCO₃ sat., then extracted with EtOAc. The organic phase was washed with brine, dried over Na₂SO₄, filtrated and evaporated to give the crude desired product 5-cyclopropyl-N²-(4-(cyclopropylsulfonyl)-2-fluorophenyl)-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (60 mg) as a yellow solid which was used in the next step without purification. UPLC-MS (+ESI) m/z=713.4 (M+H)⁺.

### Step 2/5-cyclopropyl-N²-(4-(cyclopropylsulfonyl)-2-fluorophenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To crude 5-cyclopropyl-N²-(4-(cyclopropylsulfonyl)-2-fluorophenyl)-N⁴-(4-methoxybenzyl)-N⁴-(5-methyl-1-(tetrahydro-2H-pyran-2-yl)-1H-pyrazol-3-yl)-6-(1-methyl-1H-pyrazol-4-yl)pyrimidine-2,4-diamine (60 mg) in DMF (0.5 mL) was added NaH (10 mg, 240 μmol), followed by MeI (82 μL, 253 μmol) at rt for 1 h. The crude reaction mixture was quenched with water, filtrated, and purified by preparative HPLC eluting with a gradient of MeCN in water both containing 0.1% formic acid. Appropriate fractions were combined and lyophilized to afford 5-cyclopropyl-N²-(4-(cyclopropylsulfonyl)-2-fluorophenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (23 mg, 37% yield from Step 1) as beige solid. UPLC-MS (+ESI) m/z=727.4 (M+H)⁺.

### Step 3/Compound 15

To 5-cyclopropyl-N²-(4-(cyclopropylsulfonyl)-2-fluorophenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (20.0 mg, 27.5 μmol) was added TFA (0.5 mL). The reaction mixture was stirred at 80° C. for 1 h. The crude reaction mixture was evaporated, and the residue was dissolved in DMSO and purified by preparative HPLC eluting with a gradient of MeCN in water both containing 0.1% formic acid. Appropriate fractions were combined and lyophilized to afford the desired product Compound 15 (5.2 mg, 36% yield) as a white solid. UPLC-MS (+ESI) m/z=523.2 (M+H)⁺. ¹H NMR (400 MHz, DMSO-d₆) δ 11.86 (s, 1H), 8.27 (d, J=3.9 Hz, 1H), 8.24 (s, 1H), 7.95 (s, 1H), 7.84-7.77 (m, 2H), 7.74 (dd, J=8.7, 7.0 Hz, 1H), 5.63 (s, 1H), 3.91 (s, 3H), 3.49 (s, 3H), 2.96 (td, J=8.0, 4.8 Hz, 1H), 2.04 (s, 3H), 1.82-1.70 (m, 1H), 1.19 (qd, J=4.9, 2.3 Hz, 2H), 1.16-1.03 (m, 4H), 0.20 (h, J=4.0 Hz, 2H).

### Compound 16/Method A/5-cyclopropyl-N²-(2-fluoro-4-(methylsulfonyl)phenyl)-6-(1-methyl-1H-imidazol-4-yl)-N⁴-(5-methyl-1H-pyrazol-3-yl)pyrimidine-2,4-diamine

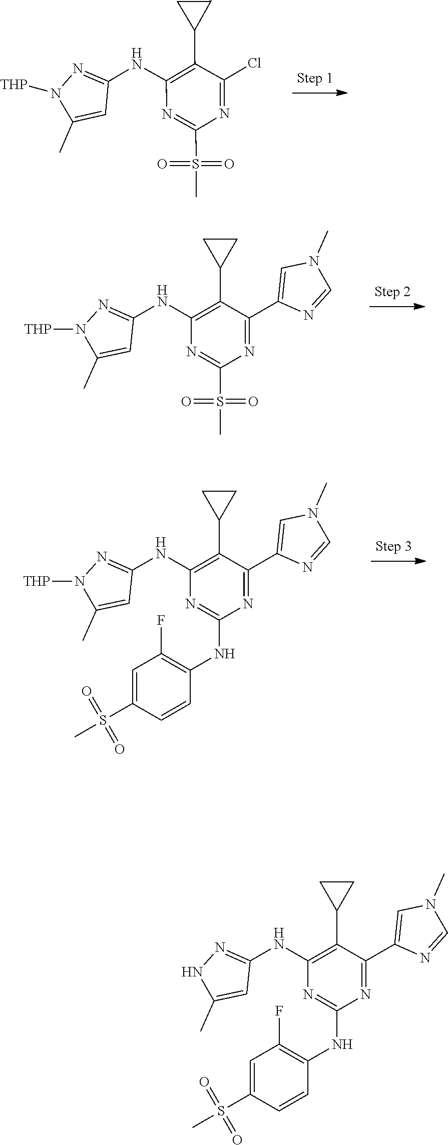

### Step 1/5-cyclopropyl-N-(5-methyl-1-(tetrahydro-2H-pyran-2-yl)-1H-pyrazol-3-yl)-6-(1-methyl-1H-imidazol-4-yl)-2-(methylsulfonyl)pyrimidin-4-amine

To solution of Intermediate 7 (200 mg, 486 μmol) in Dioxane (2 mL) was added tributyl-(1-methylimidazol-4-yl) stannane (198 mg, 534 μmol) and Pd(PPh₃)₄ (337 mg, 291 μmol). Nitrogen was bubbled in the reaction mixture then it was stirred at 100° C. for 18 hrs. The crude reaction mixture was filtrated and purified by preparative HPLC eluting with a gradient of MeCN (10 to 100%) in water both containing 0.1% formic acid. Appropriate fractions were combined and lyophilized to afford 5-cyclopropyl-N-(5-methyl-1-(tetrahydro-2H-pyran-2-yl)-1H-pyrazol-3-yl)-6-(1-methyl-1H-imidazol-4-yl)-2-(methylsulfonyl)pyrimidin-4-amine (30 mg, 14% yield). UPLC-MS (+ESI) m/z=458.3 (M+H)⁺.

### Step 2, 3/Compound 16

To a solution of 5-cyclopropyl-N-(5-methyl-1-(tetrahydro-2H-pyran-2-yl)-1H-pyrazol-3-yl)-6-(1-methyl-1H-imidazol-4-yl)-2-(methylsulfonyl)pyrimidin-4-amine (24 mg, 53 μmol) and 2-fluoro-4-methylsulfonyl-aniline (12 mg, 63 μmol) in DMPU (0.5 mL) was added NaH (4.2 mg, 115 μmol, 60% dispersion). The reaction mixture was stirred at 120° C. for 15 hrs. The crude reaction mixture was quenched with water, filtrated, and purified by preparative HPLC eluting with a gradient of MeCN in water both containing 0.1% formic acid. Appropriate fractions were combined and lyophilized to afford the intermediate 5-cyclopropyl-N²-(2-fluoro-4-(methylsulfonyl)phenyl)-N⁴-(5-methyl-1-(tetrahydro-2H-pyran-2-yl)-1H-pyrazol-3-yl)-6-(1-methyl-1H-imidazol-4-yl)pyrimidine-2,4-diamine as a yellow solid UPLC-MS (+ESI) m/z=567.3 (M+H)⁺. To the later was added TFA (0.5 mL). The reaction mixture was stirred at rt for 15 h. The crude reaction mixture was evaporated, diluted in DMSO, filtrated, and purified by preparative HPLC eluting with a gradient of MeCN in water both containing 0.1% formic acid. Appropriate fractions were combined and lyophilized to afford the desired product Compound 16 (1.0 mg, 3.8% yield for Step 2 and 3) as white solid. UPLC-MS (+ESI) m/z=483.2 (M+H)⁺.

### Compound 17/Method B/5-cyclopropyl-N²-(2-fluoro-4-(methylsulfonyl)phenyl)-N²-methyl-6-(1-methyl-1H-imidazol-4-yl)-N⁴-(5-methyl-1H-pyrazol-3-yl)pyrimidine-2,4-diamine

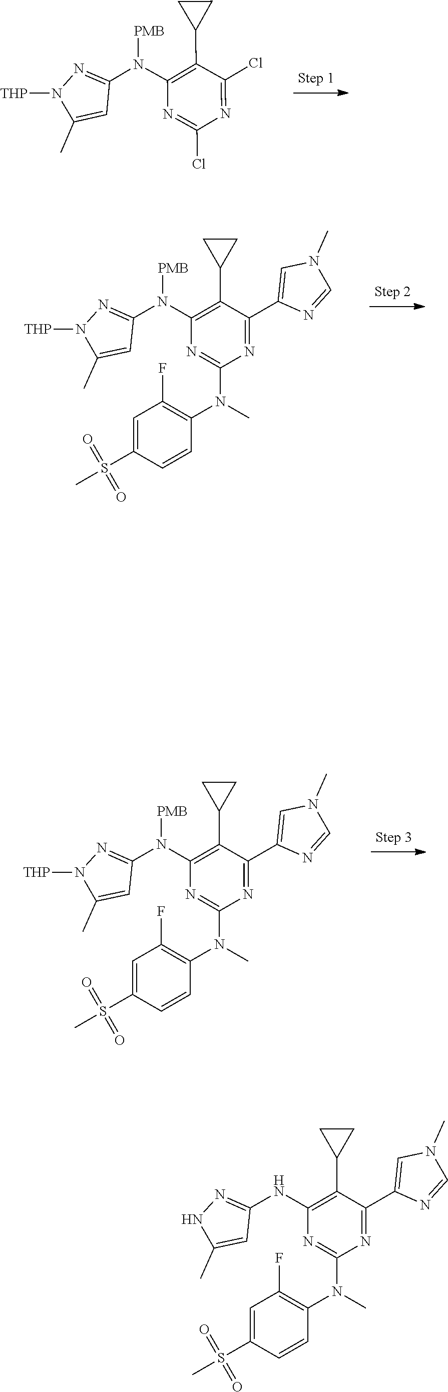

### Step 1/6-chloro-5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine

To solution of Intermediate 11 (390 mg, 798 μmol) in DMPU (3 mL) at rt was added 2-fluoro-4-methylsulfonyl-aniline (302 mg, 1.60 mmol) then NaH (96 mg, 2.4 mmol, 60% dispersion) was added. The mixture was heated to 120° C. for 4 h. The mixture was cooled down to rt and MeI (500 μL, 7.99 mmol) was added, and the mixture was stirred for 1 h at rt. MeOH (1 mL) was added and the mixture was filtered on a celite cartridge, concentrated, and purified by preparative HPLC using a gradient of MeCN (10-100%) in water both containing 0.1% formic acid. Appropriate fractions were combined and lyophilized to afford 6-chloro-5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (255 mg, 49% yield) as a 9/1 mixture of regioisomers favoring the titled compound. Used as such for the next step. UPLC-MS (+ESI) m/z=655.3 (M)⁺.

### Step 2, 3/Compound 17

To a solution of tributyl-(1-methylimidazol-4-yl) stannane (203 mg, 547 μmol) in Dioxane (2 mL) were added 6-chloro-5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (112 mg, 171 μmol) and Pd(PPh₃)₄ (99 mg, 86 μmol). The mixture was degassed (in vacuo then nitrogen) and stirred at 130° C. for 4 days. The crude reaction mixture was filtered, and the supernatant was purified by preparative HPLC eluting with a gradient of MeCN (10-100%) in water both containing 0.1% formic acid. Appropriate fractions were combined and lyophilized to afford 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine (45 mg, 38% yield). To the later was added TFA (1.5 mL) and the resulting mixture was stirred at 90° C. for 4 h. The mixture was concentrated, and the residue was purified by preparative HPLC eluting with a gradient of MeCN (10-25%) in water both containing 0.1% formic acid. Appropriate fractions were combined and lyophilized to afford Compound 17 (20 mg, 63% yield). UPLC-MS (+ESI) m/z=497.2 (M+H)⁺. ¹H NMR (400 MHz, DMSO-d₆) δ 9.33 (s, 1H), 8.25 (s, 1H), 8.11 (s, 1H), 8.07-7.98 (m, 1H), 7.83 (s, 1H), 7.78 (dd, J=8.1, 1.9 Hz, 1H), 7.71 (dd, J=8.1, 1.9 Hz, 1H), 5.48 (s, 1H), 3.97 (ddd, J=19.3, 10.2, 6.0 Hz, 3H), 3.87 (s, 3H), 3.61 (s, 3H), 3.51 (dd, J=9.0, 4.1 Hz, 2H), 3.03 (s, 3H), 2.03 (s, 3H).

### Compound 18/Method A/5-cyclopropyl-2-(2-fluoro-4-(methylsulfonyl) phenoxy)-N-(5-methyl-1H-pyrazol-3-yl)-6-(1-methyl-1H-pyrazol-4-yl)pyrimidin-4-amine

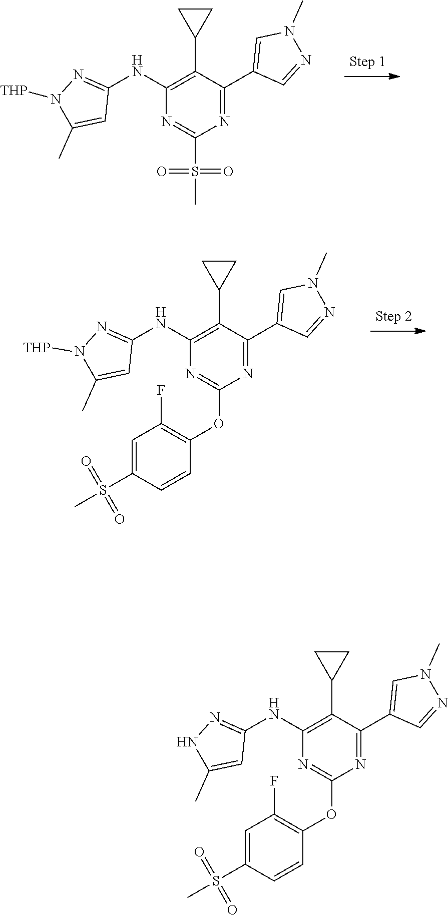

### Step 1/5-cyclopropyl-2-(2-fluoro-4-(methylsulfonyl) phenoxy)-N-(5-methyl-1-(tetrahydro-2H-pyran-2-yl)-1H-pyrazol-3-yl)-6-(1-methyl-1H-pyrazol-4-yl)pyrimidin-4-amine

To 2-fluoro-4-methylsulfonyl-phenol (42 mg, 221 μmol) in MeCN (3 mL) was added NaH (10 mg, 250 μmol, 60% dispersion) and stirred at rt for 10 min. To this mixture was added 5-cyclopropyl-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 10, Step 1) (50 mg, 110 μmol) and the reaction mixture was heated at 170° C. in the microwave for 75 min. The reaction mixture was evaporated in vacuo dissolved in DMSO and purified by preparative HPLC eluting with MeCN (40-70%) in water both containing 0.1% formic acid. The relevant fractions were combined and lyophilized to give 5-cyclopropyl-2-(2-fluoro-4-(methylsulfonyl) phenoxy)-N-(5-methyl-1-(tetrahydro-2H-pyran-2-yl)-1H-pyrazol-3-yl)-6-(1-methyl-1H-pyrazol-4-yl)pyrimidin-4-amine (9.2 mg, 15% yield). UPLC-MS (+ESI) m/z=568.2 (M+H)⁺.

### Step 2/Compound 18

To 5-cyclopropyl-2-(2-fluoro-4-(methylsulfonyl) phenoxy)-N-(5-methyl-1-(tetrahydro-2H-pyran-2-yl)-1H-pyrazol-3-yl)-6-(1-methyl-1H-pyrazol-4-yl)pyrimidin-4-amine (9.2 mg, 16 μmol) in Dioxane (1 mL) was added HCl/Dioxane (0.1 mL, 4N) and the mixture was stirred overnight at rt. The solvent was evaporated in vacuo to give Compound 18 (5.8 mg, 74% yield). UPLC-MS (+ESI) m/z=484.1 (M+H)⁺. ¹H NMR (400 MHz, DMSO-d₆) δ 8.84 (s, 1H), 8.27 (s, 1H), 7.98 (dd, J=9.7, 2.1 Hz, 1H), 7.92 (d, J=0.7 Hz, 1H), 7.87-7.81 (m, 1H), 7.66 (dd, J=8.4, 7.6 Hz, 1H), 3.87 (s, 3H), 3.29 (s, 3H), 2.01 (d, J=0.7 Hz, 3H), 1.78 (ddd, J=13.7, 8.1, 5.7 Hz, 1H), 1.25-1.08 (m, 2H), 0.19 (dt, J=6.0, 2.9 Hz, 2H).

### Compound 19/Method C/N⁶-(2-fluoro-4-(methylsulfonyl)phenyl)-3-methoxy-N⁶-methyl-N²-(5-methyl-1H-pyrazol-3-yl)-4-(1-methyl-1H-pyrazol-4-yl)pyridine-2,6-diamine

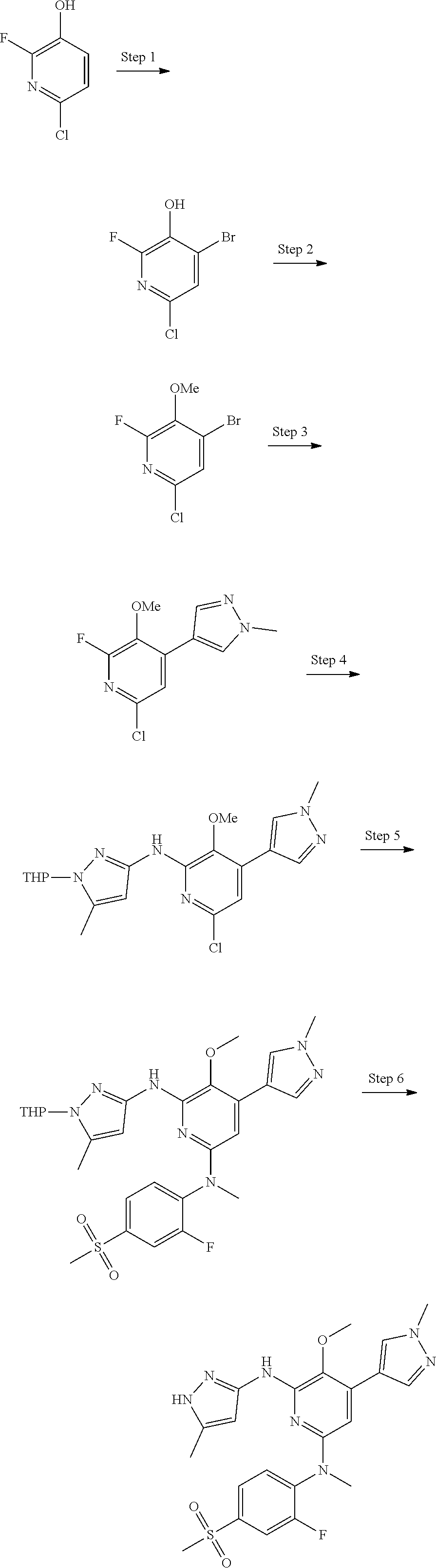

### Step 1/4-bromo-6-chloro-2-fluoropyridin-3-ol

In a round bottom flask was dissolved 6-chloro-2-fluoropyridin-3-ol (3.00 g, 20.3 mmol) in a mixture of MeCN (50 mL) and water (25 mL). Br₂ (1.04 mL, 20.3 mmol) was then added dropwise, and the reaction was stirred at rt. After 2 hrs, the crude was evaporated to dryness and directly purified by silica gel chromatography eluting with EtOAc (0 to 30%) in Heptane. Appropriate fractions were combined and concentrated to afford 4-bromo-6-chloro-2-fluoropyridin-3-ol (5.0 g, 99% yield) as an orange oil. UPLC-MS (+ESI) m/z=228.0 (M+H)⁺. ¹H NMR (400 MHz, CDCl₃) δ ppm 6.42 (br s, 1H), 7.38 (s, 1H). ¹⁹F NMR (377 MHz, CDCl₃) δ ppm −83.73 (s, 1F).

### Step 2/4-bromo-6-chloro-2-fluoro-3-methoxypyridine

In a round bottom flask, Cs₂CO₃ (411 mg, 1.26 mmol) was added to a solution of 4-bromo-6-chloro-2-fluoropyridin-3-ol (500 mg, 2.21 mmol) in DMF (14 mL). MeI (275 μL, 4.42 mmol) was then added and the reaction mixture was stirred at rt for 18 hrs. The reaction mixture was diluted in MeTHF and washed with a saturated solution of NaHCO₃. The aqueous layer was extracted with MeTHF (3×). The combined organic layers were washed with brine, dried over Na₂SO₄, filtered, and concentrated under vacuum. The crude mixture was purified by silica gel chromatography eluting with EtOAc (0 to 30%) in Heptane. The appropriate fractions were combined and concentrated to give 4-bromo-6-chloro-2-fluoro-3-methoxypyridine (500 mg, 95% yield) as a pale-yellow solid. ¹H NMR (400 MHz, CDCl₃) δ ppm 4.00 (d, J=2.2 Hz, 3H), 7.42 (s, 1H). ¹⁹F NMR (377 MHz, CDCl₃) δ ppm −77.15 (s, 1F).

### Step 3/6-chloro-2-fluoro-3-methoxy-4-(1-methyl-1H-pyrazol-4-yl)pyridine

A sealable tube was charged with 4-bromo-6-chloro-2-fluoro-3-methoxypyridine (500 mg, 2.08 mmol) in 1,4-dioxane/water (3/1). (1-Methyl-1H-pyrazol-4-yl) boronic acid (288 mg, 2.29 mmol) and Cs₂CO₃ (2.00 g, 6.24 mmol) was then added and the mixture was sparged with nitrogen for 10 min. Pd(dppf)Cl₂ (170 mg, 0.21 mmol) was added and the suspension was sparged with nitrogen for another 10 min. The reaction mixture was stirred at 90° C. for 18 h. The reaction was filtered through a pad of celite and washed with EtOAc. The combined supernatants were concentrated in vacuo. The residue was purified by preparative HPLC eluting with a gradient of MeCN (5 to 95%) in water both containing 0.1% formic acid. Appropriate fractions were combined and lyophilization to afford 6-chloro-2-fluoro-3-methoxy-4-(1-methyl-1H-pyrazol-4-yl)pyridine (375 mg, 75% yield) as yellow solid. UPLC-MS (+ESI) m/z=242.2 (M+H)⁺. ¹H NMR (400 MHz, CDCl₃) δ ppm 3.95 (d, J=2.2 Hz, 3H), 4.00 (s, 3H), 7.31 (s, 1H), 7.94 (s, 1H), 7.98 (s, 1H). ¹⁹F NMR (377 MHz, CDCl₃) δ ppm −79.93 (s, 1F).

### Step 4/6-chloro-3-methoxy-N-(5-methyl-1-(tetrahydro-2H-pyran-2-yl)-1H-pyrazol-3-yl)-4-(1-methyl-1H-pyrazol-4-yl)pyridin-2-amine

In a sealable tube a 1 M solution of NaHMDS in THF (6.21 mL, 6.21 mmol) was added to a solution of 6-chloro-2-fluoro-3-methoxy-4-(1-methyl-1H-pyrazol-4-yl)pyridine (422 mg, 2.33 mmol) in THF (39 mL) at rt. Intermediate 1 (375 mg, 1.55 mmol) was then added and the reaction was sealed and stirred at 80° C. for 3 h. The reaction was quenched with H₂O and the aqueous layer was extracted with EtOAc (3×). The combined organic layers were dried over Na₂SO₄, filtered, and concentrated under vacuum. The residue was purified by preparative HPLC eluting with a gradient of MeCN (5 to 95%) in water both containing 0.1% formic acid. Appropriate fractions were combined and lyophilization to afford 6-chloro-3-methoxy-N-(5-methyl-1-(tetrahydro-2H-pyran-2-yl)-1H-pyrazol-3-yl)-4-(1-methyl-1H-pyrazol-4-yl)pyridin-2-amine (484 mg, 77% yield) as brown solid. UPLC-MS (+ESI) m/z=403.2 (M+H)⁺. ¹H NMR (400 MHz, CDCl₃) δ ppm 1.55-1.62 (m, 1H), 1.65-1.79 (m, 2H), 1.83-1.93 (m, 1H), 2.05-2.16 (m, 1H), 2.37 (s, 3H), 2.38-2.43 (m, 1H), 3.65 (s, 3H), 3.66-3.72 (m, 1H), 3.99 (s, 3H), 4.08-4.17 (m, 1H), 5.18 (dd, J=10.3, 2.2 Hz, 1H), 6.78 (s, 1H), 6.80 (s, 1H), 7.74 (s, 1H), 7.90 (d, J=3.9 Hz, 2H).

### Step 5/N⁶-(2-fluoro-4-(methylsulfonyl)phenyl)-3-methoxy-N⁶-methyl-N²-(5-methyl-1-(tetrahydro-2H-pyran-2-yl)-1H-pyrazol-3-yl)-4-(1-methyl-1H-pyrazol-4-yl)pyridine-2,6-diamine

A flame dried sealable tube was charged with 6-chloro-3-methoxy-N-(5-methyl-1-(tetrahydro-2H-pyran-2-yl)-1H-pyrazol-3-yl)-4-(1-methyl-1H-pyrazol-4-yl)pyridin-2-amine (50.0 mg, 0.12 mmol) in toluene (1.20 mL). 2-fluoro-4-methanesulfonyl-N-methylaniline (37.8 mg, 0.190 mmol), Cs₂CO₃ (121 mg, 0.370 mmol) and +/−BINAP (31 mg, 0.050 mmol) were added and the mixture was sparged with nitrogen for 10 min. Pd(OAc)₂ (5.6 mg, 0.025 mmol) was added and the suspension was sparged with nitrogen for another 10 min. The reaction mixture was stirred at 95° C. for 18 hrs. The reaction mixture was filtered through a pad of celite and washed with EtOAc. The solvent was removed under vacuum and the crude residue was purified preparative HPLC eluting with a gradient of MeCN (5 to 95%) in water both containing 0.1% formic acid. Appropriate fractions were combined and lyophilization to afford N⁶-(2-fluoro-4-(methylsulfonyl)phenyl)-3-methoxy-N⁶-methyl-N²-(5-methyl-1-(tetrahydro-2H-pyran-2-yl)-1H-pyrazol-3-yl)-4-(1-methyl-1H-pyrazol-4-yl)pyridine-2,6-diamine (35 mg, 50% yield) as yellow oil. UPLC-MS (+ESI) m/z=570.2 (M+H)⁺. ¹H NMR (400 MHz, CDCl₃) δ ppm 1.82-1.90 (m, 2H), 2.03-2.13 (m, 2H), 2.22 (s, 3H), 2.33-2.41 (m, 1H), 3.11 (s, 3H), 3.47 (s, 3H), 3.60 (s, 3H), 3.62-3.69 (m, 1H), 3.98 (s, 3H), 4.07-4.13 (m, 1H), 5.09-5.15 (m, 1H), 6.05 (s, 1H), 6.17 (s, 1H), 7.52-7.55 (m, 2H), 7.68-7.77 (m, 2H), 7.87 (s, 2H).

### Step 6/Compound 19

A 37% HCl solution in MeOH (735 μL) was added to a solution of N⁶-(2-fluoro-4-(methylsulfonyl)phenyl)-3-methoxy-N⁶-methyl-N²-(5-methyl-1-(tetrahydro-2H-pyran-2-yl)-1H-pyrazol-3-yl)-4-(1-methyl-1H-pyrazol-4-yl)pyridine-2,6-diamine (34.0 mg, 0.060 mmol) in MeOH (1 mL). The reaction mixture was stirred at 50° C. for 4 hrs. The reaction was concentrated under vacuum and co-evaporated with MeCN (2×). The crude residue was purified preparative HPLC eluting with a gradient of MeCN (5 to 95%) in water both containing 0.1% formic acid. Appropriate fractions were combined and lyophilization to afford Compound 19 (13 mg, 45% yield) as white solid. UPLC-MS (+ESI) m/z=486.1 (M+H)⁺. ¹H NMR (400 MHz, DMSO-d₆) δ ppm 2.00 (s, 3H), 3.30 (s, 3H), 3.41 (s, 3H), 3.56 (s, 3H), 3.91 (s, 3H), 5.55 (s, 1H), 6.43 (s, 1H), 7.66 (t, J=7.9 Hz, 1H), 7.75-7.84 (m, 2H), 8.01 (s, 1H), 8.28 (s, 1H). ¹⁹F NMR (377 MHz, CDCl₃) δ ppm-114.57 (br s, 1F).

### Compound 20/Method C/3-cyclopropyl-N⁶-(2-fluoro-4-(methylsulfonyl)phenyl)-N⁶-methyl-N²-(5-methyl-1H-pyrazol-3-yl)-4-(1-methyl-1H-pyrazol-4-yl)pyridine-2,6-diamine

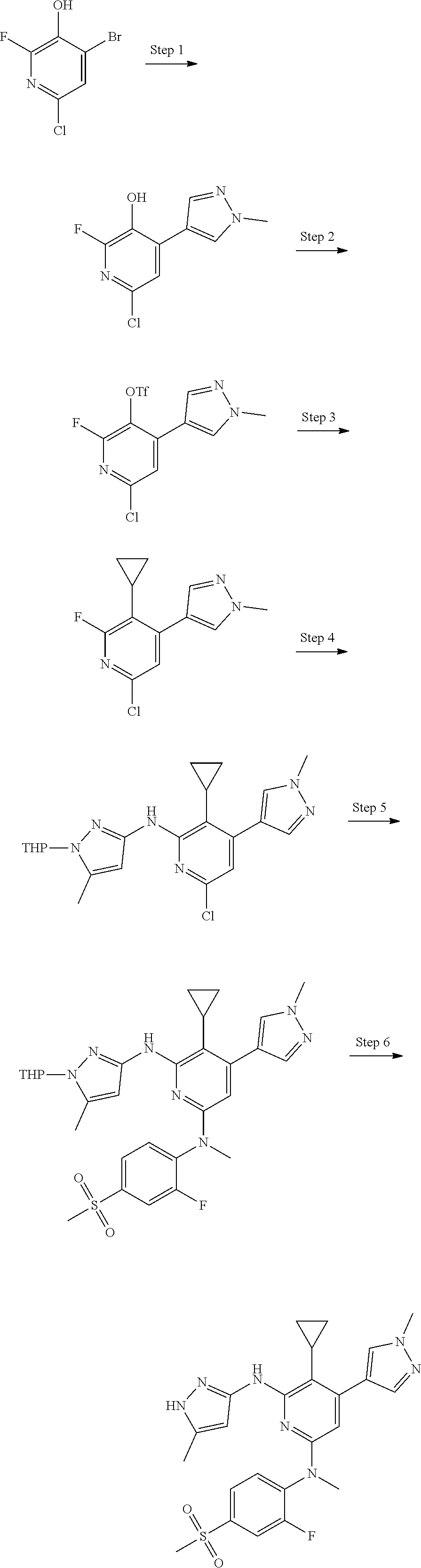

### Step 1/6-chloro-2-fluoro-4-(1-methyl-1H-pyrazol-4-yl)pyridin-3-ol

In a Schlenk flask under nitrogen was dissolved 4-bromo-6-chloro-2-fluoropyridin-3-ol (Compound 19, Step 1) (600 mg, 2.60 mmol), 1-methyl-4-(4,4,5,5-tetramethyl-1,3,2-dioxaborolan-2-yl)-1H-pyrazole (607 mg, 2.90 mmol), tri-tert-butylphosphine (530 μL, 0.50 mmol) and K₃PO₄ (1.10 g, 5.30 mmol) in Dioxane (12 mL) and water (6 mL). The solution was sparged with nitrogen for 10 min. Then Pd (dba) 3 (243 mg, 0.260 mmol) was added. The mixture was sparged with nitrogen for another 10 min and the reaction was sealed and stirred at 90° C. After 3 hrs, the reaction was cooled to rt and filtered through a pad of celite, washed with EtOAc. The organic layer was concentrated and the crude residue was purified by preparative HPLC eluting with a gradient of MeCN (5 to 100%) in water both containing 0.1% formic acid. Appropriate fractions were combined and lyophilization to afford 6-chloro-2-fluoro-4-(1-methyl-1H-pyrazol-4-yl)pyridin-3-ol (166 mg, 28% yield) as a light-yellow solid. UPLC-MS (+ESI) m/z=228.2 (M+H)⁺. ¹H NMR (400 MHz, DMSO-d₆) δ ppm 3.91 (s, 3H), 7.67 (s, 1H), 8.17 (s, 1H), 8.41 (s, 1H), 10.74 (br s, 1H). ¹⁹F NMR (377 MHz, DMSO-d₆) δ ppm −86.28 (s, 1F).

### Step 2/6-chloro-2-fluoro-4-(1-methyl-1H-pyrazol-4-yl)pyridin-3-yl trifluoromethanesulfonate

In a round bottom flask under nitrogen was dissolved 6-chloro-2-fluoro-4-(1-methyl-1H-pyrazol-4-yl)pyridin-3-ol (166 mg, 0.72 mmol) in dry DCM (5 mL). Pyridine (176 μL, 2.20 mmol) was then added, followed by Tf₂O (135 μL, 0.80 mmol) dropwise. Reaction was stirred at rt for 2 hrs, then evaporated to dryness and purified on silica gel chromatography eluting with EtOAc (0 to 80%) in Heptane. Appropriate fractions were combined and concentrated in vacuo to afford 6-chloro-2-fluoro-4-(1-methyl-1H-pyrazol-4-yl)pyridin-3-yl trifluoromethanesulfonate (126 mg, 48% yield) as a white solid. UPLC-MS (+ESI) m/z=360.0 (M+H)⁺. ¹H NMR (400 MHz, CDCl₃) δ ppm 4.02 (s, 3H), 7.39 (s, 1H), 7.90 (s, 1H), 7.96 (s, 1H). ¹⁹F NMR (377 MHz, CDCl₃) δ ppm −76.41 (q, J=15.9 Hz, 1F), −72.60 (d, J=15.0 Hz, 3F).

### Step 3/6-chloro-3-cyclopropyl-2-fluoro-4-(1-methyl-1H-pyrazol-4-yl)pyridine

In a Schlenk flask under N₂ was dissolved 6-chloro-2-fluoro-4-(1-methyl-1H-pyrazol-4-yl) pyridin-3-yl trifluoromethanesulfonate (126 mg, 0.35 mmol), Cyclopropyl-BF₃K (62.2 mg, 0.42 mmol), K₂CO₃ (145 mg, 1.05 mmol) and Ruphos (32.7 mg, 0.070 mmol) in a mixture of toluene (3 mL) and water (1.5 mL). The reaction mixture was sparged for 10 min with nitrogen, then Pd(OAc)₂ (7.9 mg, 0.030 mmol) was added and the reaction was sparged for another 10 min. The flask was sealed and heated at 90° C. After 3 hrs, the reaction was cooled to rt, filtered through a pad of celite, washed with EtOAc. The supernatant was concentrated, and the crude residue was purified preparative HPLC eluting with a gradient of MeCN (5 to 100%) in water both containing 0.1% formic acid. Appropriate fractions were combined and lyophilization to afford 6-chloro-3-cyclopropyl-2-fluoro-4-(1-methyl-1H-pyrazol-4-yl)pyridine (34 mg, 39% yield) as an off-white solid. UPLC-MS (+ESI) m/z=252.2 (M+H)⁺. ¹H NMR (400 MHz, CDCl₃) δ ppm 0.60-0.66 (m, 2H), 1.00-1.07 (m, 2H), 1.73-1.81 (m, 1H), 4.01 (s, 3H), 7.21 (s, 1H), 7.76 (s, 1H), 7.84 (s, 1H). ¹⁹F NMR (377 MHz, CDCl₃) δ ppm −66.57 (s, 1F).

### Step 4/6-chloro-3-cyclopropyl-N-(5-methyl-1-(tetrahydro-2H-pyran-2-yl)-1H-pyrazol-3-yl)-4-(1-methyl-1H-pyrazol-4-yl)pyridin-2-amine

In a vial under nitrogen was dissolved 6-chloro-3-cyclopropyl-2-fluoro-4-(1-methyl-1H-pyrazol-4-yl)pyridine (47.5 mg, 0.260 mmol) in anhydrous THF (3 mL). NaHMDS (700 μL, 0.700 mmol) was then added dropwise and stirred at rt over 5 minutes. Intermediate 1 (44.0 mg, 0.17 mmol) was then added and the reaction mixture was sealed and stirred at 80° C. The reaction was stopped after 1.5 hrs and evaporated to dryness. The crude residue was purified by preparative HPLC eluting with a gradient of MeCN (5 to 100%) in water both containing 0.1% formic acid. Appropriate fractions were combined and lyophilized to afford 6-chloro-3-cyclopropyl-N-(5-methyl-1-(tetrahydro-2H-pyran-2-yl)-1H-pyrazol-3-yl)-4-(1-methyl-1H-pyrazol-4-yl)pyridin-2-amine (53 mg, 73% yield) as a yellow solid. UPLC-MS (+ESI) m/z=413.2 (M+H)⁺. ¹H NMR (400 MHz, CDCl₃) δ ppm 0.34-0.40 (m, 2H), 1.07-1.13 (m, 2H), 1.64-1.69 (m, 4H), 1.86-1.92 (m, 1H), 2.06-2.13 (m, 1H), 2.37 (s, 3H), 2.39-2.49 (m, 1H), 3.63-3.71 (m, 1H), 3.99 (s, 3H), 4.10-4.16 (m, 1H), 5.18 (dd, J=10.4, 2.3 Hz, 1H), 6.72 (s, 1H), 6.78 (s, 1H), 7.62 (s, 1H), 7.73 (s, 2H).

### Step 5/3-cyclopropyl-N⁶-(2-fluoro-4-(methylsulfonyl)phenyl)-N⁶-methyl-N²-(5-methyl-1-(tetrahydro-2H-pyran-2-yl)-1H-pyrazol-3-yl)-4-(1-methyl-1H-pyrazol-4-yl)pyridine-2,6-diamine

In a vial was dissolved 6-chloro-3-cyclopropyl-N-(5-methyl-1-(tetrahydro-2H-pyran-2-yl)-1H-pyrazol-3-yl)-4-(1-methyl-1H-pyrazol-4-yl)pyridin-2-amine (53 mg, 0.12 mmol) and 2-fluoro-N-methyl-4-(methylsulfonyl) aniline (39 mg, 0.19 mmol) in dry toluene (1.25 mL). Cs₂CO₃ (126 mg, 0.38 mmol) and +/−BINAP (32 mg, 0.051 mmol) was then added. The solution was sparged with nitrogen for 15 min. Pd(OAc)₂ (5.8 mg, 0.02 mmol) was added and sparging continued for 5 min. The flask was sealed and heated at 95° C. After 3 hrs, the reaction mixture was cooled to rt, filtered through a pad of celite and washed with EtOAc. The supernatant was concentrated, and the crude residue was purified by preparative HPLC eluting with a gradient of MeCN (5 to 100%) in water both containing 0.1% formic acid. Appropriate fractions were combined and lyophilization to afford 3-cyclopropyl-N⁶-(2-fluoro-4-(methylsulfonyl)phenyl)-Ne-methyl-N²-(5-methyl-1-(tetrahydro-2H-pyran-2-yl)-1H-pyrazol-3-yl)-4-(1-methyl-1H-pyrazol-4-yl)pyridine-2,6-diamine (30.0 mg, 40% yield) as a light-yellow solid. UPLC-MS (+ESI) m/z=580.2 (M+H)⁺. ¹H NMR (400 MHz, CDCl₃) δ ppm 0.29-0.35 (m, 2H), 0.97-1.04 (m, 2H), 1.67-1.73 (m, J=9.5 Hz, 4H), 1.84-1.88 (m, 1H), 2.05-2.12 (m, 1H), 2.22 (s, 3H), 2.34-2.47 (m, 1H), 3.10 (s, 3H), 3.46 (s, 3H), 3.59-3.69 (m, 1H), 3.97 (s, 3H), 4.07-4.13 (m, 1H), 5.12 (dd, J=10.5, 2.2 Hz, 1H), 6.05 (s, 1H), 6.08 (s, 1H), 7.52-7.57 (m, 2H), 7.65-7.73 (m, 3H). ¹⁹F NMR (377 MHz, CDCl₃) δ ppm −113.50 (s, 1F).

### Step 6/Compound 20

3-cyclopropyl-N⁶-(2-fluoro-4-(methylsulfonyl)phenyl)-N⁶-methyl-N²-(5-methyl-1-(tetrahydro-2H-pyran-2-yl)-1H-pyrazol-3-yl)-4-(1-methyl-1H-pyrazol-4-yl)pyridine-2,6-diamine (27 mg, 0.04 mmol) was dissolved in a 3M HCl solution in MeOH (776 μL, 2.32 mmol) and stirred at rt for 1 h, then warmed to 50° C. for 4 hrs. The crude mixture was evaporated to dryness and residue was purified by preparative HPLC eluting with a gradient of MeCN (5 to 100%) in water both containing 0.1% formic acid. Appropriate fractions were combined and lyophilization to afford Compound 20 (14.7 mg, 65% yield) as a white solid. UPLC-MS (+ESI) m/z=496.1 (M+H)⁺. ¹H NMR (400 MHz, DMSO-d₆) δ ppm 0.09-0.15 (m, 2H), 1.03-1.10 (m, 2H), 1.77-1.84 (m, 1H), 2.11-2.15 (m, 3H), 3.32 (s, 3H), 3.43 (s, 3H), 3.90 (s, 3H), 5.78 (s, 1H), 6.42 (s, 1H), 7.71-7.77 (m, 1H), 7.84-7.90 (m, 3H), 8.19 (s, 1H), 8.95 (br s, 1H). ¹⁹F NMR (377 MHz, DMSO-d₆) δ ppm −115.14 (s, 1F).

### Compound 21/Method C/6-((2-fluoro-4-(methylsulfonyl)phenyl)thio)-3-methoxy-N-(5-methyl-1H-pyrazol-3-yl)-4-(1-methyl-1H-pyrazol-4-yl)pyridin-2-amine

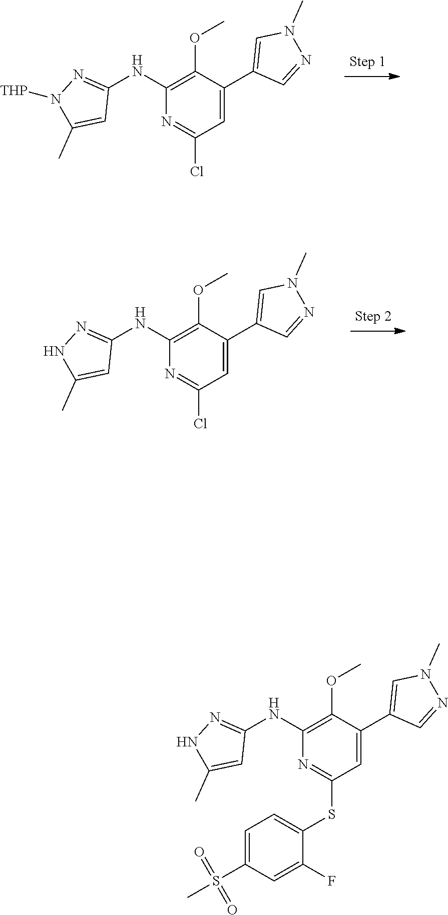

### Step 1/6-chloro-3-methoxy-N-(5-methyl-1H-pyrazol-3-yl)-4-(1-methyl-1H-pyrazol-4-yl)pyridin-2-amine

A 3 M solution of HCl in MeOH (10.7 mL, 42.7 mmol) was added to a mixture of 6-chloro-3-methoxy-N-(5-methyl-1-(tetrahydro-2H-pyran-2-yl)-1H-pyrazol-3-yl)-4-(1-methyl-1H-pyrazol-4-yl) pyridin-2-amine (Compound 19, Step 4) (344 mg, 0.85 mmol) in MeOH (8.3 mL, 0.1 M). The reaction mixture was stirred at 50° C. for 2 hrs. The reaction mixture was concentrated under vacuum and co-evaporated two times with MeCN. The residue was purified by reverse preparative HPLC eluting with a gradient of MeCN (5 to 95%) in water both containing 0.1% formic acid. Appropriate fractions were combined and lyophilized to afford 6-chloro-3-methoxy-N-(5-methyl-1H-pyrazol-3-yl)-4-(1-methyl-1H-pyrazol-4-yl)pyridin-2-amine (228 mg, 84% yield) as brown solid. UPLC-MS (+ESI) m/z=319.2 (M+H)⁺. ¹H NMR (400 MHz, DMSO-d₆) δ ppm 2.35 (s, 3H), 3.67 (s, 3H), 3.92 (s, 3H), 6.44 (s, 1H), 7.33 (s, 1H), 8.12 (s, 1H), 8.41 (s, 1H), 10.03 (s, 1H).

### Step 2/Compound 21

In a sealed tube, pivalic acid (160 mg, 1.57 mmol) was added to a solution of 6-chloro-3-methoxy-N-(5-methyl-1H-pyrazol-3-yl)-4-(1-methyl-1H-pyrazol-4-yl)pyridin-2-amine (100 mg, 0.31 mmol) in Dioxane (1 mL, 0.3 M). 2-fluoro-4-methanesulfonylbenzene-1-thiol (97.1 mg, 0.47 mmol) was then added, and the reaction mixture was stirred at 120° C. for 6 hrs. 2-fluoro-4-methanesulfonylbenzene-1-thiol (97.1 mg, 0.47 mmol) was added again, and the reaction mixture was stirred at 120° C. for another 18 hrs. The reaction was quenched with saturated aqueous NaHCO₃ and extracted with MeTHF (3×). The combined organic layers were washed with brine, dried over Na₂SO₄, filtered, and concentrated under vacuum. The residue was purified by preparative HPLC eluting with a gradient of MeCN (5 to 95%) in water both containing 0.1% formic acid. Appropriate fractions were combined and lyophilization to afford Compound 21 (17 mg, 11% yield) as white solid. UPLC-MS (+ESI) m/z=489.1 (M+H)⁺. ¹H NMR (400 MHz, DMSO-d₆) δ ppm 2.02 (s, 3H), 3.29 (br s, 3H), 3.63 (s, 3H), 3.91 (s, 3H), 5.54 (s, 1H), 7.16 (br s, 1H), 7.77-7.83 (m, 1H), 7.83-7.92 (m, 2H), 8.03 (s, 1H), 8.33 (s, 2H), 11.70-11.85 (m, 1H). ¹⁹F NMR (377 MHz, CDCl₃) δ ppm −104.75 (br s, 1F).

### Compound 22/Method D/6-((2-fluoro-4-(methylsulfonyl)phenyl)thio)-3-methoxy-5-methyl-N-(5-methyl-1H-pyrazol-3-yl)-4-(1-methyl-1H-pyrazol-4-yl)pyridin-2-amine

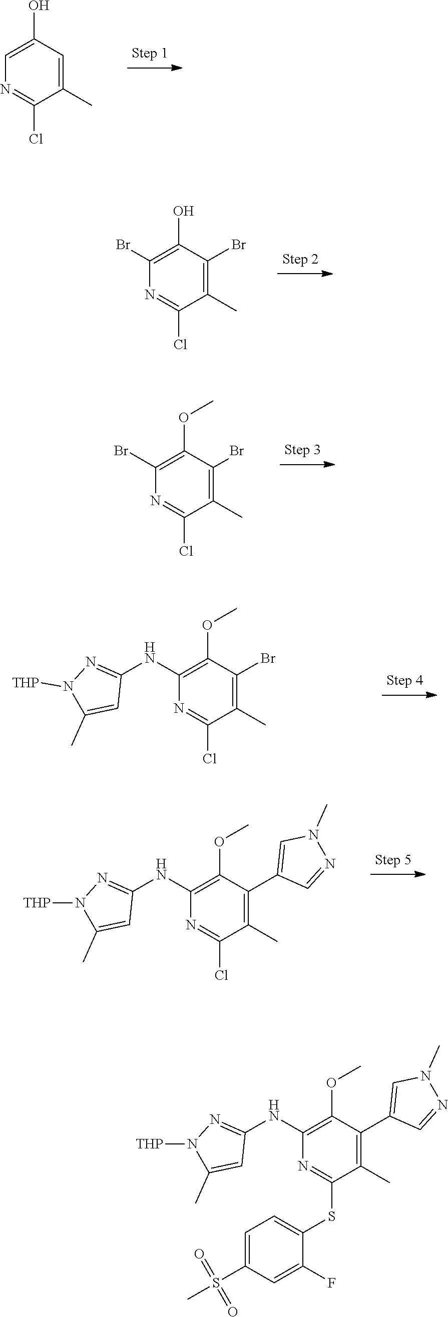

### Step 1/2,4-dibromo-6-chloro-5-methylpyridin-3-ol

To a solution of 6-chloro-5-methylpyridin-3-ol (4.67 g, 32.5 mmol) in MeCN (103 mL) and water (34 mL) was added bromine (3.74 mL, 72.9 mmol) dropwise (10 min) at rt and under nitrogen atmosphere. The reaction mixture was stirred at rt for 20 hrs. 10% aq. Na₂S₂O₃ was added until the red color disappeared, the volatiles were removed under vacuum and the resulting mixture was extracted with DCM (3×). The combined organics phases were washed with H₂O, brine, dried over Na₂SO₄, filtered, and concentrated to give 2,4-dibromo-6-chloro-5-methylpyridin-3-ol (10.39 g, quantitative, crude) as a light-yellow solid. The product was used in the next step without further purification. UPLC-MS (+ESI) m/z=301.8 (M+H)⁺. ¹H NMR (400 MHz, CDCl₃) δ ppm 2.51 (s, 3H), 5.88 (br s, 1H).

### Step 2/2,4-dibromo-6-chloro-3-methoxy-5-methylpyridine

To a suspension of 2,4-dibromo-6-chloro-5-methylpyridin-3-ol (11.24 g, 37.30 mmol) and Cs₂CO₃ (24.3 g, 74.6 mmol) in DMF (233 mL) was added MeI (4.64 mL, 74.6 mmol) dropwise at rt and under nitrogen atmosphere. The reaction mixture was stirred at rt for 15 hrs and then poured in water (1000 mL). The mixture was stirred for 5 min and the precipitate was collected by filtration. The cake was washed with water, dried on the Buchner funnel for 1 h with vacuum to give a wet solid. The latter was dissolved in DCM (200 mL). The solution was dried over Na₂SO₄, filtered, and concentrated to give 2,4-dibromo-6-chloro-3-methoxy-5-methylpyridine (9.68 g, crude) as an off-white solid. The product was used in the next step without further purification. UPLC-MS (+ESI) m/z=315.8 (M+H)⁺. ¹H NMR (400 MHz, CDCl₃) δ ppm 2.50 (s, 3H), 3.92 (s, 3H).

### Step 3/4-bromo-6-chloro-3-methoxy-5-methyl-N-(5-methyl-1-(tetrahydro-2H-pyran-2-yl)-1H-pyrazol-3-yl)pyridin-2-amine

A solution of Intermediate 1 (1.90 g, 10.5 mmol), 2,4-dibromo-6-chloro-3-methoxy-5-methylpyridine (3.00 g, 9.51 mmol), Xanthphos (1.65 g, 2.85 mmol) and K₂CO₃ (3.94 g, 28.5 mmol) in Dioxane (51 mL) was sparged with nitrogen for 10 min then Pd(OAc)₂ (320 mg, 1.43 mmol) was added to the mixture. Sparging was resumed for 5 min and the vial was sealed and stirred at 100° C. for 18 hrs). The reaction mixture was filtered on celite, concentrated to dryness and purified by silica gel chromatography eluting with EtOAc (0 to 60%) in Heptane. Appropriate fractions were combined and concentrated in vacuo to afford 4-bromo-6-chloro-3-methoxy-5-methyl-N-(5-methyl-1-(tetrahydro-2H-pyran-2-yl)-1H-pyrazol-3-yl)pyridin-2-amine (2.31 g, 58% yield) as a yellow solid. UPLC-MS (+ESI) m/z=415 (M+H)⁺. ¹H NMR (400 MHz, CDCl₃) δ ppm 1.63-1.79 (m, 2H), 1.83-1.91 (m, 1H), 2.06-2.13 (m, 1H), 2.31-2.38 (m, 4H), 2.38-2.44 (m, 4H), 3.62 (dt, J=11.5, 2.2 Hz, 1H), 3.84 (s, 3H), 4.05-4.17 (m, 1H), 5.18 (dd, J=10.5, 2.4 Hz, 1H), 6.72 (s, 1H), 7.43 (s, 1H).

### Step 4/6-chloro-3-methoxy-5-methyl-N-(5-methyl-1-(tetrahydro-2H-pyran-2-yl)-1H-pyrazol-3-yl)-4-(1-methyl-1H-pyrazol-4-yl)pyridin-2-amine

A solution of 4-bromo-6-chloro-3-methoxy-5-methyl-N-(5-methyl-1-(tetrahydro-2H-pyran-2-yl)-1H-pyrazol-3-yl)pyridin-2-amine (2.31 g, 5.56 mmol), 1-methylpyrazol-4-ylboronic acid (735 mg, 5.83 mmol), NaHCO₃ (1.87 g, 22.2 mmol) in water (11 mL) and Dioxane (32 mL) was sparged 10 min with nitrogen, then Pd(PPh₃)₄ (642 mg, 0.56 mmol) was added and sparging was resumed for 5 min. The vial was sealed and heated at 100° C. for 18 hrs. The reaction mixture was filtered on a celite pad, concentrated to dryness and the residue was purified preparative HPLC eluting with a gradient of MeCN (5 to 100%) in water both containing 0.1% formic acid. Appropriate fractions were combined and lyophilization to afford give 6-chloro-3-methoxy-5-methyl-N-(5-methyl-1-(tetrahydro-2H-pyran-2-yl)-1H-pyrazol-3-yl)-4-(1-methyl-1H-pyrazol-4-yl)pyridin-2-amine (584 mg, 25% yield) as a light-yellow solid. UPLC-MS (+ESI) m/z=417.2 (M+H)⁺. ¹H NMR (400 MHz, CDCl₃) δ ppm 1.57-1.60 (m, 1H), 1.62-1.81 (m, 2H), 1.84-1.91 (m, 1H), 2.05-2.13 (m, 1H), 2.24 (s, 3H), 2.33-2.45 (m, 4H), 3.38 (s, 3H), 3.66 (dt, J=11.2, 2.0 Hz, 1H), 4.00 (s, 3H), 4.11 (dt, J=11.6, 1.9 Hz, 1H), 5.17 (dd, J=10.3, 2.4 Hz, 1H), 6.77 (s, 1H), 7.47 (br s, 1H), 7.56 (s, 1H), 7.63 (s, 1H).

### Step 5/Compound 22

In a sealable tube, a solution of 6-chloro-3-methoxy-5-methyl-N-(5-methyl-1-(tetrahydro-2H-pyran-2-yl)-1H-pyrazol-3-yl)-4-(1-methyl-1H-pyrazol-4-yl)pyridin-2-amine (220 mg, 0.53 mmol) and 2-fluoro-4-(methylsulfonyl)benzenethiol (163 mg, 0.79 mmol) in iPrOH (1 mL) was sparged with nitrogen for 5 min. The tube was sealed, and the mixture was stirred at 120° C. for 15 hrs. The mixture was concentrated, and the residue was purified by preparative HPLC eluting with a gradient of MeCN (5 to 100%) in water both containing 0.1% formic acid. Appropriate fractions were combined and lyophilized to afford Compound 22 (48 mg, 17% yield) as a light-yellow solid. UPLC-MS (+ESI) m/z=503.1 (M+H)⁺. ¹H NMR (400 MHz, DMSO-d₆) δ ppm 1.93-2.03 (m, 3H), 2.22 (s, 3H), 3.31 (s, 3H), 3.34 (s, 3H), 3.93 (s, 3H), 5.29 (s, 1H), 7.65 (s, 1H), 7.80-7.92 (m, 3H), 7.98 (s, 1H), 8.34 (br s, 1H), 11.58 (br s, 1H). ¹⁹F NMR (377 MHz, DMSO-d₆) δ ppm −103.61 (br s, 1F).

### Compound 23/Method D/N²-(2-fluoro-4-(methylsulfonyl)phenyl)-5-methoxy-3-methyl-N⁶-(5-methyl-1H-pyrazol-3-yl)-4-(1-methyl-1H-pyrazol-4-yl)pyridine-2,6-diamine

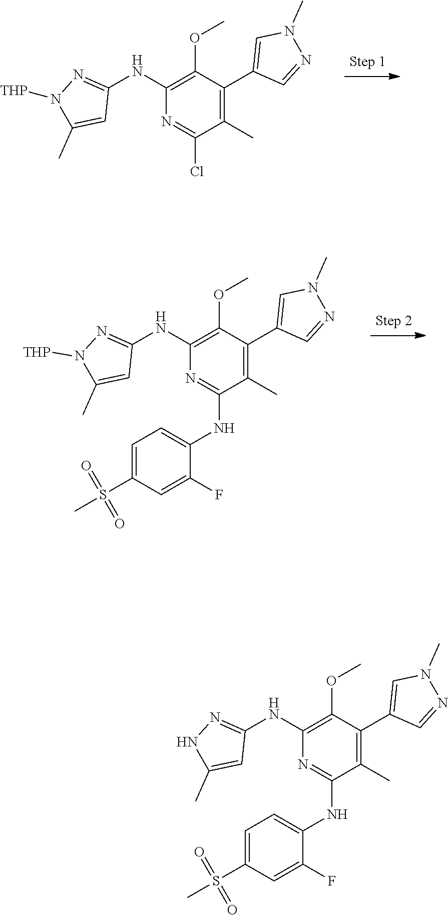

### Step 1/N²-(2-fluoro-4-(methylsulfonyl)phenyl)-5-methoxy-3-methyl-N⁶-(5-methyl-1-(tetrahydro-2H-pyran-2-yl)-1H-pyrazol-3-yl)-4-(1-methyl-1H-pyrazol-4-yl)pyridine-2,6-diamine

A solution of 6-chloro-3-methoxy-5-methyl-N-(5-methyl-1-(tetrahydro-2H-pyran-2-yl)-1H-pyrazol-3-yl)-4-(1-methyl-1H-pyrazol-4-yl)pyridin-2-amine (see Compound 22, Step 3) (100 mg, 0.24 mmol), 2-fluoro-4-(methylsulfonyl) aniline (68 mg, 0.36 mmol), Cs₂CO₃ (235 mg, 0.72 mmol), +/−BINAP (60 mg, 0.096 mmol) in Toluene (2.3 mL) was sparged with nitrogen for 15 min then Pd(OAc)₂ (11 mg, 0.048 mmol) was added and sparging was resumed for 5 min. The vial was sealed and heated at 95° C. for 18 hrs. The reaction mixture was filtered through celite pad, concentrated to dryness and the residue was purified by preparative HPLC eluting with a gradient of MeCN (5 to 100%) in water both containing 0.1% formic acid. Appropriate fractions were combined and lyophilized to afford N²-(2-fluoro-4-(methylsulfonyl)phenyl)-5-methoxy-3-methyl-N⁶-(5-methyl-1-(tetrahydro-2H-pyran-2-yl)-1H-pyrazol-3-yl)-4-(1-methyl-1H-pyrazol-4-yl)pyridine-2,6-diamine (120 mg, 85% yield) as an orange oil. UPLC-MS (+ESI) m/z=570.2 (M+H)⁺. ¹H NMR (400 MHz, CDCl₃) δ ppm 1.59-1.63 (m, 1H), 1.66-1.80 (m, 2H), 1.88-1.96 (m, 1H), 2.07-2.14 (m, 1H), 2.18 (s, 3H), 2.32 (s, 3H), 2.36-2.49 (m, 1H), 3.06 (s, 3H), 3.39 (s, 3H), 3.64-3.69 (m, 1H), 4.02 (s, 3H), 4.14-4.16 (m, 1H), 5.14-5.23 (m, 1H), 6.46 (br s, 1H), 6.57-6.62 (m, 1H), 7.41 (s, 1H), 7.56-7.68 (m, 4H), 8.43 (s, 1H).

### Step 2/Compound 23

To a solution of N²-(2-fluoro-4-(methylsulfonyl)phenyl)-5-methoxy-3-methyl-N⁶-(5-methyl-1-(tetrahydro-2H-pyran-2-yl)-1H-pyrazol-3-yl)-4-(1-methyl-1H-pyrazol-4-yl)pyridine-2,6-diamine (120 mg, 0.18 mmol) in MeOH (5.4 mL) was added HCl solution (37% in water, 1.0 mL, 12 mmol) and the reaction mixture was stirred at 50° C. until complete by UPLC-MS analysis (ca. 1.5 hrs). The reaction was quenched with NaOH (1M), concentrated to dryness, and purified by preparative HPLC eluting with a gradient of MeCN (5 to 100%) in water both containing 0.1% formic acid. Appropriate fractions were combined and lyophilization to afford Compound 23 (34 mg, 98% yield) as a light-yellow solid. UPLC-MS (+ESI) m/z=486.1 (M+H)⁺. ¹H NMR (400 MHz, DMSO) δ ppm 2.03-2.12 (m, J=2.9 Hz, 6H), 3.21 (s, 3H), 3.35 (s, 3H), 3.93 (s, 3H), 5.89 (br s, 1H), 7.40-7.66 (m, 3H), 7.70 (d, J=1.0 Hz, 1H), 7.95 (s, 1H), 8.15 (br s, 1H), 11.73 (br s, 1H). ¹⁹F NMR (377 MHz, DMSO) δ ppm −123.22 (br s, 1F).

### Compound 24/Method D/3-fluoro-N²-(2-fluoro-4-(methylsulfonyl)phenyl)-5-methoxy-N²-methyl-N⁶-(5-methyl-1H-pyrazol-3-yl)-4-(1-methyl-1H-pyrazol-4-yl)pyridine-2,6-diamine

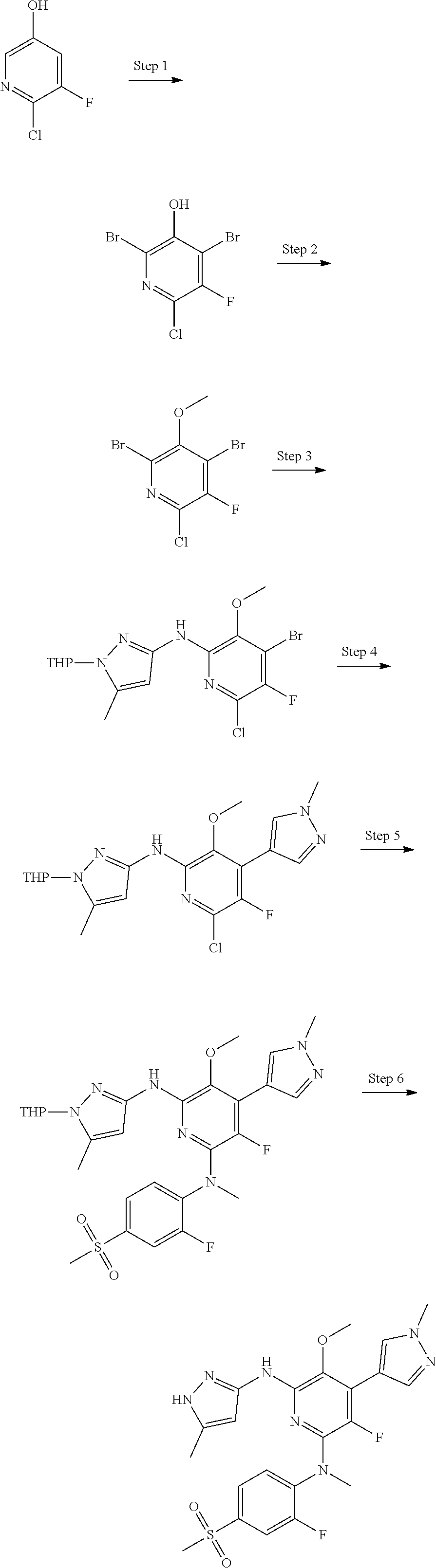

### Step 1/2,4-dibromo-6-chloro-5-fluoropyridin-3-ol

To a solution of 2-chloro-3-fluoro-5-hydroxypyridine (1.35 g, 9.12 mol) in MeCN (30 mL) and water (10 mL) was added Br₂ (1.17 mL, 22.81 mmol) at rt. The mixture was then stirred at rt overnight. The mixture was quenched with 10% Na₂S₂O₃, extracted with DCM (3×), washed with brine, dried over Na₂SO₄, filtered and concentrated. The crude was purified by silica gel chromatography eluting with EtOAc (0 to 30%) in Heptane. Appropriate fractions were combined and concentrated in vacuo to afford 2,4-dibromo-6-chloro-5-fluoropyridin-3-ol (2.18 g, 78%) as a white solid. UPLC-MS (+ESI) m/z=305.8 (M+H)⁺. ¹⁹F NMR (377 MHz, CDCl₃) δ ppm −110.55 (s, 1F).

### Step 2/2,4-dibromo-6-chloro-5-fluoro-3-methoxypyridine

To a mixture of 2,4-dibromo-6-chloro-5-fluoropyridin-3-ol (2.18 g, 7.14 mmol) and Cs₂CO₃ (4.65 g, 14.3 mmol) in DMF (45 mL) was added 12 (0.890 mL, 14.3 mmol) at rt. The mixture was then stirred at rt overnight. Water was added and the precipitate was collected by filtration using a Büchner funnel. The solid was rinsed with water then with heptane and dried under vacuum to give 2,4-dibromo-6-chloro-5-fluoro-3-methoxypyridine (2.02 g, 89% yield) as a white solid. UPLC-MS (+ESI) m/z=319.4 (M+H)⁺. ¹H NMR (400 MHz, CDCl₃) δ ppm 3.97 (s, 3H). ¹⁹F NMR (377 MHz, CDCl₃) δ ppm −109.88 (s, 1F).

### Step 3/4-bromo-6-chloro-5-fluoro-3-methoxy-N-(5-methyl-1-(tetrahydro-2H-pyran-2-yl)-1H-pyrazol-3-yl)pyridin-2-amine

A vial was charged with Intermediate 1 (1.25 g, 6.89 mmol), 2,4-dibromo-6-chloro-5-fluoro-3-methoxypyridine (2.00 g, 6.26 mmol), Xantphos (1.09 g, 1.88 mmol) and K₂CO₃ (2.60 g, 18.8 mmol) in Dioxane (33 mL) and the mixture was sparged with nitrogen for 10 min. Pd(OAc)₂ (211 mg, 0.94 mmol) was added and sparging continued for 5 min. The vial was sealed and stirred at 100° C. overnight. The reaction mixture was filtered on celite, concentrated to dryness, and purified by silica gel chromatography eluting with EtOAc (0-30%) in Heptane. Appropriate fractions were combined and concentrated in vacuo to afford 4-bromo-6-chloro-5-fluoro-3-methoxy-N-(5-methyl-1-(tetrahydro-2H-pyran-2-yl)-1H-pyrazol-3-yl)pyridin-2-amine (1.19 g, 45% yield) as a light-yellow solid. UPLC-MS (+ESI) m/z=421 (M+H)⁺. ¹H NMR (400 MHz, CDCl₃) δ ppm 1.59-1.77 (m, 3H), 1.85-1.92 (m, 1H), 2.06-2.13 (m, 1H), 2.32-2.41 (m, 4H), 3.62-3.70 (m, 1H), 3.91 (s, 3H), 4.08-4.14 (m, 1H), 5.18 (dd, J=10.3, 2.4 Hz, 1H), 6.70 (s, 1H), 7.46 (s, 1H).

### Step 4/6-chloro-5-fluoro-3-methoxy-N-(5-methyl-1-(tetrahydro-2H-pyran-2-yl)-1H-pyrazol-3-yl)-4-(1-methyl-1H-pyrazol-4-yl)pyridin-2-amine

A vial was charged with 4-bromo-6-chloro-5-fluoro-3-methoxy-N-(5-methyl-1-(tetrahydro-2H-pyran-2-yl)-1H-pyrazol-3-yl)pyridin-2-amine (1.19 g, 2.83 mmol), (1-methyl-1H-pyrazol-4-yl) boronic acid (374 mg, 2.97 mmol), NaHCO₃ (950 mg, 11.3 mmol) in Dioxane (18 mL) and water (6 mL). The mixture was sparged with N₂ for 10 min. Pd(PPh₃)₄ (490 mg, 0.42 mmol) was added and sparging continued for 5 min. The vial was sealed and stirred at 100° C. overnight. The reaction mixture was filtered on celite, concentrated to dryness and purified by silica gel chromatography eluting with EtOAc (0-70%) in Heptane. The appropriate fractions were combined and concentrated in vacuo to afford 6-chloro-5-fluoro-3-methoxy-N-(5-methyl-1-(tetrahydro-2H-pyran-2-yl)-1H-pyrazol-3-yl)-4-(1-methyl-1H-pyrazol-4-yl)pyridin-2-amine (464 mg, 39% yield) as a light-yellow solid. UPLC-MS (+ESI) m/z=421.2 (M+H)⁺. ¹H NMR (400 MHz, CDCl₃) δ ppm 1.69-1.79 (m, 3H), 1.85-1.92 (m, 1H), 2.06-2.13 (m, 1H), 2.34-2.44 (m, 4H), 3.63-3.70 (m, 4H), 4.01 (s, 3H), 4.08-4.14 (m, 1H), 5.18 (dd, J=10.3, 2.4 Hz, 1H), 6.74 (s, 1H), 7.51 (s, 1H), 7.98 (d, J=2.2 Hz, 1H), 8.10 (d, J=1.7 Hz, 1H). ¹⁹F NMR (377 MHz, CDCl₃) δ ppm −133.84 (s, 1F).

### Step 5/3-fluoro-N²-(2-fluoro-4-(methylsulfonyl)phenyl)-5-methoxy-N²-methyl-N⁶-(5-methyl-1-(tetrahydro-2H-pyran-2-yl)-1H-pyrazol-3-yl)-4-(1-methyl-1H-pyrazol-4-yl)pyridine-2,6-diamine

A vial was charged with 6-chloro-5-fluoro-3-methoxy-N-(5-methyl-1-(tetrahydro-2H-pyran-2-yl)-1H-pyrazol-3-yl)-4-(1-methyl-1H-pyrazol-4-yl)pyridin-2-amine (75 mg, 0.18 mmol), 2-fluoro-4-methanesulfonyl-N-methylaniline (54.3 mg, 0.27 mmol), Cs₂CO₃ (174 mg, 0.53 mmol) and +/−BINAP (44.4 mg, 0.070 mmol) in toluene (1.7 mL). The mixture was sparged with nitrogen for 10 min. Pd(OAc)₂ (8.0 mg, 0.040 mmol) was added and sparging continued for 5 min. The vial was sealed and stirred at 100° C. overnight. The reaction mixture was filtered on celite, concentrated to dryness and purified by preparative HPLC eluting with a gradient of MeCN (5 to 100%) in water both containing 0.1% formic acid to afford, after combining appropriate fractions and lyophilization, 3-fluoro-N²-(2-fluoro-4-(methylsulfonyl)phenyl)-5-methoxy-N²-methyl-N⁶-(5-methyl-1-(tetrahydro-2H-pyran-2-yl)-1H-pyrazol-3-yl)-4-(1-methyl-1H-pyrazol-4-yl)pyridine-2,6-diamine (36 mg, 33% yield) as a light-yellow solid. UPLC-MS (+ESI) m/z=588.2 (M+H)⁺. ¹H NMR (400 MHz, CDCl₃) δ ppm 1.55-1.60 (m, 1H), 1.64-1.69 (m, 1H), 1.84-1.90 (m, 2H), 2.05-2.12 (m, 1H), 2.25 (s, 3H), 2.31-2.43 (m, 1H), 3.08 (s, 3H), 3.52 (s, 3H), 3.60-3.69 (m, 4H), 3.98 (s, 3H), 4.07-4.13 (m, 1H), 5.14 (dd, J=10.4, 2.3 Hz, 1H), 6.24 (s, 1H), 7.23-7.26 (m, 1H), 7.51 (s, 1H), 7.60 (dd, J=11.1, 2.1 Hz, 1H), 7.69 (dd, J=8.6, 1.5 Hz, 1H), 7.94 (d, J=1.7 Hz, 1H), 8.06 (d, J=1.5 Hz, 1H).

### Step 6/Compound 24

To a solution of 3-fluoro-N2-(2-fluoro-4-(methylsulfonyl)phenyl)-5-methoxy-N2-methyl-N6-(5-methyl-1-(tetrahydro-2H-pyran-2-yl)-1H-pyrazol-3-yl)-4-(1-methyl-1H-pyrazol-4-yl)pyridine-2,6-diamine (35.0 mg, 0.060 mmol) in MeOH (600 μL) was added HCl solution (320 μL, 1.28 mmol, 4 M in dioxane). The mixture was then heated at 50° C. for 2 hrs. The solvents were evaporated, and the crude was neutralized by 5 M NaOH until pH 7-8 then purified by preparative HPLC eluting with MeCN (5-100%) in 10 mM aq. NH₄HCO₃ to give after lyophilization of the appropriate fractions Compound 24 (12 mg, 39% yield) as a light orange solid. UPLC-MS (+ESI) m/z=504.1 (M+H)⁺. ¹H NMR (400 MHz, DMSO-d₆) δ ppm 2.07 (br s, 3H), 3.24 (s, 3H), 3.42 (s, 3H), 3.59 (s, 3H), 3.92 (s, 3H), 5.89 (br s, 1H), 7.46-7.55 (m, 1H), 7.66-7.71 (m, 1H), 7.72-7.77 (m, 1H), 7.93 (s, 1H), 8.12 (br s, 1H), 8.24 (s, 1H), 11.75 (s, 1H). ¹⁹F NMR (377 MHz, DMSO-d₆) δ ppm-141.64 (s, 0.8F), −140.40 (s, 0.2F), −118.55 (s, 1F). Rotamers are observed in the ¹⁹F NMR spectrum.

### Compound 25/Method E/3-cyclopropyl-N⁶-(2,6-difluoro-4-(methylsulfonyl)phenyl)-N⁶-methyl-4-(1-methyl-1H-imidazol-4-yl)-N²-(5-methyl-1H-pyrazol-3-yl)pyridine-2,6-diamine

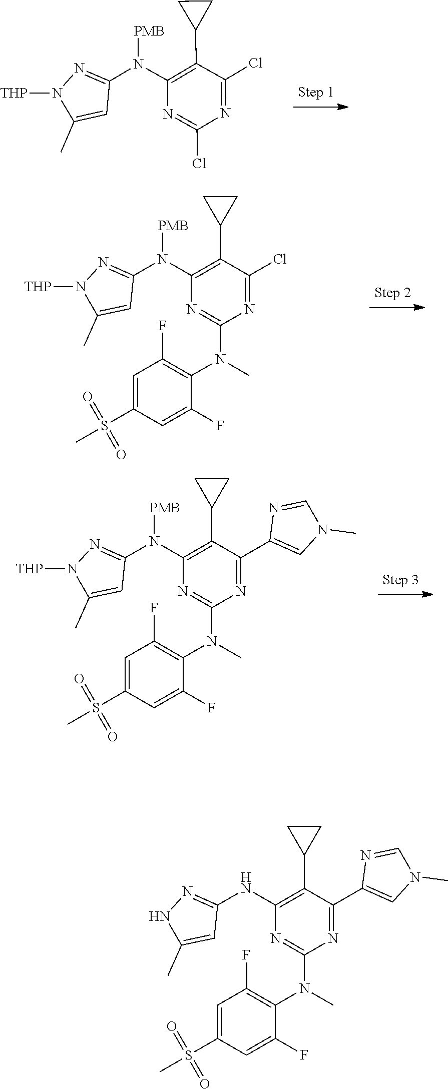

### Step 1/4-chloro-3-cyclopropyl-N⁶-(2,6-difluoro-4-(methylsulfonyl)phenyl)-N²-(4-methoxybenzyl)-N⁶-methyl-N²-(5-methyl-1-(tetrahydro-2H-pyran-2-yl)-1H-pyrazol-3-yl)pyridine-2,6-diamine

To Intermediate 12 (300 mg, 614 μmol) in DMPU (3 mL) was added 2,6-difluoro-4-methylsulfonyl-aniline (191 mg, 921 μmol) Nitrogen was bubbled for 5 minutes and then NaH (73 mg, 1.84 mmol, 60% purity) was added at rt. The vial was sealed, and the mixture was heated to 120° C. for 4 hrs. The mixture was cooled to rt and MeI (0.38 mL, 6.1 mmol,) was added and the resulting mixture was stirred for 1 h at rt. 1 mL of MeOH was added and the mixture was filtered on a celite cartridge. The volatiles were removed under reduced pressure and the residue was purified by preparative HPLC eluting with a gradient of MeCN (10 to 100%) in water both containing 0.1% formic acid. Appropriate fractions were combined and lyophilized to afford 6-chloro-5-cyclopropyl-N²-(2,6-difluoro-4-methylsulfonyl-phenyl)-N⁴-[(4-methoxyphenyl)methyl]-N²-methyl-N⁴-(5-methyl-1-tetrahydropyran-2-yl-pyrazol-3-yl)pyrimidine-2,4-diamine (210 mg, 51% yield) as a mixture of regioisomer (9/1) favoring the titled compound. UPLC-MS (+ESI) m/z=673.3 (M+H)⁺.

### Step 2/5-cyclopropyl-N²-(2,6-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine

To a solution of tributyl-(1-methylimidazol-4-yl) stannane (176 mg, 475 μmol) in Dioxane (1.00 mL) were added 6-chloro-5-cyclopropyl-N²-(2,6-difluoro-4-methylsulfonyl-phenyl)-N⁴-[(4-methoxyphenyl)methyl]-N²-methyl-N⁴-(5-methyl-1-tetrahydropyran-2-yl-pyrazol-3-yl)pyrimidine-2,4-diamine (100 mg, 149 μmol) and Pd(PPh₃)₄ (86 mg, 74 μmol). The mixture was degassed in vacuo and then backfilled with nitrogen, and finally stirred at 130° C. for 48 hrs. The crude reaction mixture was cooled to rt, filtered, and the filtrate was purified by preparative HPLC eluting with a gradient of MeCN (30 to 60%) in water both containing 0.1% formic acid. Appropriate fractions were combined and lyophilized to afford 5-cyclopropyl-N²-(2,6-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine (26 mg, 24% yield). UPLC-MS (+ESI) m/z=673.3 (M+H)⁺.

### Step 3/Compound 25

To 5-cyclopropyl-N²-(2,6-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine (26 mg, 36 μmol) was added trifluoroacetic acid (6 mL) and the resulting mixture was stirred at 90° C. for 4 hrs. The reaction mixture was concentrated, and the residue was purified by preparative HPLC eluting with a gradient of MeCN (10 to 25%) in water both containing 0.1% formic acid. Appropriate fractions were combined and lyophilized to afford Compound 25 (15 mg, 83% yield) as a white solid. UPLC-MS (+ESI) m/z=515.2 (M+H)⁺. ¹H NMR (400 MHz, DMSO-d₆) δ 11.91 (s, 1H), 8.56 (s, 1H), 8.09 (s, 1H), 7.84 (d, J=6.8 Hz, 2H), 5.25 (s, 1H), 3.86 (s, 3H), 1.98 (s, 3H), 1.74 (td, J=7.9, 3.9 Hz, 1H), 1.10 (s, 2H), 0.19 (d, J=5.0 Hz, 2H).

### Compound 26/Method B (RP-11710)/5-cyclopropyl-N²-(2-fluoro-4-((propan-2-yl-1,1,1,3,3,3-d₆)sulfonyl)phenyl)-N²-methyl-N⁴-(5-methyl-1H-pyrazol-3-yl)-6-(1-methyl-1H-pyrazol-4-yl)pyrimidine-2,4-diamine

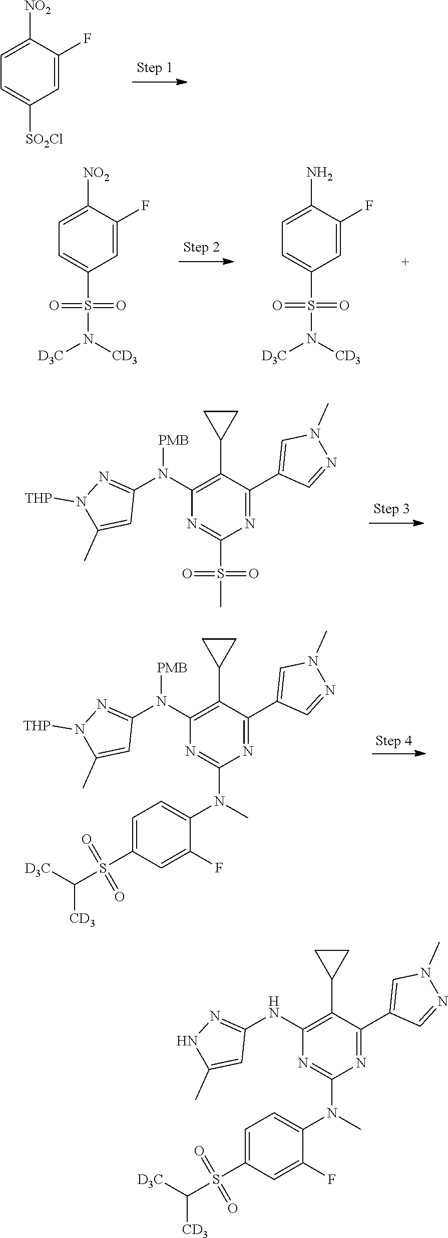

### Step 1/3-fluoro-N,N-bis(methyl-d₃)-4-nitrobenzenesulfonamide

To a mixture of dimethyl-de-amine HCl (53.7 mg, 0.607 mmol) in DCM (3 mL) at 0° C. was added triethylamine (187 μL, 1.34 mmol). The resulting reaction mixture was stirred at 0° C. for 5 minutes before the addition of 3-fluoro-4-nitrobenzenesulfonyl chloride (150 mg, 0.607 mmol). The reaction mixture was stirred 0° C. for an additional 30 minutes. The volatiles were removed in vacuo to afford 143 mg of 3-fluoro-N, N-bis(methyl-d₃)-4-nitrobenzenesulfonamide which was used as such in the subsequent step without further purification. ¹HNMR (DMSO-d₆, 400 MHz): δ 8.37 (1H, t, J=7.9 Hz), 7.98 (1H, d, J=10.4 Hz), 7.78 (1H, d, J=8.6 Hz),

### Step 2/4-amino-3-fluoro-N,N-bis(methyl-d₃)benzenesulfonamide

To a solution of 3-fluoro-N, N-bis(methyl-d₃)-4-nitrobenzenesulfonamide (143 mg, 0.562 mmol) in EtOH (4 mL) and water (2 mL) was added NH₄Cl (301 mg, 5.62 mmol) and zinc (263 mg, 3.94 mmol). The resulting reaction mixture was stirred at 80° C. for 1 h. The reaction mixture was then filtered through celite. EtOAc and aqueous saturated NaHCO₃ were added to the filtrate. The layers were partitioned and the organic layer was washed with brine, dried over anhydrous MgSO4 and concentrated in vacuo to afford 130 mg of 4-amino-3-fluoro-N, N-bis(methyl-d₃)benzenesulfonamide which was used as such in the subsequent step without further purification. UPLC-MS (+ESI): m/z=225.1 [M+H]⁺.

### Step 3/5-cyclopropyl-N²-(2-fluoro-4-((propan-2-yl-1,1,1,3,3,3-d₆) sulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine

To a solution of 5-cyclopropyl-N-(4-methoxybenzyl)-N-(5-methyl-1-(tetrahydro-2H-pyran-2-yl)-1H-pyrazol-3-yl)-6-(1-methyl-1H-pyrazol-4-yl)-2-(methylsulfonyl)pyrimidin-4-amine (see Compound 11, Step 1) (180 mg, 0.312 mmol), 4-amino-3-fluoro-N,N-bis(methyl-d₃)benzenesulfonamide (154 mg, 0.685 mmol) in NMP (1.56 mL) was added NaH (37 mg, 1.6 mmol). The resulting reaction mixture was stirred at 125° C. for 16 hrs. The conversion was not complete. Additional NaH (37 mg, 1.6 mmol) was added and the reaction mixture was stirred at 125° C. for 16 hrs. Upon cooling the reaction mixture to rt, MeI (194 μL, 3.12 mmol) was added and stirred at rt for 18 hrs. The conversion was not complete. Additional NaH (37 mg, 1.6 mmol) was added and the reaction mixture was stirred for 5 hrs. Crushed ice and EtOAc (25 mL) were added. The layers were partitioned, and the aqueous layer was extracted with EtOAc (25 mL). The combined organic layers were washed with brine, dried over MgSO₄, and concentrated in vacuo. The residue was purified by silica gel chromatography eluting with EtOAc (40-100%) in hexanes. The desired fractions were combined, concentrated to dryness to afford 5-cyclopropyl-N²-(2-fluoro-4-((propan-2-yl-1,1,1,3,3,3-d₆) sulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (71 mg, 31% yield). UPLC-MS (+ESI): m/z=736.5 [M+H]⁺.

### Step 4/Compound 26

5-cyclopropyl-N²-(2-fluoro-4-((propan-2-yl-1,1,1,3,3,3-d₆) sulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-pyrazol-4-yl)pyrimidine-2,4-diamine (71 mg, 97 μmol) was dissolved in TFA (2.2 mL, 29 mmol). The reaction mixture was heated at 80° C. for 1 h. After cooling the reaction mixture to rt, the volatiles were removed in vacuo. The residue was co-evaporated with NEt₃ (3×) and the residue was purified by silica gel chromatography eluting with MeCN (10-100%) in DCM. The desired fractions were combined, concentrated to dryness and lyophilized by redissolving in MeCN/water to afford Compound 26 (34 mg, 66% yield) as an off-white solid. 1H-NMR (400 MHz, DMSO-d₆): δ 11.85 (s; 1H); 8.22 (s; 2H); 7.91 (s; 1H); 7.72 (t; J=7.94 Hz; 1H); 7.63-7.65 (m; 2H); 5.64 (s; 1H); 3.88 (s; 3H); 3.47 (s; 3H); 2.04 (s; 3H); 1.72-1.75 (m; 1H); 1.12 (d; J=7.42 Hz; 2H); 0.18 (d; J=5.30 Hz; 2H). UPLC-MS (+ESI): m/z=532.0 [M+H]⁺.

### Compound 108/Method B/5-cyclopropyl-N²-(2-fluoro-4-(methylsulfonyl)phenyl)-N²-methyl-N⁴-(5-methyl-1H-pyrazol-3-yl)-6-(pyrazolo[1,5-a]pyrimidin-3-yl)pyrimidine-2,4-diamine

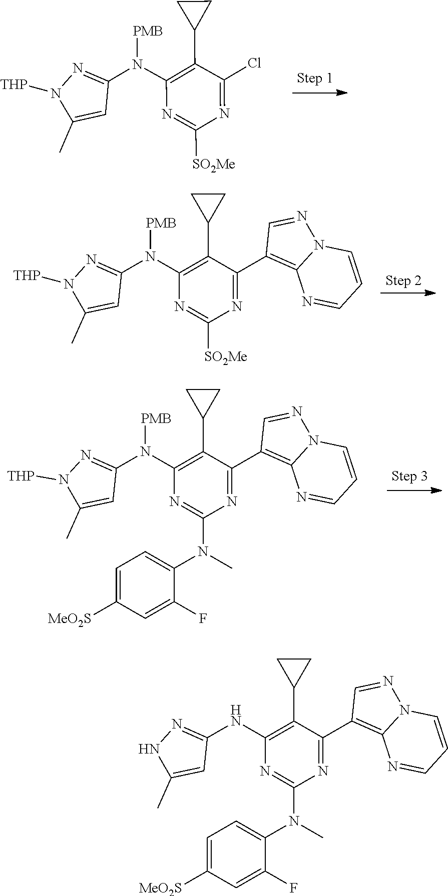

### Step 1/5-cyclopropyl-N-(4-methoxybenzyl)-N-(5-methyl-1-(tetrahydro-2H-pyran-2-yl)-1H-pyrazol-3-yl)-2-(methylsulfonyl)-6-(pyrazolo[1,5-a]pyrimidin-3-yl)pyrimidin-4-amine

The a mixture of Intermediate 8 (150 mg, 282 μmol), Cs₂CO₃ (2 M in H₂O, 423 μL, 846 umol), 3-(4,4,5,5-tetramethyl-1,3,2-dioxaborolan-2-yl) pyrazolo[1,5-a]pyrimidine (104 mg, 423 μmol) Pd(dppf)Cl₂ (26 mg, 31 μmol) in dioxane (1.5 mL) in a microwave reaction vial was degassed (3 cycles of vacuum/argon atmosphere). The reaction mixture was then heated at 85° C. for 16 hrs. After cooling the reaction mixture to rt, EtOAc was added, and the mixture was filtered on celite and washed with EtOAc. The filtrate was concentrated to dryness in vacuo. The residue was purified by silica gel flash chromatography eluting with EtOAc (0 to 100%) in hexanes. The desired fractions were combined and concentrated to dryness in vacuo to afford 5-cyclopropyl-N-(4-methoxybenzyl)-N-(5-methyl-1-(tetrahydro-2H-pyran-2-yl)-1H-pyrazol-3-yl)-2-(methylsulfonyl)-6-(pyrazolo[1,5-a]pyrimidin-3-yl)pyrimidin-4-amine (65 mg, 38% yield). UPLC-MS (+ESI): m/z=615.3 [M+H]⁺.

### Step 2/5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(pyrazolo[1,5-a]pyrimidin-3-yl)pyrimidine-2,4-diamine

Sodium hydride (60% in mineral oil, 16.9 mg, 423 μmol) was added to a solution of 5-cyclopropyl-N-(4-methoxybenzyl)-N-(5-methyl-1-(tetrahydro-2H-pyran-2-yl)-1H-pyrazol-3-yl)-2-(methylsulfonyl)-6-(pyrazolo[1,5-a]pyrimidin-3-yl)pyrimidin-4-amine (65 mg, 106 μmol) and 2-fluoro-4-(methylsulfonyl) aniline (46.3 mg, 233 μmol) in NMP (1 mL). The reaction was purged with nitrogen for 5 minutes and heated at 50° C. for 1 h. After cooling the reaction mixture to rt, iodomethane (65.8 μL, 1.06 mmol) was added and the resulting mixture was stirred at rt for 1 h. The reaction mixture was then poured in water and extracted with EtOAc (3×). The combined organic layers were washed with brine, dried over anhydrous Na₂SO₄, filtered, and concentrated to dryness in vacuo. The residue was purified by silica gel chromatography eluting with EtOAc (0 to 100%) in hexanes. The desired fractions were combined and concentrated to dryness to afford 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(pyrazolo[1,5-a]pyrimidin-3-yl)pyrimidine-2,4-diamine (27 mg, 35% yield). UPLC-MS (+ESI): m/z=738.4 [M+H]⁺.

### Step 3/Compound 108

A solution of 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(pyrazolo[1,5-a]pyrimidin-3-yl)pyrimidine-2,4-diamine (27.0 mg, 36.6 μmol) in trifluoroacetic acid (2.0 mL, 26.1 mmol) was heated at 80° C. for 1 h. After cooling the reaction mixture to rt, the volatiles were removed in vacuo. The residue was purified by preparative HPLC eluting with MeCN (40 to 60%) in 10 mM aqueous NH₄HCO₃ (pH 3.8). The desired fractions were combined, and lyophilized to afford Compound 108 (2.4 mg, 12% yield). 1H-NMR (400 MHz, DMSO-d₆): δ 9.18 (1H, d, J=7.1 Hz), 8.63 (1H, s), 8.47 (1H, s), 8.28-8.42 (1H, m), 7.77-7.87 (3H, m), 7.08-7.17 (1H, m), 5.69 (1H, s), 3.48 (3H, s), 2.05 (3H, s), 1.94 (1H, s), 0.63 (2H, d, J=4.3 Hz), −0.05 (2H, d, J=3.9 Hz). 4 protons obscured by the water peak UPLC-MS (+ESI): m/z=534.3 [M+H]⁺.

### Compound 110/Method F/5-cyclopropyl-6-(2,3-dihydro-4H-pyrido[4,3-b][1,4]oxazin-4-yl)-N²-(2-fluoro-4-(methylsulfonyl)phenyl)-N²-methyl-N⁴-(5-methyl-1H-pyrazol-3-yl)pyrimidine-2,4-diamine

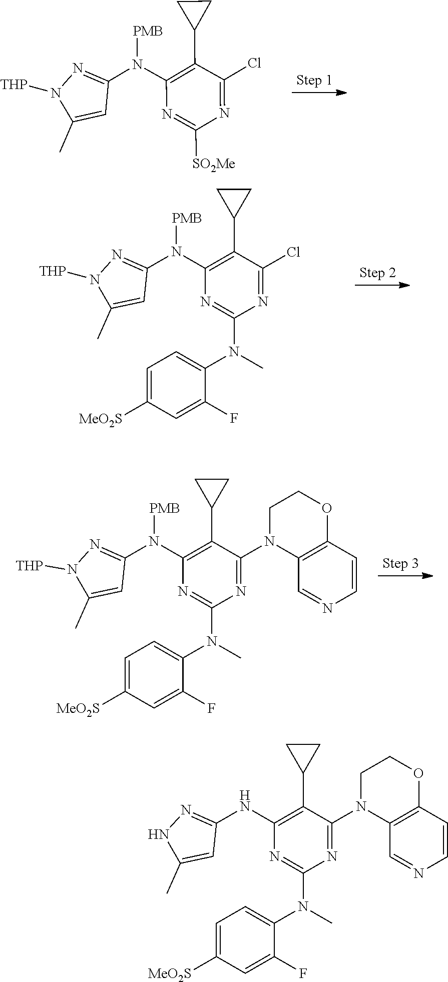

### Step 1/6-chloro-5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine

Sodium hydride (60% in mineral oil, 376 mg, 9.40 mmol) was added to a solution of Intermediate 8 (1.00 g, 1.88 mmol) and 2-fluoro-4-(methylsulfonyl) aniline (449 mg, 2.26 mmol) in NMP (2 mL). The reaction was purged with nitrogen for 5 minutes and heated at 125° C. for 16 hrs. After cooling the reaction to rt, MeI (1.23 mL, 19.7 mmol) was added and the resulting mixture was stirred at rt for 1 h. The reaction mixture was then diluted with water, EtOAc and brine. The layers were partitioned, and the organic layer was washed with brine (3×), dried over anhydrous MgSO4, filtered, and concentrated to dryness in vacuo. The residue was purified by silica gel chromatography eluting with MeCN (0 to 100%) in DCM. The desired fractions were combined, concentrated to dryness to afford 6-chloro-5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (566 mg, 46% yield). UPLC-MS (+ESI): m/z=655.3 [M+H]⁺.

### Step 2/5-cyclopropyl-6-(2,3-dihydro-4H-pyrido[4,3-b][1,4]oxazin-4-yl)-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine

Xantphos (17.7 mg, 30.5 μmol) was added to a solution of 6-chloro-5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (100 mg, 153 μmol), 3,4-dihydro-2H-pyrido[4,3-b][1,4]oxazine (54.7 mg, 382 μmol), Pd₂(dba)₃ (14.0 mg, 15.3 μmol) and Cs₂CO₃ (149 mg, 0.458 mmol) in toluene (1.5 mL). The reaction was purged with nitrogen for 5 min and heated to 115° C. for 16 hrs. After cooling the reaction mixture to rt, the volatiles were removed in vacuo. The residue was diluted with EtOAc and water. The layers were partitioned, and the organic layer was washed with brine (2×), dried over anhydrous MgSO4, filtered, and concentrated to dryness in vacuo. The residue was purified by silica gel chromatography eluting with MeCN (0 to 100%) in DCM. The desired fractions were combined and concentrated to dryness to afford 5-cyclopropyl-6-(2,3-dihydro-4H-pyrido[4,3-b][1,4]oxazin-4-yl)-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (56.1 mg, 49% yield). UPLC-MS (+ESI): m/z=755.4 [M+H]⁺.

### Step 3/Compound 110

A solution of 5-cyclopropyl-6-(2,3-dihydro-4H-pyrido[4,3-b][1,4]oxazin-4-yl)-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (56.1 mg, 74.2 μmol) in TFA (0.5 mL) was heated at 80° C. for 1 h. After cooling the reaction mixture to rt, the volatiles were removed and co-evaporated with triethylamine (3×1 mL). The residue was purified by silica gel chromatography eluting first with MeCN (0-100%) in DCM followed by 10% MeOH in DCM. The desired fractions were combined and concentrated to dryness in vacuo. The residue was then purified by preparative HPLC eluting with MeCN (35 to 55%) in 10 mM NH₄HCO₃ (pH=10). The desired fractions were combined and lyophilized to afford Compound 110 (10 mg, 24% yield). ¹H-NMR (400 MHz, DMSO-d₆): δ 11.84 (1H, s), 8.33 (1H, s), 8.01 (1H, s), 7.88 (1H, s), 7.82 (2H, t, J=9.8 Hz), 7.74 (1H, t, J=7.7 Hz), 6.86 (1H, s), 5.67 (1H, s), 4.35-4.44 (2H, m), 3.90 (2H, s), 3.39 (3H, s), 3.29 (3H, s), 2.05 (3H, s), 1.39 (1H, s), 0.65-0.80 (2H, m), 0.20-0.35 (2H, m). UPLC-MS (+ESI): m/z=551.3 [M+H]⁺.

### Compound 111/Method G/5-cyclopropyl-N²-(2-fluoro-4-(methylsulfonyl)phenyl)-N²-methyl-N⁴-(5-methyl-1H-pyrazol-3-yl)-6-(3-(methylsulfonyl) piperidin-1-yl)pyrimidine-2,4-diamine

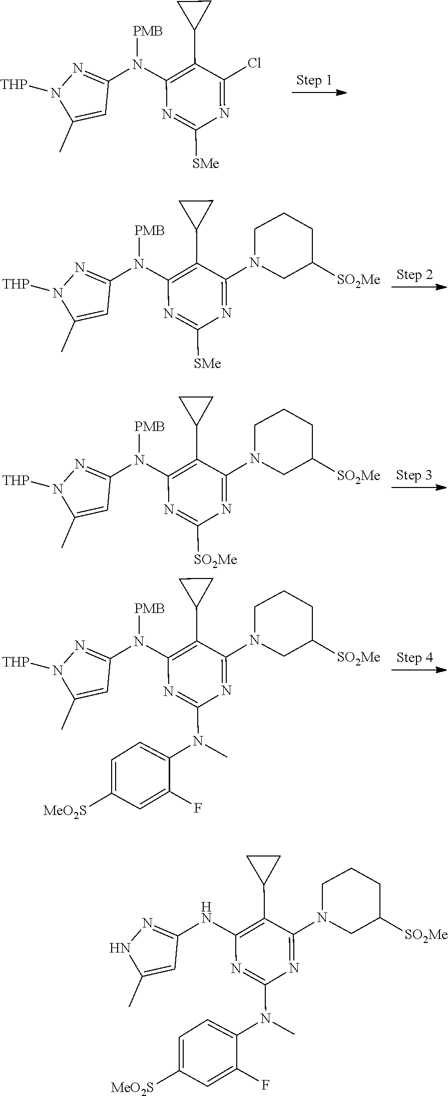

### Step 1/5-cyclopropyl-N-(4-methoxybenzyl)-N-(5-methyl-1-(tetrahydro-2H-pyran-2-yl)-1H-pyrazol-3-yl)-6-(3-(methylsulfonyl) piperidin-1-yl)-2-(methylthio)pyrimidin-4-amine

N,N-diisopropylethylamine (190 μL, 1.09 mmol) was added to a solution of 6-chloro-5-cyclopropyl-N-(4-methoxybenzyl)-N-(5-methyl-1-(tetrahydro-2H-pyran-2-yl)-1H-pyrazol-3-yl)-2-(methylthio)pyrimidin-4-amine (see Intermediate 8, Step 1) (260 mg, 520 μmol) and 3-(methylsulfonyl) piperidine (163 μL, 780 μmol) in DMSO (1 mL). The resulting reaction mixture was stirred at 140° C. for 16 hrs. After cooling to rt, the reaction mixture was diluted with EtOAc and brine. The layers were partitioned, and the organic layer was washed with brine, dried over anhydrous MgSO₄, filtered, and concentrated to dryness in vacuo. The residue was purified by silica gel chromatography eluting with EtOAc (0 to 100%) in hexanes. The desired fractions were combined, concentrated to dryness to afford 5-cyclopropyl-N-(4-methoxybenzyl)-N-(5-methyl-1-(tetrahydro-2H-pyran-2-yl)-1H-pyrazol-3-yl)-6-(3-(methylsulfonyl) piperidin-1-yl)-2-(methylthio)pyrimidin-4-amine (226 mg, 69% yield). UPLC-MS (+ESI): m/z=627.4 [M+H]⁺.

### Step 2/5-cyclopropyl-N-(4-methoxybenzyl)-N-(5-methyl-1-(tetrahydro-2H-pyran-2-yl)-1H-pyrazol-3-yl)-2-(methylsulfonyl)-6-(3-(methylsulfonyl) piperidin-1-yl)pyrimidin-4-amine

Hydrogen peroxide (258 μL, 2.52 mmol) was added to a mixture of 5-cyclopropyl-N-(4-methoxybenzyl)-N-(5-methyl-1-(tetrahydro-2H-pyran-2-yl)-1H-pyrazol-3-yl)-6-(3-(methylsulfonyl) piperidin-1-yl)-2-(methylthio)pyrimidin-4-amine (226 mg, 0.360 mmol), Na₂WO₄·2H₂O (11.9 mg, 36.0 μmol) and NBu₄(HSO₄) (19.6 mg, 57.6 μmol) in THF (7 mL) and EtOAc (7 mL). The reaction mixture was stirred at 50° C. for 3 h. After cooling to rt, the reaction mixture was quenched with 5% NaHSO3. The resulting mixture was diluted with EtOAc and brine. The layers were partitioned and the organic layer was washed with brine, dried over anhydrous MgSO₄, filtered and concentrated to dryness to afford crude 5-cyclopropyl-N-(4-methoxybenzyl)-N-(5-methyl-1-(tetrahydro-2H-pyran-2-yl)-1H-pyrazol-3-yl)-2-(methylsulfonyl)-6-(3-(methylsulfonyl) piperidin-1-yl)pyrimidin-4-amine (211 mg), which was used as such in the next step without further purification. UPLC-MS (+ESI): m/z=659.4 [M+H]⁺.

### Step 3/5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(3-(methylsulfonyl) piperidin-1-yl)pyrimidine-2,4-diamine

Sodium hydride (60% in mineral oil, 27.9 mg, 0.698 mmol) was added to a solution of 5-cyclopropyl-N-(4-methoxybenzyl)-N-(5-methyl-1-(tetrahydro-2H-pyran-2-yl)-1H-pyrazol-3-yl)-2-(methylsulfonyl)-6-(3-(methylsulfonyl) piperidin-1-yl)pyrimidin-4-amine (100 mg, 0.152 mmol) and 2-fluoro-4-(methylsulfonyl) aniline (34.5 mg, 0.182 mmol) in NMP (760 μL). The reaction mixture was purged with nitrogen for 5 minutes and heated at 125° C. for 16 hrs. After cooling the reaction mixture to rt, iodomethane (100 μL, 1.59 mmol) was added and the resulting mixture was stirred at rt for 1 h. The reaction mixture was diluted with water, EtOAc and brine. The layers were partitioned, and the organic layer was washed with brine (3×), dried over anhydrous MgSO4, filtered, and concentrated to dryness in vacuo. The residue was purified by silica gel chromatography eluting with MeCN (0 to 100%) in DCM. The desired fractions were combined, concentrated to dryness to afford of 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(3-(methylsulfonyl) piperidin-1-yl)pyrimidine-2,4-diamine (21 mg). UPLC-MS (+ESI): m/z=782.3 [M+H]+.

### Step 4/Compound 111

A solution of 5-cyclopropyl-N²-(2-fluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(3-(methylsulfonyl) piperidin-1-yl)pyrimidine-2,4-diamine (21 mg, 27 μmol) in TFA (2.0 mL) was heated at 80° C. for 1 h. After cooling the reaction mixture to rt, the volatiles were removed in vacuo. The residue was purified by preparative HPLC eluting with MeCN (35 to 75%) in H₂O both containing 0.1% formic acid. The desired fractions were combined and lyophilized to afford of Compound 111 (2.5 mg, 16% yield). 1H-NMR (400 MHz, DMSO-d₆): δ 8.06 (1H, s), 7.80 (2H, t, J=8.5 Hz), 7.71 (1H, t, J=7.8 Hz), 5.66 (1H, s), 4.45 (1H, d, J=12.3 Hz), 4.09 (1H, d, J=12.9 Hz), 3.41 (3H, s), 3.29 (3H, s), 2.86-2.94 (6H, m), 2.16-2.19 (1H, m), 2.04 (3H, s), 1.78 (1H, d, J=11.9 Hz), 1.53-1.67 (3H, m), 1.00-1.02 (2H, m), 0.35-0.40 (2H, m). One proton obscured by the water peak. UPLC-MS (+ESI): m/z=578.3 [M+H]⁺.

### Compound 121/Method H/5-cyclopropyl-N²-(2,6-difluoro-4-(methylsulfonyl)phenyl)-6-(1,3-dimethyl-1H-pyrazol-4-yl)-N²-methyl-N⁴-(5-methyl-1H-pyrazol-3-yl)pyrimidine-2,4-diamine

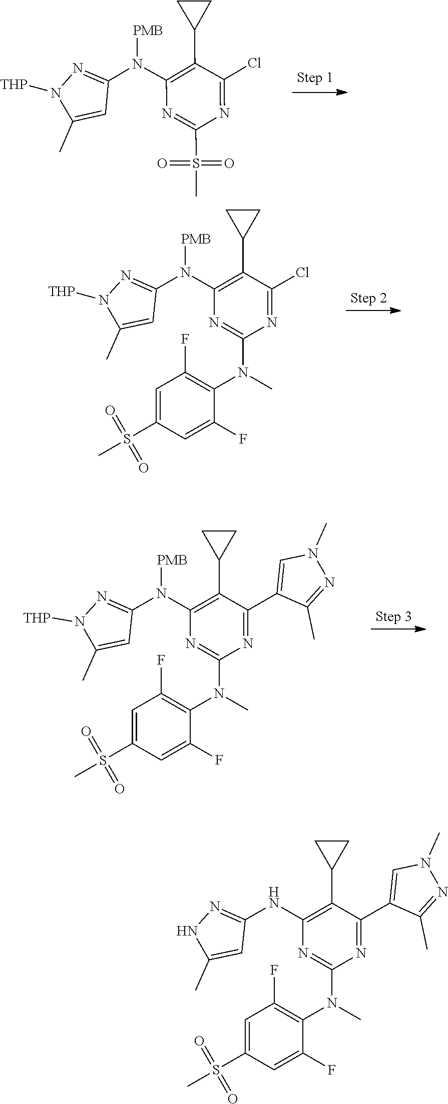

### Step 1/6-chloro-5-cyclopropyl-N²-(2,6-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine

To a nitrogen purged solution of 2,6-difluoro-4-methylsulfonyl-aniline (2.34 g, 11.3 mmol) and Intermediate 8 (5.62 g, 10.3 mmol in DMPU (48 mL) was added NaHMDS (1 M, 22.5 mL, 22.5 mmol) at rt. The reaction was heated to 160° C. for 30 minutes. The reaction mixture was cooled down to rt and iodomethane (3.19 mL, 51.2 mmol,) was slowly added. The residual mixture was stirred at rt for 30 min. The resulting mixture was poured over a mixture of water (800 mL) and ethyl acetate (200 mL). Phases were separated then the aqueous phase was back extracted twice with ethyl acetate (2×200 mL). The pooled organic phases were washed with brine (200 mL), dried over MgSO₄, filtered, and concentrated in vacuo. Crude product was purified by silica gel chromatography eluting with EtOAc (5 to 10%) in heptane. Appropriate fractions were pooled and concentrated to dryness. The residue was then purified by reverse phase flash chromatography (HP C18 RediSep® Rf gold) eluting with a gradient of CH₃CN (0 to 100%) in water both containing 0.1% formic acid. Appropriate fractions were combined and concentrated under reduced pressure to a volume of approximately 20 mL. Poured in aqueous saturated NaHCO₃ and extracted with EtOAc (3×). The combined organic layers were washed with brine, dried over Na₂SO₄, filtered, and concentrated in vacuo then dried on vacuum pump to afford 6-chloro-5-cyclopropyl-N²-(2,6-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (3.50 g, 51% yield). ¹H NMR (400 MHz, DMSO-d₆) δ 7.76 (d, J=8.0 Hz, 2H), 6.64 (s, 4H), 5.92 (s, 1H), 5.21 (d, J=9.4 Hz, 1H), 4.55 (s, 1H), 3.80 (d, J=11.5 Hz, 1H), 3.65 (s, 3H), 3.58-3.44 (m, 1H), 3.27 (s, 3H), 3.26 (s, 3H), 2.18 (s, 3H), 2.13-1.97 (m, 1H), 1.89 (d, J=12.5 Hz, 1H), 1.68 (d, J=12.9 Hz, 1H), 1.56 (d, J=11.1 Hz, 1H), 1.44 (s, 2H), 0.94 (ddd, J=13.8, 8.3, 5.5 Hz, 1H), 0.43 (p, J=9.2 Hz, 2H), 0.26-−0.01 (m, 2H). UPLC-MS (+ESI): m/z=673.7 [M+H]⁺.

### Step 2/5-cyclopropyl-N²-(2,6-difluoro-4-(methylsulfonyl)phenyl)-6-(1,3-dimethyl-1H-pyrazol-4-yl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine

To 1,3-dimethyl-4-(4,4,5,5-tetramethyl-1,3,2-dioxaborolan-2-yl) pyrazole (85.8 mg, 386 umol) in Dioxane (1 mL) and H₂O (250 μL) were added 6-chloro-5-cyclopropyl-N²-(2,6-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (200 mg, 297 μmol), Cs₂CO₃ (242 mg, 743 μmol) and PdCl₂(dppf) (43.5 mg, 59.4 μmol). The mixture was degassed in vacuo and then backfilled with N₂, sealed, and stirred at 120° C. for 1 h. The crude reaction mixture was filtered, and the filtrate was purified by preparative HPLC eluting with a gradient of CH₃CN (55 to 85%) in water both containing 0.1% formic acid. Appropriate fractions were combined and lyophilized to afford 5-cyclopropyl-N²-(2,6-difluoro-4-(methylsulfonyl)phenyl)-6-(1,3-dimethyl-1H-pyrazol-4-yl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (73.0 mg, 34% yield). UPLC-MS (+ESI): m/z=733.0 [M+H]⁺.

### Step 3/Compound 121

To 5-cyclopropyl-N²-(2,6-difluoro-4-(methylsulfonyl)phenyl)-6-(1,3-dimethyl-1H-pyrazol-4-yl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (73.0 mg, 99.6 μmol) in DCM (1 mL) was added trifluoroacetic acid (115 uL, 1.49 mmol,). The resulting mixture was heated to 90° C. for 6 hrs. The volatiles were removed in vacuo and the residue was co-evaporated with DCM twice. The residue was dissolved in DCM washed with NaHCO₃, dried over Na₂SO₄, filtered and concentrated. The residue was purified by reverse phase flash chromatography eluting with a gradient of CH₃CN (15 to 60%) in water both containing 0.1% formic acid. Appropriate fractions were combined and lyophilized to afford Compound 121 (16.0 mg, 30% yield). ¹H NMR (400 MHz, DMSO-d₆) δ 11.83 (br s; 1H); 8.22 (br s; 1H); 8.01 (s; 1H); 7.79 (d; J=6.80 Hz; 2H); 5.27 (br s; 1H); 3.75 (s; 3H); 3.33 (s; 3H); 3.30 (s; 3H); 2.00 (br s; 3H); 1.59-1.67 (m; 1H); 0.96 (d; J=7.60 Hz; 2H); 0.05 (d; J=5.20 Hz; 2H). UPLC-MS (+ESI): m/z=528.8 [M+H]⁺.

### Compound 125/Method H/5-cyclopropyl-N²-(2,5-difluoro-4-(methylsulfonyl)phenyl)-N²-methyl-6-(1-methyl-1H-imidazol-4-yl)-N⁴-(5-methyl-1H-pyrazol-3-yl)pyrimidine-2,4-diamine

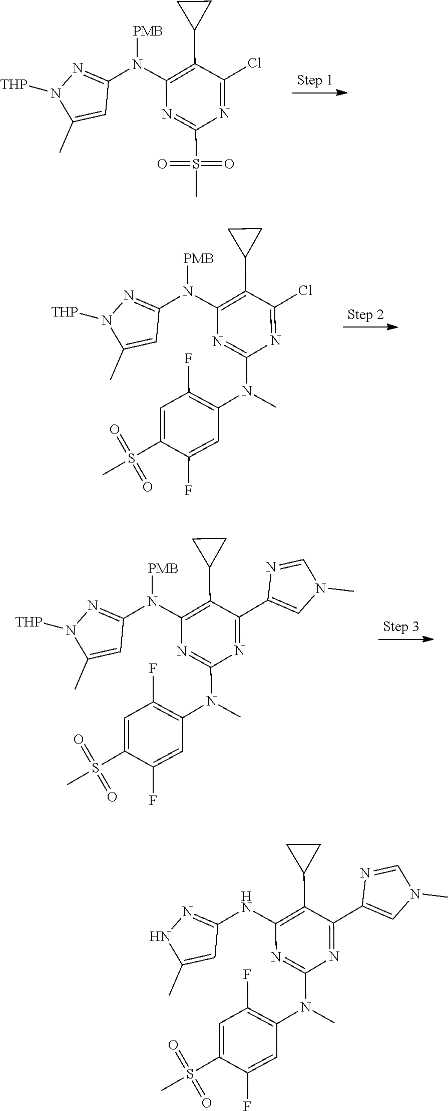

### Step 1/6-chloro-5-cyclopropyl-N²-(2,5-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine

To Intermediate 8 (5.00 g, 9.40 mmol), and 2,5-difluoro-4-methylsulfonyl-aniline (2.92 g, 14.1 mmol) in DMPU (60 mL) was added NaHMDS (1 M, 21.1 mL, 21.4 mmol) and the reaction mixture was heated to 110° C. for 10 minutes. The resulting mixture was cooled to rt then MeI (5.9 mL, 94 mmol,) was added over 15 minutes and the reaction was stirred at rt for 45 minutes. The reaction mixture was poured over a mixture of water (400 mL) and ethyl acetate (200 mL). Organic phase were separated then aqueous phase was back extracted twice with EtOAc (2×100 ml mL). Pooled organic phases were washed with brine (200 mL) then dried over anhydrous MgSO₄, filtered over a fritted glass Buchner and the cake was rinsed with ethyl acetate (100 mL). Filtrate was concentrated and the residue was purified by silica gel chromatography eluting with EtOAc (5 to 10%) in Hex followed by a second purification by silica gel chromatography eluting with MeOH (10 to 20%) in DCM. Appropriate fractions were combined and the residue was further purified reverse phase flash chromatography (HP C18 RediSep® Rf gold) eluting with a gradient of CH₃CN (10 to 100%) in water both containing 0.1% formic acid. Appropriate fractions were combined and lyophilized to afford 6-chloro-5-cyclopropyl-N²-(2,5-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (4.00 g, 63% yield). UPLC-MS (+ESI): m/z=674.2 [M+H]⁺.

### Step 2/5-cyclopropyl-N²-(2,5-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine

Nitrogen was bubbled through a solution of 6-chloro-5-cyclopropyl-N²-(2,5-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)pyrimidine-2,4-diamine (600 mg, 891 μmol), tributyl-(1-methylimidazol-4-yl) stannane (700 mg, 1.89 mmol) in 1,4-dioxane (10 mL) while sonicating for 15 minutes. XPhosPdG₂ (210 mg, 223 μmol) was then added, and Nitrogen was bubbled in the resulting suspension under sonication for another 10 min. The final reaction mixture was sealed and the stirred at 130° C. for 2 hours. The resulting suspension was diluted with 10 mL EtOAc and 3 mL of 1M KF, filtered through celite, and the organic phase was separated, washed with brine, dried over Na₂SO₄, filtered and concentrated. The residue was purified by silica gel chromatography eluting with a gradient of MeOH (1 to 8%) in DCM. Appropriate fractions were combined and concentrated in vacuo to afford 5-cyclopropyl-N²-(2,5-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine (525 mg, 79% yield).

### Step 3/Compound 125

5-cyclopropyl-N²-(2,5-difluoro-4-(methylsulfonyl)phenyl)-N⁴-(4-methoxybenzyl)-N²-methyl-N⁴-(5-methyl-1-(tetrahydro-2H-pyran-2-yl)-1H-pyrazol-3-yl)-6-(1-methyl-1H-imidazol-4-yl)pyrimidine-2,4-diamine (525 mg, 730 μmol) and L-Cysteine (180 mg, 1.49 mmol) were dissolved in DCE (5 mL) to which was added TFA (2.25 mL, 29.21 mmol,). The mixture was heated for 1 h at 80° C. under reflux. Volatiles were removed in vacuo, co-evaporated twice with CH₃CN, dissolved DCM and neutralized with saturated aqueous NaHCO₃. The organic layer was separated and concentrated in vacuo. The residue was dissolved in DMSO and purified by preparative HPLC eluting with a gradient of CH₃CN (15 to 45%) in water containing 10 mM NH₄HCO₃ (pH adjusted to 10 with NH₄OH). Appropriate fractions were combined and lyophilized to afford Compound 125 (71 mg, 19% yield) as a white solid. ¹H NMR (400 MHz, DMSO-d₆) δ 12.45-11.67 (m, 1H), 8.23 (s, 1H), 7.79 (dd, J=10.9, 5.9 Hz, 1H), 7.72-7.64 (m, 2H), 7.60 (d, J=1.4 Hz, 1H), 5.72 (s, 1H), 3.73 (s, 3H), 3.54-3.43 (m, 3H), 3.39 (s, 3H), 2.08 (d, J=4.9 Hz, 3H), 1.82 (p, J=7.5 Hz, 1H), 0.96 (h, J=4.3 Hz, 2H), 0.09 (td, J=6.0, 4.2 Hz, 2H). UPLC-MS (+ESI): m/z=515.2 [M+H]⁺.

### Compound 144/Method I/2-(5-cyclopropyl-4-((5-methyl-1H-pyrazol-3-yl)amino)-6-(1-methyl-1H-pyrazol-4-yl)pyrimidin-2-yl)-2-(4-(methylsulfonyl)phenyl) acetonitrile

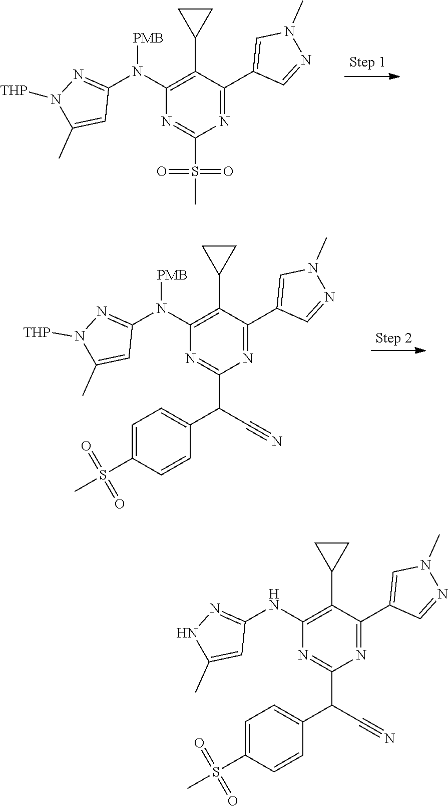

### Step 1/2-(5-cyclopropyl-4-((4-methoxybenzyl)(5-methyl-1-(tetrahydro-2H-pyran-2-yl)-1H-pyrazol-3-yl)amino)-6-(1-methyl-1H-pyrazol-4-yl)pyrimidin-2-yl)-2-(4-(methylsulfonyl)phenyl) acetonitrile

A 4 mL vial was charged with 5-cyclopropyl-N-[(4-methoxyphenyl)methyl]-6-(1-methylpyrazol-4-yl)-2-methylsulfonyl-N-(5-methyl-1-tetrahydropyran-2-yl-pyrazol-3-yl)pyrimidin-4-amine (see Compound 11, Step 1) (100 mg, 173 μmol), 2-(4-methylsulfonylphenyl) acetonitrile (50.0 mg, 256 μmol) and DMPU (500 μL). NaHMDS (1 M, 350 μL, 350 μmol) was added and the mixture was stirred at 120° C. for 30 min. The cooled reaction mixture was diluted with EtOAc and washed with water and brine, dried over Na₂SO₄, filtered and concentrated. The residue was purified by silica gel chromatography eluting with a gradient of EtOAc (0 to 10%) in Heptane then with a gradient of MeOH (0 to 20%) in DCM. Appropriate fractions were combined and concentrated in vacuo to afford 2-(5-cyclopropyl-4-((4-methoxybenzyl)(5-methyl-1-(tetrahydro-2H-pyran-2-yl)-1H-pyrazol-3-yl)amino)-6-(1-methyl-1H-pyrazol-4-yl)pyrimidin-2-yl)-2-(4-(methylsulfonyl)phenyl) acetonitrile (28 mg, 23% yield). UPLC-MS (+ESI): m/z=693.0 [M+H]⁺.

### Step 2/Compound 144

2-(5-cyclopropyl-4-((4-methoxybenzyl)(5-methyl-1-(tetrahydro-2H-pyran-2-yl)-1H-pyrazol-3-yl)amino)-6-(1-methyl-1H-pyrazol-4-yl)pyrimidin-2-yl)-2-(4-(methylsulfonyl)phenyl) acetonitrile (28.0 mg, 40.4 μmol) was dissolved in DCM (1 mL) and TFA (1 mL). L-Cysteine (10.0 mg, 82.5 umol) was added and the mixture was stirred at 50° C. for 90 min. The mixture was concentrated and then dissolved in DMSO and neutralized with Et₃N. The reaction mixture was filtered, and the filtrate was purified by preparative HPLC eluting with a gradient of CH₃CN (25 to 55%) in water containing 10 mM NH₄HCO₃ (pH adjusted to 10 with NH₄OH). Appropriate fractions were combined and lyophilized to afford Compound 144 (6.0 mg, 30% yield) ¹H NMR (DMSO-d₆) δ: 11.98 (s, 1H), 8.65 (s, 1H), 8.25 (s, 1H), 7.99-7.93 (m, 3H), 7.75 (d, J=8.2 Hz, 2H), 6.33 (s, 1H), 5.90 (s, 1H), 3.88 (s, 3H), 3.16 (s, 3H), 2.17 (s, 3H), 1.85-1.75 (m, 1H), 1.20-1.11 (m, 2H), 0.23-0.15 (m, 2H). UPLC-MS (+ESI): m/z=489.3 [M+H]⁺.

### Compound 158/Method I/5-cyclopropyl-N-(5-methyl-1H-pyrazol-3-yl)-6-(1-methyl-1H-pyrazol-4-yl)-2-(4-(methylsulfonyl)benzyl)pyrimidin-4-amine

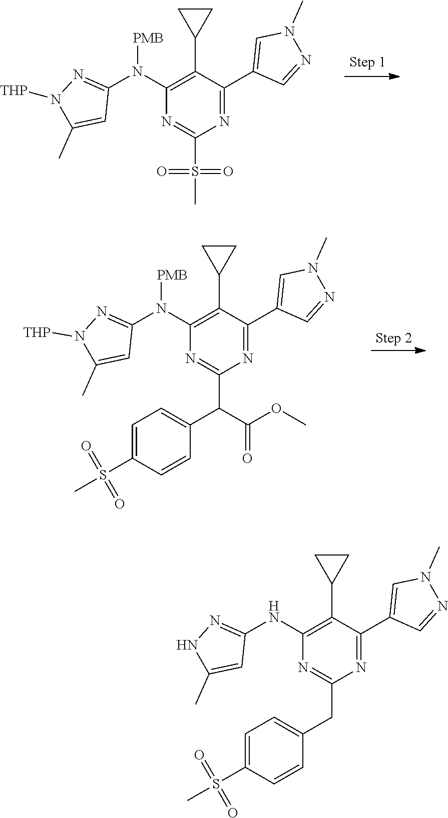

### Step 1/methyl 2-(5-cyclopropyl-4-((4-methoxybenzyl)(5-methyl-1-(tetrahydro-2H-pyran-2-yl)-1H-pyrazol-3-yl)amino)-6-(1-methyl-1H-pyrazol-4-yl)pyrimidin-2-yl)-2-(4-(methylsulfonyl)phenyl)acetate

A 4 mL vial was charged with 5-cyclopropyl-N-[(4-methoxyphenyl)methyl]-6-(1-methylpyrazol-4-yl)-2-methylsulfonyl-N-(5-methyl-1-tetrahydropyran-2-yl-pyrazol-3-yl)pyrimidin-4-amine (see Compound 11, Step 1) (100 mg, 173 μmol), methyl 2-(4-methylsulfonylphenyl)acetate (60.0 mg, 263 μmol) and DMPU (500 μL). NaHMDS (1 M, 350 μL, 350 μmol) was added and the mixture was stirred at 90° C. for 90 min. The mixture was stirred overnight at rt. The mixture was diluted with EtOAc and washed with water and brine, dried over Na₂SO₄, filtered and concentrated. The residue was purified by silica gel chromatography eluting with a gradient of EtOAc (30 to 100%) in heptane then with MeOH (0 to 20%) in DCM to provide methyl 2-(5-cyclopropyl-4-((4-methoxybenzyl)(5-methyl-1-(tetrahydro-2H-pyran-2-yl)-1H-pyrazol-3-yl)amino)-6-(1-methyl-1H-pyrazol-4-yl)pyrimidin-2-yl)-2-(4-(methylsulfonyl)phenyl)acetate (62.0 mg, 49% yield). UPLC-MS (+ESI): m/z=727.0 [M+H]⁺.

### Step 2/Compound 158

Methyl 2-(5-cyclopropyl-4-((4-methoxybenzyl)(5-methyl-1-(tetrahydro-2H-pyran-2-yl)-1H-pyrazol-3-yl)amino)-6-(1-methyl-1H-pyrazol-4-yl)pyrimidin-2-yl)-2-(4-(methylsulfonyl)phenyl)acetate (62.0 mg, 85.4 μmol) was dissolved in Methanol (250 μL) and THF (750 μL) and then treated with LiOH (1 M, 250 μL, 250 μmol). The mixture was stirred at rt for 2 hrs. The reaction mixture was neutralized with 250 μL of 10% aqueous solution HCl and then concentrated to dryness in vacuo. L-Cysteine (21.0 mg, 173 μmol) was added to the crude product followed by DCM (1 mL) and TFA (1 mL). The mixture was stirred at 60° C. overnight under reflux. The volatiles were evaporated, and the crude residue was redissolved in DMSO and basified with a few drops of Et₃N. The reaction mixture was filtered and the filtrate was purified by preparative HPLC eluting with a gradient of CH₃CN (25 to 55%) in water containing 10 mM ammonium bicarbonate (pH adjusted to 10 with NH₄OH). Appropriate fractions were combined and lyophilized to afford Compound 158 (8 mg, 20% yield). ¹H NMR (DMSO-d₆) δ: 11.88 (s, 1H), 8.34 (s, 1H), 8.23 (s, 1H), 7.94 (s, 1H), 7.86-7.80 (m, 2H), 7.62-7.54 (m, 2H), 6.11 (s, 1H), 4.09 (s, 2H), 3.87 (s, 3H), 3.13 (s, 3H), 2.12 (s, 3H), 1.78 (h, J=6.2, 5.4 Hz, 1H), 1.16-1.08 (m, 2H), 0.20-0.13 (m, 2H). UPLC-MS (+ESI): m/z=464.6 [M+H]⁺.

### Compound 228/Method H/5-cyclopropyl-N⁴-(5-cyclopropyl-1H-pyrazol-3-yl)-N²-(2,6-difluoro-4-(methylsulfonyl)phenyl)-N²-methyl-6-(1-methyl-1H-imidazol-4-yl)pyrimidine-2,4-diamine

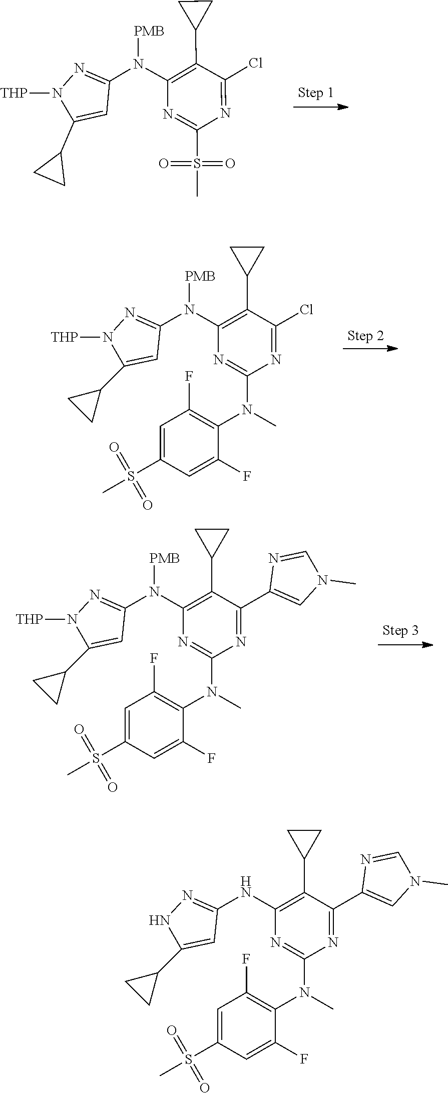

### Step 1/6-chloro-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine

To a solution of 2,6-difluoro-4-methylsulfonyl-aniline (100 mg, 483 μmol) in DMPU (1.5 mL) was added NaHMDS (1 M, 900 μL, 900 μmol) at rt. Then Intermediate 13 (223 mg, 400 μmol) was added and the resulting reaction mixture was stirred at rt for 1 hr. CH₃I (125 μL, 2.01 mmol,) was added dropwise and the resulting mixture was stirred at rt for 30 min. The reaction was diluted with EtOAc, washed with water, brine (10 mL), dried over Na₂SO₄, filtered, and concentrated to dryness. The residue was purified on silica gel column eluting with a gradient of EtOAc (50-100%) in heptane. Appropriate fractions were combined and concentrated and the residue was further purified by preparative HPLC (Phenomenex Gemini) eluting with a gradient of CH₃CN (25 to 100%) in water containing 10 mM ammonium bicarbonate (pH adjusted to 10 with NH₄OH). Appropriate fractions were combined and lyophilized to afford 6-chloro-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine (128 mg, 46% yield) as an off-white solid. UPLC-MS (+ESI): m/z=699.8 [M+H]⁺.

### Step 2/5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N-methyl-6-(1-methyl-1H-imidazol-4-yl)pyrimidine-2,4-diamine

To a solution of 6-chloro-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine (60.0 mg, 85.8 μmol) in dioxane (2 mL) were added tributyl-(1-methylimidazol-4-yl) stannane (65.0 mg, 175 μmol) and XPhosPdG₃ (16.0 mg, 17.0 μmol). The mixture was degassed in vacuo and backfilled with N₂ three time then the resulting mixture was stirred at 120° C. for 1 hr. Additional XPhosPdG₃ (16 mg, 16.99 μmol) was added the mixture was stirred for another 1 hr at 120° C. The resulting mixture was cooled to rt, and concentrated. The residue was purified on silica gel column eluting with a gradient of EtOAc (60-100%) in hexane. A second purification on silica gel column eluting with a gradient of MeOH (0-10%) in DCM. Appropriate fractions were combined and concentrated to afford 5-cyclopropyl-N⁴-(5-cyclopropyl-1-(tetrahydro-2H-pyran-2-yl)-1H-pyrazol-3-yl)-N²-(2,6-difluoro-4 (methylsulfonyl)phenyl)-N⁴-(4-methoxybenzyl)-N-methyl-6-(1-methyl-1H-imidazol-4-yl)pyrimidine-2,4-diamine (31 mg, 49% yield) as an off-white solid. UPLC-MS (+ESI): m/z=746.0 [M+H]⁺.

### Step 3/Compound 228

To a solution of 5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N-methyl-6-(1-methyl-1H-imidazol-4-yl)pyrimidine-2,4-diamine (31.0 mg, 41.6 μmol) in DCE (500 μL) was added TFA (500 μL, 6.49 mmol,) and L-Cysteine (11.0 mg, 90.8 μmol). The resulting mixture was stirred at 80° C. under N₂ for 4 hrs. After cooling to rt, the volatiles were removed in vacuo and the residue was co-evaporated with MeOH then co-evaporated with MeOH and TEA. The residue was purified by reverse phase flash chromatography (HP C18 RediSep® Rf gold) eluting with a gradient of CH₃CN (25 to 100%) in water containing 10 mM ammonium bicarbonate (pH adjusted to 10 with NH₄OH). Appropriate fractions were combined and lyophilized to afford Compound 228 (18.0 mg, 80% yield) as an off-white solid. ¹H NMR (400 MHz, DMSO-d₆) δ 11.82 (s, 1H), 8.17 (s, 1H), 7.81 (d, J=6.9 Hz, 2H), 7.60 (2s, 2H), 5.58 (bs, 1H), 3.67 (s, 3H), 3.34 (s, 3H), 3.30 (s, 3H), 1.76 (m, 1H), 1.66 (m, 1H), 1.04-0.82 (m, 2H), 0.79 (m, 2H), 0.44 (m, 2H), 0.06 (m, 2H). UPLC-MS (+ESI): m/z=541.3 [M+H]⁺.

### Compound 287/Method H/4-(5-cyclopropyl-6-((5-cyclopropyl-1H-pyrazol-3-yl)amino)-2-((2,6-difluoro-4-(methylsulfonyl)phenyl)(methyl)amino)pyrimidin-4-yl)-1-methyl-1H-imidazole-2-carbonitrile

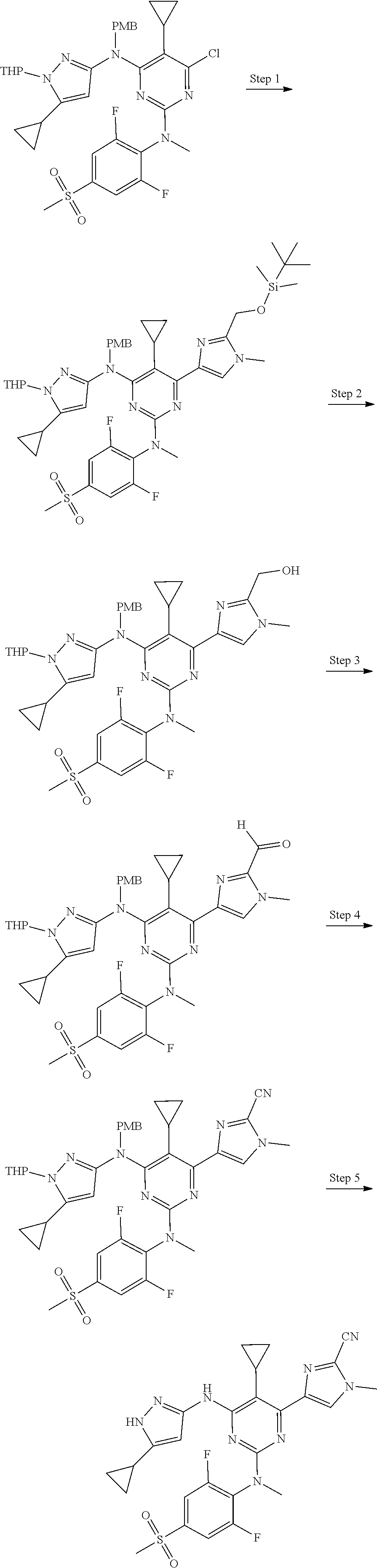

### Step 1/6-(2-(((tert-butyldimethylsilyl)oxy)methyl)-1-methyl-1H-imidazol-4-yl)-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine

In a 2-5 mL microwave vial equipped with a stirring bar, 6-chloro-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine (see Compound 228, Step 1) (170 mg, 243 μmol) was dissolved in 1,4-dioxane (2.5 mL). tert-butyl-dimethyl-[(1-methyl-4-tributylstannyl-imidazol-2-yl)methoxy]silane (360 mg, 489 μmol) was added to the reaction mixture. The reaction mixture was degassed with a stream of N₂ for 10 minutes, Pd(PPh₃)₄ (70.2 mg, 60.8 μmol) was added. The mixture was degassed again with a stream of nitrogen for 15 minutes, finally the vial was sealed and the reaction was heated at 130° C. for 16 hrs. The resulting mixture was cooled to rt, diluted with EtOAc and washed with 1M KF. The organic layer was separated, dried over Na₂SO₄, filtered on celite and concentrated. The residue was purified by silica gel chromatography eluting with a gradient of EtOAc (0 to 75%) in Heptane. Appropriate fractions were combined and concentrated in vacuo to afford 6-(2-(((tert-butyldimethylsilyl)oxy)methyl)-1-methyl-1H-imidazol-4-yl)-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine (124 mg, 57% yield). UPLC-MS (+ESI): m/z=889.5 [M+H]⁺.

### Step 2/(4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazol-2-yl) methanol

6-(2-(((tert-butyldimethylsilyl)oxy)methyl)-1-methyl-1H-imidazol-4-yl)-5-cyclopropyl-N⁴-(5-cyclopropyl-1-(tetrahydro-2H-pyran-2-yl)-1H-pyrazol-3-yl)-N²-(2,6-difluoro-4-(methylsulfonyl)phenyl)-N⁴-(4-methoxybenzyl)-N²-methylpyrimidine-2,4-diamine (123 mg, 138 μmol) was dissolved in THF (1.4 mL) cooled to 0° C. TBAF Solution (1 M, 166 μL, 166 μmol) was added and the reaction mixture was warmed to rt and stirred for 1 hr. The reaction mixture was poured in H₂O and extracted with EtOAc (2×). The combined organic layers were washed with brine, dried over Na₂SO₄, filtered, and concentrated in vacuo and dried with high vacuum for 1 hr to afford (4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-methylsulfonyl)phenyl)(methyl)amino)pyrimidin-4-yl)-1-methyl-1H-imidazol-2-yl) methanol (108 mg), which was used as is in next step without further purification. UPLC-MS (+ESI): m/z=775.2 [M+H]⁺.

### Step 3/4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazole-2-carbaldehyde

(4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-methylsulfonyl)phenyl)(methyl)amino)pyrimidin-4-yl)-1-methyl-1H-imidazol-2-yl) methanol (108 mg, 139 μmol) was dissolved in DCM (1.5 mL) and cooled to 0° C. Dess-Martin periodinane (70.9 mg, 167 μmol) was added and the resulting mixture was stirred for 1 hr. To complete the reaction, additional Dess-Martin periodinane (29.6 mg, 69.7 μmol) was added and the final mixture was stirred for an additional 1 hr. The reaction mixture was quenched with addition of iPrOH (250 μL) and stirred for 5 minutes. Aqueous Na₂S₂O₃ (10 mL) was added to the reaction mixture and then extracted with CH₂Cl₂ (3×). The combined organic layers were dried over Na₂SO₄, filtered, and concentrated in vacuo and dried with high vacuum to afford 4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazole-2-carbaldehyde (107 mg), which was used in next step without further purification. UPLC-MS (+ESI): m/z=773.2 [M+H]⁺.

### Step 4/4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazole-2-carbonitrile

4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazole-2-carbaldehyde (107 mg, 139 μmol) was dissolved in MeOH (1 mL). HONH₂·HCl (20 mg, 288 μmol) and NaOAc (25.0 mg, 305 μmol) were added in single portions. The final mixture was vigorously stirred for 1 hr. The resulting mixture was concentrated under reduced pressure. The crude was dissolved in DCM (1.45 mL), pyridine (55.8 μL, 692.23 μmol,) was added, followed by (2,2,2-trifluoroacetyl) 2,2,2-trifluoroacetate (78.06 μL, 554 μmol,). The reaction mixture was stirred at rt for 16 hrs. Additional Pyridine (22.3 μL, 277 μmol,) was added, and the reaction mixture was heated to 35° C. for 2 hrs but the reaction did not progress further. The volatiles were evaporated under reduced pressure, the crude residue was dissolved in DMSO and purified by reverse phase flash chromatography (HP C18 RediSep® Rf gold) eluting with a gradient of CH₃CN (0 to 100%) in water both containing 0.1% formic acid. Appropriate fractions were combined, and lyophilized to afford 4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazole-2-carbonitrile (45.0 mg, 42% yield). UPLC-MS (+ESI): m/z=770.4 [M+H]⁺.

### Step 5/Compound 287

4-(5-cyclopropyl-6-((5-cyclopropyl-1-(tetrahydro-2H-pyran-2-yl)-1H-pyrazol-3-yl) (4-methoxybenzyl)amino)-2-((2,6-difluoro-4-(methylsulfonyl)phenyl)(methyl)amino)pyrimidin-4-yl)-1-methyl-1H-imidazole-2-carbonitrile (45.0 mg, 58.5 μmol) was dissolved in 1,2-dichloroethane (1.5 mL). L-cysteine (21.3 mg, 175 μmol) was added, followed by TFA (313 μL, 4.09 mmol). The reaction mixture was stirred at 80° C. for 6 hrs. Volatiles were evaporated to dryness. The reaction mixture was neutralized with aqueous saturated NaHCO₃ and extracted with DCM (3×). The combined organic layers were dried over Na₂SO₄, filtered, and concentrated in vacuo. The residue was purified by preparative HPLC (Phenomenex Gemini) eluting with a gradient of CH₃CN (10 to 55%) in water both containing 0.1% formic acid. Appropriate fractions were combined and lyophilized to afford Compound 287 (24 mg, 73% yield). ¹H NMR (400 MHz, DMSO) δ 11.85 (s, 1H), 8.34 (s, 1H), 8.02 (s, 1H), 7.82 (d, J=6.9 Hz, 2H), 5.40 (s, 1H), 3.87 (s, 3H), 3.35 (s, 3H), 3.31 (s, 3H), 1.85-1.72 (m, 1H), 1.66 (s, 1H), 1.02-0.86 (m, 2H), 0.87-0.65 (m, 2H), 0.61-0.28 (m, 2H), 0.02 (dd, J=6.0, 4.2 Hz, 2H). ¹⁹F NMR (376 MHz, DMSO) δ −113.26. 2F. UPLC-MS (+ESI): m/z=566.2 [M+H]⁺.

### Compound 344/Method G/5-cyclopropoxy-N²-(2-fluoro-4-(methylsulfonyl)phenyl)-N²-methyl-N⁴-(5-methyl-1H-pyrazol-3-yl)-6-(1-methyl-1H-pyrazol-4-yl)pyrimidine-2,4-diamine

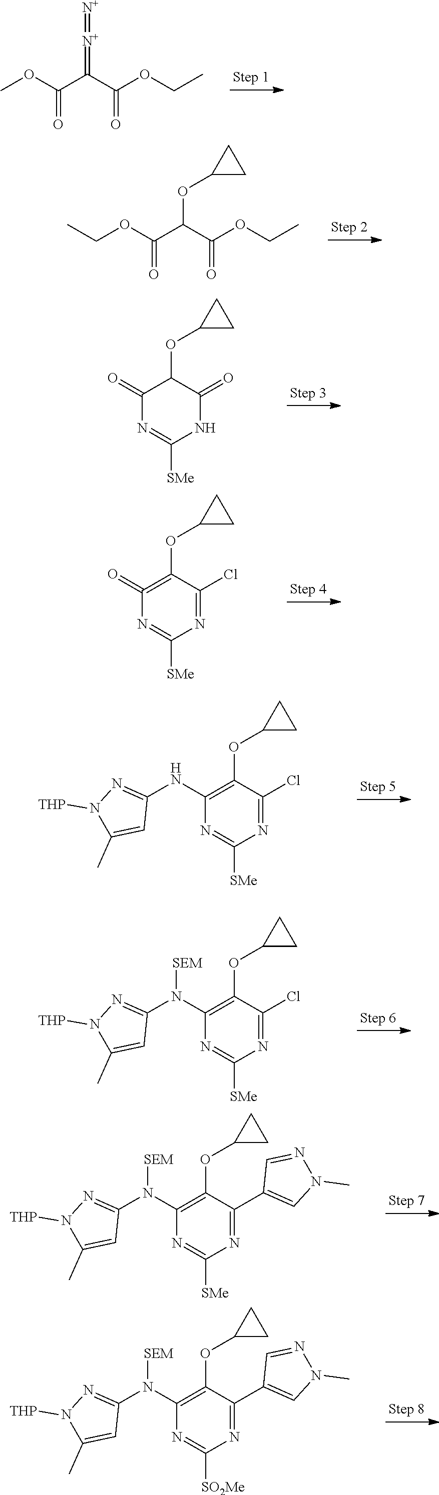

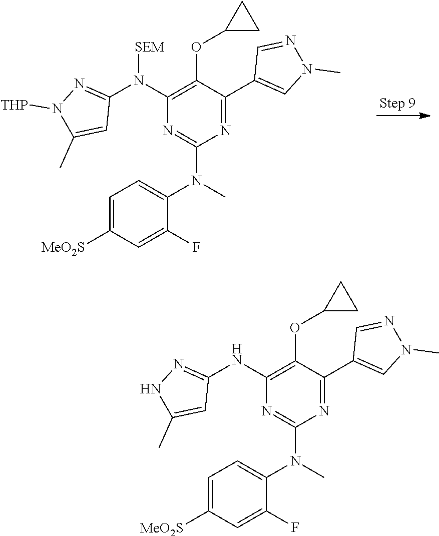

### Step 1/diethyl 2-cyclopropoxymalonate

Rhodium (II) acetate dimer (563 mg, 1.27 mmol) was added to a solution of diethyl 2-diazomalonate (4.74 g, 25.5 mmol) in DCM (100 mL), followed by cyclopropanol (8.41 mL, 129 mmol). The reaction mixture was stirred at 50° C. for 24 hrs. The volatiles were concentrated, and the crude material was purified by silica gel chromatography eluting with EtOAc (0 to 50%) in hexanes. The desired fractions were combined and concentrated to dryness in vacuo to afford diethyl 2-cyclopropoxymalonate (3.37 g, 61% yield) as a colorless oil. ¹H-NMR (400 MHz, CDCl₃): δ 4.56 (s; 1H); 4.21-4.31 (m; 4H); 3.55-3.60 (m; 1H); 1.27-1.31 (m; 6H); 0.74-0.75 (m; 2H); 0.49-0.54 (m; 2H). UPLC-MS (+ESI): m/z=217.1 [M+H]⁺.

### Step 2/5-cyclopropoxy-2-(methylthio)pyrimidine-4,6(1H,5H)-dione

Sodium methoxide (25% w/w in MeOH, 3.56 mL, 15.6 mmol) was added to a solution of thiourea (1.19 g, 15.6 mmol) and diethyl 2-cyclopropoxymalonate (3.37 g, 15.6 mmol) in MeOH (62 mL). The reaction mixture was stirred under reflux for 1 h. The reaction mixture was cooled down to rt, and MeI (1.46 mL, 23.4 mmol) was added dropwise. After stirring at rt for 5 hrs, the reaction mixture was quenched with water (minimum amount), and the solvent was removed under reduced pressure to afford 3.34 g of crude 5-cyclopropoxy-2-(methylthio)pyrimidine-4,6(1H,5H)-dione (yellow foamy solid) was used as such in the next step without further purification. ¹H-NMR (400 MHz, DMSO-d₆): δ 3.96-3.99 (m; 1H); 2.35 (s; 3H); 0.62-0.67 (m; 2H); 0.27-0.32 (m; 2H). UPLC-MS (+ESI): m/z=215.1 [M+H]⁺.

### Step 3/4,6-dichloro-5-cyclopropoxy-2-(methylthio)pyrimidine

A suspension of 5-cyclopropoxy-2-(methylthio)pyrimidine-4,6(1H,5H)-dione (3.30 g, 15.4 mmol) in POCl₃ (29.0 mL, 308 mmol) was stirred at 80° C. for 3 hrs. After cooling the reaction mixture to rt, the volatiles were removed under reduced pressure, and the residue was dissolved in DCM. This mixture was slowly added to ice water while stirring (exotherm observed). The mixture was extracted with DCM (3×75 mL), the combined organic layers were washed with brine, dried over anhydrous Na₂SO₄, filtered, and concentrated to afford the crude product. The crude material was purified by silica gel chromatography eluting with EtOAc (0 to 40%) in hexanes. The desired fractions were combined and concentrated to dryness in vacuo. The purified residue was redissolved in DCM and washed with 20% aqueous Na₂S₂O₃ (20 mL). The organic layer was washed with brine, dried over anhydrous Na₂SO₄, filtered, and concentrated to afford 4,6-dichloro-5-cyclopropoxy-2-(methylthio)pyrimidine (1.40 g) as a yellow/brown oil. UPLC-MS (+ESI): m/z=251.0 [M+H]⁺.

### Step 4/6-chloro-5-cyclopropoxy-N-(5-methyl-1-(tetrahydro-2H-pyran-2-yl)-1H-pyrazol-3-yl)-2-(methylthio)pyrimidin-4-amine

To a solution of 4,6-dichloro-5-cyclopropoxy-2-(methylthio)pyrimidine (1.15 g, 4.58 mmol) and 5-methyl-1-(tetrahydro-2H-pyran-2-yl)-1H-pyrazol-3-amine (913 mg, 5.04 mmol) at rt was added NaHMDS (1 M in THF, 5.50 mL, 5.50 mmol) dropwise. The resulting mixture was stirred at 50° C. for 2 hrs. At this point, additional NaHMDS (1 M in THF, 3.66 mL, 3.66 mmol) was added, and the reaction was stirred at 50° C. for another hour. Saturated aqueous NH₄Cl (10 mL) was added, and the aqueous layer was extracted with EtOAc (3×50 mL). The combined organic layers were washed with brine, dried over anhydrous Na₂SO₄, filtered, and concentrated under reduced pressure. The crude material was purified by silica gel chromatography eluting with EtOAc (0 to 100%) in hexanes. The desired fractions were combined and concentrated in vacuo to afford 6-chloro-5-cyclopropoxy-N-(5-methyl-1-(tetrahydro-2H-pyran-2-yl)-1H-pyrazol-3-yl)-2-(methylthio)pyrimidin-4-amine (1.00 g, 55% yield) as a white solid. 1H-NMR (400 MHz, CDCl₃): δ 7.58 (s; 1H); 6.66 (s; 1H); 5.17 (dd; J=10.36; 2.36 Hz; 1H); 4.15-4.20 (m; 1H); 4.07-4.13 (m; 1H); 3.62-3.68 (m; 1H); 2.55 (s; 3H); 2.34 (s; 3H); 2.04-2.12 (m; 1H); 1.82-1.90 (m; 1H); 1.63-1.78 (m; 2H); 0.90-0.98 (m; 2H); 0.62-0.68 (m; 2H). UPLC-MS (+ESI): m/z=396.2 [M+H]⁺.

### Step 5/6-chloro-5-cyclopropoxy-N-(5-methyl-1-(tetrahydro-2H-pyran-2-yl)-1H-pyrazol-3-yl)-2-(methylthio)-N-((2-(trimethylsilyl)ethoxy)methyl)pyrimidin-4-amine

To a mixture of sodium hydride (60% in mineral oil, 40.0 mg, 1.00 mmol) in THF (1 mL) was added a solution of 6-chloro-5-cyclopropoxy-N-(5-methyl-1-(tetrahydro-2H-pyran-2-yl)-1H-pyrazol-3-yl)-2-(methylthio)pyrimidin-4-amine (170 mg, 429 μmol) in THF (2 mL) dropwise at rt, followed by 2-(trimethylsilyl)ethoxymethyl chloride (200 μL, 1.13 mmol). The resulting solution was stirred at rt for 2 hrs. The reaction was quenched with H₂O (1 mL) and diluted with EtOAc (3 mL). The layers were separated, and the aqueous layer was back extracted with EtOAc (3×5 mL). The combined organic layers were washed with brine, dried over anhydrous Na₂SO₄, filtered, and concentrated under reduced pressure. The crude material was purified by silica gel chromatography eluting with EtOAc (0 to 40%) in hexanes. The desired fractions were combined and concentrated to dryness to afford 6-chloro-5-cyclopropoxy-N-(5-methyl-1-(tetrahydro-2H-pyran-2-yl)-1H-pyrazol-3-yl)-2-(methylthio)-N-((2-(trimethylsilyl)ethoxy)methyl)pyrimidin-4-amine (180 mg, 80% yield) as a viscous oil. UPLC-MS (+ESI): m/z=548.2 [M+Na]⁺.

### Step 6/5-cyclopropoxy-N-(5-methyl-1-(tetrahydro-2H-pyran-2-yl)-1H-pyrazol-3-yl)-6-(1-methyl-1H-pyrazol-4-yl)-2-(methylthio)-N-((2-(trimethylsilyl)ethoxy)methyl)pyrimidin-4-amine

In a vial, a mixture of 6-chloro-5-cyclopropoxy-N-(5-methyl-1-(tetrahydro-2H-pyran-2-yl)-1H-pyrazol-3-yl)-2-(methylthio)-N-((2-(trimethylsilyl)ethoxy)methyl)pyrimidin-4-amine (175 mg, 333 umol), (1-methyl-1H-pyrazol-4-yl) boronic acid (50.3 mg, 399 μmol), NaHCO₃ (55.9 mg, 665 μmol), and Pd(PPh₃)₄ (28.8 mg, 24.9 μmol) in a solvent mixture of DME (1.4 mL) and H₂O (200 μL) was degassed first and heated at 85° C. for 18 h. After cooling to rt, the reaction was diluted with a mixture of 1:1 brine and water (2 mL), and the aqueous mixture was extracted with EtOAc (3×5 mL). The combined organic layers were washed with brine, dried over anhydrous Na₂SO₄, filtered, and concentrated under reduced pressure. The residue was purified by silica gel chromatography eluting with EtOAc (0 to 80%) in hexanes. The desired fractions were combined and concentrated to dryness to afford 5-cyclopropoxy-N-(5-methyl-1-(tetrahydro-2H-pyran-2-yl)-1H-pyrazol-3-yl)-6-(1-methyl-1H-pyrazol-4-yl)-2-(methylthio)-N-((2-(trimethylsilyl)ethoxy)methyl)pyrimidin-4-amine (100 mg, 53% yield) as a yellow oil. UPLC-MS (+ESI): m/z=572.3 [M+H]⁺.

### Step 7/5-cyclopropoxy-N-(5-methyl-1-(tetrahydro-2H-pyran-2-yl)-1H-pyrazol-3-yl)-6-(1-methyl-1H-pyrazol-4-yl)-2-(methylsulfonyl)-N-((2-(trimethylsilyl)ethoxy)methyl)pyrimidin-4-amine

To a mixture of 5-cyclopropoxy-N-(5-methyl-1-(tetrahydro-2H-pyran-2-yl)-1H-pyrazol-3-yl)-6-(1-methyl-1H-pyrazol-4-yl)-2-(methylthio)-N-((2-(trimethylsilyl)ethoxy)methyl)pyrimidin-4-amine (100 mg, 175 μmol), NEt4(HSO₄) (9.50 mg, 28.0 μmol) and Na₂WO₄·2H₂O (5.77 mg, 17.5 μmol) in EtOAc (1.5 mL) and THF (1.5 mL) at rt was added H₂O₂ (179 μL, 1.75 mmol). The reaction mixture was heated at 50° C. for 2 hrs. After cooling to rt, the reaction mixture was quenched with 5% NaHSO3 (2 mL). The layers were partitioned, and the aqueous layer was extracted with EtOAc (3×5 mL). The combined organic layers were washed with water (5 mL), brine, dried over anhydrous Na₂SO₄, filtered, and concentrated to dryness under reduced pressure to afford 105 mg of crude 5-cyclopropoxy-N-(5-methyl-1-(tetrahydro-2H-pyran-2-yl)-1H-pyrazol-3-yl)-6-(1-methyl-1H-pyrazol-4-yl)-2-(methylsulfonyl)-N-((2-(trimethylsilyl)ethoxy)methyl)pyrimidin-4-amine (foamy solid), which was used in the step without further purification. UPLC-MS (+ESI): m/z=604.3 [M+H]⁺.

### Step 8/5-cyclopropoxy-N²-(2-fluoro-4-(methylsulfonyl)phenyl)-N²-methyl-N⁴-(5-methyl-1-(tetrahydro-2H-pyran-2-yl)-1H-pyrazol-3-yl)-6-(1-methyl-1H-pyrazol-4-yl)-N⁴-((2-(trimethylsilyl)ethoxy)methyl)pyrimidine-2,4-diamine

To a solution of 5-cyclopropoxy-N-(5-methyl-1-(tetrahydro-2H-pyran-2-yl)-1H-pyrazol-3-yl)-6-(1-methyl-1H-pyrazol-4-yl)-2-(methylsulfonyl)-N-((2-(trimethylsilyl)ethoxy)methyl)pyrimidin-4-amine (105 mg, 174 μmol) and 2-fluoro-4-(methylsulfonyl) aniline (34.6 mg, 174 μmol) in dry NMP (900 μL) was added LiHMDS (1 M in THF, 522 μL, 522 μmol) under inert atmosphere, and the resulting mixture was stirred at 80° C. for 3 hrs. Then, the reaction was cooled down to rt, and MeI (54 μL, 869 μmol) was added. The resulting mixture was stirred at rt for 1 h. The reaction was quenched with saturated aqueous NH₄Cl (0.5 mL), and the aqueous layer was extracted with EtOAc (3×5 mL). The combined organic layers were washed with water (5 mL), brine, dried over anhydrous Na₂SO₄, filtered, and concentrated to dryness under reduced pressure. The crude material was purified by silica gel chromatography eluting with EtOAc (0 to 100%) in hexanes. The desired fractions were combined and concentrated to dryness to afford 5-cyclopropoxy-N²-(2-fluoro-4-(methylsulfonyl)phenyl)-N²-methyl-N⁴-(5-methyl-1-(tetrahydro-2H-pyran-2-yl)-1H-pyrazol-3-yl)-6-(1-methyl-1H-pyrazol-4-yl)-N⁴-((2-(trimethylsilyl)ethoxy)methyl)pyrimidine-2,4-diamine (60.0 mg, 48% yield) as a yellow oil. UPLC-MS (+ESI): m/z=727.4 [M+H]⁺.

### Step 9/Compound 344

A solution of 5-cyclopropoxy-N²-(2-fluoro-4-(methylsulfonyl)phenyl)-N²-methyl-N⁴-(5-methyl-1-(tetrahydro-2H-pyran-2-yl)-1H-pyrazol-3-yl)-6-(1-methyl-1H-pyrazol-4-yl)-N⁴-((2-(trimethylsilyl)ethoxy)methyl)pyrimidine-2,4-diamine (60.0 mg, 82.5 μmol) in TFA (400 μL) and DCM (400 μL) was stirred at rt for 1 h. After completion, MeCN (1 mL) was added to the reaction mixture, and the volatiles were removed under reduced pressure. The crude residue was purified by preparative HPLC eluting with MeCN (30 to 100%) in H₂O both containing 0.1% FA. The desired fractions were combined, and lyophilized to afford Compound 344 (15 mg, 36% yield) as a white solid. 1H-NMR (400 MHz, CDCl₃): δ 8.15 (s; 1H); 8.03 (s; 1H); 7.76-7.82 (m; 2H); 7.64 (t; J=7.70 Hz; 1H); 7.56 (s; 1H); 5.80 (s; 1H); 3.98 (s; 3H); 3.75-3.78 (m; 1H); 3.59 (s; 3H); 3.18 (s; 3H); 2.19 (s; 3H); 0.87-0.92 (s; 2H); 0.53-0.60 (m; 2H). UPLC-MS (+ESI): m/z=513.3 [M+H]⁺.

### Binding Assay.

To determine the affinity of Compounds described herein in the PLK4 NanoBRET target engagement assay, HEK293 T cells (ATCC Product Number CRL-3216) are first transfected with the PLK4 NanoLuc fusion vector DNA and Transfection carrier DNA using the Fugene HD Transfection reagent in Opti-MEM No Phenol Red buffer. After an overnight incubation in a 37° C./5% CO₂ incubator, the transfected HEK293 T cells are trypsinized, counted, and resuspended in Opti-MEM No Phenol Red buffer at a concentration of 200000 cells/mL. White 96-well plates are then plated with 85 μL of cells (17000 cells/well) to which 5 μL of the 20×K-5 tracer solution diluted in tracer dilution buffer is added. Finally, 10 μL of the 10× compounds are added and the plates and then incubated in a 37° C./5% CO₂ incubator for 2 hrs. After the incubation, a 50 uL 3× solution of the substrate/inhibitor mix is added to the cells. The plate is then transferred in the Envision plate reader where the Acceptor emission (610 nm) and the Donor emission (450 nm) are measured.

The IC₅₀ of compounds is determined as follows:
1. The ratio between the Acceptor emission (610 nm)/Donor emission (450 nm) is calculated.
2. This ratio is then multiplied by 1000 to obtain the mBRET units (multiplying by 1000 is an arbitrary step that simply makes the data easier to interpret; it has no impact on the data set itself)
3. The mBRET units are then used in Scigilian Analyze software (https://analyze.scigilian.io) to determine the IC₅₀ value using a 4-parameter fit analysis.

Exemplary prepared compounds and their activities were shown in Table 3 below. The Compounds were prepared according to Method A, B, C, D, E, F, G previously described using Intermediates described herein and commercially available reagents or readily prepared from commercially available reagents supported by literature precedents. The m/z (M+H)⁺ column indicates the positive ion mass observed by UPLC-MS (+ESI).

TABLE 3

| | | | PLK4 NanoBRET |
|---|---|---|---|
| Compound | Method | m/z (M + H)⁺ | IC₅₀ (μM) |
| 1 | A | 473.1 | 0.22 |
| 2 | B | 487.2 | 0.053 |
| 3 | B | 501.2 | 0.068 |
| 4 | B | 581.3 | 0.72 |
| 5 | A | 481.2 | 1.1 |
| 6 | B | 487.2 | 0.049 |
| 7 | B | 513.2 | 0.065 |
| 8 | B | 505.2 | 0.006 |
| 9 | A | 474.1 | 0.40 |
| 10 | A | 483.2 | 0.095 |
| 11 | B | 497.2 | 0.014 |
| 12 | B | 497.2 | 0.016 |
| 13 | B | 515.2 | 0.005 |
| 14 | B | 515.2 | 0.005 |
| 15 | B | 523.2 | 0.021 |
| 16 | A | 483.2 | 0.20 |
| 17 | E | 497.2 | 0.020 |
| 18 | A | 484.1 | 0.25 |
| 19 | C | 486.1 | 0.056 |
| 20 | C | 496.1 | 0.069 |
| 21 | C | 489.1 | 0.053 |
| 22 | D | 503.1 | 0.096 |
| 23 | D | 486.1 | 0.21 |
| 24 | D | 504.1 | 1.1 |
| 25 | E | 515.2 | 0.003 |
| 26 | B | 532.0 | 0.005 |
| 27 | B | 498.2 | 0.018 |
| 28 | A | 503.6 | 3.7 |
| 29 | A | 494.0 | 1.3 |
| 30 | A | 539.2 | 0.40 |
| 31 | A | 485.2 | 0.62 |
| 32 | A | 471.2 | 2.8 |
| 33 | A | 536.1 | 1.1 |
| 34 | A | 490.1 | 0.90 |
| 35 | A | 473.1 | 3.1 |
| 36 | A | 483.0 | 7.0 |
| 37 | A | 455.1 | 0.55 |
| 38 | A | 474.1 | 1.2 |
| 39 | A | 456.1 | 1.2 |
| 40 | B | 501.2 | 0.010 |
| 41 | B | 515.2 | 0.68 |
| 42 | B | 471.2 | 0.014 |
| 43 | B | 498.2 | 0.18 |
| 44 | B | 514.2 | 0.24 |
| 45 | B | 514.2 | 0.14 |
| 46 | B | 490.1 | 0.11 |
| 47 | B | 483.1 | 2.3 |
| 48 | B | 513.1 | 0.050 |
| 49 | B | 505.1 | 0.054 |
| 50 | B | 556.2 | 0.77 |
| 51 | B | 497.1 | 0.22 |
| 52 | B | 556.2 | 0.29 |
| 53 | B | 559.0 | 0.063 |
| 54 | B | 484.1 | 2.1 |
| 55 | B | 511.2 | 0.32 |
| 56 | B | 493.2 | 0.13 |
| 57 | B | 513.2 | 0.029 |
| 58 | B | 470.1 | 1.1 |
| 59 | B | 471.1 | 0.28 |
| 60 | B | 499.3 | >5.0 |
| 61 | B | 458.1 | 0.10 |
| 62 | B | 616.1 | 0.028 |
| 63 | B | 434.1 | 0.28 |
| 64 | B | 489.1 | 0.059 |
| 65 | B | 453.1 | 0.82 |
| 66 | B | 470.1 | 1.6 |
| 67 | B | 527.2 | 0.27 |
| 68 | B | 481.1 | 1.0 |
| 69 | B | 490.1 | 0.70 |
| 70 | B | 483.1 | 0.17 |
| 71 | B | 480.1 | 0.74 |
| 72 | B | 473.2 | 0.82 |
| 73 | B | 511.0 | 0.086 |
| 74 | B | 439.1 | 1.2 |
| 75 | B | 489.1 | 0.60 |
| 76 | B | 529.2 | 0.046 |
| 77 | B | 544.3 | 0.58 |
| 78 | B | 469.1 | 4.5 |
| 79 | B | 453.1 | 2.3 |
| 80 | B | 474.1 | 0.91 |
| 81 | B | 507.3 | 0.073 |
| 82 | B | 521.3 | 0.065 |
| 83 | B | 469.1 | 0.19 |
| 84 | B | 515.2 | 0.14 |
| 85 | B | 544.2 | 9.2 |
| 86 | B | 511.3 | 0.12 |
| 87 | B | 497.2 | 0.16 |
| 88 | B | 531.3 | 0.29 |
| 89 | B | 479.2 | 1.7 |
| 90 | B | 545.2 | 0.37 |
| 91 | B | 531.2 | 5.4 |
| 92 | C | 514.1 | 0.053 |
| 93 | C | 496.1 | 0.078 |
| 94 | C | 499.1 | 0.11 |
| 95 | D | 507.0 | 0.17 |
| 96 | D | 482.1 | 0.67 |
| 97 | D | 532.0 | 1.2 |
| 98 | D | 511.1 | 2.0 |
| 99 | D | 500.1 | 4.8 |
| 100 | D | 523.0 | 0.24 |
| 101 | D | 514.0 | 0.12 |
| 102 | D | 504.2 | 0.84 |
| 103 | E | 501.1 | 0.009 |
| 104 | E | 516.2 | 0.015 |
| 105 | F | 483.9 | 0.74 |
| 106 | B | 501.1 | 0.006 |
| 107 | G | 497.2 | 0.12 |
| 108 | B | 534.3 | 0.016 |
| 109 | B | 498.2 | 0.049 |
| 110 | F | 551.3 | 0.16 |
| 111 | G | 578.3 | 0.043 |
| 112 | G | 497.2 | 0.11 |
| 113 | G | 497.2 | 0.15 |
| 114 | B | 529.2 | 0.010 |
| 115 | A | 540.2 | 0.022 |
| 116 | B | 521.1 | 0.068 |
| 117 | B | 521.1 | 0.22 |
| 118 | B | 531.1 | >5 |
| 119 | B | 533.2 | 0.009 |
| 120 | B | 549.1 | 0.019 |
| 12 | H | 529.2 | 0.003 |
| 122 | H | 515.2 | 0.008 |
| 123 | H | 538.2 | 0.12 |
| 124 | H | 529.2 | 0.002 |
| 125 | H | 515.2 | 0.007 |
| 126 | H | 541.2 | 0.005 |
| 127 | H | 533.1 | 0.006 |
| 128 | H | 551.2 | 0.006 |
| 129 | H | 529.2 | 0.057 |
| 130 | H | 527.2 | 0.024 |
| 131 | H | 551.1 | 0.009 |
| 132 | H | 513.2 | 0.015 |
| 133 | H | 515.2 | 0.018 |
| 134 | H | 515.2 | 0.007 |
| 135 | H | 513.2 | 0.018 |
| 136 | H | 529.2 | 0.005 |
| 137 | H | 543.2 | 0.003 |
| 138 | H | 475.1 | 0.16 |
| 139 | H | 559.2 | 0.004 |
| 140 | H | 541.2 | 0.003 |
| 141 | H | 545.2 | 0.009 |
| 142 | H | 515.0 | 0.013 |
| 143 | H | 565.5 | 0.012 |
| 144 | I | 489.6 | 5.0 |
| 145 | H | 540.5 | 0.018 |
| 146 | H | 513.2 | 0.021 |
| 147 | H | 545.2 | 0.005 |
| 148 | H | 541.2 | 0.004 |
| 149 | H | 565.2 | 0.006 |
| 150 | H | 559.5 | 0.015 |
| 151 | H | 530.6 | 0.007 |
| 152 | H | 532.6 | 0.005 |
| 153 | H | 532.6 | 0.008 |
| 154 | H | 545.2 | 0.010 |
| 155 | H | 551.6 | 0.010 |
| 156 | H | 516.6 | 0.011 |
| 157 | H | 532.5 | 0.009 |
| 158 | I | 464.6 | 1.6 |
| 159 | H | 529.6 | 0.007 |
| 160 | H | 573.6 | 0.002 |
| 161 | H | 573.6 | 0.004 |
| 162 | I | 552.1 | >5.0 |
| 163 | H | 565.2 | 0.011 |
| 164 | H | 559.2 | 0.008 |
| 165 | H | 565.2 | 0.004 |
| 166 | H | 559.2 | 0.004 |
| 167 | H | 545.2 | 0.030 |
| 168 | H | 529.2 | 0.041 |
| 169 | H | 545.6 | 0.004 |
| 170 | H | 586.2 | 0.011 |
| 171 | H | 572.2 | 0.023 |
| 172 | H | 607.2 | 0.006 |
| 173 | H | 540.6 | 0.36 |
| 174 | H | 557.6 | 0.003 |
| 175 | H | 541.2 | 0.004 |
| 176 | H | 569.2 | 0.013 |
| 177 | H | 589.2 | 0.038 |
| 178 | H | 545.2 | 0.011 |
| 179 | H | 478.1 | 0.26 |
| 180 | H | 571.2 | 0.004 |
| 181 | H | 530.6 | 0.029 |
| 182 | H | 557.2 | 0.011 |
| 183 | H | 541.2 | 0.013 |
| 184 | F | 547.2 | 0.033 |
| 185 | H | 599.2 | 0.021 |
| 186 | H | 605.2 | 0.017 |
| 187 | H | 528.6 | 0.013 |
| 188 | H | 573.2 | 0.008 |
| 189 | H | 501.2 | 0.15 |
| 190 | H | 545.2 | 0.021 |
| 191 | F | 520.2 | 0.016 |
| 192 | H | 621.2 | 0.61 |
| 193 | H | 599.2 | 0.003 |
| 194 | H | 551.1 | 0.063 |
| 195 | H | 523.6 | 0.028 |
| 196 | H | 539.6 | 0.044 |
| 197 | H | 541.5 | 0.024 |
| 198 | H | 555.6 | 0.004 |
| 199 | H | 525.6 | 0.017 |
| 200 | H | 543.6 | 0.008 |
| 201 | H | 559.2 | 0.007 |
| 202 | H | 559.2 | 0.006 |
| 203 | H | 557.2 | 0.006 |
| 204 | H | 540.2 | 0.008 |
| 205 | H | 542.2 | 0.022 |
| 206 | H | 540.6 | 0.004 |
| 207 | H | 541.6 | 0.006 |
| 208 | H | 523.6 | 0.009 |
| 209 | H | 541.2 | 0.005 |
| 210 | H | 557.5 | 0.008 |
| 211 | H | 556.2 | 0.040 |
| 212 | H | 598.2 | 0.053 |
| 213 | H | 573.3 | 0.006 |
| 214 | H | 555.2 | 0.008 |
| 215 | H | 516.1 | 0.069 |
| 216 | H | 516.2 | 0.19 |
| 217 | H | 559.2 | 0.019 |
| 218 | H | 545.2 | 0.053 |
| 219 | H | 555.2 | 0.018 |
| 220 | H | 555.2 | 0.038 |
| 221 | H | 573.2 | 0.012 |
| 222 | H | 573.2 | 0.015 |
| 223 | H | 599.2 | 0.009 |
| 224 | H | 573.2 | 0.013 |
| 225 | H | 545.2 | 0.089 |
| 226 | H | 621.2 | 0.006 |
| 227 | H | 621.2 | 0.005 |
| 228 | H | 541.6 | 0.003 |
| 229 | H | 561.6 | 0.003 |
| 230 | H | 545.6 | 0.026 |
| 231 | H | 558.6 | 0.004 |
| 232 | H | 559.7 | 0.026 |
| 233 | H | 556.2 | 0.045 |
| 234 | H | 557.2 | 0.049 |
| 235 | I | 528.1 | 1.1 |
| 236 | I | 556.2 | 0.087 |
| 237 | H | 562.7 | 0.005 |
| 238 | H | 545.6 | 0.034 |
| 239 | H | 599.2 | 0.004 |
| 240 | H | 557.6 | 0.004 |
| 241 | H | 555.6 | 0.004 |
| 242 | H | 555.7 | 0.005 |
| 243 | H | 557.6 | 0.016 |
| 244 | H | 573.2 | 0.010 |
| 245 | H | 582.2 | 0.005 |
| 246 | H | 529.2 | 0.020 |
| 247 | H | 573.2 | 0.006 |
| 248 | H | 558.2 | 0.033 |
| 249 | H | 559.2 | 0.002 |
| 250 | I | 556.2 | 0.021 |
| 251 | H | 558.7 | 0.31 |
| 252 | H | 586.7 | 0.048 |
| 253 | H | 571.7 | 0.007 |
| 254 | H | 571.7 | 0.004 |
| 255 | H | 585.6 | 0.002 |
| 256 | H | 584.6 | 0.004 |
| 257 | H | 571.6 | 0.033 |
| 258 | H | 529.6 | 0.005 |
| 259 | H | 627.6 | 0.007 |
| 260 | H | 586.7 | 0.005 |
| 261 | H | 572.6 | 0.008 |
| 262 | B | 495.2 | 0.018 |
| 263 | H | 598.7 | 0.009 |
| 264 | H | 634.7 | 0.025 |
| 265 | H | 571.7 | 0.005 |
| 266 | H | 541.2 | 0.006 |
| 267 | H | 552.6 | 0.006 |
| 268 | I | 560.2 | 0.048 |
| 269 | I | 570.2 | 0.014 |
| 270 | H | 518.6 | 0.006 |
| 271 | I | 556.2 | 0.060 |
| 272 | H | 585.7 | 0.003 |
| 273 | H | 583.7 | 0.002 |
| 274 | H | 586.7 | 3.1 |
| 275 | H | 516.2 | 0.006 |
| 276 | H | 599.3 | 0.004 |
| 277 | H | 599.3 | 0.003 |
| 278 | H | 569.6 | 0.060 |
| 279 | H | 551.1 | 0.046 |
| 280 | H | 612.2 | 0.001 |
| 281 | H | 558.2 | 0.34 |
| 282 | I | 570.2 | 0.006 |
| 283 | H | 529.2 | 0.045 |
| 284 | H | 501.2 | 0.096 |
| 285 | H | 541.2 | 0.15 |
| 286 | H | 568.2 | 0.024 |
| 287 | H | 566.2 | 0.006 |
| 288 | H | 552.2 | 0.022 |
| 289 | H | 559.1 | 0.66 |
| 290 | H | 535.2 | 0.030 |
| 291 | H | 553.3 | 0.007 |
| 292 | H | 531 | 0.024 |
| 293 | H | 586.2 | 0.014 |
| 294 | H | 573.1 | 0.009 |
| 295 | H | 526.2 | 0.075 |
| 296 | H | 557.2 | 0.12 |
| 297 | H | 599.3 | 0.014 |
| 298 | H | 541.2 | 0.011 |
| 299 | I | 588.2 | 0.18 |
| 300 | I | 588.2 | 0.15 |
| 301 | H | 545.2 | 0.006 |
| 302 | H | 557.2 | 0.008 |
| 303 | H | 583 | 0.017 |
| 304 | H | 598.2 | 0.008 |
| 305 | H | 613.2 | 0.016 |
| 306 | H | 629.2 | 0.094 |
| 307 | H | 557.2 | 0.019 |
| 308 | H | 554.2 | 0.002 |
| 309 | H | 581.2 | 0.012 |
| 310 | H | 569.2 | 0.031 |
| 311 | H | 587.2 | 0.014 |
| 312 | H | 559.2 | 0.015 |
| 313 | I | 530.2 | 0.018 |
| 314 | H | 518.3 | 0.007 |
| 315 | H | 523.3 | 0.023 |
| 316 | H | 541.2 | 0.005 |
| 317 | H | 526.2 | 0.027 |
| 318 | H | 565.2 | 0.011 |
| 319 | H | 586.2 | 0.005 |
| 320 | H | 558.2 | 0.004 |
| 321 | H | 558.2 | 0.011 |
| 322 | H | 585.3 | 0.012 |
| 323 | H | 572.2 | 0.010 |
| 324 | H | 585.2 | 0.003 |
| 325 | I | 530.2 | 0.052 |
| 326 | H | 552.2 | 0.009 |
| 327 | H | 525.2 | 0.005 |
| 328 | I | 528.2 | 0.051 |
| 329 | H | 554.1 | 0.004 |
| 330 | H | 587.2 | 0.009 |
| 331 | H | 567.1 | 0.014 |
| 332 | I | 529.2 | 0.033 |
| 333 | H | 612.3 | 0.011 |
| 334 | I | 530.0 | 0.037 |
| 335 | B | 514.9 | 0.043 |
| 336 | H | 587.2 | 0.008 |
| 337 | B | 471.1 | 0.062 |
| 338 | B | 467.1 | 0.077 |
| 339 | B | 462.1 | 0.024 |
| 340 | H | 554.2 | 0.004 |
| 341 | H | 545.1 | 0.42 |
| 342 | H | 600.2 | 0.004 |
| 343 | B | 477.1 | 0.026 |
| 344 | G | 512.6 | 0.073 |
| 345 | H | 539.2 | 0.033 |
| 346 | I | 547.3 | 0.012 |
| 347 | B | 541.2 | 0.005 |
| 348 | B | 544.3 | 0.046 |
| 349 | B | 562.3 | 0.046 |
| 350 | B | 521.3 | 0.031 |
| 351 | B | 481.1 | 0.052 |
| 352 | H | 543.3 | 0.003 |
| 353 | B | 522.3 | 0.006 |
| 354 | B | 480.3 | 0.017 |
| 355 | B | 508.3 | 0.19 |
| 356 | B | 550.3 | 0.26 |
| 357 | B | 570.3 | 0.30 |
| 358 | B | 519.6 | 0.027 |
| 359 | B | 520.3 | 0.065 |
| 360 | B | 482.2 | 0.010 |
| 361 | B | 522.3 | >5.00 |
| 362 | B | 502.0 | 0.004 |
| 363 | B | 534.3 | >5 |
| 364 | B | 564.3 | >5 |
| 365 | H | 585.3 | 0.004 |

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of formula (I): 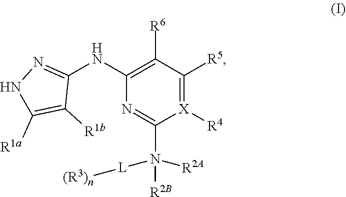
or a pharmaceutically acceptable salt thereof,
wherein
n is 0, 1, 2, 3, or 4;
m is 0, 1, or 2;
L is optionally substituted C₂₋₉ heterocyclyl, optionally substituted C₂₋₉ heteroaryl, optionally substituted C₆₋₁₀ aryl, or optionally substituted C₃₋₈ cycloalkyl, wherein L is further optionally substituted by n occurrences of R³;
R^{1a} is hydrogen, halogen, optionally substituted C₁₋₆ alkyl, optionally substituted C₁₋₆ alkoxy, optionally substituted C₁₋₆ heteroalkyl, optionally substituted C₂₋₆ alkenyl, optionally substituted C₂₋₆ alkynyl, optionally substituted C₃₋₈ cycloalkyl, or nitrile;
R^{1b} is hydrogen; or
R^{1a} and R^{1b}, together with the atoms to which they are attached, are a 3-5-membered cycloalkyl, cycloakylene, cycloalkylyne, heterocycloalkyl, aryl, or heteroaryl;
A is O or S, and R^{2A} and R^{2B} are both absent; or A is N, R^{2A} is absent, and R^{2B} is hydrogen, optionally substituted C₁₋₆ alkyl, optionally substituted C₁₋₆ heteroalkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl, or R^{2B} and L, together with the atom to which they are attached, combine to form an optionally substituted C₂₋₉ heterocyclyl or optionally substituted C₂₋₉ heteroaryl; or A is C, and each of R^{2A} and R^{2B} are independently hydrogen, optionally substituted C₁₋₆ alkyl, optionally substituted C₁₋₆ heteroalkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl;
each R³ is independently halogen, cyano, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₆₋₁₀ aryl C₁₋₆ alkyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, —S(O)ₘR^{3A}, —N(R^{3B})₂, or —OR^{3B};
R^{3A} is optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₆₋₁₀ aryl, optionally substituted C₂₋₉ heterocyclyl, optionally substituted C₁₋₉ heteroaryl, —OR^{3B} or —N(R^{3B})₂.
each R^{3B} is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl; or two R^{3B} groups, together with the atom to which both are attached, combine to form an optionally substituted C₂₋₉ heterocyclyl;
X is N, and R⁴ is absent; or X is C, and R⁴ is hydrogen, halogen, cyano, optionally substituted amino, optionally substituted acyl, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, or optionally substituted C₁₋₉ heteroaryl;
R⁵ is optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, optionally substituted C₁₋₉ heteroaryl, —CONH₂, or —Z—R^{5A};
Z is optionally substituted amino, optionally substituted C₂₋₉ heterocyclylene, optionally substituted C₂₋₉ heteroarylene, optionally substituted C₆₋₁₀ arylene, or optionally substituted C₃₋₈ cycloalkylene;
R^{5A} is hydrogen, halogen, cyano, optionally substituted C₁₋₆ alkylsulfonyl, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₆₋₁₀ aryl, optionally substituted C₂₋₉ heterocyclyl, or optionally substituted C₁₋₉ heteroaryl;
R⁶ is hydrogen, halogen, cyano, optionally substituted C₁₋₆ alkyl, optionally substituted C₂₋₆ alkenyl, optionally substituted C₂₋₆ alkynyl, optionally substituted C₃₋₈ cycloalkyl, or —OR^{6A}; and
R^{6A} is hydrogen, optionally substituted C₁₋₆ alkyl, optionally substituted C₂₋₆ alkenyl, optionally substituted C₂₋₆ alkynyl, or optionally substituted C₃₋₈ cycloalkyl.

2. The compound of claim 1, or a pharmaceutically acceptable salt thereof, wherein the compound is of formula (II):

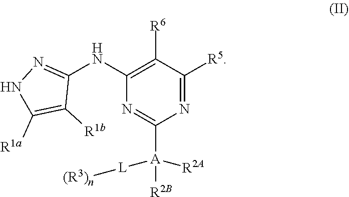

3. The compound of claim 1, or a pharmaceutically acceptable salt thereof, wherein the compound is of formula (III):

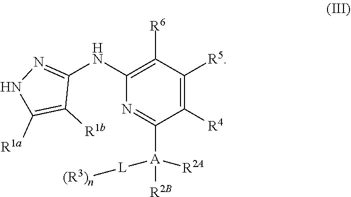

4. The compound of claim 1, or a pharmaceutically acceptable salt thereof, wherein the compound is of formula (II-A):

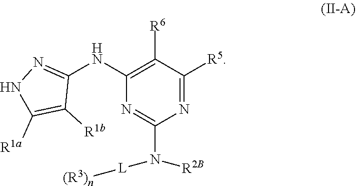

5. The compound of claim 1, or a pharmaceutically acceptable salt thereof, wherein the compound is of formula (III-A):

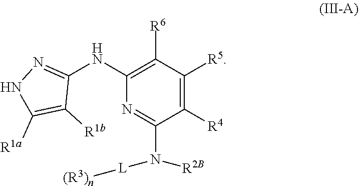

6. The compound of claim 1, or a pharmaceutically acceptable salt thereof, wherein the compound is of formula (II-B):

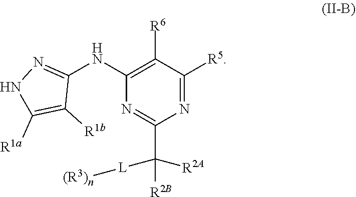

7. The compound of claim 1, or a pharmaceutically acceptable salt thereof, wherein the compound is of formula (III-B):

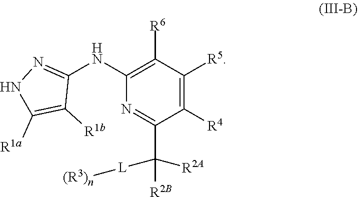

8. The compound of any one of claims 1 to 7, or a pharmaceutically acceptable salt thereof, wherein one of R^{2A} and R^{2B} is hydrogen.

9. The compound of any one of claims 1 to 7, or a pharmaceutically acceptable salt thereof, wherein one of R^{2A} and R^{2B} is optionally substituted C₁₋₆ alkyl.

10. The compound of any one of claims 1 to 7, or a pharmaceutically acceptable salt thereof, wherein one of R^{2A} and R^{2B} is optionally substituted C₁₋₆ heteroalkyl.

11. The compound of claim 9 or claim 10, or a pharmaceutically acceptable salt thereof, wherein R^{2B} is:

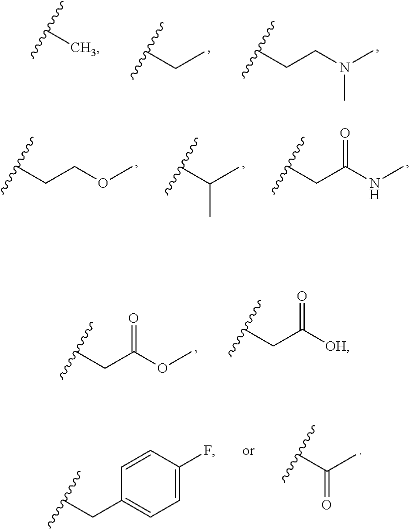

12. The compound of claim 1, or a pharmaceutically acceptable salt thereof, wherein the compound is of formula (II-C):

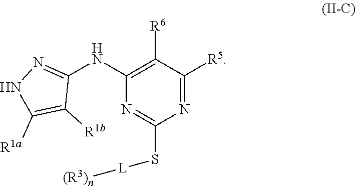

13. The compound of claim 1, or a pharmaceutically acceptable salt thereof, wherein the compound is of formula (III-C):

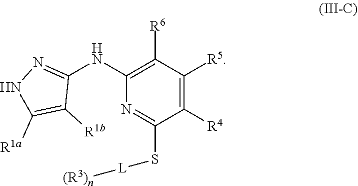

14. The compound of claim 1, or a pharmaceutically acceptable salt thereof, wherein the compound is of formula (II-D):

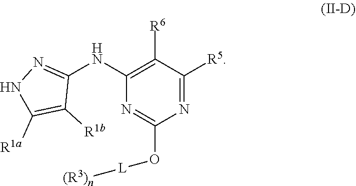

15. The compound of claim 1, or a pharmaceutically acceptable salt thereof, wherein the compound is of formula (III-D):

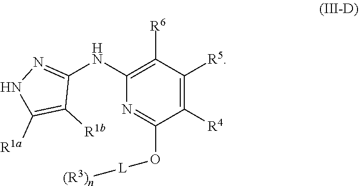

16. The compound of any one of claims 1 to 15, or a pharmaceutically acceptable salt thereof, wherein R^{1a} and R^{1b} are independently are optionally substituted C₁₋₆ alkyl, halo, optionally substituted C₁₋₆ alkoxy, optionally substituted alkynyl, or optionally substituted C₃₋₆ cycloalkyl.

17. The compound of claim 16, or a pharmaceutically acceptable salt thereof, wherein R^{1a} and R^{1b} are independently —CH₃—Cl, —OMe, —CH₂OMe, —CN, —CF₂H, —CF₃, —CHF₂, cyclopropyl, or cyclobutyl.

18. The compound of claim 15, wherein R^{1a} and R^{1b} together with the atoms to which they are attached are a cycloalkyl, cycloalkylene, cycloalkylyne, aryl, heterocyclyl, or heteroaryl.

19. The compound of claim 18, wherein R^{1a} and R^{1b} together with the atoms to which they are attached are:

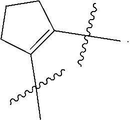 [SMILES: *C1=C(*)CCC1]

20. The compound of any one of claims 1 to 19, or a pharmaceutically acceptable salt thereof, wherein L is optionally substituted C₆₋₁₀ aryl.

21. The compound of claim 20, or a pharmaceutically acceptable salt thereof, wherein the optionally substituted C₆₋₁₀ aryl is optionally substituted phenyl.

22. The compound of any one of claims 1 to 19, or a pharmaceutically acceptable salt thereof, wherein L is optionally substituted C₂₋₉ heteroaryl.

23. The compound of claim 22, or a pharmaceutically acceptable salt thereof, wherein L is optionally substituted C₅ heteroaryl.

24. The compound of claim 23, or a pharmaceutically acceptable salt thereof, wherein the optionally substituted C₅ heteroaryl contains one N.

25. The compound of claim 24, or a pharmaceutically acceptable salt thereof, wherein -L-(R³)ₙ is:

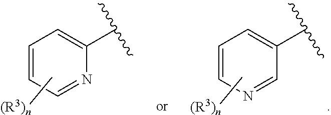

26. The compound of any one of claims 1 to 25, or a pharmaceutically acceptable salt thereof, wherein at least one R³ is halogen.

27. The compound of claim 26, or a pharmaceutically acceptable salt thereof, wherein at least one R³ is F.

28. The compound of claim 26, or a pharmaceutically acceptable salt thereof, wherein at least one R³ is Cl.

29. The compound of claim 26, or a pharmaceutically acceptable salt thereof, wherein at least one R³ is Br.

30. The compound of any one of claims 1 to 25, or a pharmaceutically acceptable salt thereof, wherein at least one R³ is —S(O)ₘR^{3A}.

31. The compound of any one of claims 1 to 30, or a pharmaceutically acceptable salt thereof, wherein m is 1.

32. The compound of any one of claims 1 to 30, or a pharmaceutically acceptable salt thereof, wherein m is 2.

33. The compound of any one of claims 30 to 32, or a pharmaceutically acceptable salt thereof, wherein R^{3A} is optionally substituted C₁₋₆ alkyl.

34. The compound of claim 33, or a pharmaceutically acceptable salt thereof, wherein R^{3A} is —CH₃.

35. The compound of any one of claims 30 to 32, or a pharmaceutically acceptable salt thereof, wherein R^{3A} is optionally substituted C₃₋₈ cycloalkyl.

36. The compound of claim 35, or a pharmaceutically acceptable salt thereof, wherein R^{3A} is optionally substituted cyclopropyl.

37. The compound of any one of claims 1 to 36, or a pharmaceutically acceptable salt thereof, wherein at least one R³ is optionally substituted C₂₋₉ heteroaryl.

38. The compound of any one of claims 1 to 37, or a pharmaceutically acceptable salt thereof, wherein at least one R³ is —N(R^{3B})₂.

39. The compound of any one of claims 1 to 38, or a pharmaceutically acceptable salt thereof, wherein at least one R³ is —OR^{3B}.

40. The compound of any one of claims 1 to 21, or a pharmaceutically acceptable salt thereof, wherein -L-(R³)ₙ is:

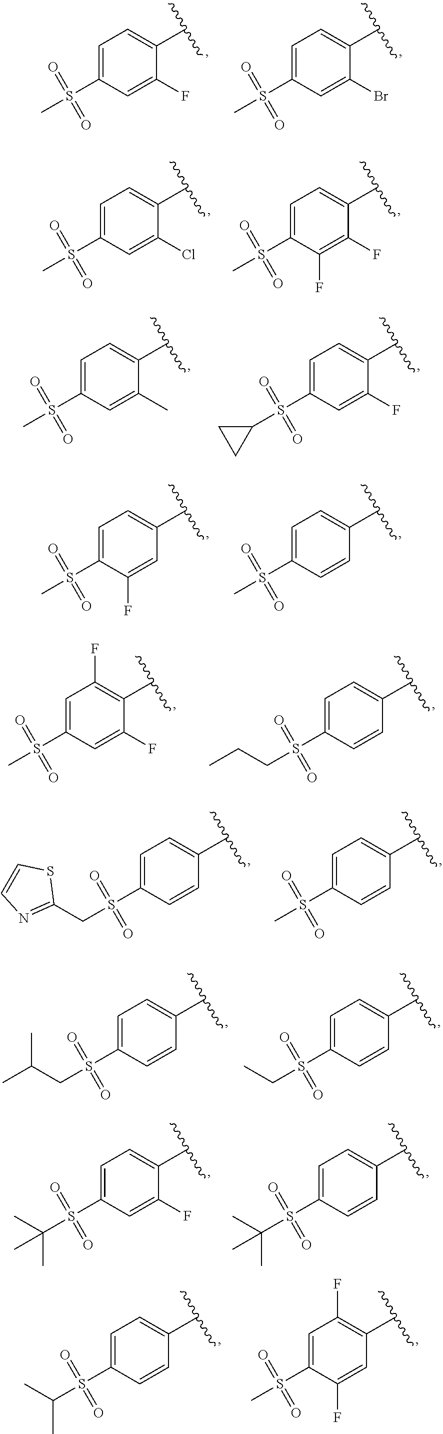

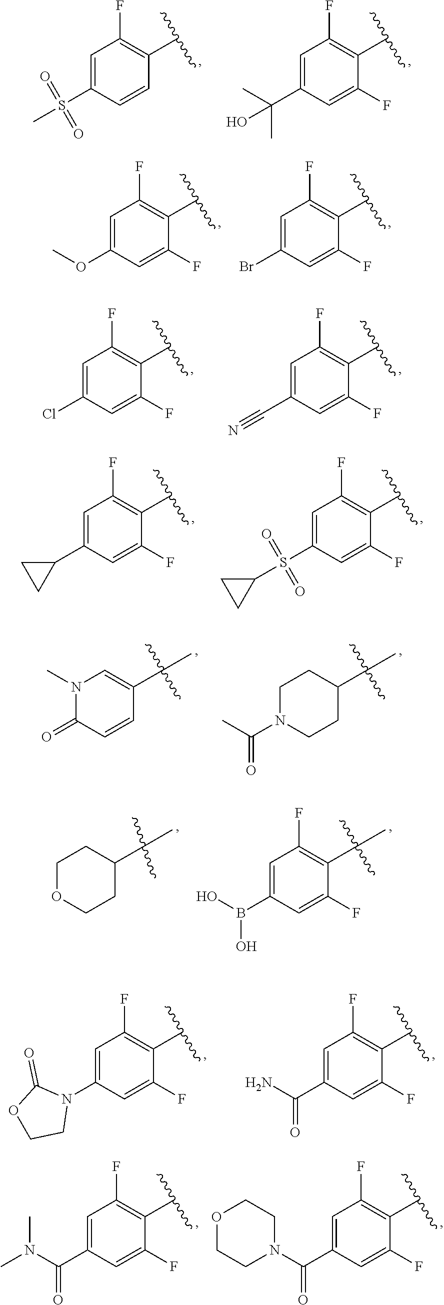

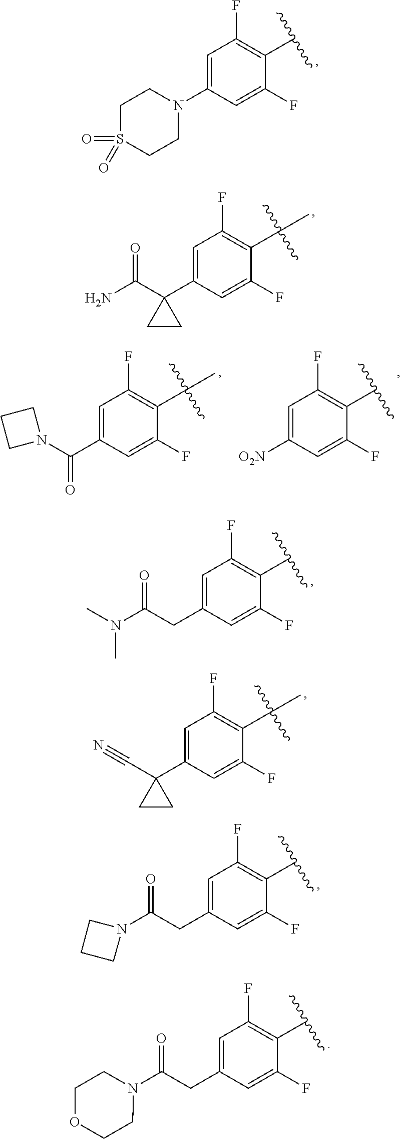

41. The compound of claim 40, or a pharmaceutically acceptable salt thereof, wherein -L-(R³)ₙ is:

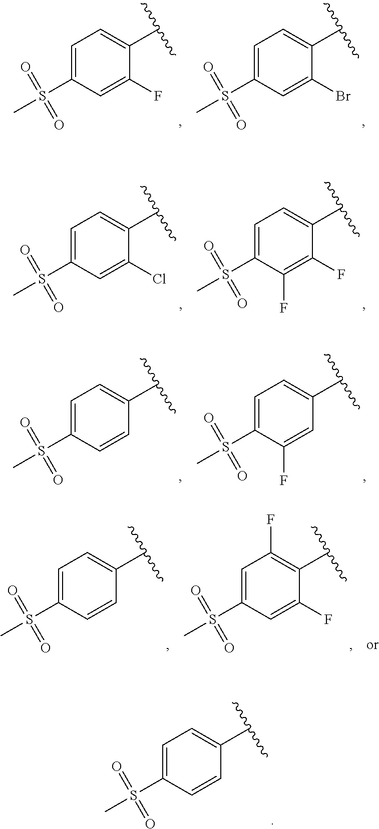

42. The compound of any one of claims 1 to 21, or a pharmaceutically acceptable salt thereof, wherein -L-(R³)ₙ is:

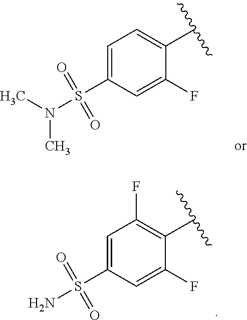 [SMILES: *c1ccc(S(=O)(=O)N(C)C)cc1F ; *c1c(F)cc(S(N)(=O)=O)cc1F]

43. The compound of any one of claims 1 to 21, or a pharmaceutically acceptable salt thereof, wherein -L-(R³)ₙ is:

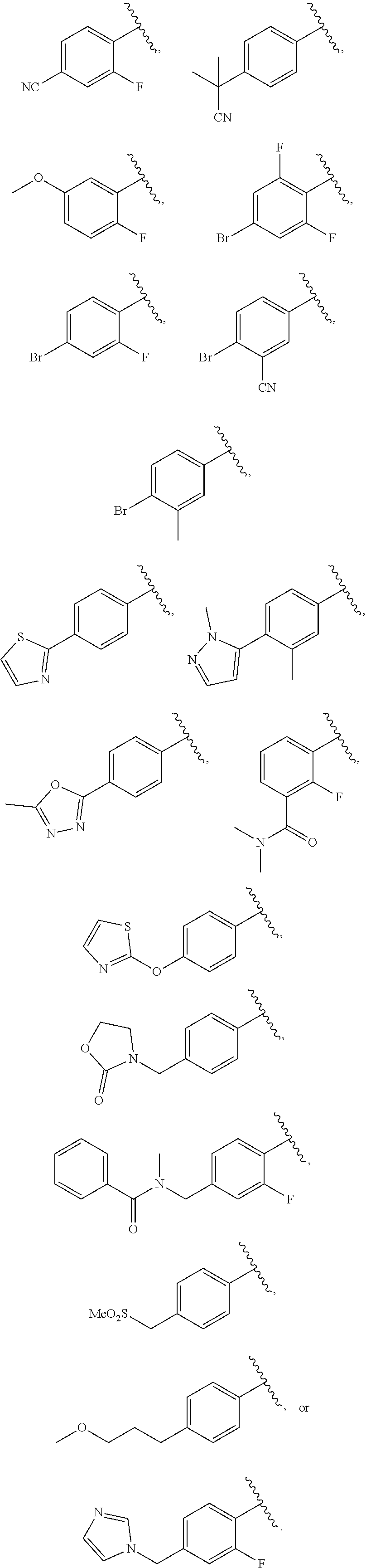

44. The compound of any one of claims 1 to 19, or a pharmaceutically acceptable salt thereof, wherein -L-(R³)ₙ is:

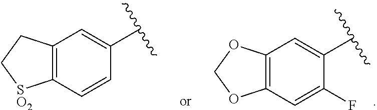 [SMILES: *c1ccc2c(c1)CCS2(=O)=O ; *c1cc2c(cc1F)OCO2]

45. The compound of any one of claims 1 to 21, or a pharmaceutically acceptable salt thereof, wherein -L-(R³)ₙ is:

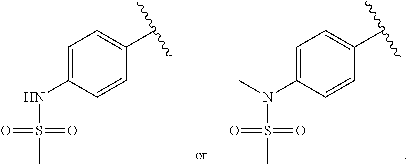 [SMILES: *c1ccc(NS(C)(=O)=O)cc1 ; *c1ccc(N(C)S(C)(=O)=O)cc1]

46. The compound of any one of claims 1 to 19 and 22 to 25, or a pharmaceutically acceptable salt thereof, wherein -L-(R³)ₙ is:

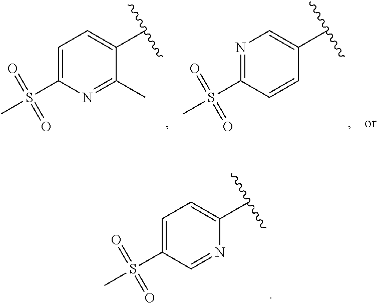

47. The compound of any one of claims 1, 3, 5, 7 to 11, 13, 15, and 16 to 46, or a pharmaceutically acceptable salt thereof, wherein R⁴ is halogen.

48. The compound of claim 47, or a pharmaceutically acceptable salt thereof, wherein R⁴ is F.

49. The compound of claim 47, or a pharmaceutically acceptable salt thereof, wherein R⁴ is Cl.

50. The compound of any one of claims 1, 3, 5, 7 to 11, 13, 15, and 16 to 46, or a pharmaceutically acceptable salt thereof, wherein R⁴ is cyano.

51. The compound of any one of claims 1, 3, 5, 7 to 11, 13, 15, and 16 to 46, or a pharmaceutically acceptable salt thereof, wherein R⁴ is optionally substituted amino.

52. The compound of claim 51, or a pharmaceutically acceptable salt thereof, wherein R⁴ is-NH₂ or —N(CH₃)₂.

53. The compound of any one of claims 1, 3, 5, 7 to 11, 13, 15, and 16 to 46, or a pharmaceutically acceptable salt thereof, wherein R⁴ is hydrogen.

54. The compound of any one of claims 1, 3, 5, 7 to 11, 13, 15, and 16 to 46, or a pharmaceutically acceptable salt thereof, wherein R⁴ is —CH₃.

55. The compound of any one of claims 1 to 54, or a pharmaceutically acceptable salt thereof, wherein R⁵ is optionally substituted C₁₋₉ heteroaryl.

56. The compound of claim 55, or a pharmaceutically acceptable salt thereof, wherein R⁵ is optionally substituted C₃-C₄ heteroaryl or optionally substituted C₄ heterocycle.

57. The compound of claim 56, or a pharmaceutically acceptable salt thereof, wherein the optionally substituted C₃-C₄ heteroaryl or optionally substituted C₄ heterocycle comprises 1 to 2 N atoms.

58. The compound of claim 57, or a pharmaceutically acceptable salt thereof, wherein R⁵ is:

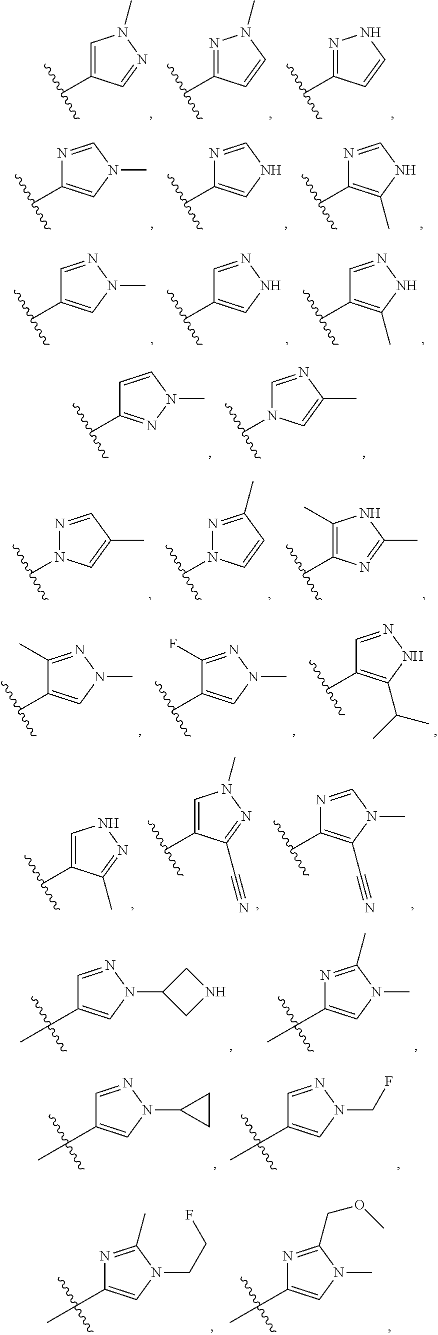

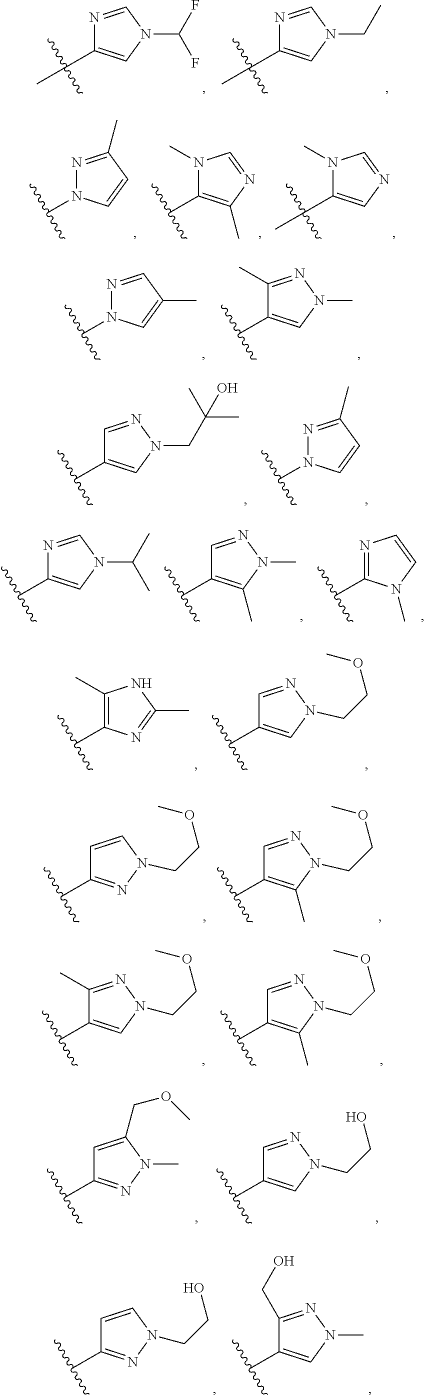

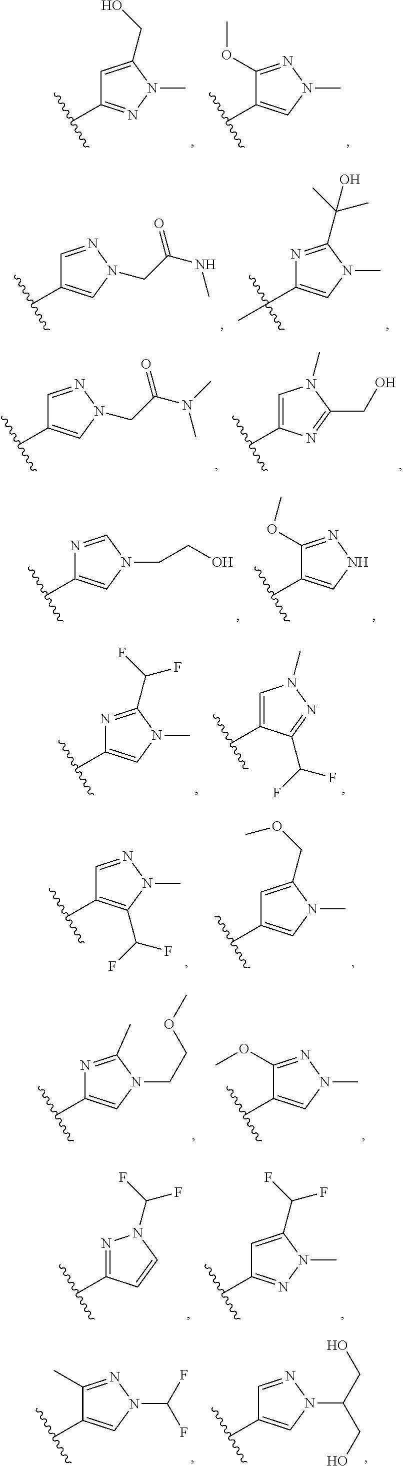

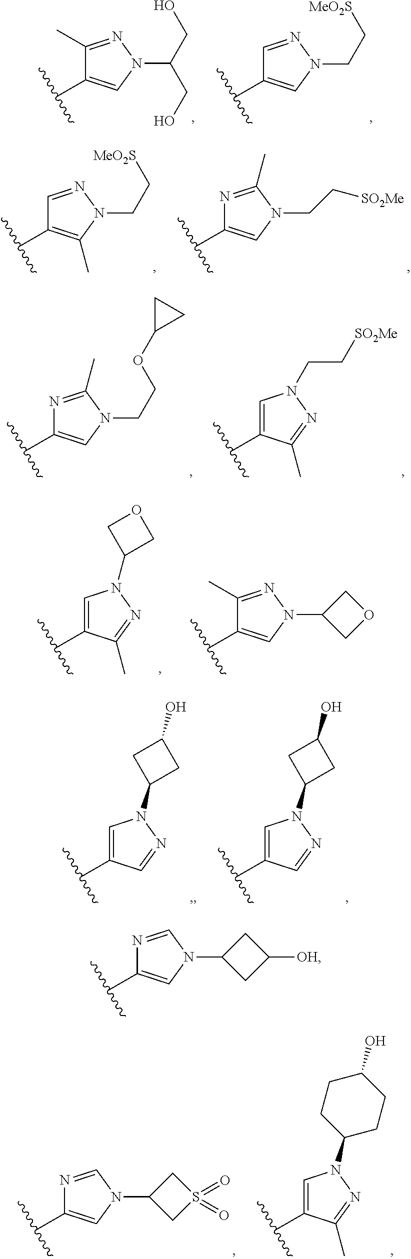

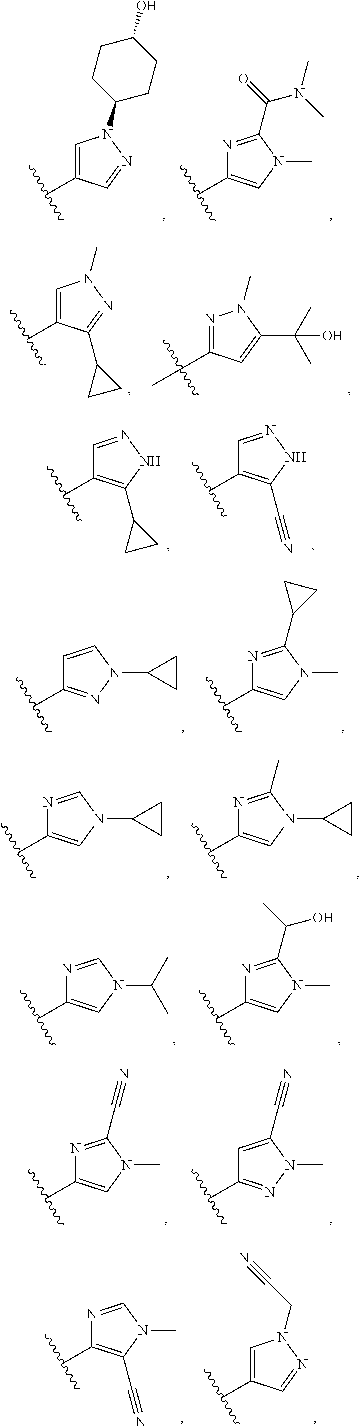

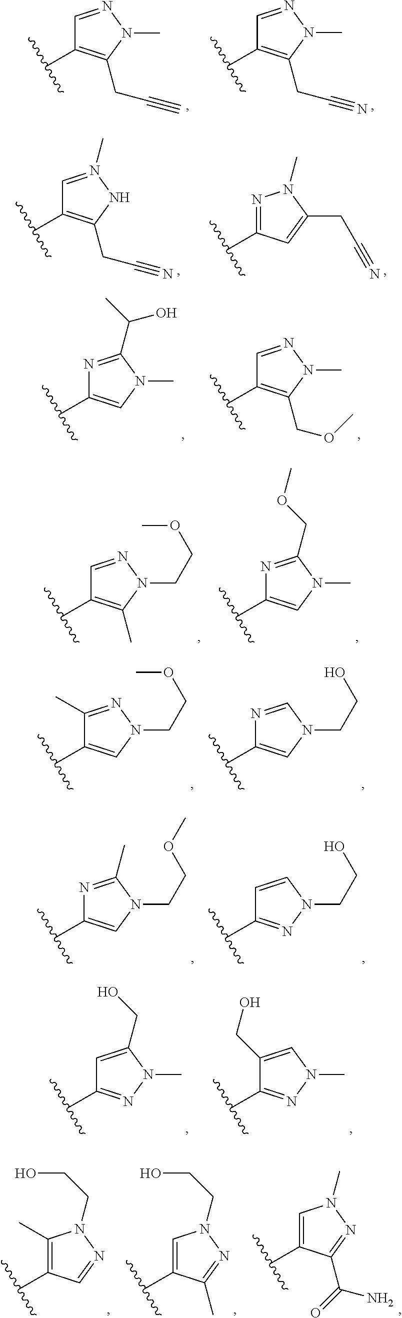

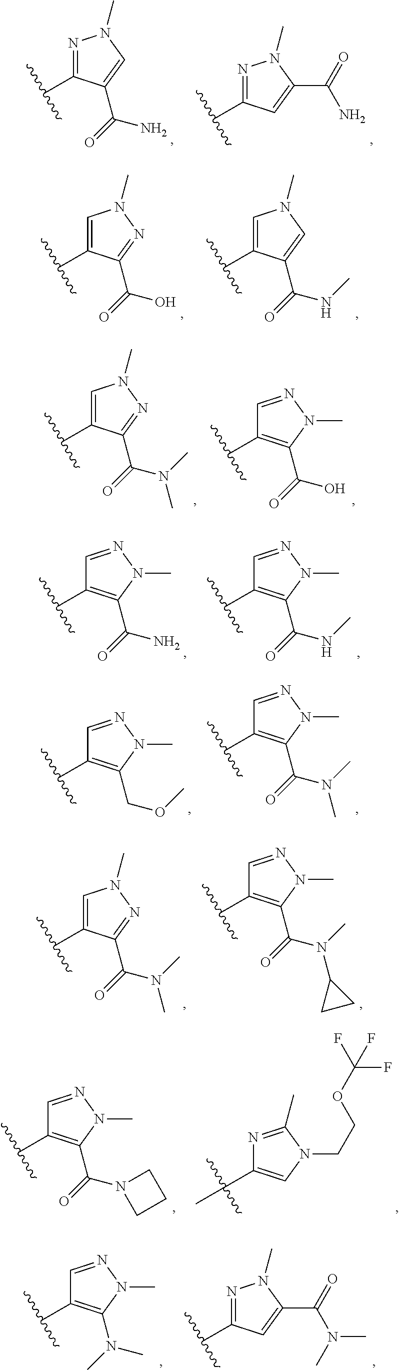

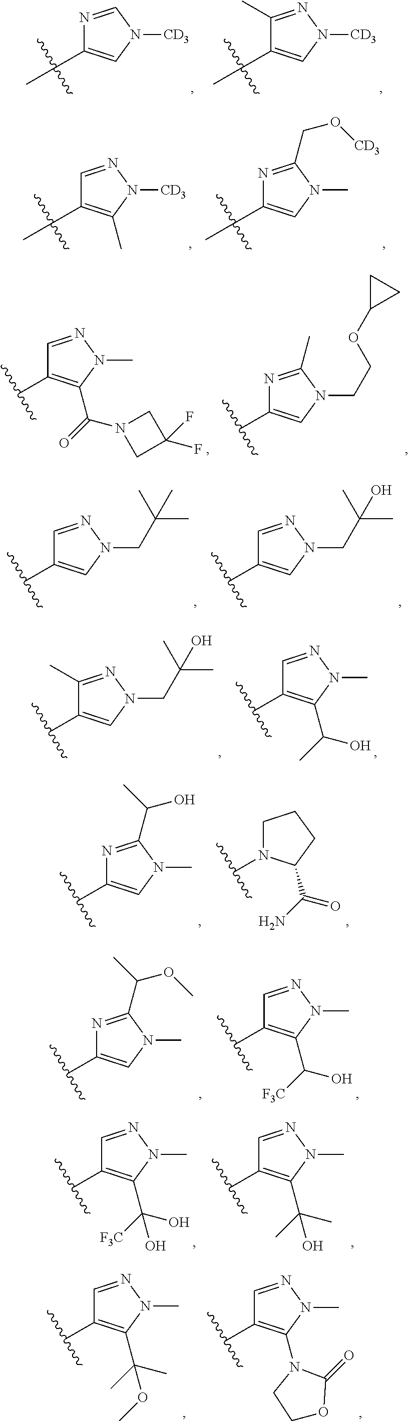

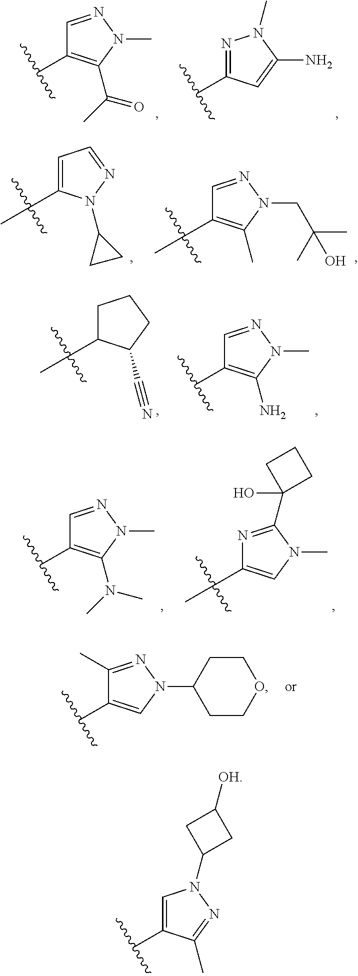

59. The compound of claim 58, or a pharmaceutically acceptable salt thereof, wherein R⁵ is

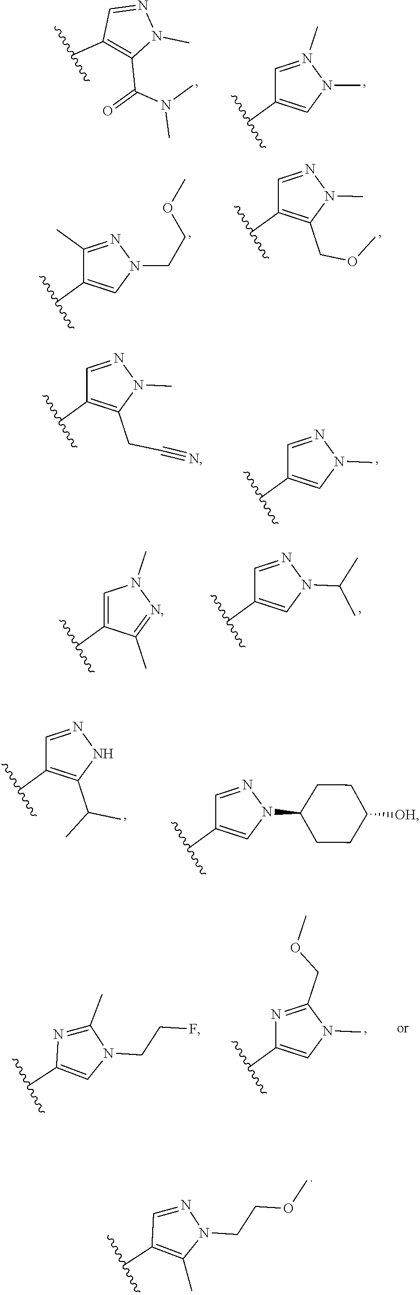

60. The compound of claim 56, or a pharmaceutically acceptable salt thereof, wherein the optionally substituted C₃ heteroaryl comprises 1 N atom and 1 S atom or 1 O atom.

61. The compound of claim 60, or a pharmaceutically acceptable salt thereof, wherein R⁵ is:

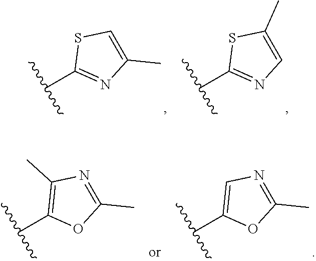

62. The compound of any one of claims 1 to 54, or a pharmaceutically acceptable salt thereof, wherein R⁵ is —Z—R^{5A}.

63. The compound of claim 62, or a pharmaceutically acceptable salt thereof, wherein Z is an optionally substituted amino and R^{5A} is

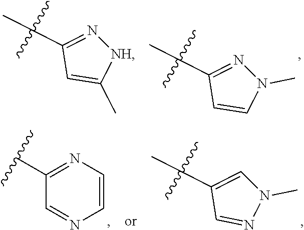

64. The compound of claim 63, or a pharmaceutically acceptable salt thereof, wherein Z is optionally substituted C₂₋₉ heteroarylene.

65. The compound of any one of claims 1 to 54, or a pharmaceutically acceptable salt thereof, wherein R⁵ is optionally substituted C₂₋₉ heterocyclyl.

66. The compound of claim 65, or a pharmaceutically acceptable salt thereof, wherein R⁵ is:

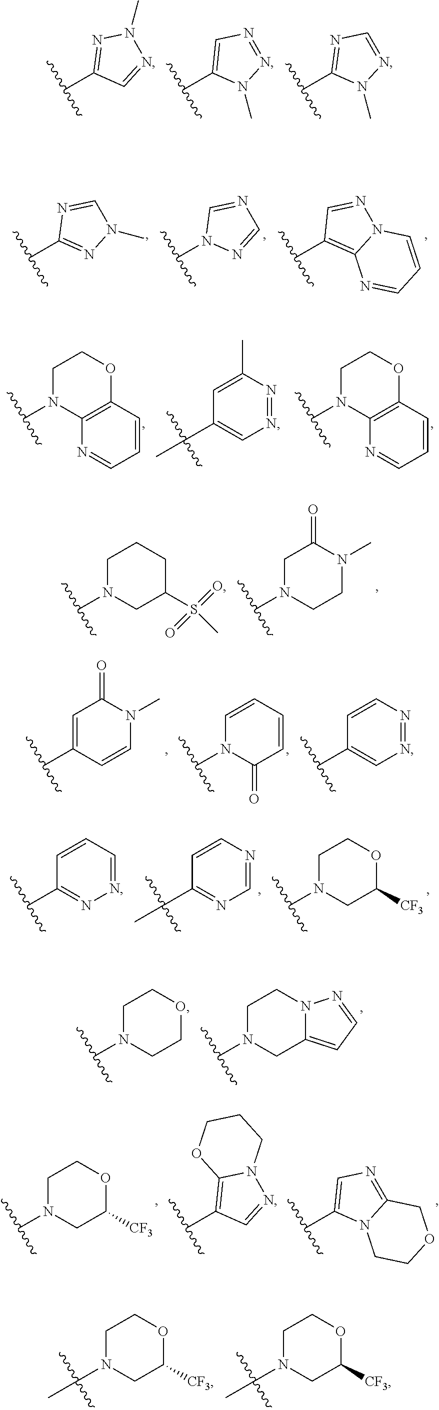

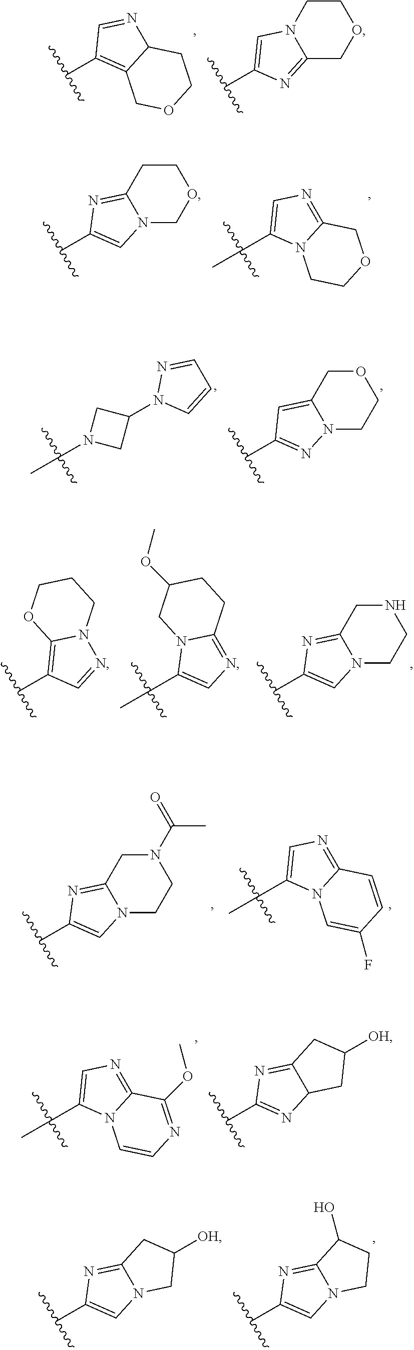

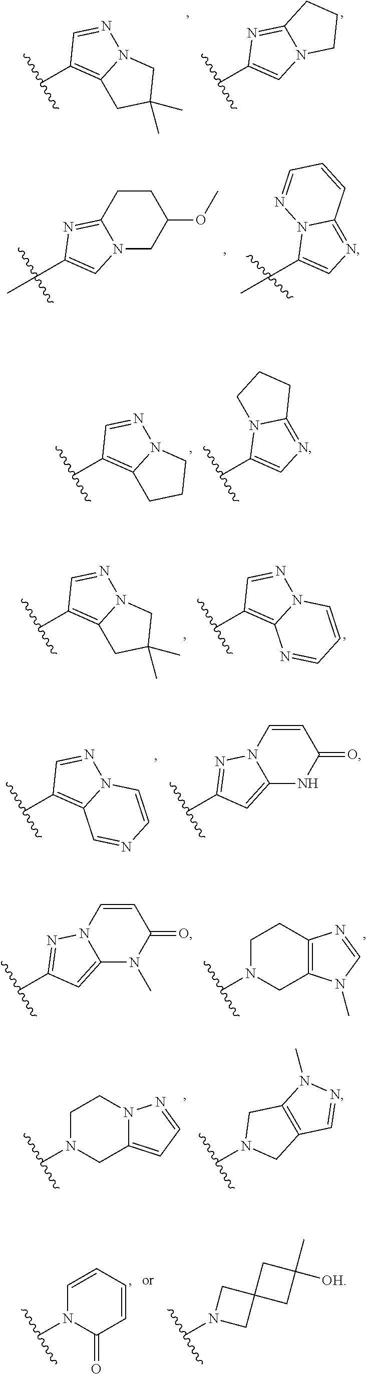

67. The compound of claim 65, or a pharmaceutically acceptable salt thereof, wherein R⁵ is:

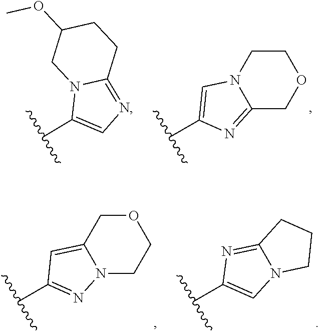 [SMILES: *c1cnc2n1CC(OC)CC2 ; *c1cn2c(n1)COCC2 ; *c1cc2n(n1)CCOC2 ; *c1cn2c(n1)CCC2]

68. The compound of claim 64, or a pharmaceutically acceptable salt thereof, wherein R^{5A} is:

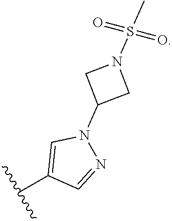 [SMILES: *c1cnn(C2CN(S(C)(=O)=O)C2)c1]

69. The compound of any one of claims 1-54, or a pharmaceutically acceptable salt thereof, wherein R⁵ is:

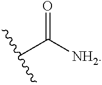 [SMILES: *C(N)=O]

70. The compound of any one of claims 1 to 69, or a pharmaceutically acceptable salt thereof, wherein R⁶ is optionally substituted C₁₋₆ alkyl.

71. The compound of claim 70, or a pharmaceutically acceptable salt thereof, wherein R⁶ is:

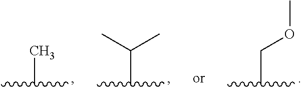

72. The compound of any one of claims 1 to 69, or a pharmaceutically acceptable salt thereof, wherein R⁶ is —OR^{6A}.

73. The compound of claim 72, or a pharmaceutically acceptable salt thereof, wherein R^{6A} is —CH₃.

74. The compound of any one of claims 1 to 69, or a pharmaceutically acceptable salt thereof, wherein R⁶ is optionally substituted C₃₋₈ cycloalkyl.

75. The compound of claim 74 or a pharmaceutically acceptable salt thereof, wherein Re is optionally substituted cyclopropyl.

76. The compound of claim 75, or a pharmaceutically acceptable salt thereof, wherein R⁶ is:

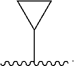 [SMILES: *C1CC1]

77. The compound of claim 1, or a pharmaceutically acceptable salt thereof, wherein the compound is of formula (IV):

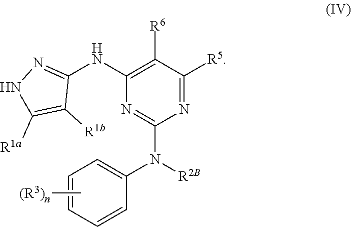

78. The compound of claim 1, or a pharmaceutically acceptable salt thereof, wherein the compound is of formula (V):

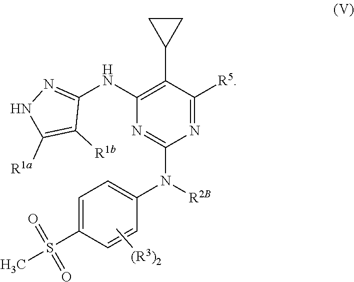

79. A compound selected from the group consisting of compounds 1 to 365 and pharmaceutically acceptable salts thereof.

80. A pharmaceutical composition comprising the compound of any one of claims 1 to 79, or a pharmaceutically acceptable salt thereof, and a pharmaceutically acceptable excipient.

81. The pharmaceutical composition of claim 80, wherein the composition is isotopically enriched in deuterium.

82. A method of inhibiting PLK4 expression in a cell, the method comprising contacting the cell with the compound of any one of claims 1 to 79, or a pharmaceutically acceptable salt thereof.

83. The method of claim 82, wherein the cell is overexpressing TRIM37 or has a TRIM37 amplification.

84. The method of claim 82 or claim 83, wherein the cell is in a subject.

85. A method of treating a subject in need thereof, the method comprising administering to the subject the compound of any one of claims 1 to 79, or a pharmaceutically acceptable salt thereof, or the pharmaceutical composition of claim 80 or claim 81.

86. The method of claim 85, wherein the subject is suffering from, and is in need of treatment for, a disease or condition having the symptom of cell hyperproliferation.

87. The method of claim 86, wherein the disease is cancer.

88. The method of claim 87, wherein the cancer is a cancer overexpressing TRIM37 or a cancer with TRIM37 amplification.

89. A method of treating cancer in a subject, the method comprising administering to the subject a therapeutically effective amount of the compound of any one of claims 1 to 79, or a pharmaceutically acceptable salt thereof, or the pharmaceutical composition of claim 80 or claim 81, wherein the cancer has been previously identified as a cancer overexpressing TRIM37 or a cancer with TRIM37 amplification.

90. A method of treating cancer in a subject, the method comprising administering to the subject a therapeutically effective amount of the compound of any one of claims 1 to 79, or the pharmaceutical composition of claim 80 or claim 81, wherein the cancer is a cancer overexpressing TRIM37 or a cancer with TRIM37 amplification.

91. The method of any one of claims 87 to 90, wherein the cancer is uterine cancer, ovarian cancer, breast cancer, stomach cancer, esophageal cancer, lung cancer, or endometrial cancer.

92. A method of inducing cell death in a cancer cell overexpressing TRIM37 or a cancer with TRIM37 amplification, the method comprising contacting the cell with an effective amount of a PLK4 inhibitor.

93. The method of claim 92, wherein the PLK4 inhibitor is the compound of any one of claims 1 to 75, or a pharmaceutically acceptable salt thereof.

94. The method of claim 92 or claim 93, wherein the cell is in a subject.
